(19) United States
(12) Patent Application Publication
(10) Pub. No.: US 2005/0118117 A1
(43) Pub. Date: Jun. 2, 2005

(54) Methods for identifying risk of melanoma and treatments thereof

(76) Inventors: Richard B. Roth, La Jolla, CA (US); Matthew Roberts Nelson, San Marcos, CA (US); Stefan M. Kammerer, San Diego, CA (US); Andreas Braun, San Diego, CA (US)

Correspondence Address: BIOTECHNOLOGY LAW GROUP, c/o PORTFOLIO IP, P.O. BOX 52050, MINNEAPOLIS, MN, 55402, US

(21) Appl. No.: 10/703,817

(22) Filed: Nov. 6, 2003

Related U.S. Application Data
(60) Provisional application No. 60/489,703, filed on Jul. 23, 2003.
(60) Provisional application No. 60/424,475, filed on Nov. 6, 2002.

(51) Int. Cl. C12Q 1/68; A61K 38/16; A61K 48/00; A61K 33/24; A61K 31/4745

(52) U.S. Cl. 424/59; 514/44; 435/6; 514/8; 514/283; 424/649

(57) ABSTRACT

Provided herein are methods for identifying risk of melanoma in a subject and/or subjects at risk of melanoma, reagents and kits for carrying out the methods, methods for identifying candidate therapeutics for treating melanoma, therapeutic methods for treating melanoma in a subject and compositions comprising one or more melanoma cells and one or more NRP1, NID2 or ENDO180 directed agents. These embodiments are based upon an analysis of polymorphic variations in a NRP1, NID2 or ENDO180 nucleic acid, exemplified by nucleotide sequences of SEQ ID NO: 1, 2 or 3.

### RELATED PATENT APPLICATIONS

This patent application claims the benefit of provisional patent application 60/424,475 filed Nov. 6, 2002 and provisional patent application 60/489,703 filed Jul. 23, 2003, having attorney docket number 524593004000 and 524593004001, respectively. Each of these provisional patent applications names Richard B. Roth et al. as inventors. Each of these provisional patent applications is hereby incorporated herein by reference in its entirety, including all drawings and cited documents.

### FIELD OF THE INVENTION

The invention relates to genetic methods for identifying risk of melanoma and treatments that specifically target the disease.

### BACKGROUND

In some parts of the world, especially among western countries, the number of people who develop melanoma is increasing faster than any other cancer. In the United States, for example, the number of new cases of melanoma has more than doubled in the past twenty years. The probability of developing melanoma increases with age, but this disease effects people of all age groups. Melanoma is one of the most common cancers in young adults.

Melanoma occurs when melanocytes (pigment cells) become malignant. Most pigment cells are in skin, and when melanoma begins its etiology in the skin it is referred to as coetaneous melanoma. Melanoma may also occur in the eye and is called ocular melanoma or intraocular melanoma. Rarely, melanoma arises in the meninges, the digestive tract, lymph nodes or other areas where melanocytes are found. Within the skin, melanocytes are located throughout the lower part of the epidermis, the latter being the surface layer of the skin. Melanocytes produce melanin, which is the pigment that gives skin its natural color. When skin is exposed to the sun, melanocytes produce more pigment, causing the skin to tan or darken.

Sometimes, clusters of melanocytes and surrounding tissue form benign growths referred to as moles or nevi (singular form is nevus). Cells in or near the nevi can divide without control or order and form malignant tumors. When melanoma spreads, cancer cells often are found in the lymph nodes. If the cancer has reached the lymph nodes, it may mean that cancer cells have spread to other parts of the body such as the liver, lungs or brain, giving rise to metastatic melanoma.

Melanoma is currently diagnosed by assessing risk factors and by performing biopsies. Risk factors for melanoma are a family history of melanoma, the presence of dysplastic nevi, patient history of melanoma, weakened immune system, many ordinary nevi, exposure levels to ultraviolet radiation, exposure to severe sunburns especially as a child or teenager, and fair skin. In a biopsy, a pathologist typically examines the biopsied tissue under a microscope to identify cancer cells. Depending upon the thickness of a tumor, if one exists, a physician may order chest x-ray, blood tests, liver scans, bone scans, and brain scans to determine whether the cancer spread to other tissues. Also, a test that identifies p16 nucleotide sequences is sold.

Upon a diagnosis of melanoma, the standard treatment is surgery. Side effects of surgery typically are pain and scarring. Surgery is generally not effective, however, in controlling melanoma that is known to have spread to other parts of the body. In such cases, physicians may utilize other methods of treatment, such as chemotherapy, biological therapy, radiation therapy, or a combination of these methods. Chemotherapy agents for treating melanoma include cisplatin, vinblastine, and dacarbazine. Chemotherapy can lead to side effects such as an increased probability of infection, bruising and bleeding, weakness and fatigue, hair loss, poor appetite, nausea and vomiting, and mouth and lip sores. Side effects of radiation therapy include fatigue and hair loss in the treated area. Biological therapies currently utilized for treatment of melanoma include interferon and interleuken-2. Side effects caused by biological therapies include flu-like symptoms, such as chills, fever, muscle aches, weakness, loss of appetite, nausea, vomiting, and diarrhea; bleeding and bruising skin; rashes, and swelling.

Certain melanoma therapeutics are in clinical trials. For example, canvaxin, which is a whole cell allogenic vaccine developed by irradiating tumor cells from two different patients, is under study. In addition, MAGE-1 and 3 minigenes and peptides and gp 100 peptides are being tested. Upcoming studies include testing of agents such as dacarbazine with a bcl-2 antisense oligonucleotide, and paclitaxel in combination with a matrix metalloprotease inhibitor.

### SUMMARY

It has been discovered that polymorphic variations of NRP1, NID2 and ENDO180 loci in human genomic DNA are associated with occurrence of melanoma. Thus, featured herein are methods for identifying a subject at risk of melanoma and/or determining risk of melanoma in a subject, which comprise detecting the presence or absence of one or more polymorphic variations associated with melanoma in a nucleic acid sample from the subject. The one or more polymorphic variations of ten are detected in or near the NRP1, NID2 and/or ENDO180 nucleotide sequence, which are set forth as SEQ ID NOs: 1, 2 and 3 respectively, or a substantially identical nucleotide sequence thereof.

Also featured are nucleic acids that encode a NRP1, NID2 or ENDO180 polypeptide, and include one or more polymorphic variations associated with melanoma, and oligonucleotides which hybridize to those nucleic acids. Also provided are polypeptides encoded by nucleic acids having a NRP1, NID2 or ENDO180 nucleotide sequence, which include the full-length polypeptide, isoforms and fragments thereof. In an embodiment, an NID2 protein is encoded which includes a serine at amino acid position 656 (i.e., position 656 in FIG. 6A). In addition, featured are methods for identifying candidate therapeutic molecules for treating melanoma and related disorders, as well as methods of treating melanoma in a subject by administering a therapeutic molecule.

Also provided are compositions comprising a melanoma cell and/or a NRP1, NID2 or ENDO180 nucleic acid, or a fragment or substantially identical nucleic acid thereof, with a RNAi, siRNA, antisense DNA or RNA, or ribozyme nucleic acid designed from a NRP1, NID2 or ENDO180 nucleotide sequence. In an embodiment, the nucleic acid is designed from a NRP1, NID2 or ENDO180 nucleotide sequence that includes one or more melanoma associated polymorphic variations, and in some instances, specifically interacts with such a nucleotide sequence. Further, provided are arrays of nucleic acids bound to a solid surface, in which one or more nucleic acid molecules of the array are NRP1, NID2 or ENDO180 nucleic acids, or a fragment or substantially identical nucleic acid thereof, or a complementary nucleic acid of the foregoing. Featured also are compositions comprising a melanoma cell and/or a protein, polypeptide or peptide encoded by a NRP1, NID2 or ENDO180 nucleic acid with an antibody that specifically binds to the protien, polypeptide or peptide. In an embodiment, the antibody specifically binds to an epitope in a NRP1, NID2 or ENDO180 protein, polypeptide or peptide that includes a non-synonymous amino acid modification associated with melanoma, such as a serine at position 656 of a NID2 protein, polypeptide or peptide.

### BRIEF DESCRIPTION OF THE DRAWINGS

FIGS. 1A to 1W show a genomic sequence of neuropilin 1 (NRP1) with the polymorphic variants in IUPAC format. The genomic sequence set forth in FIGS. 1A to 1W correspond to SEQ ID NO: 1. The following nucleotide representations are used throughout the specification and figures: “A” or “a” is adenosine, adenine, or adenylic acid; “C” or “c” is cytidine, cytosine, or cytidylic acid; “G” or “g” is guanosine, guanine, or guanylic acid; “T” or “t” is thymidine, thymine, or thymidylic acid; and “I” or “i” is inosine, hypoxanthine, or inosinic acid. SNPs are designated by the following convention: “R” represents A or G, “M” represents A or C; “W” represents A or T; “Y” represents C or T; “S” represents C or G; “K” represents G or T; “V” represents A, C or G; “H” represents A, C, or T; “D” represents A, G, or T; “B” represents C, G, or T; and “N” represents A, G, C, or T.

FIG. 2 shows a human cDNA structure for NRP1.

FIG. 3 shows a human cDNA structure for nidogen 2 (NID2).

FIGS. 4A and 4B show a human cDNA structure for mannose receptor, C type 2 (MRC2), also known as ENDO180. Throughout the specification and claims, this gene and gene product are referred to as ENDO180.

FIGS. 5A and 5B show a human polypeptide sequences for NRP1.

FIGS. 6A and 6B show a human polypeptide sequences for NID2.

FIGS. 7A and 7B show a human polypeptide sequences for ENDO180.

FIGS. 8, 9 and 10 show proximal SNPs in and around the NRP1 gene for males and females combined, females alone, and males alone, respectively. The position of each SNP on the chromosome is shown on the x-axis and the y-axis provides the negative logarithm of the p-value comparing the estimated allele to that of the control group. Also shown in FIGS. 8-10 are the exons and introns of the genes in the approximate chromosomal positions.

FIG. 11 depicts effects of NRP1 directed siRNA on melanoma M14 cell line proliferation according to a WST-1 assay.

FIG. 12 shows effects of NRP1 directed siRNA on melanoma A375 cell line proliferation according to a WST-1 assay.

FIGS. 13A to 13O depict a genomic nucleotide sequence of NRP1, which corresponds to SEQ ID NO: 2. SNP positions designated as rs3742536 and rs753050 herein are located at positions 32929 and 34983, respectively, in these figures.

FIGS. 14A to 14P depict a genomic nucleotide sequence of ENDO180, which corresponds to SEQ ID NO: 3. The SNP position designated as rs729647 herein is located at position 43763 in these figures.

### DETAILED DESCRIPTION

It has been discovered that polymorphic variants in and around NRP1, NID2 or ENDO180 nucleotide sequences are associated with occurrence of melanoma in subjects. Thus, detecting genetic determinates associated with an increased risk of melanoma occurrence can lead to early identification of melanoma or susceptibility to melanoma and early prescription of preventative measures and treatments. Also, associating NRP1, NID2 and ENDO180 polymorphic variants with melanoma has provided new targets for screening molecules useful in melanoma prognostics/diagnostics and melanoma treatments.

Melanoma and Sample Selection

Melanoma is typically described as a malignant neoplasm derived from cells capable of forming melanin. Melanomas arise most commonly in the skin of any part of the body, or in the eye, and rarely, in the mucous membranes of the genitalia, anus, oral cavity, or other sites. Melanoma occurs mostly in adults and may originate de novo or from a pigmented nevus or lentigo maligna. Melanomas frequently metastasize widely to regions such as lymph-nodes, skin, liver, lungs, and brain.

In the early phases, the cutaneous form is characterized by proliferation of cells at the dermal-epidermal junction that soon invade adjacent tissues. The cells vary in amount and pigmentation of cytoplasm; the nuclei are relatively large and irregular in shape, with prominent acidophilic nucleoli; and mitotic figures tend to be numerous. Other criteria for melanomas are asymmetry, irregular borders, heterogeneous color, large diameter, and a recent change in shape, size or pigmentation. Excised melanoma skin samples are often subjected to the following analyses: diagnosis of the melanocytic nature of the lesion and confirmation of its malignancy; maximum tumor thickness in millimeters (according to Breslow's method); assessment of completeness of excision of invasive and in situ components and microscopic measurements of the shortest extent of clearance; level of invasion (Clark); presence and extent of regression; presence and extent of ulceration; histological type and special variants; pre-existing lesion; mitotic rate; vascular invasion; neurotropism; cell type; tumor lymphocyte infiltration; and growth phase, vertical or radial.

Based in part upon selection criteria set forth above, individuals having melanoma can be selected for genetic studies. Also, individuals having no history of cancer or melanoma often are selected for genetic studies. Other selection criteria can include: a tissue or fluid sample is derived from an individual characterized as Caucasian; a sample is derived from an individual of German paternal and maternal descent; and relevant phenotype information is available for the individual. Phenotype information corresponding to each individual can include sex of the individual, number of nevi (e.g., actual number or relative number (e.g., few, moderate, numerous)), hair color (e.g., black, brown, blond, red), diagnosis of melanoma (e.g., tumor thickness, date of primary diagnosis, age of individual as of primary diagnosis, post-operative tumor classification, presence of nodes, occurrence of metastases, subtype, location), country or origin of mother and father, presence of certain conditions for each individual (e.g., coronary heart disease, cardiomyopathy, arteriosclerosis, abnormal blood clotting/thrombosis, emphysema, asthma, diabetes type 1, diabetes type 2, Alzheimer's disease, epilepsy, schizophrenia, manic depression/bipolar disorder, autoimmune disease, thyroid disorder, and hypertension), presence of cancer in the donor individual or blood relative (e.g., melanoma, basaliom/spinaliom/lentigo malignant/mycosis fungoides, breast cancer, colon cancer, rectum cancer, lung cancer, lung cancer, bronchus cancer, prostate cancer, stomach cancer, leukemia, lymphoma, or other cancer in donor, donor parent, donor aunt or uncle, donor offspring or donor grandparent).

Provided herein is a set of blood samples and a set of corresponding nucleic acid samples isolated from the blood samples, where the blood samples are donated from individuals diagnosed with melanoma. The sample set often includes blood samples or nucleic acid samples from 100 or more, 150 or more, or 200 or more individuals having melanoma, and sometimes from 250 or more, 300 or more, 400 or more, or 500 or more individuals. The individuals can have parents from any place of origin, and in an embodiment, the set of samples are extracted from individuals of German paternal and German maternal ancestry. The samples in each set may be selected based upon five or more criteria and/or phenotypes set forth above.

Polymorphic Variants Associated with Melanoma

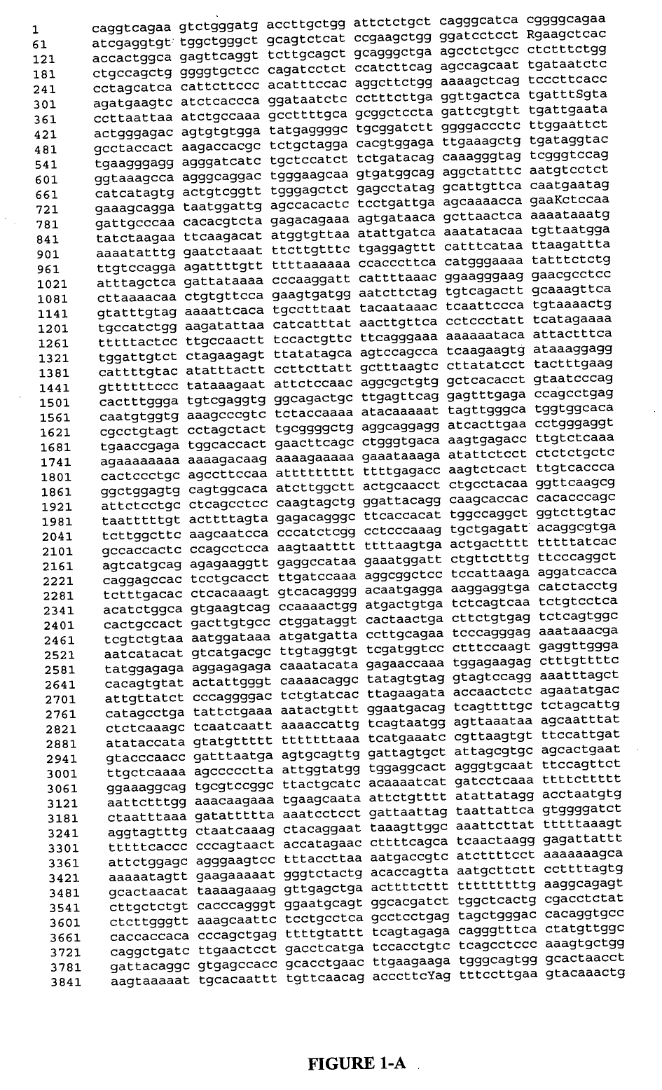

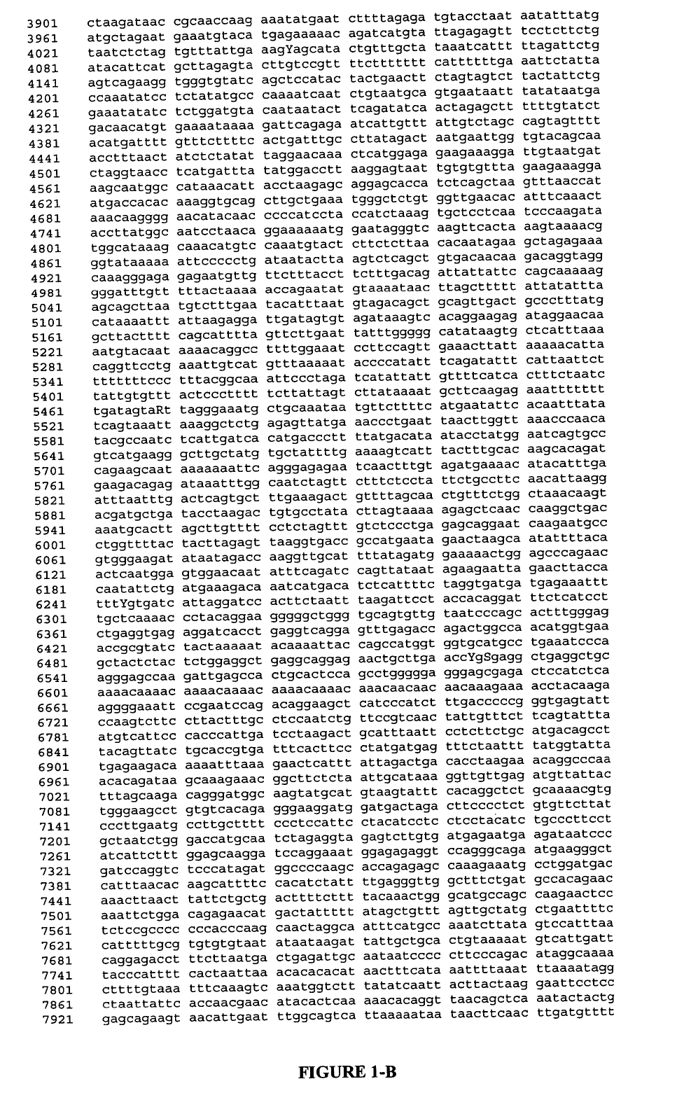

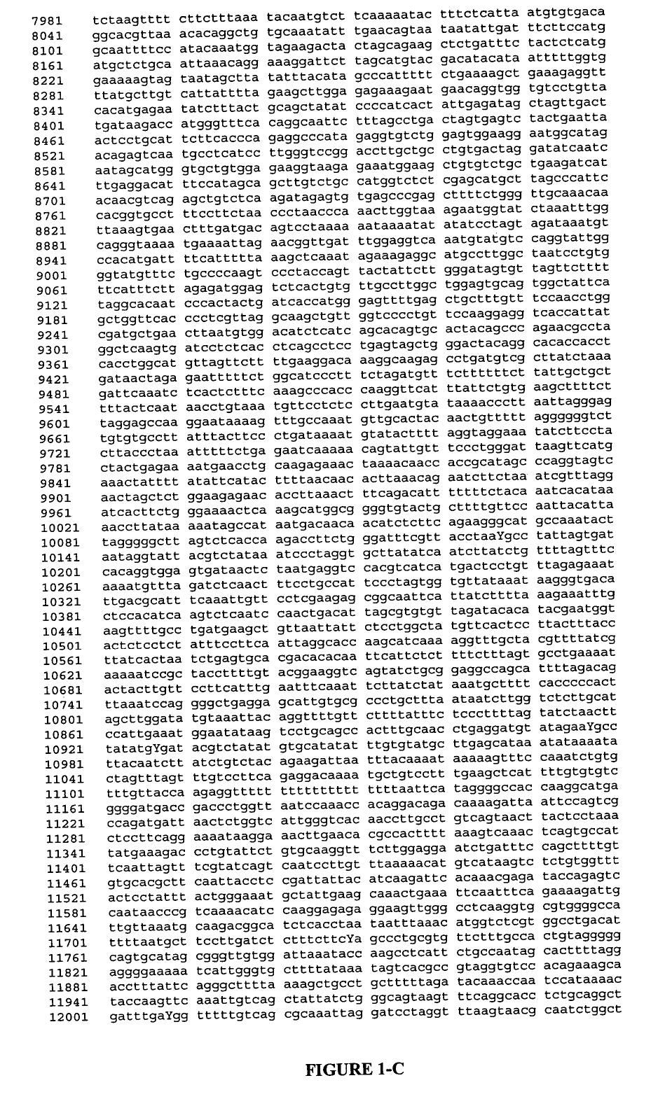

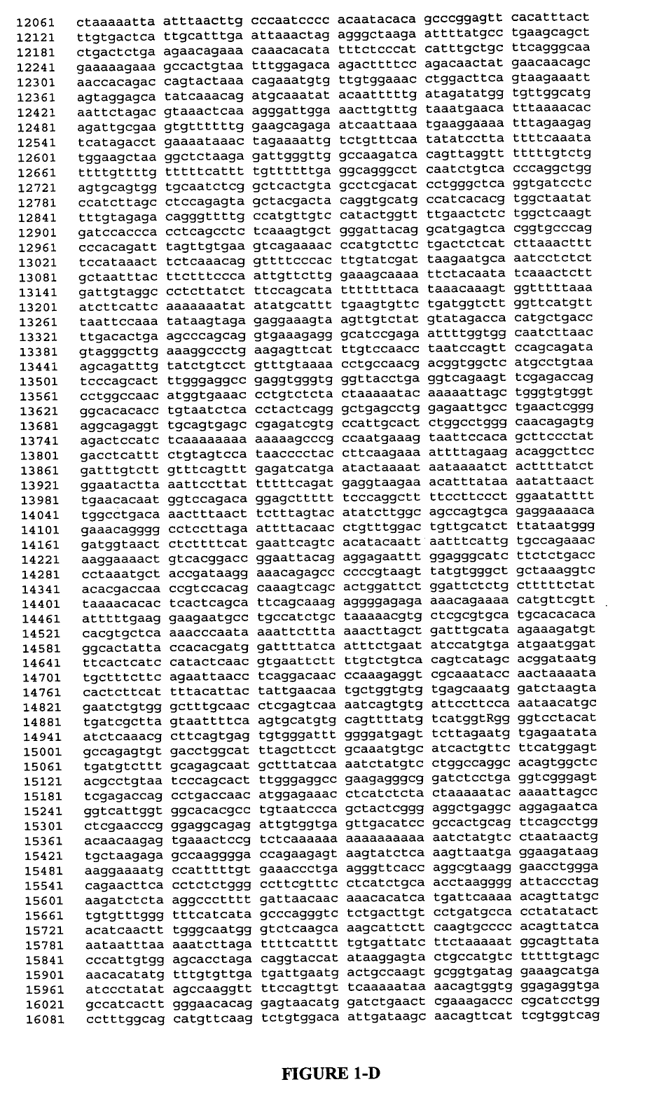

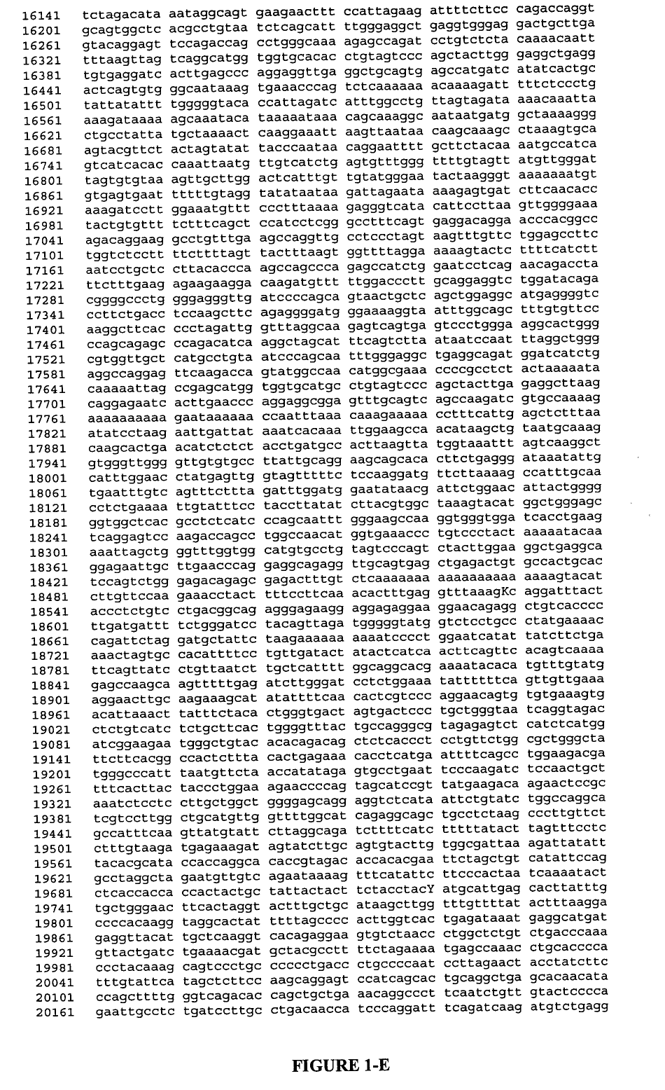

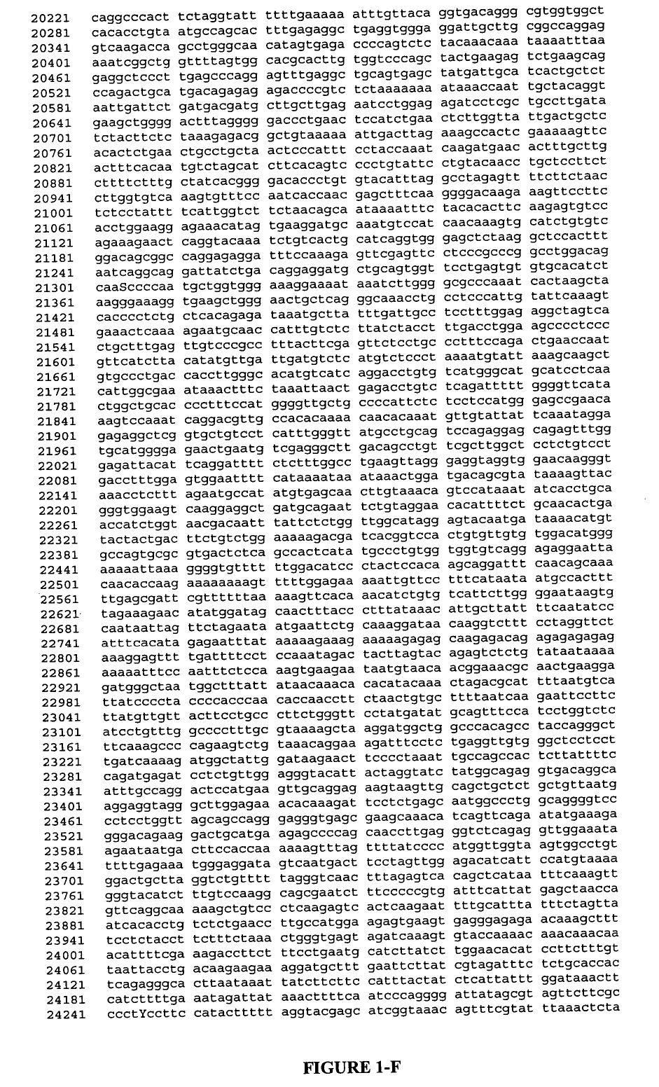

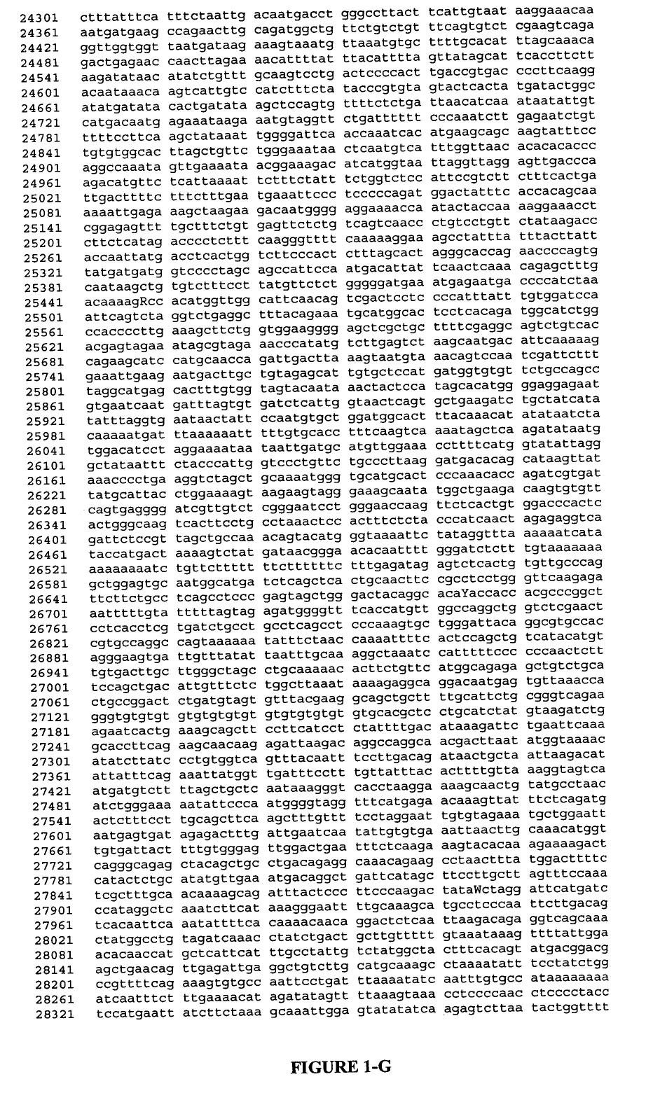

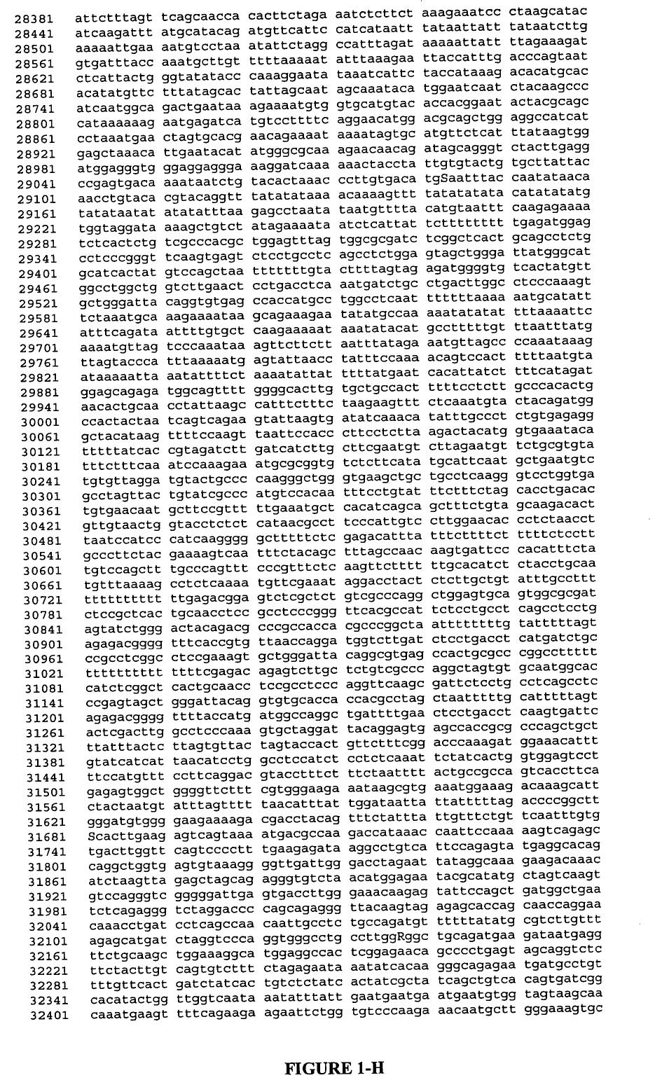

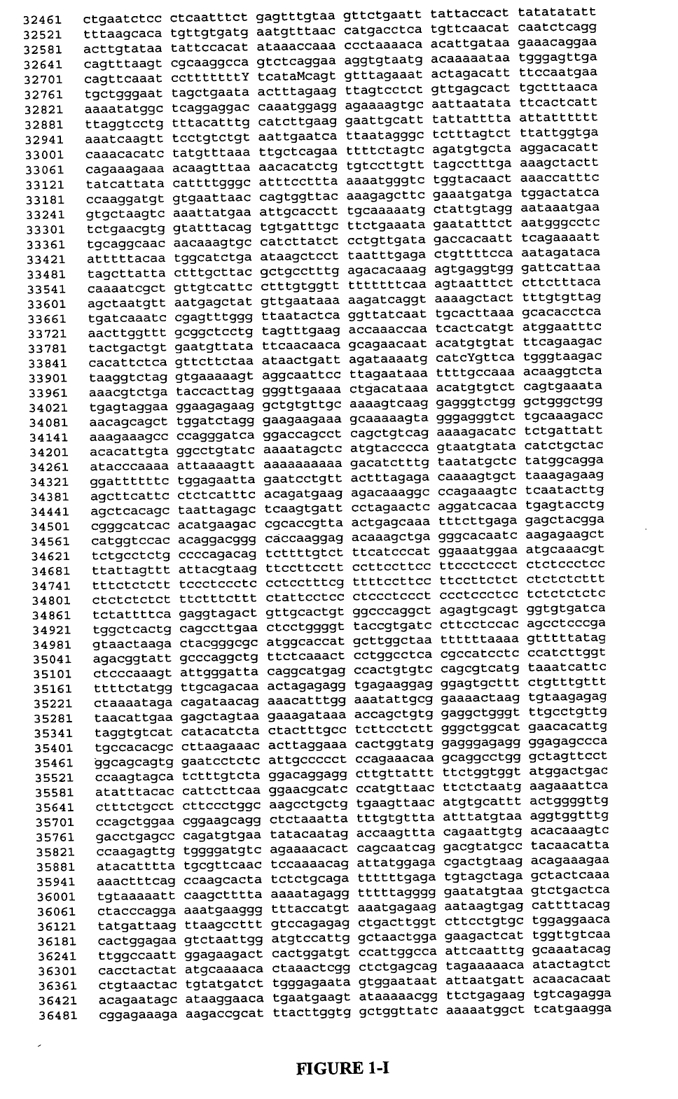

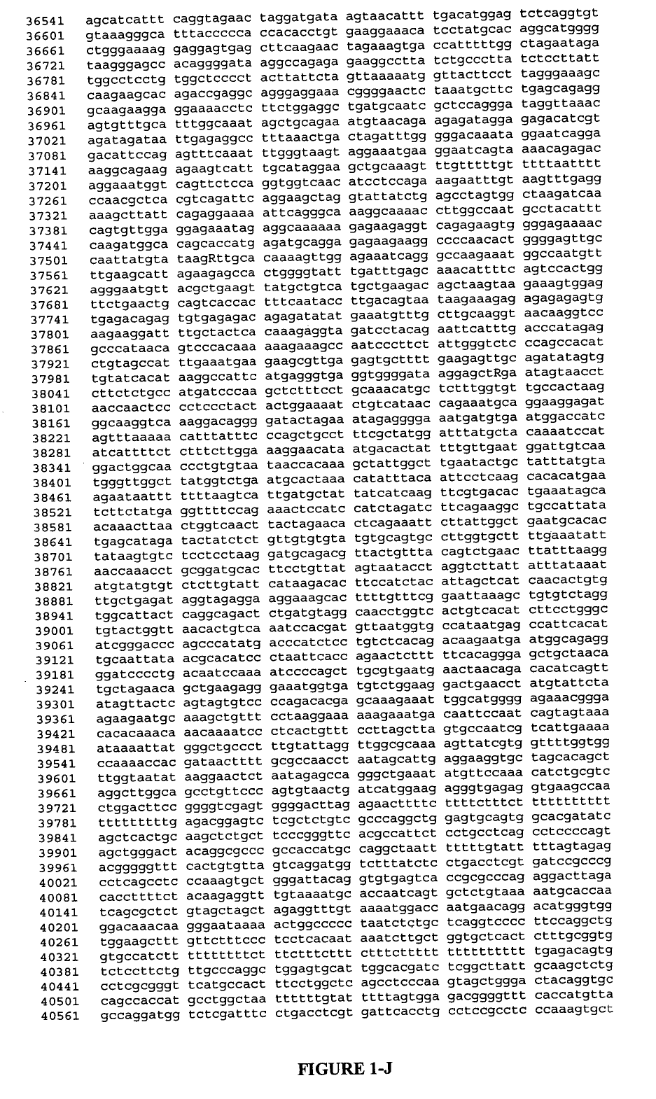

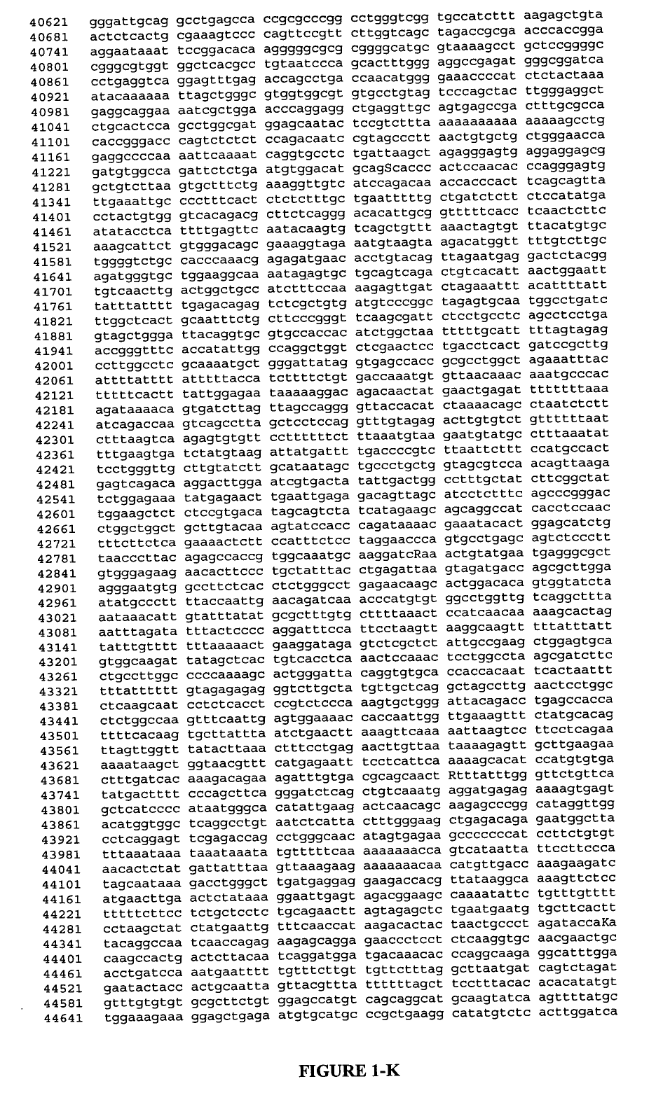

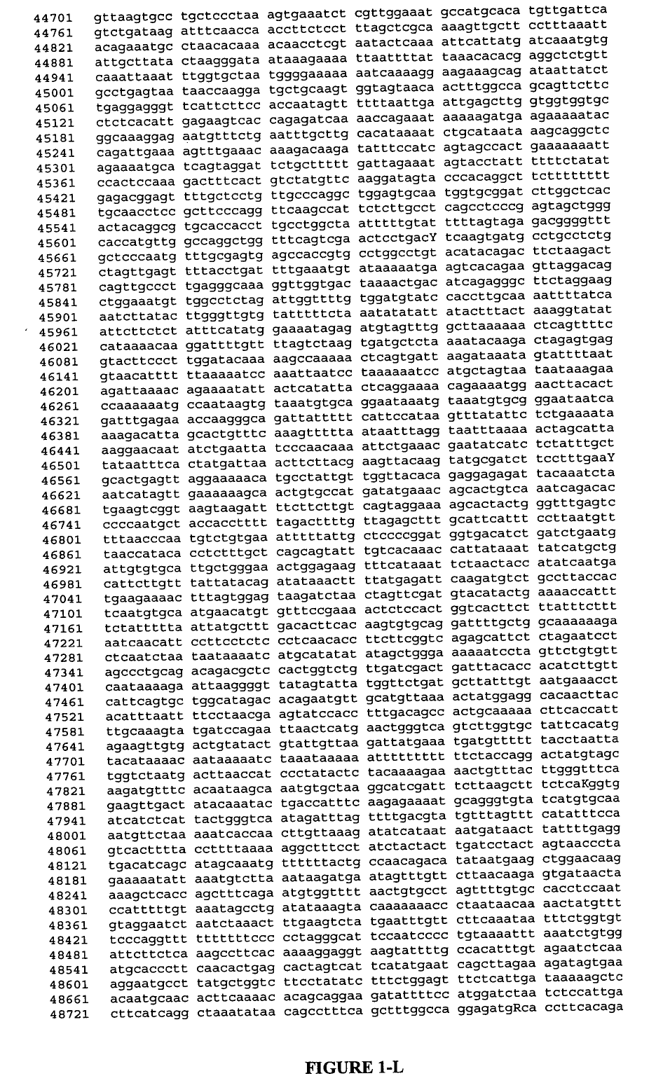

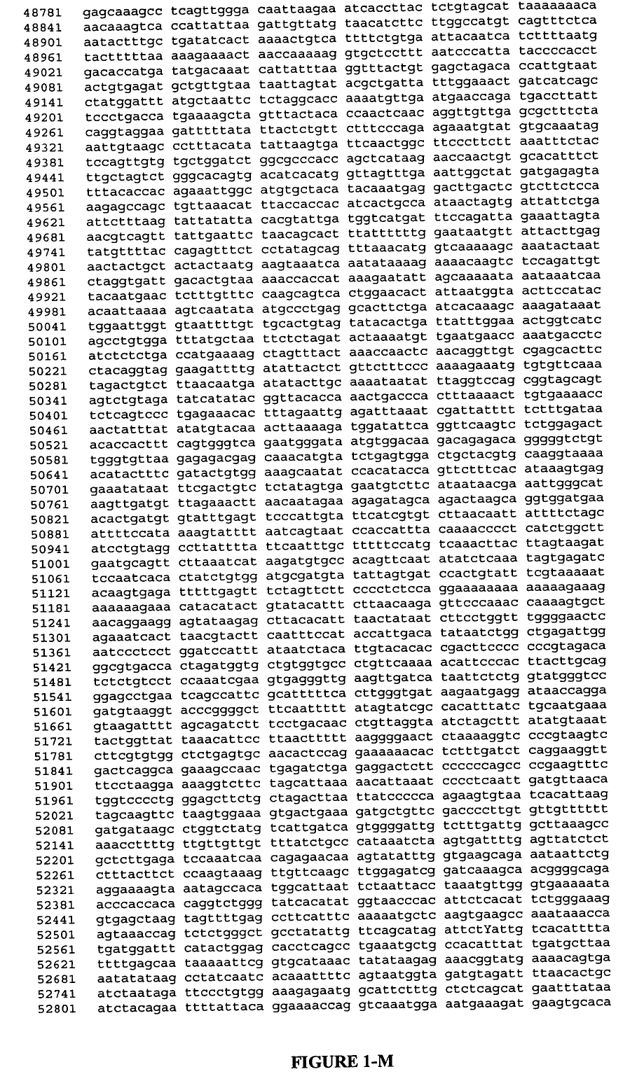

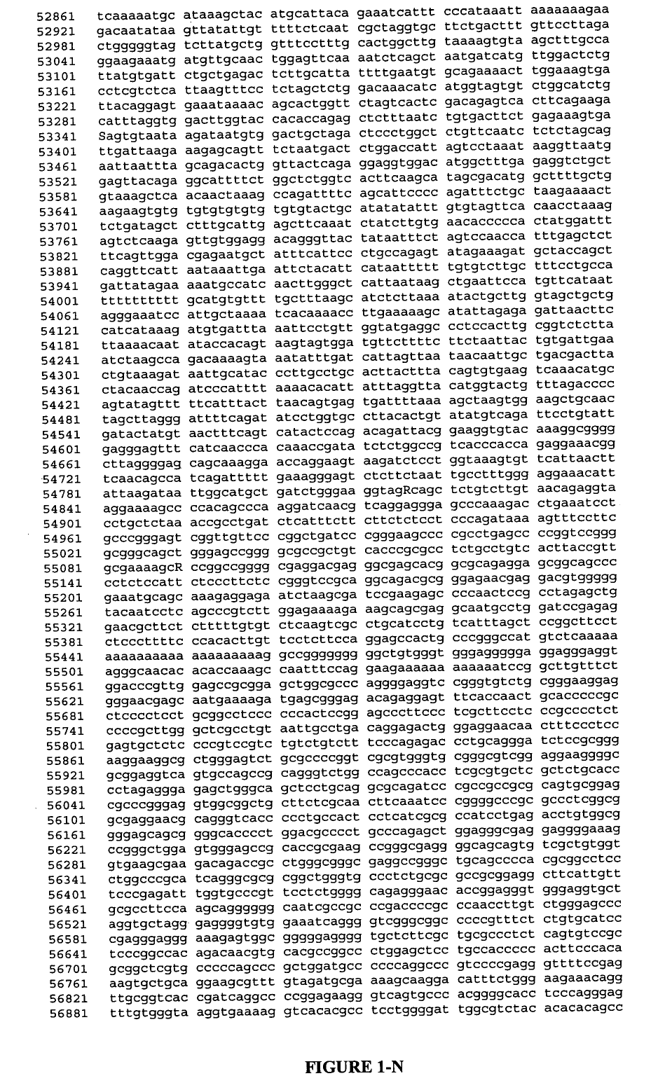

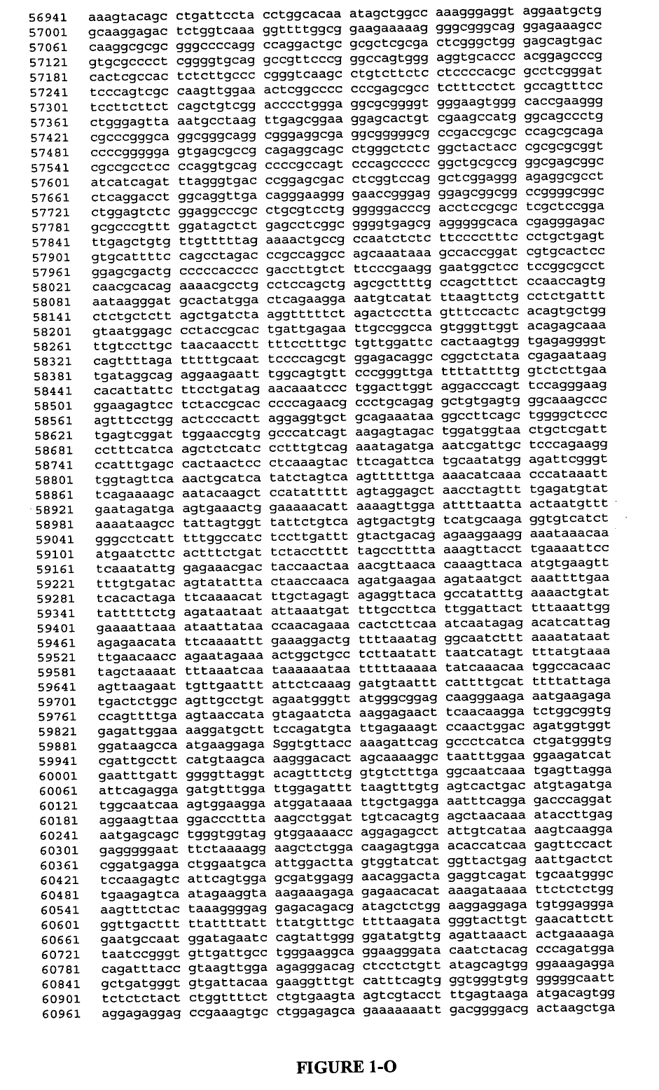

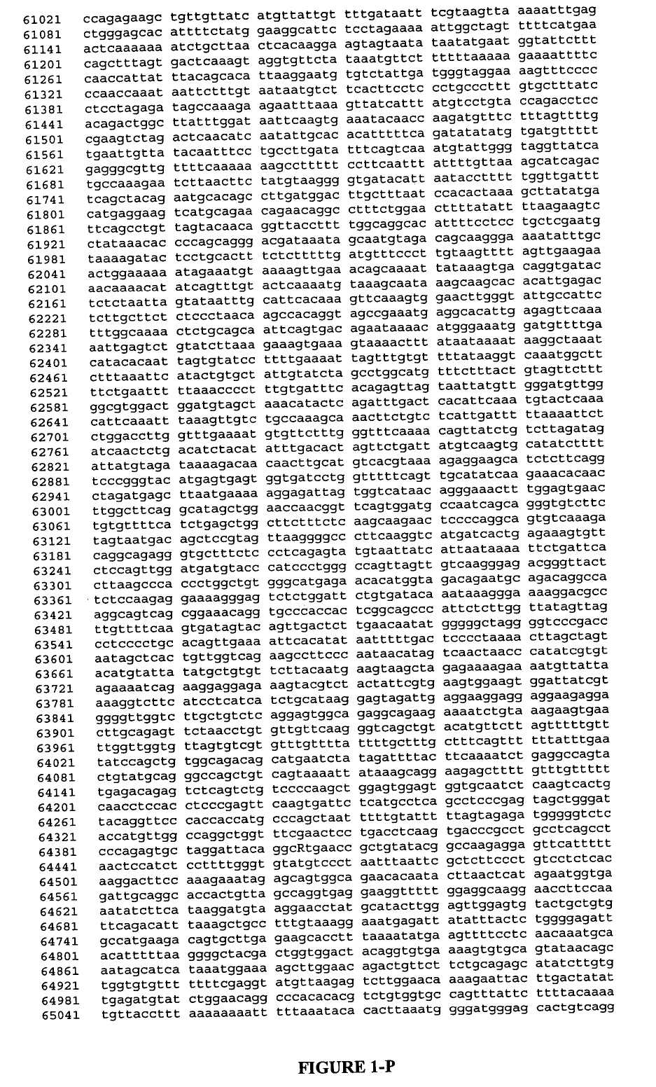

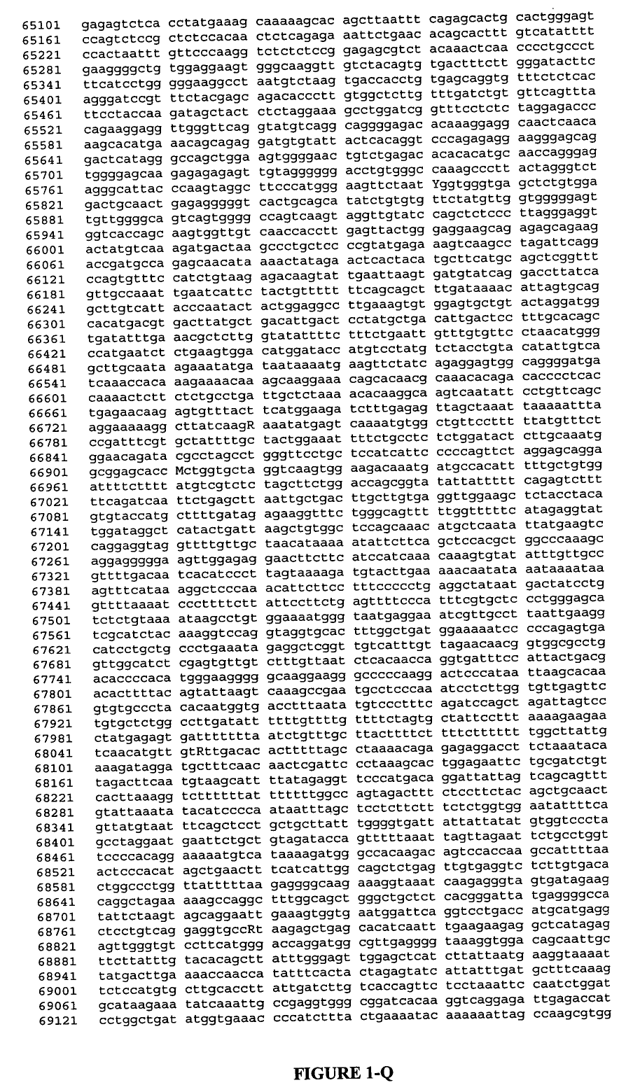

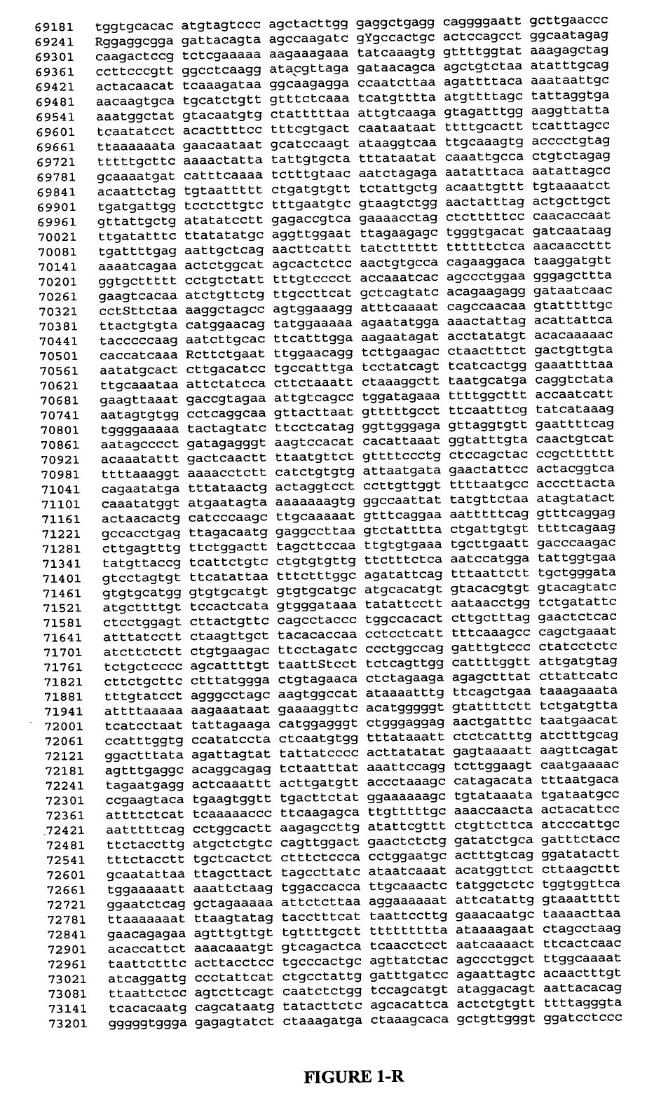

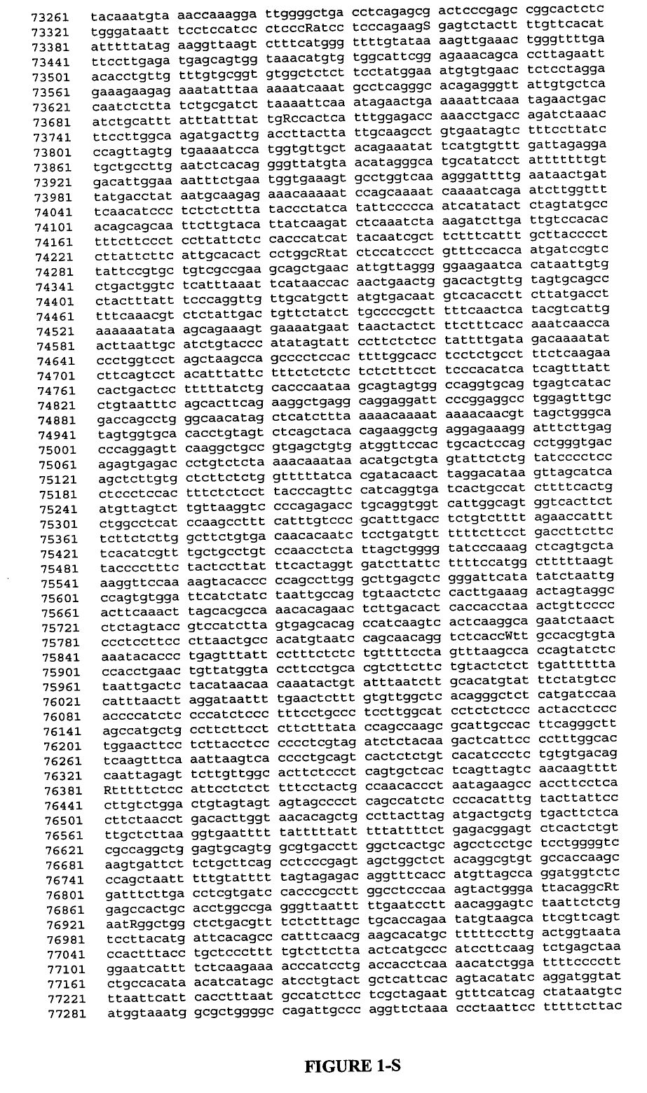

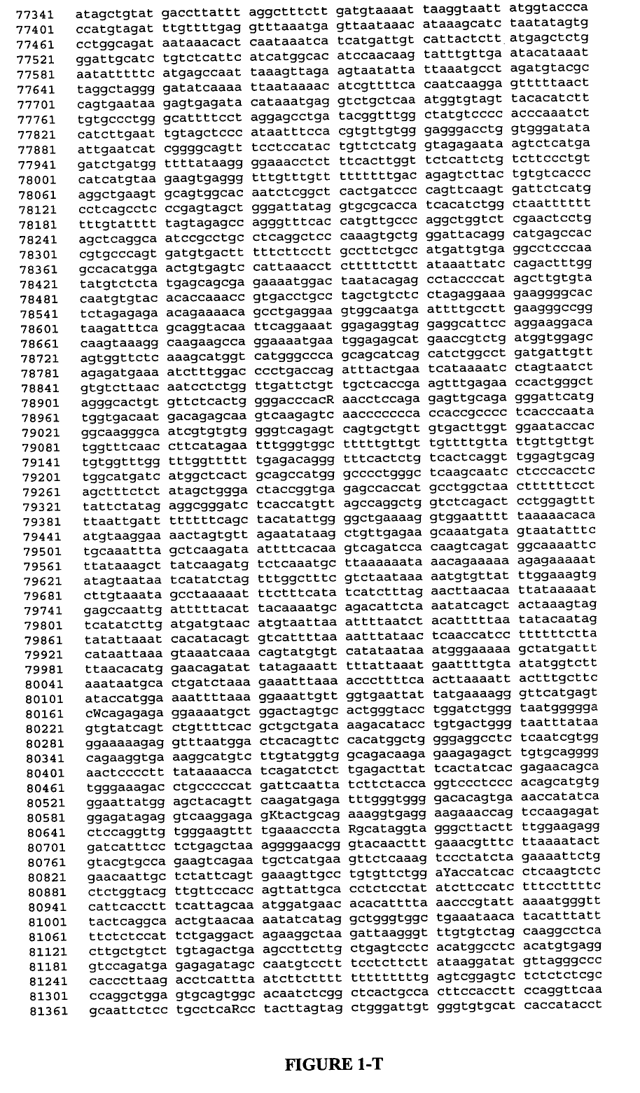

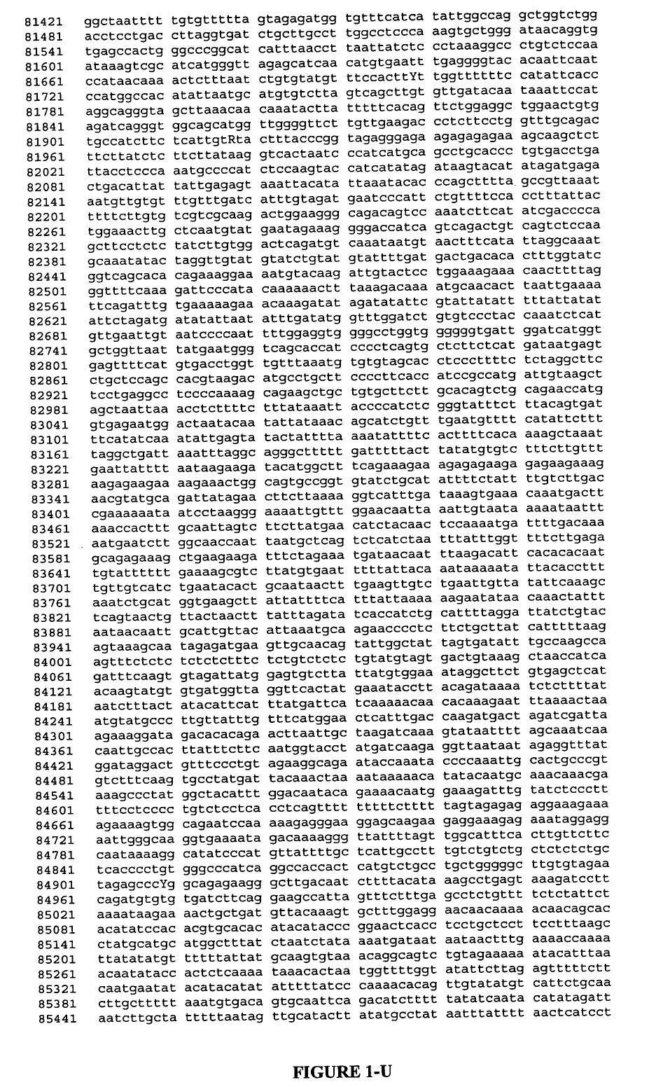

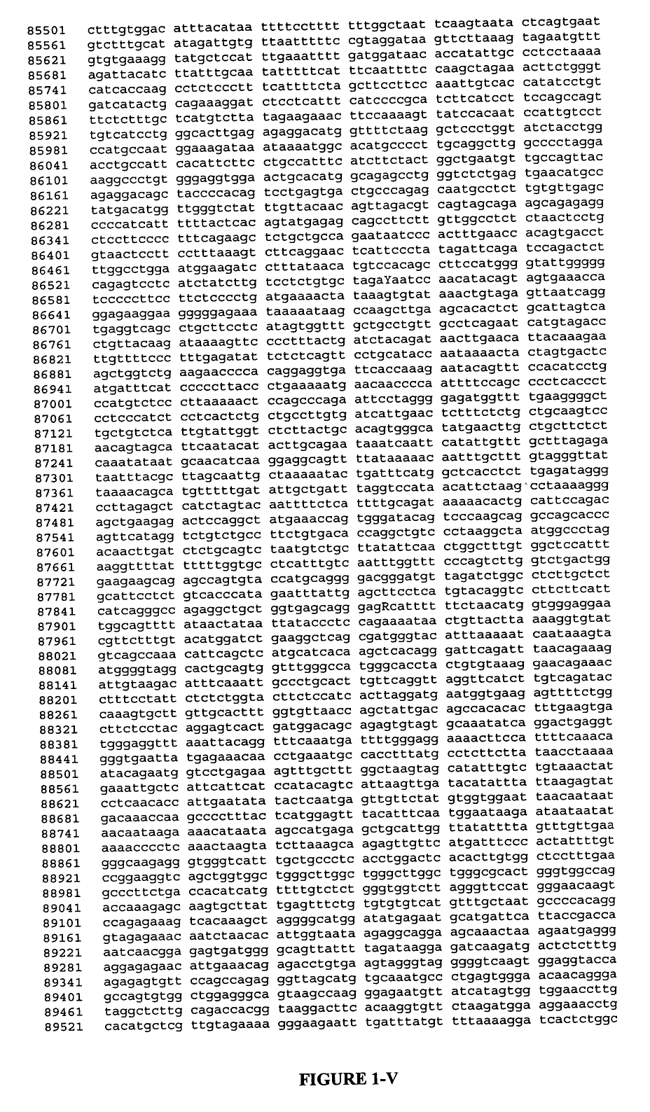

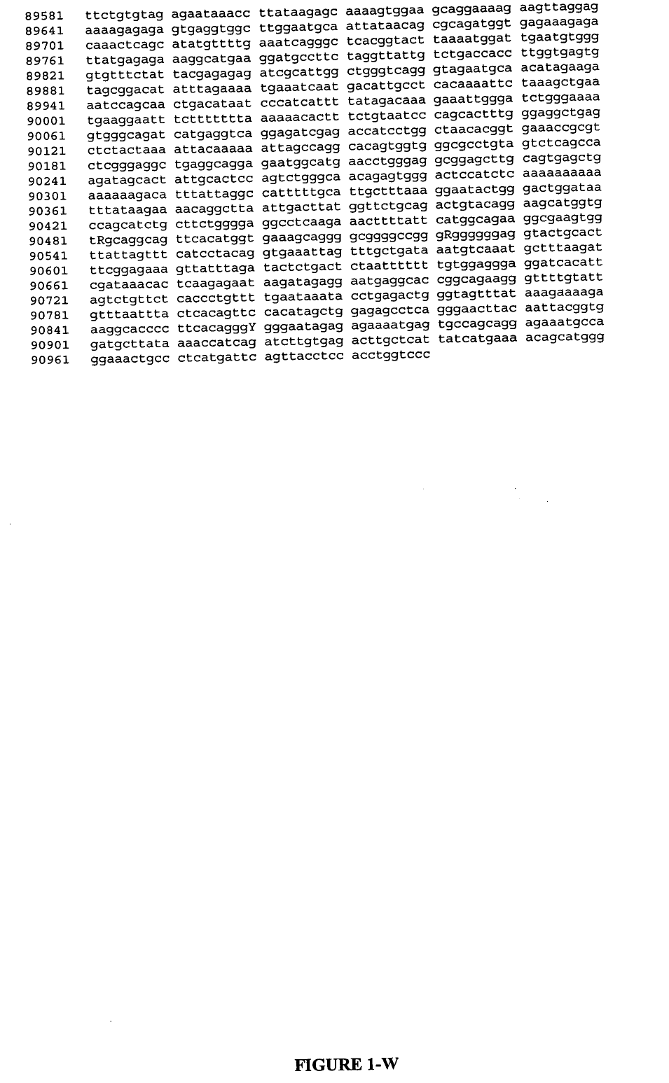

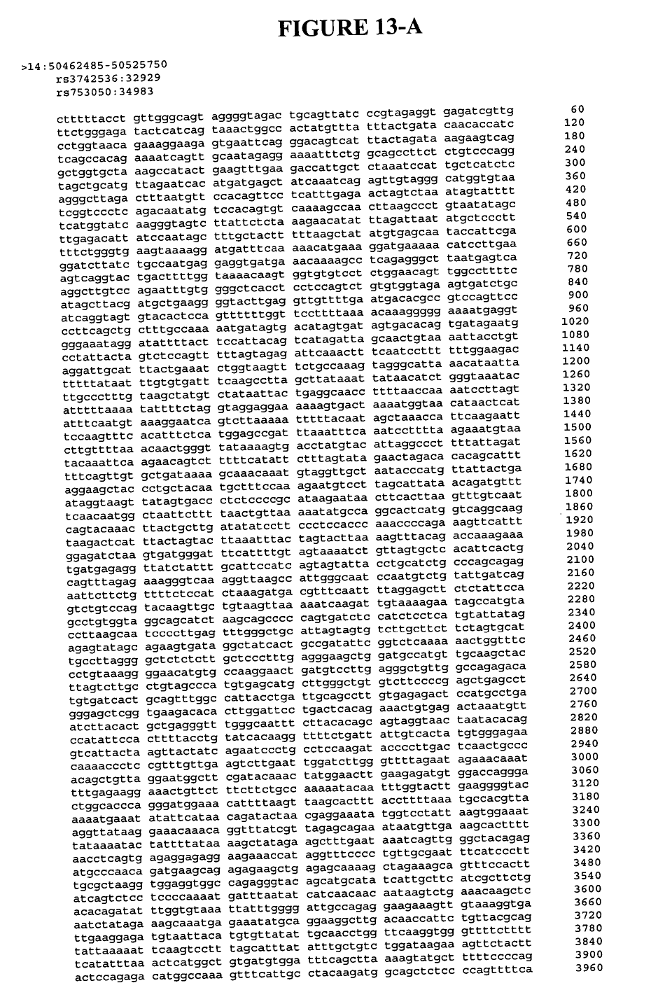

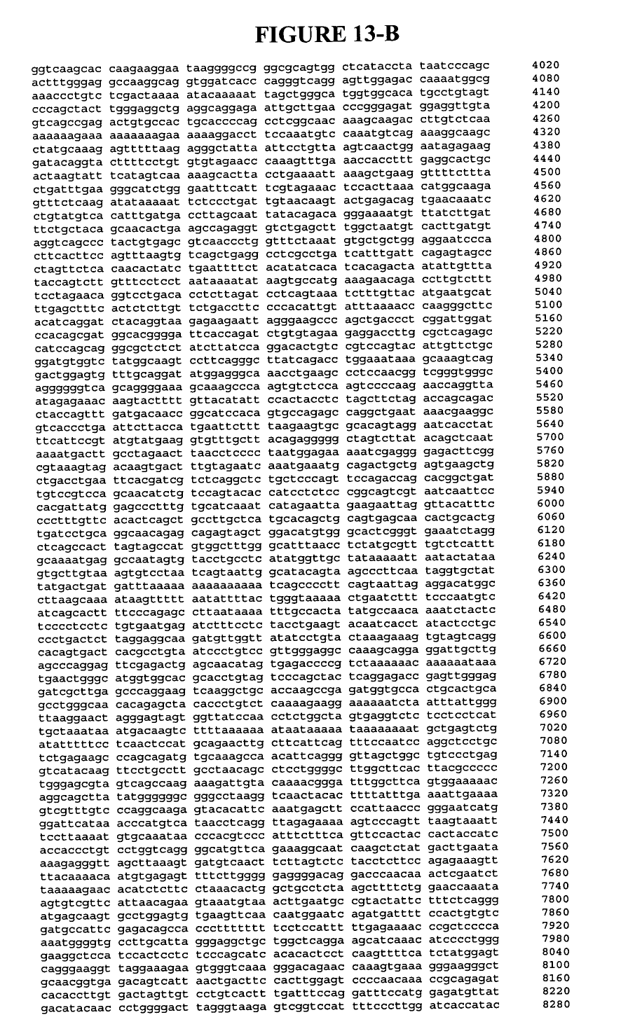

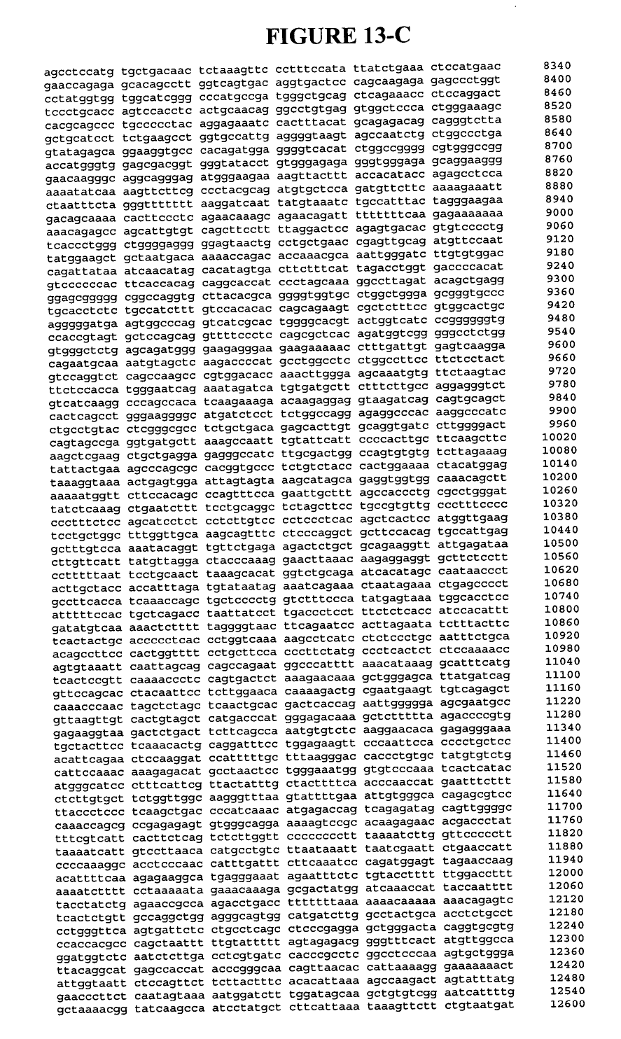

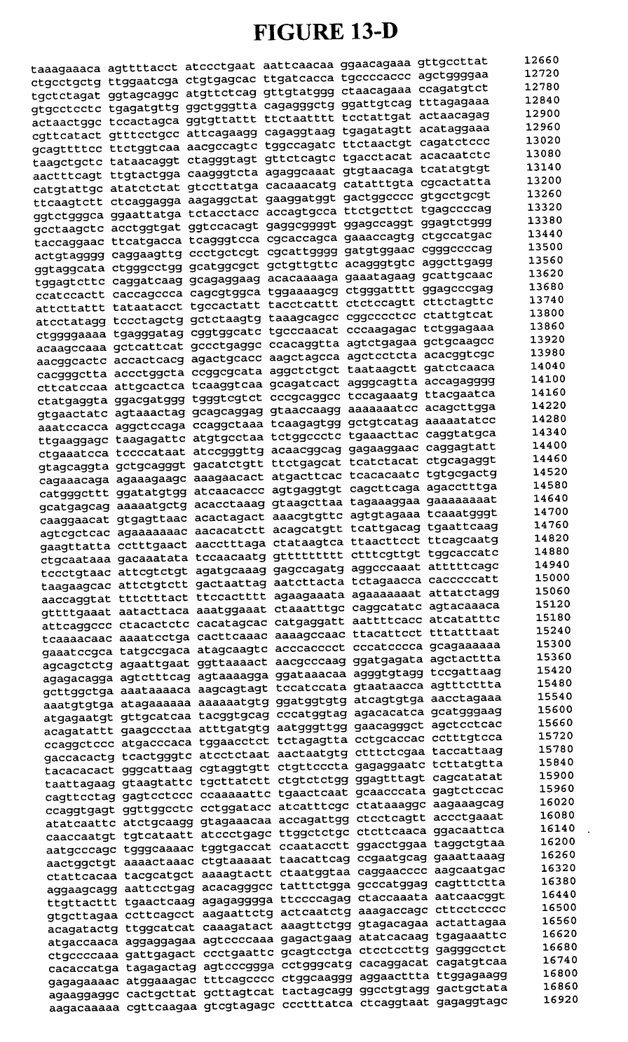

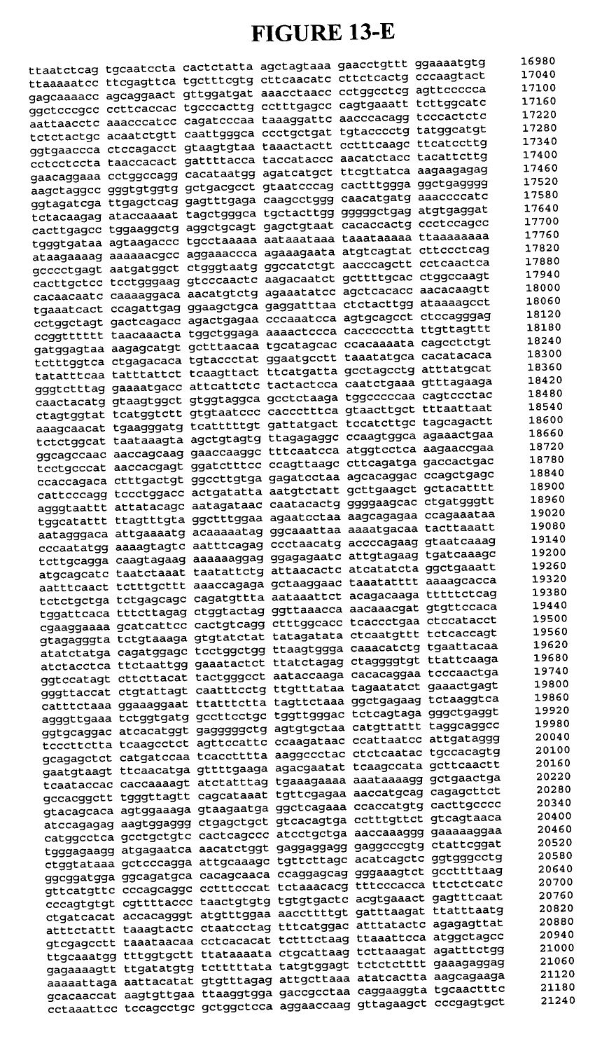

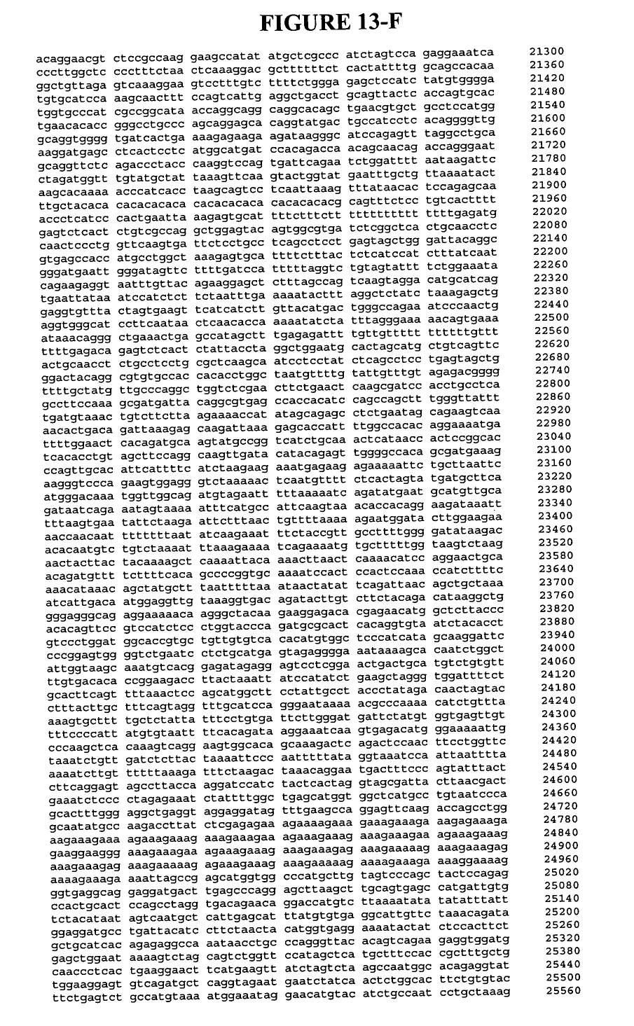

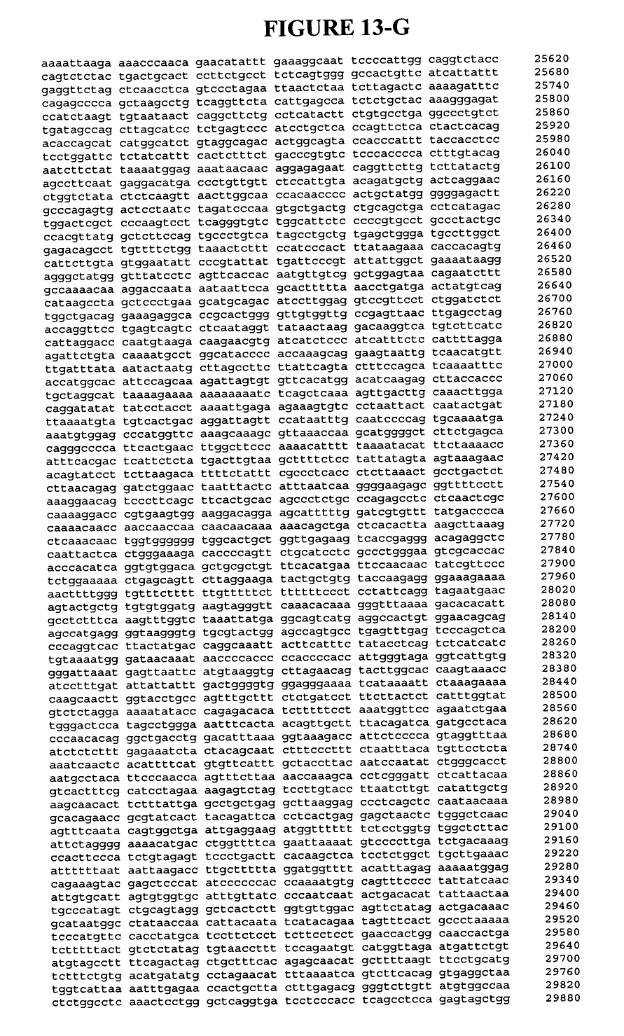

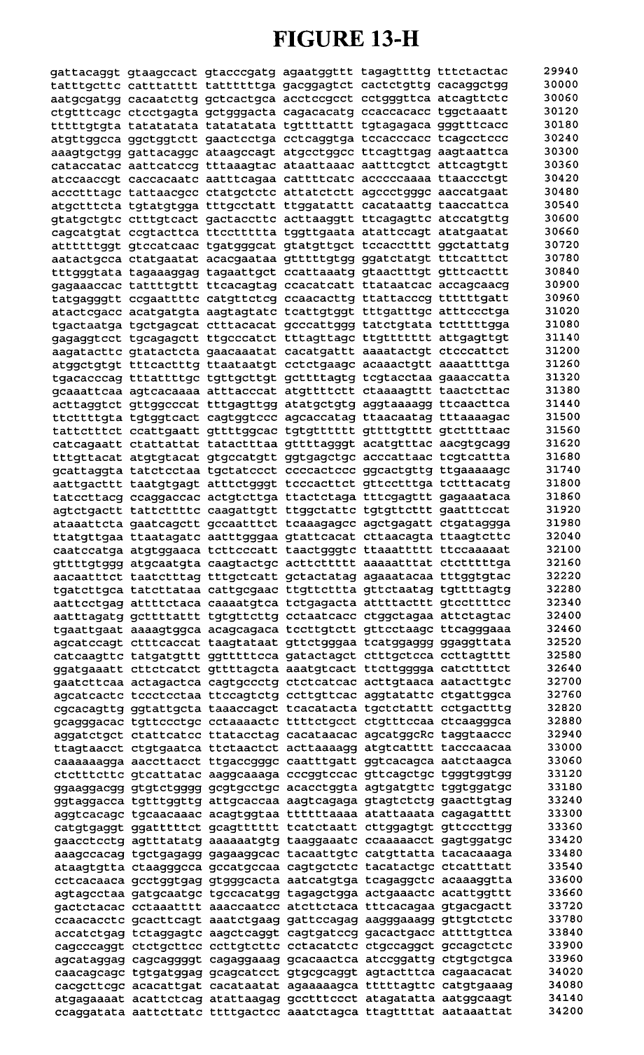

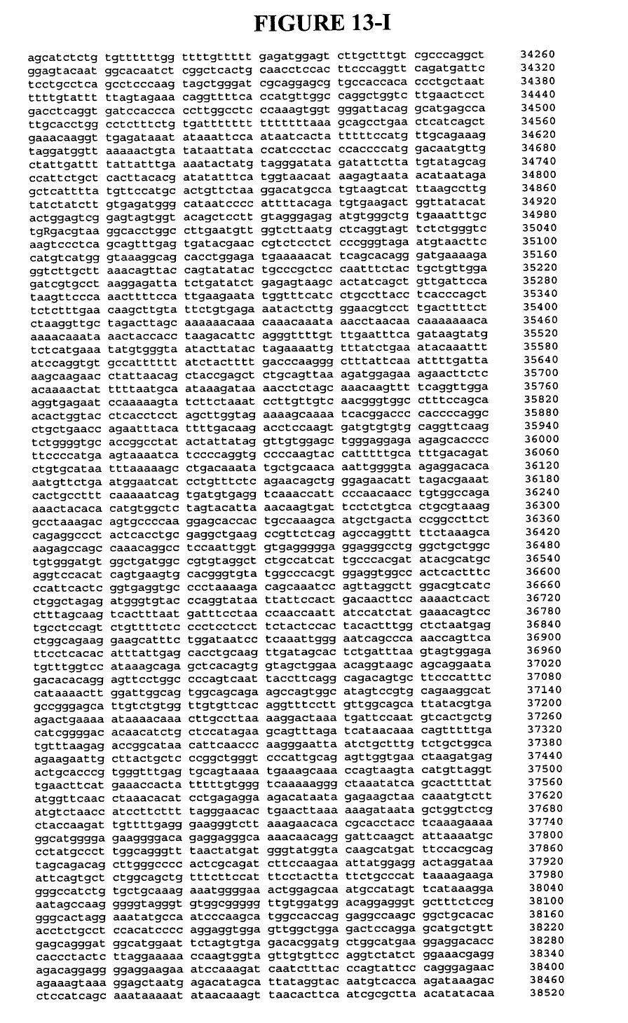

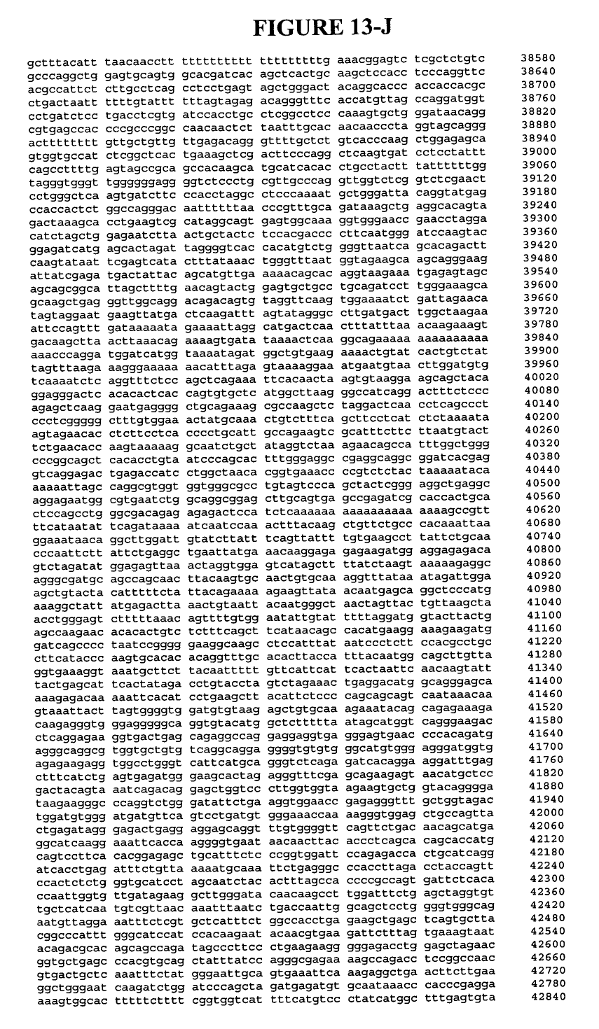

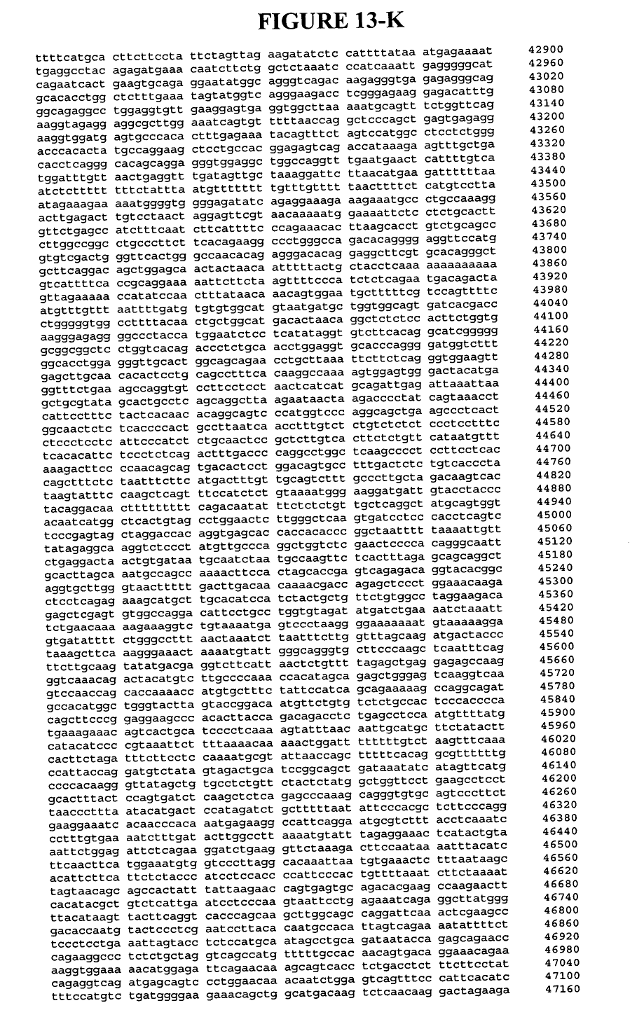

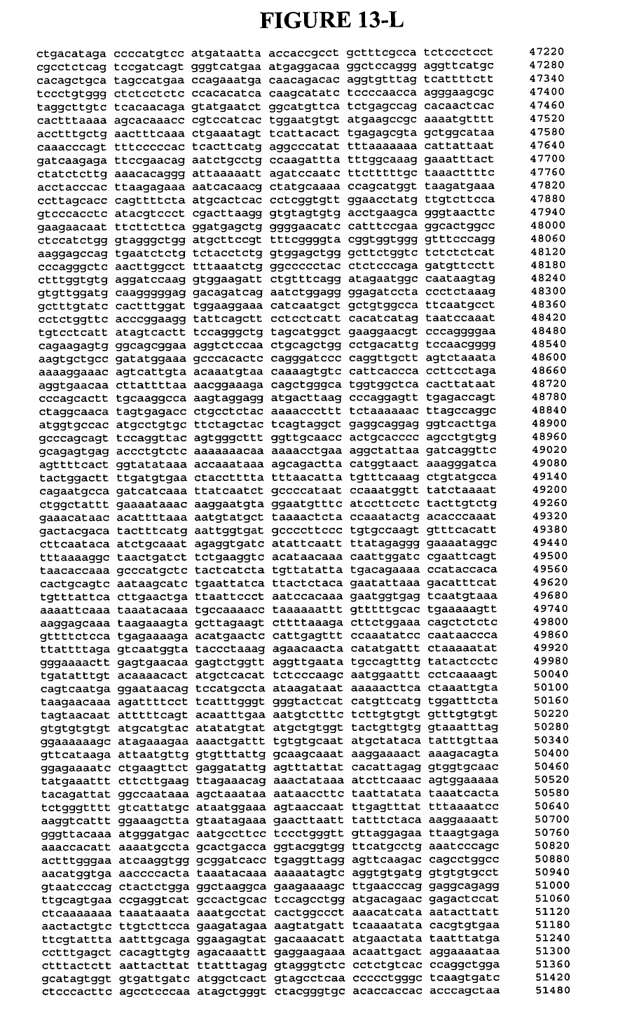

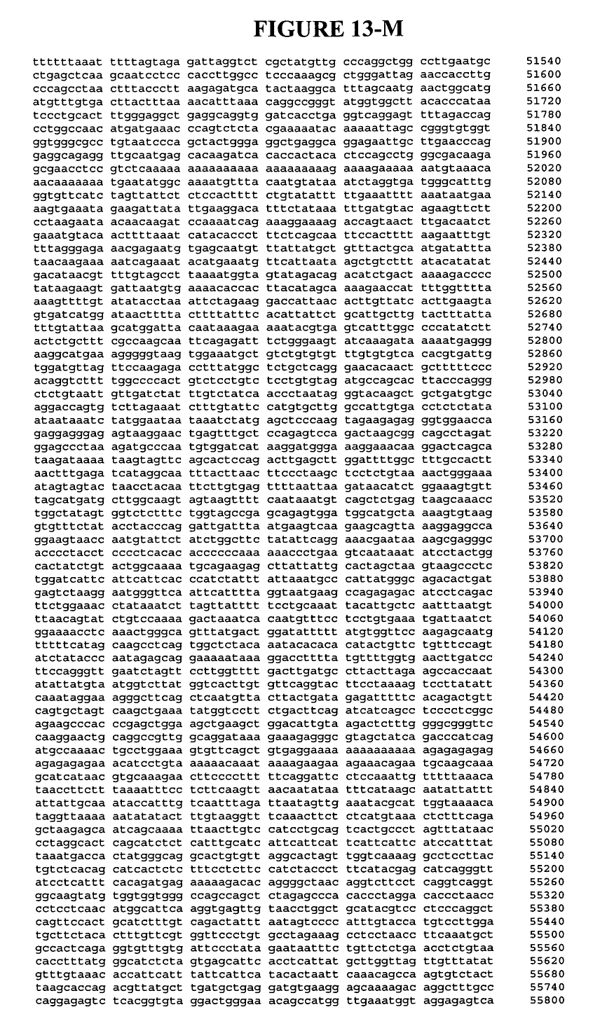

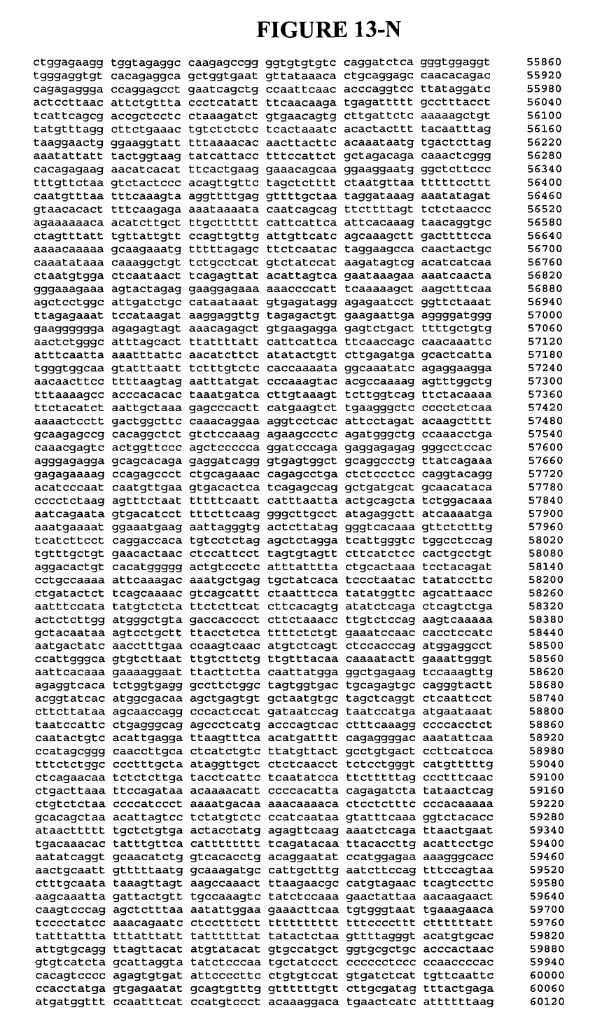

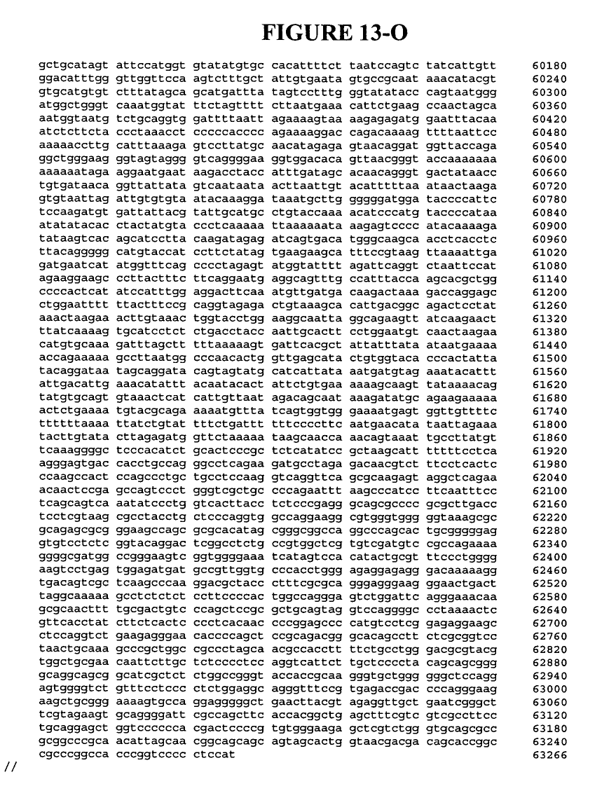

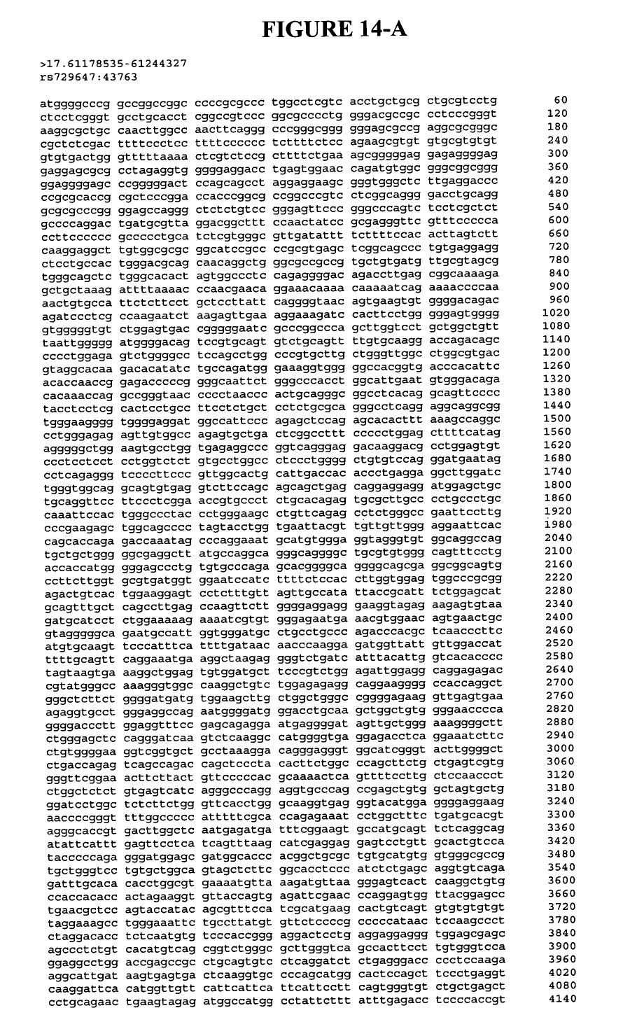

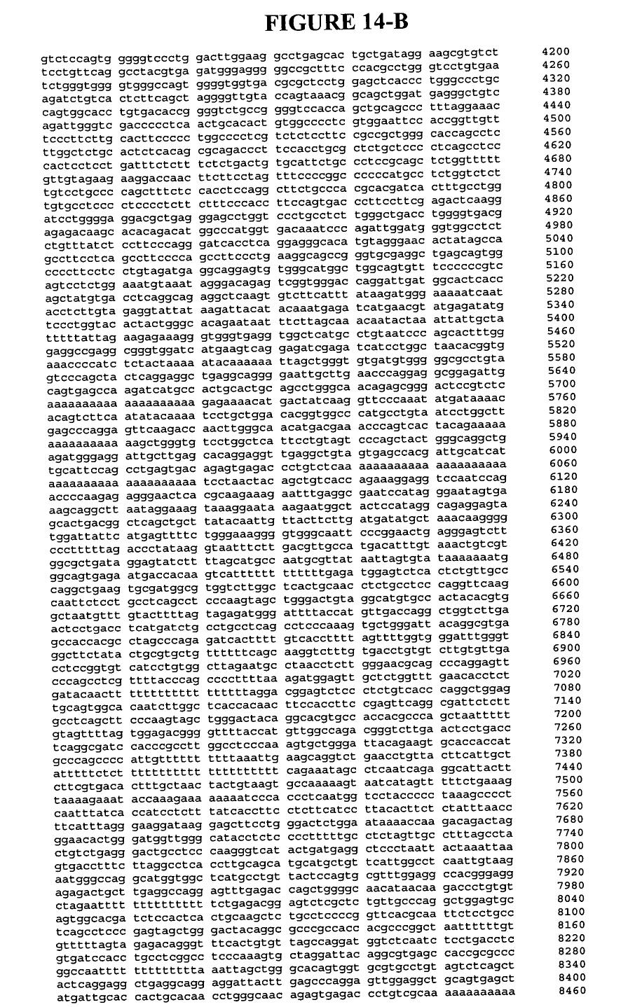

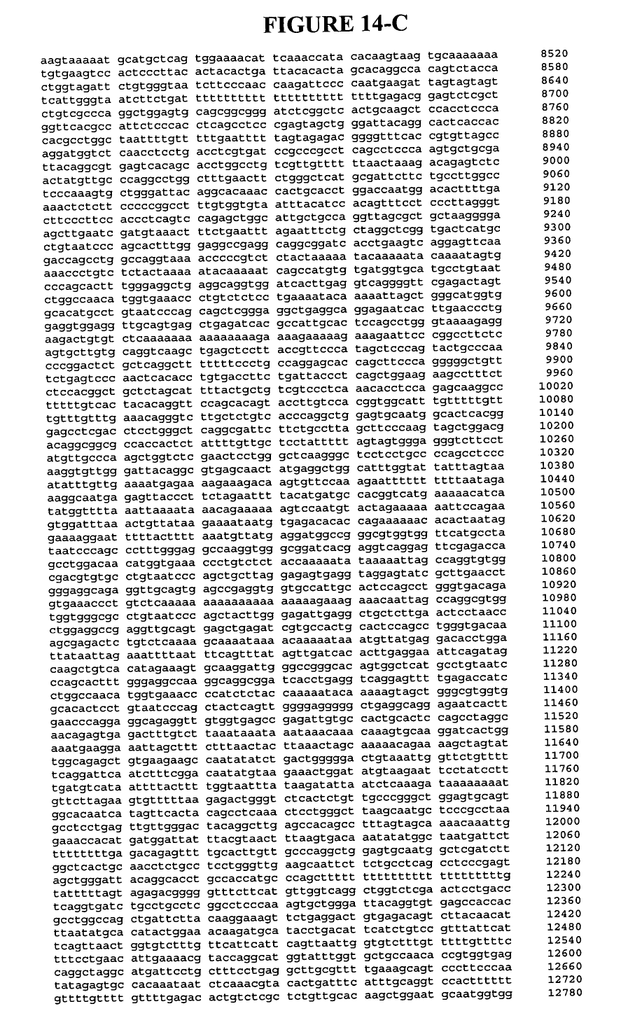

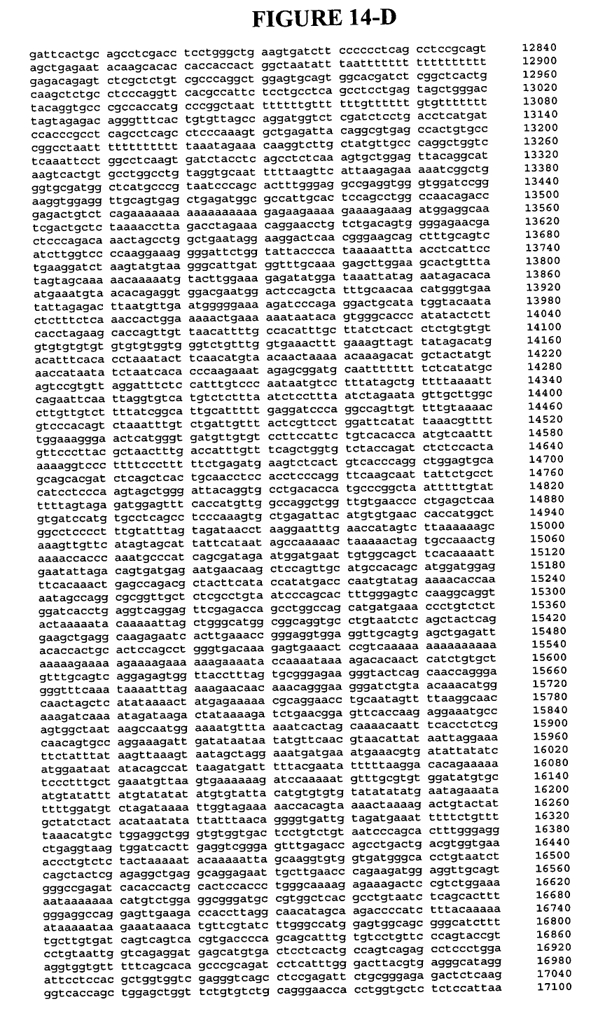

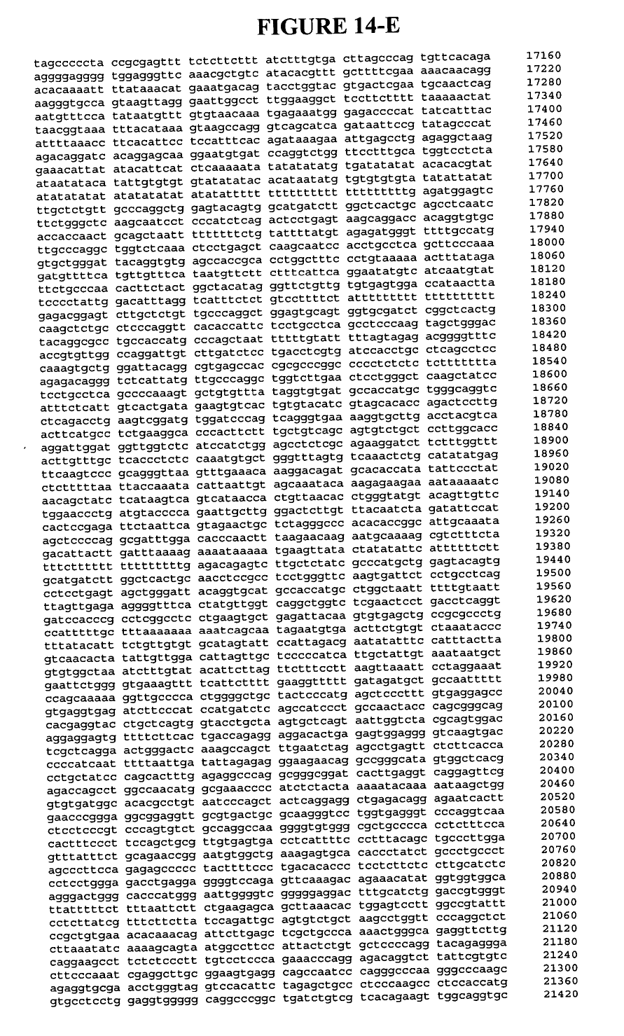

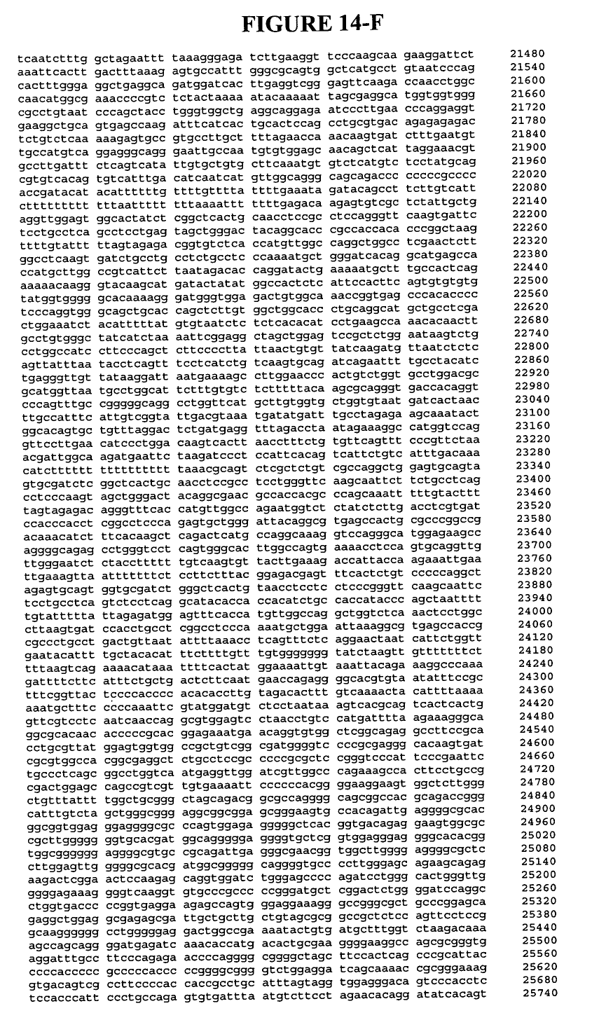

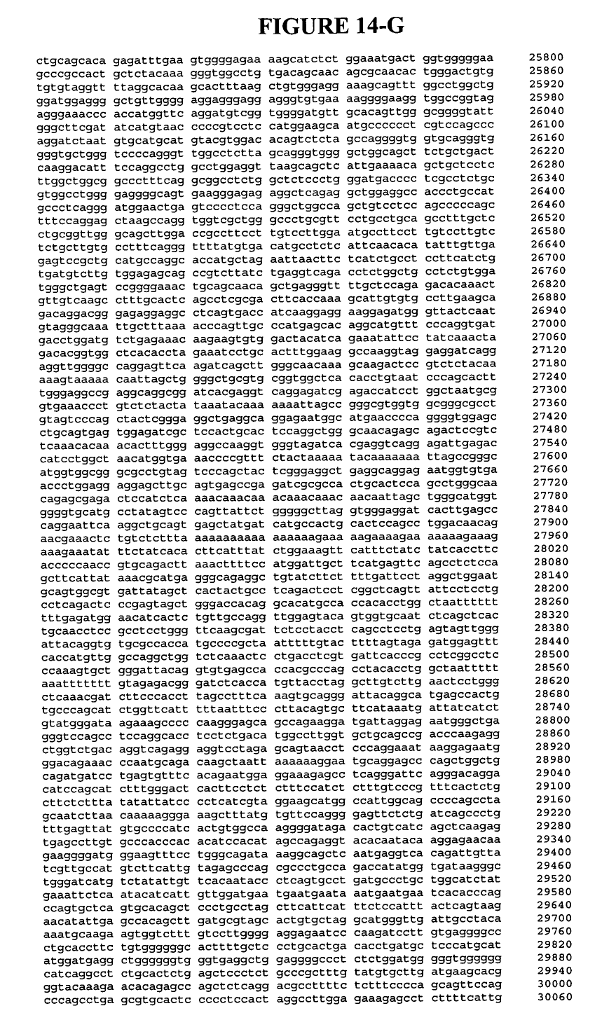

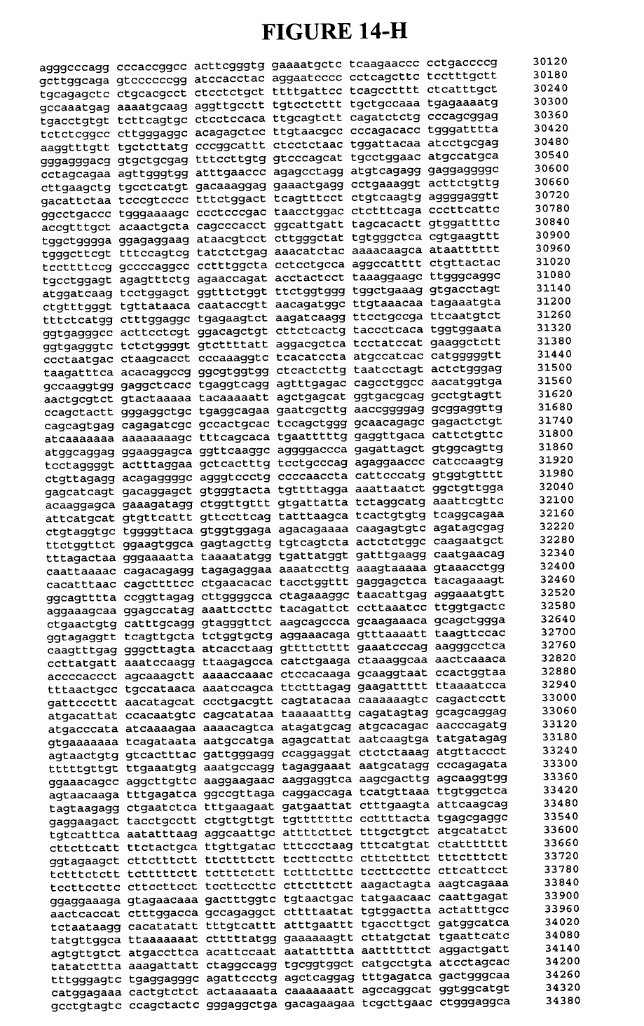

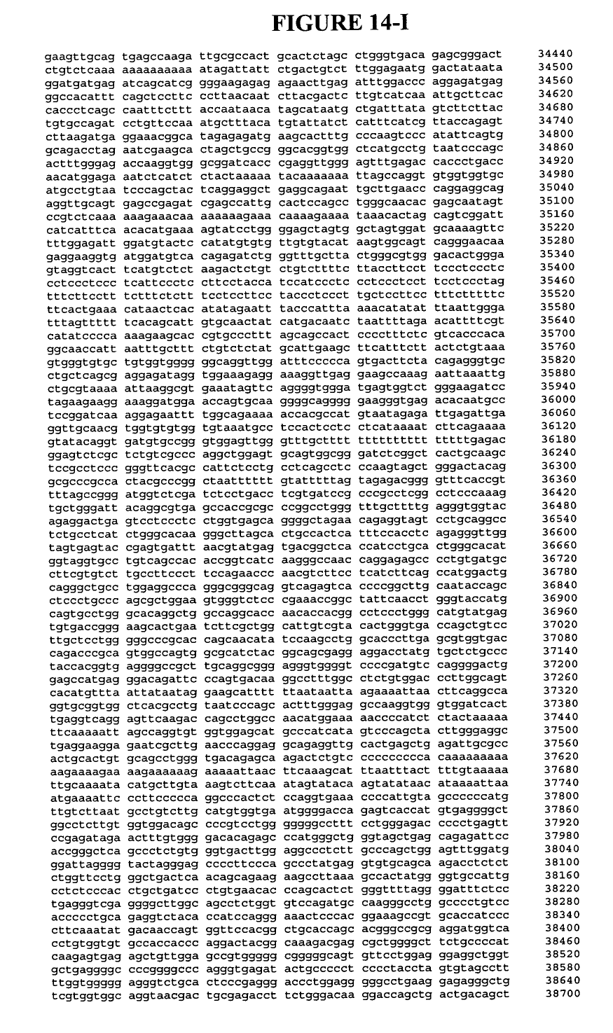

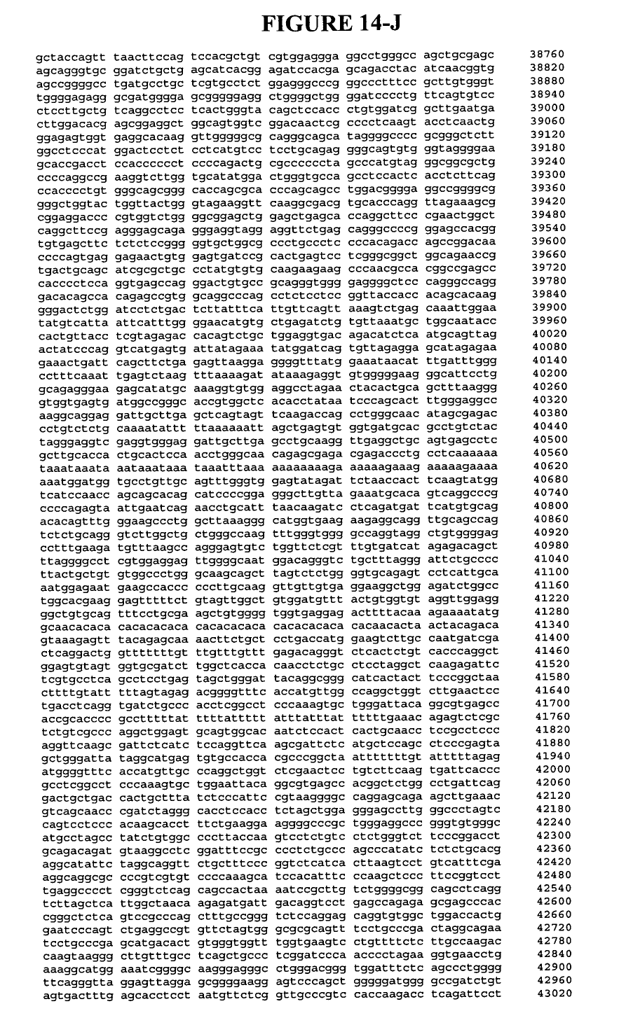

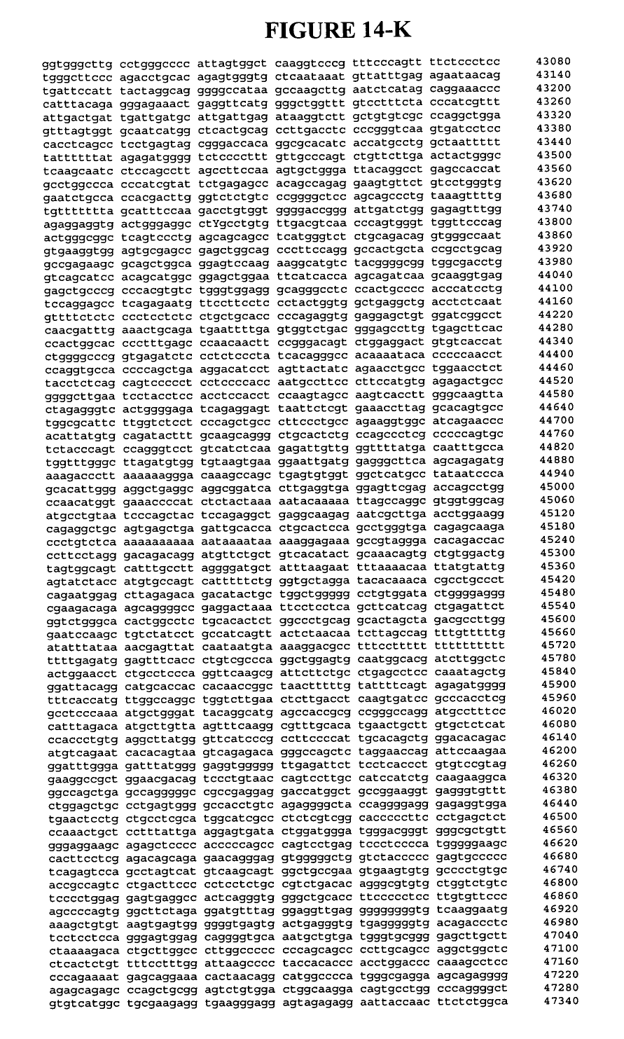

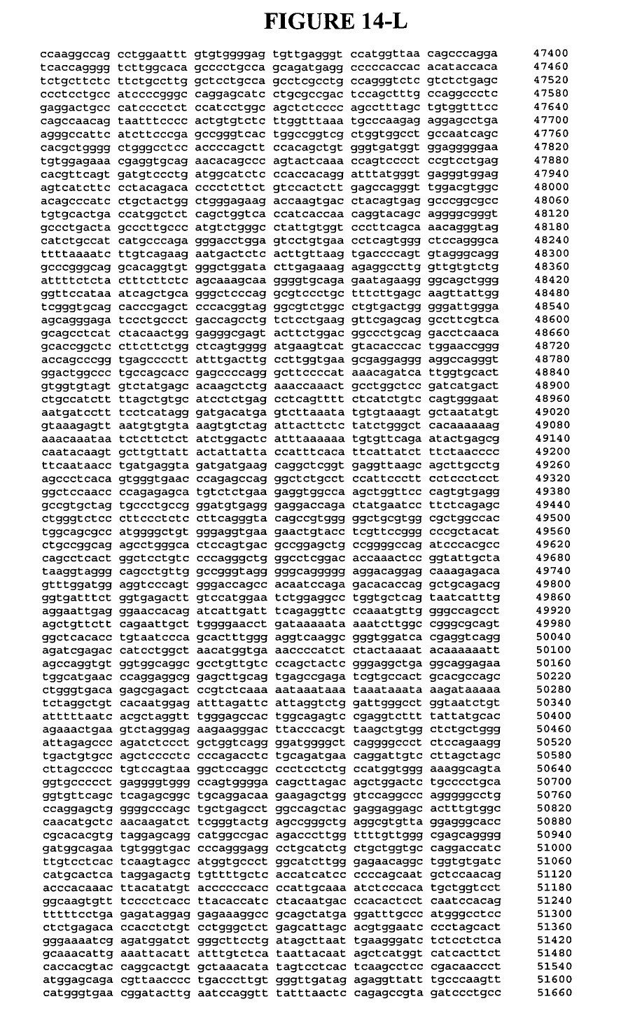

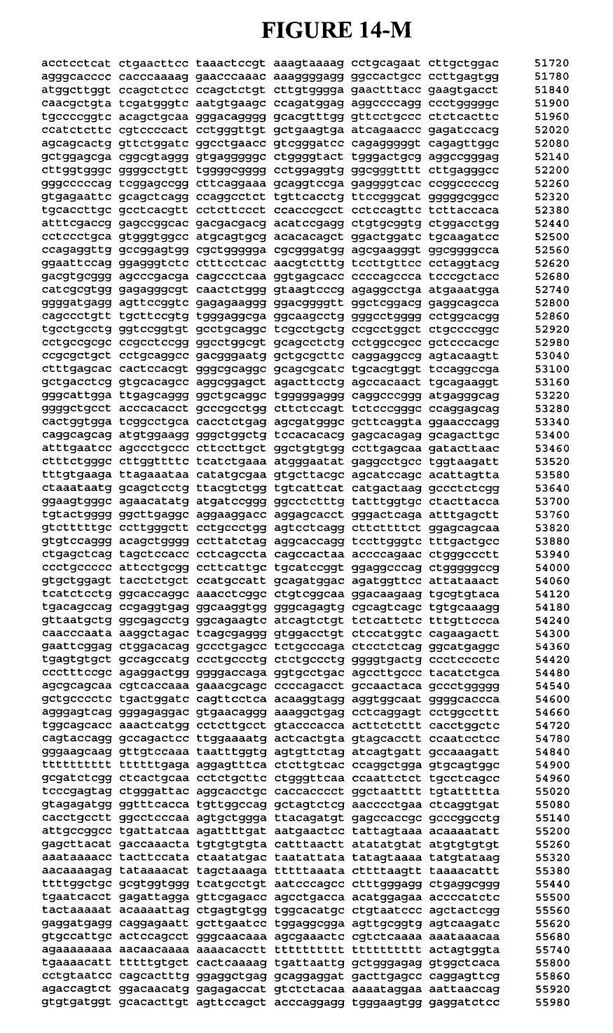

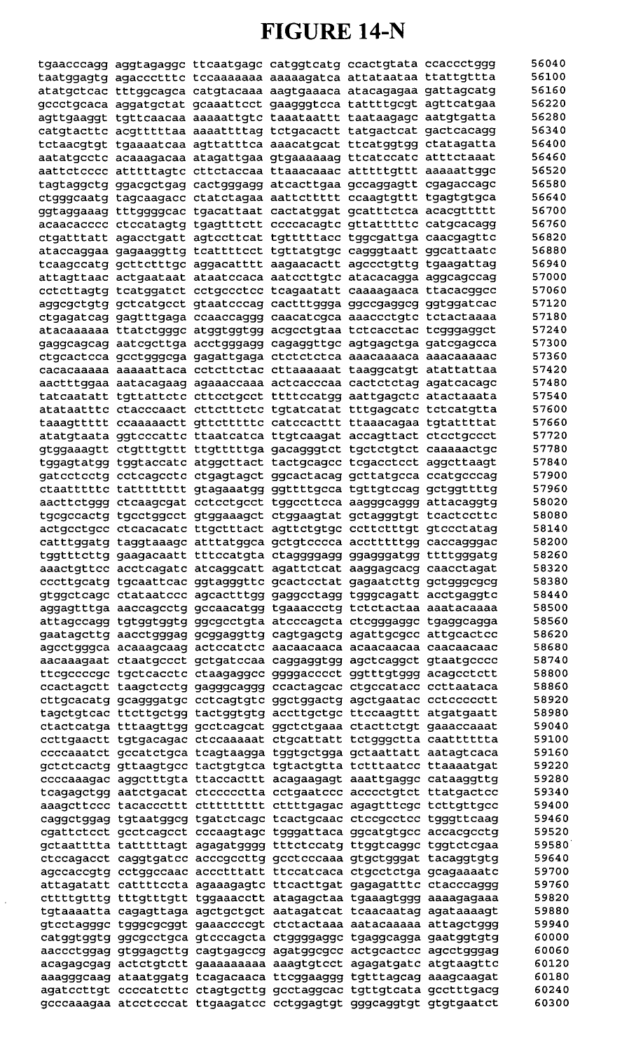

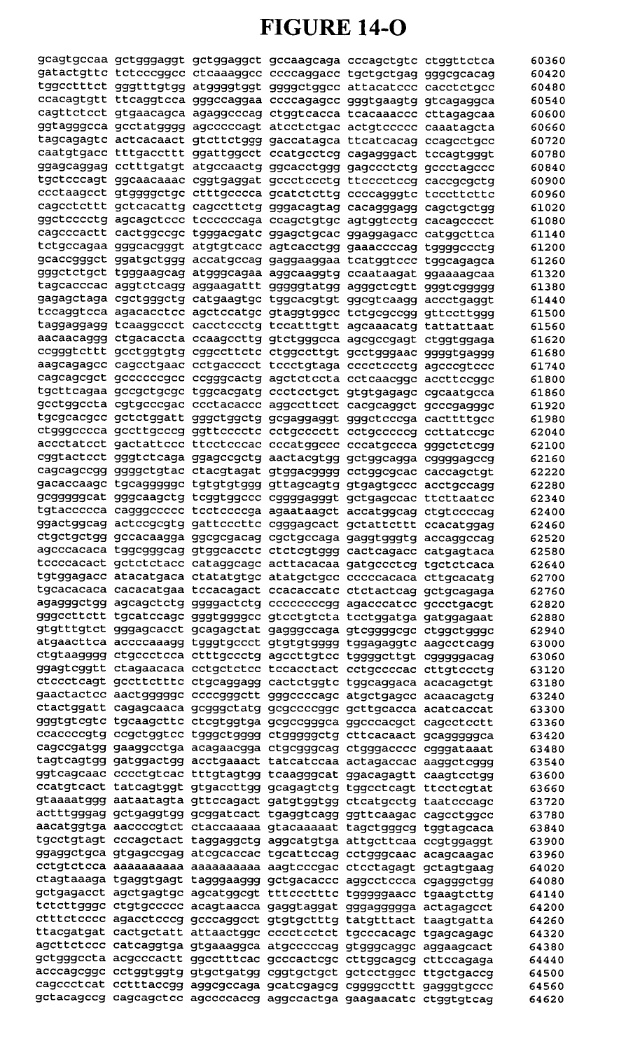

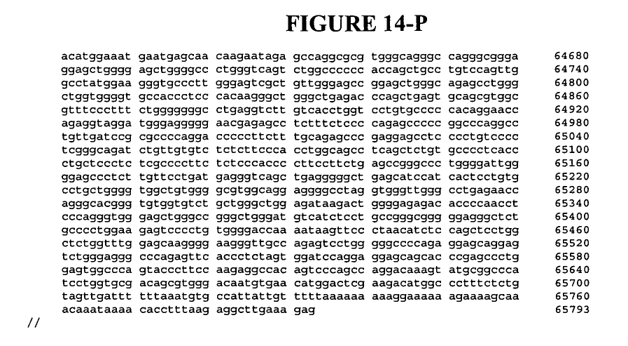

A genetic analysis described hereafter linked melanoma with polymorphic variants of NRP1, NID2 and ENDO180 from human subjects. Nucleotide sequences representative of NRP1 , NID2 and ENDO180 nucleic acids are set forth in FIGS. 1A-1W (SEQ ID NO: 1), FIGS. 13A-13O (SEQ ID NO: 2) and FIGS. 14A-14P (SEQ ID NO: 3), respectively, and are incorporated herein by reference from published database entries (see Examples section and http address at www.ncbi.nlm.nih.gov/LocusLink/). The following is a description of NRP1, NID2 and END180 molecules.

NRP1

NRP1 is known as neuropilin 1, NRP, VEGF165R (Vascular Endothelial Growth Factor-165 Receptor). NRP1 protein is 923 amino acids in length and the corresponding genomic nucleotide sequence has been mapped to chromosomal position 10p12.

Neuropilin is a type I transmembrane protein and is an epitope recognized by a monoclonal antibody that labels specific subsets of axons in the developing Xenopus nervous system. Neuropilin comprises in its extracellular domain several distinctive motifs; its cytoplasmic domain is short (40 amino acids) and is highly conserved among Xenopus, mouse, and chick. An aberrant retinal pathway and visual centers in Xenopus tadpoles share a common cell surface molecule, A5 antigen (Dev. Biol. 135: 231-240, 1989). The gene encoding human neuropilin was cloned and the structure of the protein product was characterized (He & Tessier-Lavigne, Neuropilin is a receptor for the axonal chemorepellent semaphorin III. Cell 90: 739-751, 1997).

A VEGF receptor (NCBI MIM 192240) was discovered which reportedly binds VEGF165 but not VEGF121. This isoform-specific VEGF receptor (VEGF165R) is identical to human neuropilin-1, which is also a receptor for the collapsin/semaphorin family that mediates neuronal cell guidance (Soker et al., Neuropilin-1 is expressed by endothelial and tumor cells as an isoform-specific receptor for vascular endothelial growth factor. Cell 92: 735-745, 1998). It also was shown that when coexpressed in cells with KDR (VEGFR2; NCBI MIM 191306), neuropilin-1 enhances the binding of VEGF165 to KDR and VEGF165-mediated chemotaxis. Conversely, inhibition of VEGF165 binding to neuropilin-1 inhibits its binding to KDR and its mitogenic activity for endothelial cells. It was proposed that neuropilin-1 is a novel VEGF receptor that modulates VEGF binding to KDR and subsequent bioactivity and therefore may regulate VEGF-induced angiogenesis.

It was concluded that NRP1-mediated interactions are a necessary element in the initiation of the primary immune response and offer another example, like that of agrin (NCBI MIM 103320), of a molecule shared by neurologic and immunologic synapses (Tordjman et al., A neuronal receptor, neuropilin-1, is essential for the initiation of the primary immune response. Nature Immun. 3: 477-482, 2002). It was shown that VEGF selectively upregulates NRP1 but not NRP2 via the VEGF receptor 2-dependent pathway. In a murine model of VEGF-dependent angioproliferative retinopathy, intense NRP1 mRNA expression was observed in the newly formed vessels. Furthermore, selective NRP1 inhibition in this model suppressed neovascular formation substantially. These results suggested that VEGF can not only activate endothelial cells directly but also contribute to robust angiogenesis in vivo by a mechanism that involves upregulation of its cognate receptor expression (Oh et al., Selective induction of neuropilin-1 by vascular endothelial growth factor (VEGF): a mechanism contributing to VEGF-induced angiogenesis. Proc. Nat. Acad. Sci. 99: 383-388, 2002).

Transgenic mice died in utero at embryonic day 8.5 when both NRP1 and NRP2, which they called Np1 and Np2, respectively, were knocked out. The yolk sacs of these mice were avascular. Mice deficient for NRP2 but heterozygous for NRP1 or deficient for NRP1 but heterozygous for NRP2 were also embryonic lethal and survived to embryonic days 10 to 10.5. Other details of the abnormal vascular phenotype resembled those of VEGF and Vefgr2 knockouts. The results suggested that neuropilins are early genes in embryonic vessel development and that both NRP1 and NRP2 are required (Takashima et al., Targeting of both mouse neuropilin-1 and neuropilin-2 genes severely impairs developmental yolk sac and embryonic angiogenesis. Proc. Nat. Acad. Sci. 99: 3657-3662, 2002).

It was shown in zebrafish the NRP1 protein was a functional receptor for human VEGF 165. Whole-mount in situ hybridization showed that transcripts of the zebrafish NRP1 gene during embryonic and early larval development were detected mainly in neuronal and vascular tissues. A knockdown of the gene in embryos resulted in vascular defects. Embryos treated with VEGFR2 kinase inhibitor had a similar vessel defect, suggesting that knockdown of zebrafish NRP1 reduces VEGF activity. To determine whether NRP1 and VEGF activities are interdependent in vivo, zebrafish NRP1 and VEGF morpholinos were coinjected into embryos at concentrations that individually did not significantly inhibit blood vessel development. The result was a potent inhibition of blood cell circulation via both intersegmental and axial vessels, demonstrating that VEGF and NRP1 act synergistically to promote a functional circulatory system. These results provided the first physiologic demonstration that NRP1 regulated angiogenesis through a VEGF-dependent pathway (Lee et al., Neuropilin-1 is required for vascular development and is a mediator of VEGF-dependent angiogenesis in zebrafish. Proc. Nat. Acad. Sci. 99: 10470-10475, 2002).

NID2

The protein Nidogen 2 is also known as NID2 or osteonidogen. NID2 protein is 1376 amino acids in length and has a predicted molecular weight of 151,153 daltons (but see below). NID2 is a member of the nidogen superfamily and has EGF homology; LDL receptor YWTD-containing repeat homology; and thyroglobulin type I repeat homology. The NID2 genomic sequence has been mapped to chromosomal position 14q21-q22.

Basement membranes, which are composed of type IV collagens (see NCBI MIM 120130), laminins (see LAMC1; NCBI MIM 150290), perlecan (HSPG2; NCBI MIM 142461), and nidogen (see NID1; NCBI MIM 131390), are thin pericellular protein matrices that control a large number of cellular activities, including adhesion, migration, differentiation, gene expression, and apoptosis. By sequencing several overlapping cDNA clones in both directions, a cDNA encoding NID2 was obtained Kohfeldt et al., Nidogen-2: a new basement membrane protein with diverse binding properties. J. Molec. Biol. 282: 99-109, 1998). Sequence analysis predicted that the 1,375-amino acid NID2 protein, which is 46% identical to NID1, contains a 30-residue signal peptide, 49 primarily central cys residues, 5 potential N-linked glycosylation sites, 2 tyr residues in a potential O-sulfation sequence, and a YGD rather than an RGD cell adhesion sequence. Electron microscopy confirmed the presence of 3 deduced globular domains connected by a link and a rod-like region. Additional predicted structures similar to those found in NID1 include 5 epidermal growth factor (EGF; NCBI MIM 131530)-like modules, 2 of which have potential calcium-binding sequences; 2 thyroglobulin (NCBI MIM 188450) type I modules; and 5 low density lipoprotein (LDL) receptor (NCBI MIM 606945) modules. SDS-PAGE analysis showed that NID2 is expressed as a 200-kD protein, larger than the calculated mass of 148 kD, presumably due to oligosaccharide substitution as indicated by hexosamine analysis. Northern blot analysis revealed ubiquitous expression of a 5.5-kb NID2 transcript that was strongest in heart and placenta, moderate in pancreas, kidney, and skeletal muscle, and weakest in brain. Immunoblot analysis detected expression of NID2 in muscle, heart, placenta, kidney, skin, and testis, with weaker expression in liver and brain. Immunofluorescence analysis of mouse tissues showed staining of basement membranes usually in close colocalization with NID1.

It was determined that the mouse NID2 gene contains 21 exons (Schymeinsky et al., Gene structure and functional analysis of the mouse nidogen-2 gene: nidogen-2 is not essential for basement membrane formation in mice. Molec. Cell. Biol. 22: 6820-6830, 2002). The promoter region contains several putative SP1 (NCBI MIM 189906) and CAAT recognition sites, but lacks a TATA box. Mice carrying a phenotypic null mutation in the NID2 gene have been developed. NID2-deficient mice showed no overt abnormalities, and their basement membranes appeared normal by ultrastructural analysis and immunostaining. NID2 deficiency did not lead to hemorrhages, and NID2 did not appear essential for basement membrane formation or maintenance.

ENDO180

The ENDO180 nucleotide sequence encodes a protein also known as UPARAP (urokinase plasminogen activator receptor-associated protein), KIAA0709, MRC2 (mannose receptor, C type 2), endocytic receptor (macrophage mannose receptor family) and is described as a novel mesenchymally expressed member of the macrophage mannose receptor family of endocytic receptors. ENDO180 is 1479 amino acids in length and has a molecular weight of about 180 kDa. ENDO180 has been mapped to chromosomal position 17q24.1.

ENDO180 is a member of the mannose receptor family (East et al., Biochim Biophys Acta. Sep. 19, 2002;1572(2-3):364-86. The mannose receptor family comprises four glycoproteins each of which is a type I transmembrane receptor with an N-terminal cysteine-rich domain, a single fibronectin type II (FNII) domain and eight to ten C-type lectin-like domains (CTLDs). Characteristically, these proteins are able to recycle between the plasma membrane and the endosomal apparatus due to discrete motifs present within their cytoplasmic domains. The family includes mannose receptor (MR), M-type receptor for secretory phospholipases A(2) (PLA(2)R), DEC-205/gp200-MR6 and ENDO180/uPARAP. Despite their overall structural similarity, these four receptors have evolved to use different domains to interact with discrete ligands. In addition, they differ in their ability to mediate endocytic and phagocytic events and in their intracellular destinations. Together, they represent a unique group of multidomain, multifunctional receptors.

Members of this receptor family are unusual in that they contain 8-10 C-type lectin carbohydrate recognition domains in a single polypeptide backbone, however, only the macrophage mannose receptor had been shown to function as a lectin (Sheikh et al, J Cell Sci. March 2000;113 (Pt 6): 1021-32). Sequence analysis of ENDO180 reveals that the second carbohydrate recognition domain has retained key conserved amino acids found in other functional C-type lectins. The protein also displays Ca(2+)-dependent binding to N-acetylglucosamine but not mannose affinity columns. ENDO180 predominantly is expressed in vivo and in vitro on fibroblasts, endothelial cells and macrophages, and the distribution and post-translational processing in these cells is consistent with ENDO180 functioning to internalize glycosylated ligands from the extracellular milieu for release in an endosomal compartment.

The uptake and lysosomal degradation of collagen by fibroblasts constitute a major pathway in the turnover of connective tissue (Engelholm, et al., J Cell Biol. Mar. 31, 2003;160(7):1009-15). It was reported that ENDO180 is a key player in this process. Fibroblasts from mice with a targeted deletion in the ENDO180 gene displayed a near to complete abrogation of collagen endocytosis. These cells had diminished initial adhesion to a range of different collagens, as well as impaired migration on fibrillar collagen.

As used herein, “polymorphic site” refers to a region in a nucleic acid at which two or more alternative nucleotide sequences are observed in a significant number of nucleic acid samples from a population of individuals. A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site is often one nucleotide in length, which is referred to herein as a “single nucleotide polymorphism” or a “SNP.”

Where there are two, three, or four alternative nucleotide sequences at a polymorphic site, each nucleotide sequence is referred to as a “polymorphic variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele. As shown hereafter, certain polymorphic variants of NRP1, NID2 or ENDO180 nucleotide sequences are associated with melanoma.

A genotype or polymorphic variant may be expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

As used herein, “phenotype” refers to a trait which can be compared between individuals, such as presence or absence of a condition, a visually observable difference in appearance between individuals, metabolic variations, physiological variations, variations in the function of biological molecules, and the like. An example of a phenotype is occurrence of melanoma.

Researchers sometimes report a polymorphic variant in a database without determining whether the variant is represented in a significant fraction of a population. Because a subset of these reported polymorphic variants are not represented in a statistically significant portion of the population, some of them are sequencing errors and/or not biologically relevant. Thus, it often is not known whether a reported polymorphic variant is statistically significant or biologically relevant until the presence of the variant is detected in a population of individuals and the frequency of the variant is determined. Methods for detecting a polymorphic variant in a population are described herein, such as in Example 2. A polymorphic variant is statistically significant and often biologically relevant if it is represented in 5% or more of a population, sometimes 10% or more, 15% or more, or 20% or more of a population, and often 25% or more, 30% or more, 35% or more, 40% or more, 45% or more, or 50% or more of a population.

A polymorphic variant may be detected on either or both strands of a double-stranded nucleic acid. Also, a polymorphic variant may be located within an intron or exon of a gene or within a portion of a regulatory region such as a promoter, a 5′ untranslated region (UTR), a 3′ UTR, and in DNA (e.g., genomic DNA (gDNA) and complementary DNA (cDNA)), RNA (e.g., mRNA, tRNA, and rRNA), or a polypeptide. Polymorphic variations may or may not result in detectable differences in gene expression, polypeptide structure, or polypeptide function.

For duplex DNA, a polymorphic variation may be reported from one strand or its complementary strand. For example, a thymine at a particular position in SEQ ID NO: 1, 2 or 3 can be reported as an adenine from the complementary strand. Also, while polymorphic variations at all positions within a haplotype often are reported from the same strand orientation, polymorphic variations at certain positions within a haplotype sometimes are reported from one strand orientation while others are reported from the other. The latter sometimes occurs even though it is understood by the person of ordinary skill in the art that polymorphic variants in a haplotype occur within one strand in a nucleic acid. Where a haplotype is reported from mixed strand orientations, a person of ordinary skill in the art can determine the orientation of each polymorphic variation in the haplotype by analyzing the orientation of each extension oligonucleotide utilized to identify each polymorphic variation.

In the genetic analyses that associated polymorphic variations in NRP1, NID2 or ENDO180 with melanoma, samples from individuals having melanoma and individuals not having cancer were allelotyped and genotyped. The term “allelotype” as used herein refers to a process for determining the allele frequency for a polymorphic variant in pooled DNA samples from cases and controls. By pooling DNA from each group, an allele frequency for each SNP in each group is calculated. These allele frequencies are then compared to one another. Particular SNPs are considered as being associated with a particular disease when allele frequency differences calculated between case and control pools are statistically significant. The term “genotyped” as used herein refers to a process for determining a genotype of one or more individuals, where a “genotype” is a representation of one or more polymorphic variants in a population. It was determined that SNPs associated with an increased risk of melanoma existed in NRP1, NID2 or ENDO180 nucleotide sequences at chromosome positions 33767168, 50495413 and 61089738. Another polymorphic variation associated with an increased risk of melanoma was identified at chromosome position 50497467, which coded for a non-synonymous amino acid change at amino acid position 656 in a NID2 amino acid sequence (a proline or serine existed at this amino acid position with serine being associated with an increased risk of melanoma). Polymorphic variations associated with an increased risk of melanoma also were detected at the following positions in SEQ ID NO: 1: 12008, 32137, 32720, 43721, 44339, 45640, 48768, 74247, 75828, 76381, and 84909. Of these, variations at postitions 12008, 32720, 43721, 44339, 45640, 48768, 74247, 75828, 76381 and 84909 were in particular associated with an increased risk of melanoma in females, and variations at positions 32137 and 45640 were in particular associated with an increased risk of melanoma in males. At these positions, a tymine at chromosome position 33767168, an adenine at chromosome position 50495413, a guanine at chromosome position 61089738, a thymine at chromosome position 50497467, a guanine at position 32137 in SEQ ID NO: 1, a cytosine at position 32720 in SEQ ID NO: 1, a thymine at position 43721 in SEQ ID NO: 1, a guanine at position 44339 in SEQ ID NO: 1, a thymine at position 45640 in SEQ ID NO: 1, a thymine at position 48768 in SEQ ID NO: 1, a guanine at position 74247 in SEQ ID NO: 1, an adenine at position 75828 in SEQ ID NO: 1, a thymine at position 76381 in SEQ ID NO: 1, and a guanine at position 84909 in SEQ ID NO: 1 were in particular associated with an increased risk of melanoma.

Additional Polymorphic Variants Associated with Melanoma

Also provided is a method for identifying polymorphic variants proximal to an incident, founder polymorphic variant associated with melanoma. Thus, featured herein are methods for identifying a polymorphic variation associated with melanoma that is proximal to an incident polymorphic variation associated with melanoma, which comprises identifying a polymorphic variant proximal to the incident polymorphic variant associated with melanoma, where the incident polymorphic variant is in a nucleotide sequence set forth in SEQ ID NO: 1, 2 or 3. The nucleotide sequence often comprises a polynucleotide sequence selected from the group consisting of (a) a polynucleotide sequence set forth in SEQ ID NO: 1, 2 or 3; (b) a polynucleotide sequence that encodes a polypeptide having an amino acid sequence encoded by a nucleotide sequence set forth in SEQ ID NO: 1, 2 or 3; and (c) a polynucleotide sequence that encodes a polypeptide having an amino acid sequence that is 90% or more identical to an amino acid sequence encoded by a nucleotide sequence set forth in SEQ ID NO: 1, 2 or 3 or a polynucleotide sequence 90% or more identical to the polynucleotide sequence set forth in SEQ ID NO: 1, 2 or 3. The presence or absence of an association of the proximal polymorphic variant with NIDDM then is determined using a known association method, such as a method described in the Examples hereafter. In an embodiment, the incident polymorphic variant is described in SEQ ID NO: 1, 2 or 3 or in the Examples below. In another embodiment, the proximal polymorphic variant identified sometimes is a publicly disclosed polymorphic variant, which for example, sometimes is published in a publicly available database. In other embodiments, the polymorphic variant identified is not publicly disclosed and is discovered using a known method, including, but not limited to, sequencing a region surrounding the incident polymorphic variant in a group of nucleic samples. Thus, multiple polymorphic variants proximal to an incident polymorphic variant are associated with melanoma using this method.

The proximal polymorphic variant often is identified in a region surrounding the incident polymorphic variant. In certain embodiments, this surrounding region is about 50 kb flanking the first polymorphic variant (e.g. about 50 kb 5′ of the first polymorphic variant and about 50 kb 3′ of the first polymorphic variant), and the region sometimes is composed of shorter flanking sequences, such as flanking sequences of about 40 kb, about 30 kb, about 25 kb, about 20 kb, about 15 kb, about 10 kb, about 7 kb, about 5 kb, or about 2 kb 5′ and 3′ of the incident polymorphic variant. In other embodiments, the region is composed of longer flanking sequences, such as flanking sequences of about 55 kb, about 60 kb, about 65 kb, about 70 kb, about 75 kb, about 80 kb, about 85 kb, about 90 kb, about 95 kb, or about 100 kb 5′ and 3′ of the incident polymorphic variant.

In certain embodiments, polymorphic variants associated with melanoma are identified iteratively. For example, a first proximal polymorphic variant is associated with melanoma using the methods described above and then another polymorphic variant proximal to the first proximal polymorphic variant is identified (e.g., publicly disclosed or discovered) and the presence or absence of an association of one or more other polymorphic variants proximal to the first proximal polymorphic variant with melanoma is determined.

The methods described herein are useful for identifying or discovering additional polymorphic variants that may be used to further characterize a gene, region or loci associated with a condition, a disease (e.g., melanoma), or a disorder. For example, allelotyping or genotyping data from the additional polymorphic variants may be used to identify a functional mutation or a region of linkage disequilibrium. In certain embodiments, polymorphic variants identified or discovered within a region comprising the first polymorphic variant associated with melanoma are genotyped using the genetic methods and sample selection techniques described herein, and it can be determined whether those polymorphic variants are in linkage disequilibrium with the first polymorphic variant. The size of the region in linkage disequilibrium with the first polymorphic variant also can be assessed using these genotyping methods. Thus, provided herein are methods for determining whether a polymorphic variant is in linkage disequilibrium with a first polymorphic variant associated with melanoma, and such information can be used in prognosis methods described herein.

Isolated Nucleic Acids and Variants Thereof

Featured herein are isolated NRP1, NID2 or ENDO180 nucleic acids, which include the nucleotide sequence of SEQ ID NO: 1, 2 or 3, NRP1, NID2 or ENDO180 nucleic acid variants, and substantially identical nucleic acids and fragments of the foregoing. Nucleotide sequences of NRP1, NID2 or ENDO180 nucleic acids sometimes are referred to herein as “NRP1, NID2 or ENDO180 nucleotide sequences.” A “NRP1, NID2 or ENDO180 nucleic acid variant” refers to one allele that may have different polymorphic variations as compared to another allele in another subject or the same subject. A polymorphic variation in the NRP1, NID2 or ENDO180 nucleic acid variant may be represented on one or both strands in a double-stranded nucleic acid or on one chromosomal complement (heterozygous) or both chromosomal complements (homozygous). A NRP1, NID2 or ENDO180 nucleic acid may comprise one or more polymorphic variations associated with melanoma described herein.

As used herein, the term “nucleic acid” includes DNA molecules (e.g., a complementary DNA (cDNA) and genomic DNA (gDNA)) and RNA molecules (e.g., mRNA, rRNA, siRNA and tRNA) and analogs of DNA or RNA, for example, by use of nucleotide analogs. The nucleic acid molecule can be single-stranded and it is often double-stranded. The term “isolated or purified nucleic acid” refers to nucleic acids that are separated from other nucleic acids present in the natural source of the nucleic acid. For example, with regard to genomic DNA, the term “isolated” includes nucleic acids which are separated from the chromosome with which the genomic DNA is naturally associated. An “isolated” nucleic acid is often free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used herein, the term “NRP1, NID2 or ENDO180 gene” refers to a nucleotide sequence that encodes a NRP1, NID2 or ENDO180 polypeptide.

Also included herein are nucleic acid fragments. These fragments are typically a nucleotide sequence identical to a nucleotide sequence in SEQ ID NO: 1, 2 or 3, a nucleotide sequence substantially identical to a nucleotide sequence in SEQ ID NO: 1, 2 or 3, or a nucleotide sequence that is complementary to the foregoing. The nucleic acid fragment may be identical, substantially identical or homologous to a nucleotide sequence in an exon or an intron in SEQ ID NO: 1, 2 or 3 and may encode a full-length or mature polypeptide, or may encode a domain or part of a domain of a NRP1, NID2 or ENDO180 polypeptide. Sometimes, the fragment will comprises one or more of the polymorphic variations described herein as being associated with melanoma. The nucleic acid fragment is often 50, 100, or 200 or fewer base pairs in length, and is sometimes about 300, 400, 500, 600, 700, 800, 900, 1000, 1100, 1200, 1300, or 1400 base pairs in length. A nucleic acid fragment that is complementary to a nucleotide sequence identical or substantially identical to the nucleotide sequence of SEQ ID NO: 1, 2 or 3 and hybridizes to such a nucleotide sequence under stringent conditions is often referred to as a “probe.” Nucleic acid fragments often include one or more polymorphic sites, or sometimes have an end that is adjacent to a polymorphic site as described hereafter.

An example of a nucleic acid fragment is an oligonucleotide. As used herein, the term “oligonucleotide” refers to a nucleic acid comprising about 8 to about 50 covalently linked nucleotides, often comprising from about 8 to about 35 nucleotides, and more often from about 10 to about 25 nucleotides. The backbone and nucleotides within an oligonucleotide may be the same as those of naturally occurring nucleic acids, or analogs or derivatives of naturally occurring nucleic acids, provided that oligonucleotides having such analogs or derivatives retain the ability to hybridize specifically to a nucleic acid comprising a targeted polymorphism. Oligonucleotides described herein may be used as hybridization probes or as components of prognostic or diagnostic assays, for example, as described herein.

Oligonucleotides are typically synthesized using standard methods and equipment, such as the ABI™3900 High Throughput DNA Synthesizer and the EXPEDITE™ 8909 Nucleic Acid Synthesizer, both of which are available from Applied Biosystems (Foster City, Calif.). Analogs and derivatives are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and related publications. Methods for synthesizing oligonucleotides comprising such analogs or derivatives are disclosed, for example, in the patent publications cited above and in U.S. Pat. Nos. 5,614,622; 5,739,314; 5,955,599; 5,962,674; 6,117,992; in WO 00/75372; and in related publications.

Oligonucleotides may also be linked to a second moiety. The second moiety may be an additional nucleotide sequence such as a tail sequence (e.g., a polyadenosine tail), an adapter sequence (e.g., phage M1 3 universal tail sequence), and others. Alternatively, the second moiety may be a non-nucleotide moiety such as a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the oligonucleotide, provided the oligonucleotide can hybridize to the nucleic acid comprising the polymorphism.

Uses for Nucleic Acids

Nucleic acid coding sequences depicted in SEQ ID NO: 1, 2 or 3, or substantially identical sequences thereof, may be used for diagnostic purposes for detection and control of polypeptide expression. Also, included are oligonucleotide sequences such as antisense nucleic acids (e.g., DNA, RNA or PNA), inhibitory RNA and small-interfering RNA (siRNA), and ribozymes that function to inhibit translation of a polypeptide. Antisense techniques and RNA interference techniques are known in the art and are described herein.

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. For example, hammerhead motif ribozyme molecules may be engineered that specifically and efficiently catalyze endonucleolytic cleavage of RNA sequences encoded by a nucleotide sequence set forth in SEQ ID NO: 1, 2 or 3 or a substantially identical sequence thereof. Specific ribozyme cleavage sites within any potential RNA target are initially identified by scanning the target molecule for ribozyme cleavage sites which include the following sequences, GUA, GUU and GUC. Once identified, short RNA sequences of between fifteen (15) and twenty (20) ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Antisense RNA and DNA molecules, siRNA and ribozymes may be prepared by any method known in the art for the synthesis of RNA molecules. These include techniques for chemically synthesizing oligodeoxyribonucleotides well known in the art such as solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

DNA encoding a polypeptide also may have a number of uses for the diagnosis of diseases, including melanoma, resulting from aberrant expression of a target gene described herein. For example, the nucleic acid sequence may be used in hybridization assays of biopsies or autopsies to diagnose abnormalities of expression or function (e.g, Southern or Northern blot analysis, in situ hybridization assays).

In addition, the expression of a polypeptide during embryonic development may also be determined using nucleic acid encoding the polypeptide. As addressed, infra, production of functionally impaired polypeptide is the cause of various disease states, melanoma. In situ hybridizations using polypeptide as a probe may be employed to predict problems related to melanoma. Further, as indicated, infra, administration of human active polypeptide, recombinantly produced as described herein, may be used to treat disease states related to functionally impaired polypeptide. Alternatively, gene therapy approaches may be employed to remedy deficiencies of functional polypeptide or to replace or compete with dysfunctional polypeptide.

Expression Vectors, Host Cells, and Genetically Engineered Cells

Provided herein are nucleic acid vectors, often expression vectors, which contain a NRP1, NID2 or ENDO180 nucleic acid. As used herein, the term “vector” refers to a nucleic acid molecule capable of transporting another nucleic acid to which it has been linked and can include a plasmid, cosmid, or viral vector. The vector can be capable of autonomous replication or it can integrate into a host DNA. Viral vectors may include replication defective retroviruses, adenoviruses and adeno-associated viruses for example.

A vector can include a NRP1, NID2 or ENDO180 nucleic acid in a form suitable for expression of the nucleic acid in a host cell. The recombinant expression vector typically includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of polypeptide desired, and the like. Expression vectors can be introduced into host cells to produce NRP1, NID2 or ENDO180 polypeptides, including fusion polypeptides, encoded by NRP1, NID2 or ENDO180 nucleic acids.

Recombinant expression vectors can be designed for expression of NRP1, NID2 or ENDO180 polypeptides in prokaryotic or eukaryotic cells. For example, NRP1, NID2 or ENDO180 polypeptides can be expressed in E. coli, insect cells (e.g., using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith & Johnson, Gene 67: 31-40 (1988)), pMAL (New England Biolabs, Beverly, Mass.) and pRIT5 (Pharmacia, Piscataway, N.J.) which fuse glutathione S-transferase (GST), maltose E binding polypeptide, or polypeptide A, respectively, to the target recombinant polypeptide.

Purified fusion polypeptides can be used in screening assays and to generate antibodies specific for NRP1, NID2 or ENDO180 polypeptides. In a therapeutic embodiment, fusion polypeptide expressed in a retroviral expression vector is used to infect bone marrow cells that are subsequently transplanted into irradiated recipients. The pathology of the subject recipient is then examined after sufficient time has passed (e.g., six (6) weeks).

Expressing the polypeptide in host bacteria with an impaired capacity to proteolytically cleave the recombinant polypeptide is often used to maximize recombinant polypeptide expression (Gottesman, S., Gene Expression Technology: Methods in Enzymology, Academic Press, San Diego, Calif. 185: 119-128 (1990)). Another strategy is to alter the nucleotide sequence of the nucleic acid to be inserted into an expression vector so that the individual codons for each amino acid are those preferentially utilized in E. coli (Wada et al., Nucleic Acids Res. 20: 2111-2118 (1992)). Such alteration of nucleotide sequences can be carried out by standard DNA synthesis techniques.

When used in mammalian cells, the expression vector's control functions are often provided by viral regulatory elements. For example, commonly used promoters are derived from polyoma, Adenovirus 2, cytomegalovirus and Simian Virus 40. Recombinant mammalian expression vectors are often capable of directing expression of the nucleic acid in a particular cell type (e.g., tissue-specific regulatory elements are used to express the nucleic acid). Non-limiting examples of suitable tissue-specific promoters include an albumin promoter (liver-specific; Pinkert et al., Genes Dev. 1: 268-277 (1987)), lymphoid-specific promoters (Calame & Eaton, Adv. Immunol. 43: 235-275 (1988)), promoters of T cell receptors (Winoto & Baltimore, EMBO J. 8: 729-733 (1989)) promoters of immunoglobulins (Banerji et al., Cell 33: 729-740 (1983); Queen & Baltimore, Cell 33: 741-748 (1983)), neuron-specific promoters (e.g., the neurofilament promoter; Byrne & Ruddle, Proc. Natl. Acad. Sci. USA 86: 5473-5477 (1989)), pancreas-specific promoters (Edlund et al., Science 230: 912-916 (1985)), and mammary gland-specific promoters (e.g., milk whey promoter; U.S. Pat. No. 4,873,316 and European Application Publication No.264,166). Developmentally-regulated promoters are sometimes utilized, for example, the murine hox promoters (Kessel & Gruss, Science 249: 374-379 (1990)) and the α-fetopolypeptide promoter (Campes & Tilghman, Genes Dev. 3: 537-546 (1989)).

A NRP1, NID2 or ENDO180 nucleic acid may also be cloned into an expression vector in an antisense orientation. Regulatory sequences (e.g., viral promoters and/or enhancers) operatively linked to a NRP1, NID2 or ENDO180 nucleic acid cloned in the antisense orientation can be chosen for directing constitutive, tissue specific or cell type specific expression of antisense RNA in a variety of cell types. Antisense expression vectors can be in the form of a recombinant plasmid, phagemid or attenuated virus. For a discussion of the regulation of gene expression using antisense genes see Weintraub et al., Antisense RNA as a molecular tool for genetic analysis, Reviews—Trends in Genetics, Vol. 1(1) (1986).

Also provided herein are host cells that include a NRP1, NID2 or ENDO180 nucleic acid within a recombinant expression vector or NRP1, NID2 or ENDO180 nucleic acid sequence fragments which allow it to homologously recombine into a specific site of the host cell genome. The terms “host cell” and “recombinant host cell” are used interchangeably herein. Such terms refer not only to the particular subject cell but rather also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a NRP1, NID2 or ENDO180 polypeptide can be expressed in bacterial cells such as E. coli, insect cells, yeast or mammalian cells (such as Chinese hamster ovary cells (CHO) or COS cells). Other suitable host cells are known to those skilled in the art.

Vectors can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, transduction/infection, DEAE-dextran-mediated transfection, lipofection, or electroporation.

A host cell provided herein can be used to produce (i.e., express) a NRP1, NID2 or ENDO180 polypeptide. Accordingly, further provided are methods for producing a NRP1, NID2 or ENDO180 polypeptide using the host cells described herein. In one embodiment, the method includes culturing host cells into which a recombinant expression vector encoding a NRP1, NID2 or ENDO180 polypeptide has been introduced in a suitable medium such that a NRP1, NID2 or ENDO180 polypeptide is produced. In another embodiment, the method further includes isolating a NRP1, NID2 or ENDO180 polypeptide from the medium or the host cell.

Also provided are cells or purified preparations of cells which include a NRP1, NID2 or ENDO180 transgene, or which otherwise misexpress NRP1, NID2 or ENDO180 polypeptide. Cell preparations can consist of human or non-human cells, e.g., rodent cells, e.g., mouse or rat cells, rabbit cells, or pig cells. In embodiments, the cell or cells include a NRP1, NID2 or ENDO180 transgene (e.g., a heterologous form of a NRP1, NID2 or ENDO180 such as a human gene expressed in non-human cells). The NRP1, NID2 or ENDO180 transgene can be misexpressed, e.g., overexpressed or underexpressed. In other embodiments, the cell or cells include a gene which misexpress an endogenous NRP1, NID2 or ENDO180 polypeptide (e.g., expression of a gene is disrupted, also known as a knockout). Such cells can serve as a model for studying disorders which are related to mutated or mis-expressed NRP1, NID2 or ENDO180 alleles or for use in drug screening. Also provided are human cells (e.g., a hematopoietic stem cells) transformed with a NRP1, NID2 or ENDO180 nucleic acid.

Also provided are cells or a purified preparation thereof (e.g., human cells) in which an endogenous NRP1, NID2 or ENDO180 nucleic acid is under the control of a regulatory sequence that does not normally control the expression of the endogenous NRP1, NID2 or ENDO180 gene. The expression characteristics of an endogenous gene within a cell (e.g., a cell line or microorganism) can be modified by inserting a heterologous DNA regulatory element into the genome of the cell such that the inserted regulatory element is operably linked to the endogenous NRP1, NID2 or ENDO180 gene. For example, an endogenous NRP1, NID2 or ENDO180 gene (e.g., a gene which is “transcriptionally silent,” not normally expressed, or expressed only at very low levels) may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

Transgenic Animals

Non-human transgenic animals that express a heterologous NRP1, NID2 or ENDO180 polypeptide (e.g., expressed from a NRP1, NID2 or ENDO180 nucleic acid isolated from another organism) can be generated. Such animals are useful for studying the function and/or activity of a NRP1, NID2 or ENDO180 polypeptide and for identifying and/or evaluating modulators of NRP1, NID2 or ENDO180 nucleic acid and NRP1, NID2 or ENDO180 polypeptide activity. As used herein, a “transgenic animal” is a non-human animal such as a mammal (e.g., a non-human primate such as chimpanzee, baboon, or macaque; an ungulate such as an equine, bovine, or caprine; or a rodent such as a rat, a mouse, or an Israeli sand rat), a bird (e.g., a chicken or a turkey), an amphibian (e.g., a frog, salamander, or newt), or an insect (e.g., Drosophila melanogaster), in which one or more of the cells of the animal includes a NRP1, NID2 or ENDO180 transgene. A transgene is exogenous DNA or a rearrangement (e.g., a deletion of endogenous chromosomal DNA) that is often integrated into or occurs in the genome of cells in a transgenic animal. A transgene can direct expression of an encoded gene product in one or more cell types or tissues of the transgenic animal, and other transgenes can reduce expression (e.g., a knockout). Thus, a transgenic animal can be one in which an endogenous NRP1, NID2 or ENDO180 gene has been altered by homologous recombination between the endogenous gene and an exogenous DNA molecule introduced into a cell of the animal (e.g., an embryonic cell of the animal) prior to development of the animal.

Intronic sequences and polyadenylation signals can also be included in the transgene to increase expression efficiency of the transgene. One or more tissue-specific regulatory sequences can be operably linked to a NRP1, NID2 or ENDO180 transgene to direct expression of a NRP1, NID2 or ENDO180 polypeptide to particular cells. A transgenic founder animal can be identified based upon the presence of a NRP1, NID2 or ENDO180 transgene in its genome and/or expression of NRP1, NID2 or ENDO180 mRNA in tissues or cells of the animals. A transgenic founder animal can then be used to breed additional animals carrying the transgene. Moreover, transgenic animals carrying a transgene encoding a NRP1, NID2 or ENDO180 polypeptide can further be bred to other transgenic animals carrying other transgenes.

NRP1, NID2 or ENDO180 polypeptides can be expressed in transgenic animals or plants by introducing, for example, a nucleic acid encoding the polypeptide into the genome of an animal. In embodiments the nucleic acid is placed under the control of a tissue specific promoter, e.g., a milk or egg specific promoter, and recovered from the milk or eggs produced by the animal. Also included is a population of cells from a transgenic animal.

NRP1, NID2 or ENDO180 Polypeptides

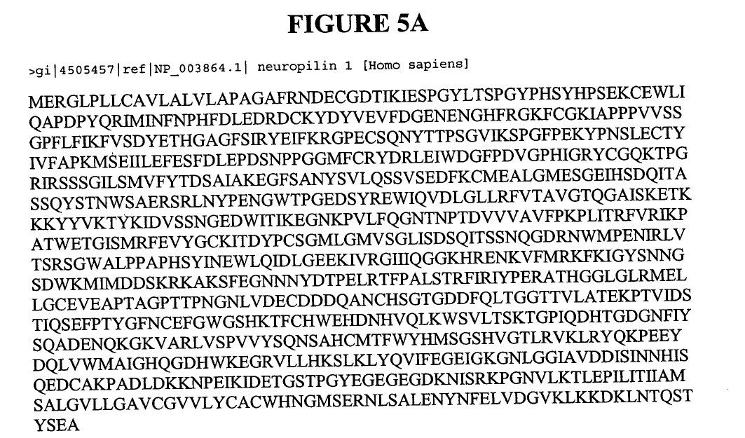

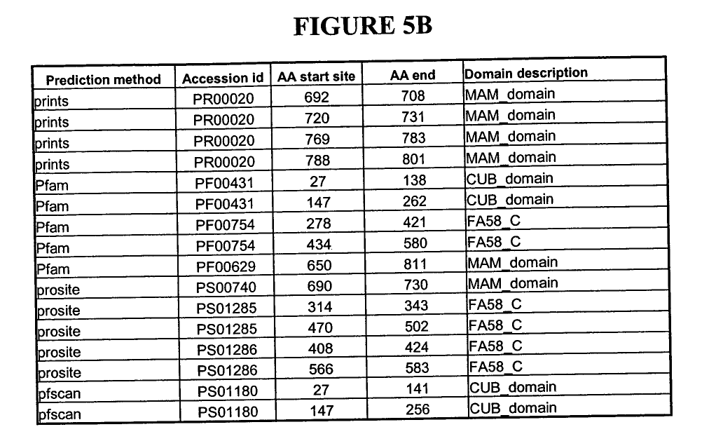

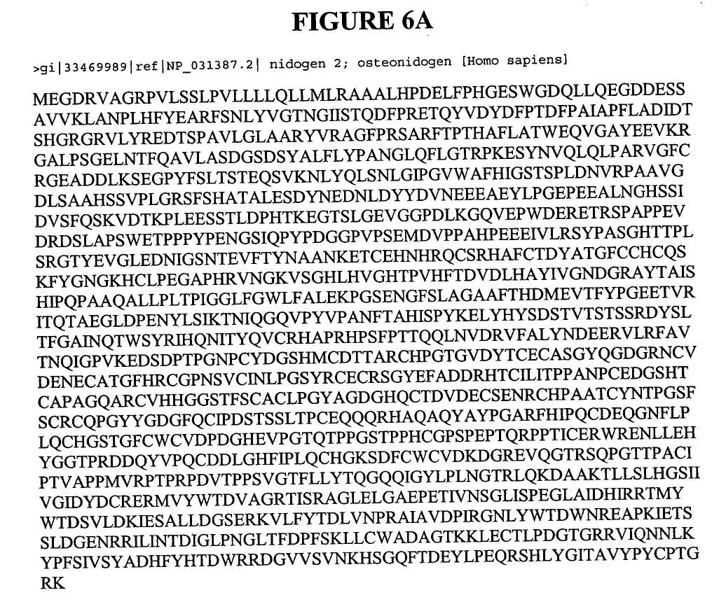

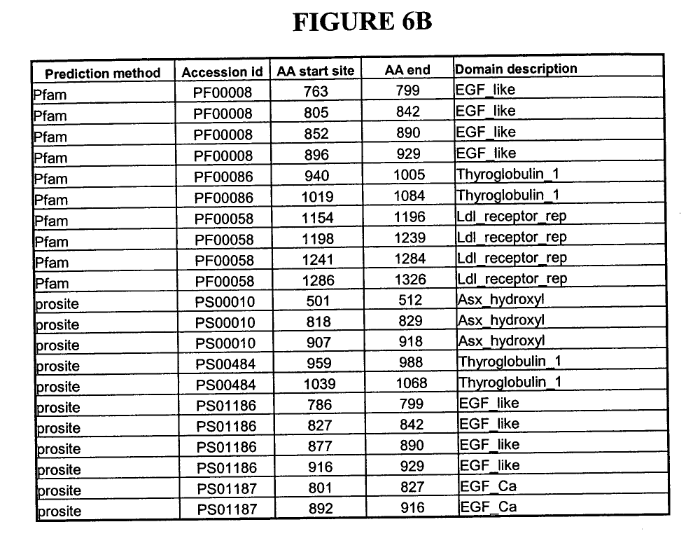

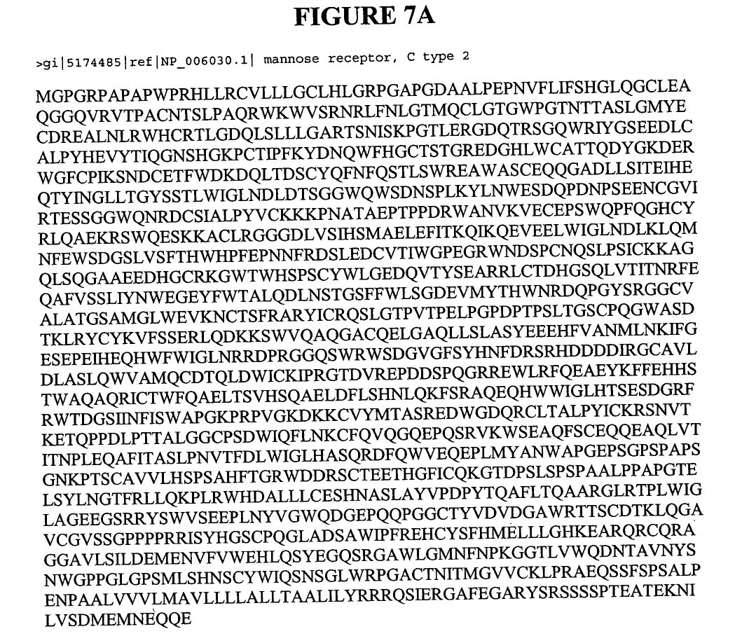

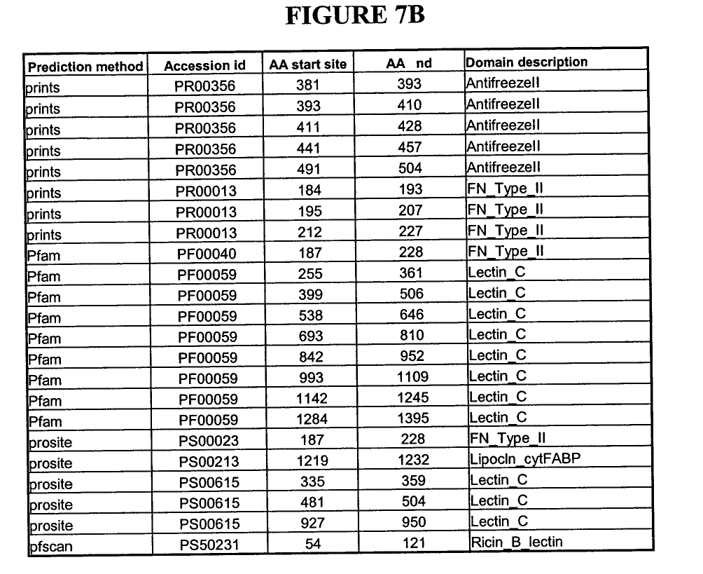

Also featured herein are isolated NRP1, NID2 or ENDO180 polypeptides, including proteins and peptides, that include an amino acid sequence set forth in FIGS. 5A-5B, 6A-6B and 7A-7B, respectively, or a substantially identical sequence thereof or variant thereof. In an embodiment, an NID2 polypeptide includes a serine at amino acid position 656. Isolated NRP1, NID2 or ENDO180 polypeptides featured herein include both the full-length polypeptide and the mature polypeptide (i.e., the polypeptide minus the signal sequence or propeptide domain). A NRP1, NID2 or ENDO180 polypeptide is a polypeptide encoded by a NRP1, NID2 or ENDO180 nucleic acid, where one nucleic acid can encode one or more different polypeptides. An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a NRP1, NID2 or ENDO180 polypeptide or NRP1, NID2 or ENDO180 polypeptide variant having less than about 30%, 20%, 10% and more preferably 5% (by dry weight), of non-NRP1, NID2 or ENDO180 polypeptide (also referred to herein as a “contaminating protein”), or of chemical precursors or non-NRP1, NID2 or ENDO180 chemicals. When the NRP1, NID2 or ENDO180 polypeptide or a biologically active portion thereof is recombinantly produced, it is also preferably substantially free of culture medium, specifically, where culture medium represents less than about 20%, sometimes less than about 10%, and often less than about 5% of the volume of the polypeptide preparation. Isolated or purified NRP1, NID2 or ENDO180 polypeptide preparations are sometimes 0.01 milligrams or more or 0.1 milligrams or more, and often 1.0 milligrams or more and 10 milligrams or more in dry weight.

Further included herein are NRP1, NID2 or ENDO180 polypeptide fragments. The polypeptide fragment may be a domain or part of a domain of a NRP1, NID2 or ENDO180 polypeptide. The polypeptide fragment may have increased, decreased or unexpected biological activity. The polypeptide fragment is often 50 or fewer, 100 or fewer, or 200 or fewer amino acids in length, and is sometimes 300, 400, 500, 600, or 700, or fewer amino acids in length.

Substantially identical polypeptides may depart from the amino acid sequences set forth in FIGS. 5A-5B, 6A-6B and 7A-7B in different manners. For example, conservative amino acid modifications may be introduced at one or more positions in the amino acid sequences of FIGS. 5A-5B, 6A-6B and 7A-7B. A “conservative amino acid substitution” is one in which the amino acid is replaced by another amino acid having a similar structure and/or chemical function. Families of amino acid residues having similar structures and functions are well known. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Also, essential and non-essential amino acids may be replaced. A “non-essential” amino acid is one that can be altered without abolishing or substantially altering the biological function of a NRP1, NID2 or ENDO180 polypeptide, whereas altering an “essential” amino acid abolishes or substantially alters the biological function of a NRP1, NID2 or ENDO180 polypeptide. Amino acids that are conserved among NRP1, NID2 or ENDO180 polypeptides are typically essential amino acids.

Also, NRP1, NID2 or ENDO180 polypeptides and polypeptide variants may exist as chimeric or fusion polypeptides. As used herein, a NRP1, NID2 or ENDO180 “chimeric polypeptide” or “fusion polypeptide” includes a NRP1, NID2 or ENDO180 polypeptide linked to a non-NRP1, NID2 or ENDO180 polypeptide. A “non-NRP1, NID2 or ENDO180 polypeptide” refers to a polypeptide having an amino acid sequence corresponding to a polypeptide which is not substantially identical to the NRP1, NID2 or ENDO180 polypeptide, which includes, for example, a polypeptide that is different from the NRP1, NID2 or ENDO180 polypeptide and derived from the same or a different organism. The NRP1, NID2 or ENDO180 polypeptide in the fusion polypeptide can correspond to an entire or nearly entire NRP1, NID2 or ENDO180 polypeptide or a fragment thereof. The non-NRP1, NID2 or ENDO180 polypeptide can be fused to the N-terminus or C-terminus of the NRP1, NID2 or ENDO180 polypeptide.

Fusion polypeptides can include a moiety having high affinity for a ligand. For example, the fusion polypeptide can be a GST-NRP1, NID2 or ENDO180 fusion polypeptide in which the NRP1, NID2 or ENDO180 sequences are fused to the C-terminus of the GST sequences, or a polyhistidine-NRP1, NID2 or ENDO180 fusion polypeptide in which the NRP1, NID2 or ENDO180 polypeptide is fused at the N- or C-terminus to a string of histidine residues. Such fusion polypeptides can facilitate purification of recombinant NRP1, NID2 or ENDO180. Expression vectors are commercially available that already encode a fusion moiety (e.g., a GST polypeptide), and a NRP1, NID2 or ENDO180 nucleic acid can be cloned into an expression vector such that the fusion moiety is linked in-frame to the NRP1, NID2 or ENDO180 polypeptide. Further, the fusion polypeptide can be a NRP1, NID2 or ENDO180 polypeptide containing a heterologous signal sequence at its N-terminus. In certain host cells (e.g., mammalian host cells), expression, secretion, cellular internalization, and cellular localization of a NRP1, NID2 or ENDO180 polypeptide can be increased through use of a heterologous signal sequence. Fusion polypeptides can also include all or a part of a serum polypeptide (e.g., an IgG constant region or human serum albumin).

NRP1, NID2 or ENDO180 polypeptides or fragments thereof can be incorporated into pharmaceutical compositions and administered to a subject in vivo. Administration of these NRP1, NID2 or ENDO180 polypeptides can be used to affect the bioavailability of a NRP1, NID2 or ENDO180 substrate and may effectively increase NRP1, NID2 or ENDO180 biological activity in a cell. NRP1, NID2 or ENDO180 fusion polypeptides may be useful therapeutically for the treatment of disorders caused by, for example, (i) aberrant modification or mutation of a gene encoding a NRP1, NID2 or ENDO180 polypeptide; (ii) mis-regulation of the NRP1, NID2 or ENDO180 gene; and (iii) aberrant post-translational modification of a NRP1, NID2 or ENDO180 polypeptide. Also, NRP1, NID2 or ENDO180 polypeptides can be used as immunogens to produce anti-NRP1, NID2 or ENDO180 antibodies in a subject, to purify NRP1, NID2 or ENDO180 ligands or binding partners, and in screening assays to identify molecules which inhibit or enhance the interaction of NRP1, NID2 or ENDO180 with a NRP1, NID2 or ENDO180 substrate.

In addition, polypeptides can be chemically synthesized using techniques known in the art (See, e.g., Creighton, 1983 Proteins. New York, N.Y.: W. H. Freeman and Company; and Hunkapiller et al., (1984) Nature July 12-18;310(5973):105-1 1). For example, a relative short fragment can be synthesized by use of a peptide synthesizer. Furthermore, if desired, nonclassical amino acids or chemical amino acid analogs can be introduced as a substitution or addition into the fragment sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

Polypeptides and polypeptide fragments sometimes are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; and the like. Additional post-translational modifications include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptide fragments may also be modified with a detectable label, such as an enzymatic, fluorescent, isotopic or affinity label to allow for detection and isolation of the polypeptide.

Also provided are chemically modified derivatives of polypeptides that can provide additional advantages such as increased solubility, stability and circulating time of the polypeptide, or decreased immunogenicity (see e.g., U.S. Pat. No: 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the molecular weight often utilized is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polymers should be attached to the polypeptide with consideration of effects on functional or antigenic domains of the polypeptide. There are a number of attachment methods available to those skilled in the art (e.g., EP 0 401 384 (coupling PEG to G-CSF) and Malik et al. (1992) Exp Hematol. September;20(8):1028-35 (pegylation of GM-CSF using tresyl chloride)). For example, polyethylene glycol may be covalently bound through amino acid residues via a reactive group, such as a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. For therapeutic purposes, the attachment sometimes is at an amino group, such as attachment at the N-terminus or lysine group.

Proteins can be chemically modified at the N-terminus. Using polyethylene glycol as an illustration of such a composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus may be accomplished by reductive alkylation, which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

Substantially Identical NRP1, NID2 or ENDO180 Nucleic Acids and Polypeptides

NRP1, NID2 or ENDO180 nucleotide sequences and NRP1, NID2 or ENDO180 polypeptide sequences that are substantially identical to the nucleotide sequence of SEQ ID NO: 1, 2 or 3 and the polypeptide sequences of FIGS. 5A-5B, 6A-6B and 7A-7B, respectively, are included herein. The term “substantially identical” as used herein refers to two or more nucleic acids or polypeptides sharing one or more identical nucleotide sequences or polypeptide sequences, respectively. Included are nucleotide sequences or polypeptide sequences that are 55%, 60%, 65%, 70%, 75%, 80%, 85%, 90%, 95% or more (each often within a 1%, 2%, 3% or 4% variability) identical to the NRP1, NID2 or ENDO180 nucleotide sequence in SEQ ID NO: 1, 2 or 3 or the NRP1, NID2 or ENDO180 polypeptide sequences of FIGS. 5A-5B, 6A-6B and 7A-7B. One test for determining whether two nucleic acids are substantially identical is to determine the percent of identical nucleotide sequences or polypeptide sequences shared between the nucleic acids or polypeptides.

Calculations of sequence identity are often performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70%, 80%, 90%, 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, I% SDS at 65° C.

An example of a substantially identical nucleotide sequence to SEQ ID NO: 1, 2 or 3 is one that has a different nucleotide sequence and still encodes a polypeptide sequence set forth in FIGS. 5A-5B, 6A-6B and 7A-7B. Another example is a nucleotide sequence that encodes a polypeptide having a polypeptide sequence that is more than 70% identical to, sometimes more than 75%, 80%, or 85% identical to, and often more than 90% and 95% or more identical to the polypeptide sequences set forth in FIGS. 5A-5B, 6A-6B and 7A-7B.

NRP1, NID2 or ENDO180 nucleotide sequences and polypeptide sequences can be used as “query sequences” to perform a search against public databases to identify other family members or related sequences, for example. Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to NRP1, NID2 or ENDO180 nucleic acid molecules. BLAST polypeptide searches can be performed with the XBLAST program, score=50, wordlength=3 to obtain amino acid sequences homologous to NRP1, NID2 or ENDO180 polypeptides. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NBLAST) can be used (see the http address www.ncbi.nlm.nih.gov).

A nucleic acid that is substantially identical to the nucleotide sequence of SEQ ID NO: 1, 2 or 3 may include polymorphic sites at positions equivalent to those described herein (e.g., position 146311 in SEQ ID NO: 1, 2 or 3) when the sequences are aligned. For example, using the alignment procedures described herein, SNPs in a sequence substantially identical to the sequence of SEQ ID NO: 1, 2 or 3 can be identified at nucleotide positions that match (i.e., align) with nucleotides at SNP positions in SEQ ID NO: 1, 2 or 3. Also, where a polymorphic variation is an insertion or deletion, insertion or deletion of a nucleotide sequence from a reference sequence can change the relative positions of other polymorphic sites in the nucleotide sequence.

Substantially identical NRP1, NID2 or ENDO180 nucleotide and polypeptide sequences include those that are naturally occurring, such as allelic variants (same locus), splice variants, homologs (different locus), and orthologs (different organism) or can be non-naturally occurring. Non-naturally occurring variants can be generated by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Orthologs, homologs, allelic variants, and splice variants can be identified using methods known in the art. These variants normally comprise a nucleotide sequence encoding a polypeptide that is 50%, about 55% or more, often about 70-75% or more, more often about 80-85% or more, and typically about 90-95% or more identical to the amino acid sequences shown in FIGS. 5A-5B, 6A-6B and 7A-7B or a fragment thereof. Such nucleic acid molecules can readily be identified as being able to hybridize under stringent conditions to the nucleotide sequence shown in SEQ ID NO: 1, 2 or 3 or a fragment of this sequence. Nucleic acid molecules corresponding to orthologs, homologs, and allelic variants of the NRP1, NID2 or ENDO180 nucleotide sequence can further be identified by mapping the sequence to the same chromosome or locus as the NRP1, NID2 or ENDO180 nucleotide sequence or variant.

Also, substantially identical NRP1, NID2 or ENDO180 nucleotide sequences may include codons that are altered with respect to the naturally occurring sequence for enhancing expression of a NRP1, NID2 or ENDO180 polypeptide or polypeptide variant in a particular expression system. For example, the nucleic acid can be one in which one or more codons are altered, and often 10% or more or 20% or more of the codons are altered for optimized expression in bacteria (e.g., E. coli.), yeast (e.g., S. cervesiae), human (e.g., 293 cells), insect, or rodent (e.g., hamster) cells.

Methods for Identifying Subjects at Risk of Melanoma and Risk of Melanoma

Methods for determining whether a subject is at risk of melanoma are provided herein. These methods include detecting the presence or absence of one or more polymorphic variations associated with melanoma in a NRP1, NID2 or ENDO180 nucleotide sequence, or substantially identical sequence thereof, in a sample from a subject, where the presence of such a polymorphic variation is indicative of the subject being at risk of melanoma. These genetic tests are useful for prognosing and/or diagnosing melanoma and often are useful for determining whether an individual is at an increased, intermediate or decreased risk of developing or having melanoma.

Thus, featured herein is a method for identifying a subject at risk of melanoma, which comprises detecting in a nucleic acid sample from the subject the presence or absence of a polymorphic variation associated with melanoma at a polymorphic site in a NRP1, NID2 or ENDO180 nucleotide sequence. The nucleotide sequence often is selected from the group consisting of: (a) a nucleotide sequence of SEQ ID NO: 1, 2 or 3; (b) a nucleotide sequence which encodes a polypeptide consisting of the amino acid sequence encoded by the nucleotide sequence of SEQ ID NO: 1, 2 or 3; (c) a nucleotide sequence which encodes a polypeptide that is 90% or more identical to the amino acid sequence encoded by the nucleotide sequence of SEQ ID NO: 1, 2 or 3; and (d) a fragment of a nucleotide sequence of (a), (b), or (c), where the fragment often comprises a polymorphic site; whereby the presence of the polymorphic variation is indicative of the subject being at risk of melanoma. A polymorphic variation assayed in the genetic test often is located in an intron, sometimes in a region surrounding the NRP1, NID2 or ENDO180 open reading frame (e.g., within 50 kilobases (kb), 40 kb, 30 kb, 20 kb, 15, kb, 10 kb, 5 kb, 4 kb, 3 kb, 2 kb, or 1 kb of the open reading frame initiation site or termination site), and sometimes in an exon. Sometimes the polymorphic variation is not located in an exon (e.g., it sometimes is located in an intron or region upstream or downstream of a terminal intron or exon).

Results from such genetic tests may be combined with other test results to diagnose melanoma. For example, genetic test results may be gathered, a patient sample may be ordered based on a determined predisposition to melanoma (e.g., a skin biopsy), the patient sample is analyzed, and the results of the analysis may be utilized to diagnose melanoma. Also, melanoma diagnostic tests are generated by stratifying populations into subpopulations having different progressions of melanoma and detecting polymorphic variations associated with different progressions of the melanoma, as described in further detail hereafter. In another embodiment, genetic test results are gathered, a patient's risk factors for developing melanoma are analyzed (e.g., exposure to sun and skin pigmentation), and a patient sample may be ordered based on a determined risk of melanoma.

Risk of melanoma sometimes is expressed as a probability, such as an odds ratio, percentage, or risk factor. The risk assessment is based upon the presence or absence of one or more polymorphic variants described herein, and also may be based in part upon phenotypic traits of the individual being tested. Methods for calculating risks based upon patient data are well known (see, e.g., Agresti, Categorical Data Analysis, 2nd Ed. 2002. Wiley). Allelotyping and genotyping analyses may be carried out in populations other than those exemplified herein to enhance the predictive power of the prognostic method. These further analyses are executed in view of the exemplified procedures described herein, and may be based upon the same polymorphic variations or additional polymorphic variations.

The nucleic acid sample typically is isolated from a biological sample obtained from a subject. For example, nucleic acid can be isolated from blood, saliva, sputum, urine, cell scrapings, and biopsy tissue. The nucleic acid sample can be isolated from a biological sample using standard techniques, such as the technique described in Example 2. As used herein, the term “subject” refers primarily to humans but also refers to other mammals such as dogs, cats, and ungulates (e.g., cattle, sheep, and swine). Subjects also include avians (e.g., chickens and turkeys), reptiles, and fish (e.g., salmon), as embodiments described herein can be adapted to nucleic acid samples isolated from any of these organisms. The nucleic acid sample may be isolated from the subject and then directly utilized in a method for determining the presence of a polymorphic variant, or alternatively, the sample may be isolated and then stored (e.g., frozen) for a period of time before being subjected to analysis.

The presence or absence of a polymorphic variant is determined using one or both chromosomal complements represented in the nucleic acid sample. Determining the presence or absence of a polymorphic variant in both chromosomal complements represented in a nucleic acid sample from a subject having a copy of each chromosome is useful for determining the zygosity of an individual for the polymorphic variant (i.e., whether the individual is homozygous or heterozygous for the polymorphic variant). Any oligonucleotide-based diagnostic may be utilized to determine whether a sample includes the presence or absence of a polymorphic variant in a sample. For example, primer extension methods, ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), microarray sequence determination methods, restriction fragment length polymorphism (RFLP), single strand conformation polymorphism detection (SSCP) (e.g., U.S. Pat. Nos. 5,891,625 and 6,013,499), PCR-based assays (e.g., TAQMAN® PCR System (Applied Biosystems)), and nucleotide sequencing methods may be used.

Oligonucleotide extension methods typically involve providing a pair of oligonucleotide primers in a polymerase chain reaction (PCR) or in other nucleic acid amplification methods for the purpose of amplifying a region from the nucleic acid sample that comprises the polymorphic variation. One oligonucleotide primer is complementary to a region 3′ of the polymorphism and the other is complementary to a region 5′ of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GENEAMP® Systems available from Applied Biosystems. Also, those of ordinary skill in the art will be able to design oligonucleotide primers based upon the nucleotide sequence of SEQ ID NO: 1, 2 or 3 without undue experimentation using knowledge readily available in the art.

Also provided are extension oligonucleotides that hybridize to the amplified fragment adjacent to the polymorphic variation. As used herein, the term “adjacent” refers to the 3′ end of the extension oligonucleotide being sometimes 1 nucleotide from the 5′ end of the polymorphic site, often 2 or 3, and at times 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, often 2 or 3 nucleotides, and the number and/or type of nucleotides that are added to the extension oligonucleotide determine whether the polymorphic variant is present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. Oligonucleotide extension methods using mass spectrometry are described, for example, in U.S. Pat. Nos. 5,547,835; 5,605,798; 5,691,141; 5,849,542; 5,869,242; 5,928,906; 6,043,031; and 6,194,144, and a method often utilized is described herein in Example 2.

A microarray can be utilized for determining whether a polymorphic variant is present or absent in a nucleic acid sample. A microarray sometimes includes an oligonucleotides described herein, and methods for making and using oligonucleotide microarrays suitable for prognostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and the oligonucleotides sometimes are linked to the solid support by covalent or non-covalent interactions. The oligonucleotides sometimes are linked to the solid support directly or by a spacer molecule. A microarray sometimes comprise one or more oligonucleotides complementary to a portion of SEQ ID NO: 1, 2 or 3, or complementary to a variant described herein.

A kit may also be utilized for determining whether a polymorphic variant is present or absent in a nucleic acid sample. A kit often comprises one or more pairs of oligonucleotide primers useful for amplifying a fragment of SEQ ID NO: 1, 2 or 3 or a substantially identical sequence thereof, where the fragment includes a polymorphic site. The kit sometimes comprises a polymerizing agent, for example, a thermostable nucleic acid polymerase such as one disclosed in U.S. Pat. Nos. 4,889,818 or 6,077,664. Also, the kit often comprises an elongation oligonucleotide that hybridizes to a NRP1, NID2 or ENDO180 nucleic acid in a nucleic acid sample adjacent to the polymorphic site. Where the kit includes an elongation oligonucleotide, it also often comprises chain elongating nucleotides, such as dATP, dTTP, dGTP, dCTP, and dITP, including analogs of dATP, dTTP, dGTP, dCTP and dITP, provided that such analogs are substrates for a thermostable nucleic acid polymerase and can be incorporated into a nucleic acid chain elongated from the extension oligonucleotide. Along with chain elongating nucleotides would be one or more chain terminating nucleotides such as ddATP, ddTTP, ddGTP, ddCTP, and the like. In an embodiment, the kit comprises one or more oligonucleotide primer pairs, a polymerizing agent, chain elongating nucleotides, at least one elongation oligonucleotide, and one or more chain terminating nucleotides. Kits optionally include buffers, vials, microtitre plates, and instructions for use. NRP1, NID2 or ENDO180 directed hits may be utilized to prognose or diagnose melanoma for a significant fraction of melanoma occurrences, such as in 50% or more melanoma occurrences, or sometimes 60% or more, 70% or more, 80% or more, 90% or more, or 95% or more melanoma occurrences.

Using a polymorphism detection technology (e.g., a technique described above or below in Example 2), mutations and polymorphisms in or around the NRP1, NID2 or ENDO180 locus may be detected in melanocytic lesions, which include nevi, radial growth phase (RGP) melanomas, vertical growth phase (VGP) melanomas, and melanoma metastases. The mutations can be detected within 50 kilobases (kb), 40 kb, 30 kb, 20 kb, 15, kb, 10 kb, 5 kb, 4 kb, 3 kb, 2 kb, or 1 kb from the NRP1, NID2 or ENDO180 open reading frame initiation or termination site. Therefore, provided herein are methods for genotyping NRP1, NID2 or ENDO180 mutations in melanocytic lesions and metastases (e.g., described in Example 2). Mutations in or around the NRP1, NID2 or ENDO180 loci present in later stage melanomas, such as VGP melanomas and melanoma metastases, are indicative of melanomas particularly likely to continue to progress and/or metastasize (e.g., from RGP to VGP melanoma or melanoma metastases), i.e., aggressive melanomas. Thus, provided herein are methods for identifying subjects at risk of a progressive or aggressive melanoma by determining the presence or absence of one or more NRP1, NID2 or ENDO180 mutations in the DNA sample of a subject that exist in melanocytic lesions and/or metastases. Identifying the presence of one or more of these mutations is useful for identifying subjects in need of aggressive treatments of melanoma, and once identified using such methods, a subject often is given information concerning preventions and treatments of the disease, and sometimes is treated with an aggressive melanoma treatment method (e.g., surgery or administration of drugs), as described in more detail hereafter.

Determining the presence of a polymorphic variant, or a combination of two or more polymorphic variants, in a nucleic acid set forth in SEQ ID NOs: 1, 2 and/or 3 of the sample often is indicative of a predisposition to melanoma. In certain embodiments, nucleic acid variants of other loci, such as the BRAF locus described in U.S. application Ser. No. 10/661,966 filed Sep. 11, 2003 and any loci described in the concurrently filed applications directed to melanoma (e.g., CDK10, PCLO, FPGT and REPS2), are detected in combination with one or more nucleic acid variants in the NRP1, NID2 or ENDO180 loci.

As noted above, a tymine at chromosome position 33767168, an adenine at chromosome position 50495413, a guanine at chromosome position 61089738, a thymine at chromosome position 50497467, a thymine at position 12008 in SEQ ID NO: 1, a guanine at position 32137 in SEQ ID NO: 1, a cytosine at position 32720 in SEQ ID NO: 1, an adenine at position 43721 in SEQ ID NO: 1, a guanine at position 44339 in SEQ ID NO: 1, a thymine at position 45640 in SEQ ID NO: 1, an adenine at position 48768 in SEQ ID NO: 1, a guanine at position 74247 in SEQ ID NO: 1, a thymine at position 75828 in SEQ ID NO: 1, an adenine at position 76381 in SEQ ID NO: 1, and a cytosine at position 84909 in SEQ ID NO: 1 are associated with an increased risk of melanoma. An individual identified as having a predisposition to melanoma may be heterozygous or homozygous with respect to the allele associated with melanoma. A subject homozygous for an allele associated with an increased risk of melanoma (e.g., a thymine at position 45640 in SEQ ID NO: 1) is at a comparatively high risk of melanoma, a subject heterozygous for an allele associated with an increased risk of melanoma is at a comparatively intermediate risk of melanoma, and a subject homozygous for an allele associated with a decreased risk of melanoma (e.g., a cytosine at position 45640 in SEQ ID NO: 1, see Examples section below) is at a comparatively low risk of melanoma. A genotype may be assessed for a complementary strand, such that the complementary nucleotide at a particular position is detected (e.g., if a thymine or cytosine is detected at position 45640 in SEQ ID NO: 1, the complementary strand would yield an adenine or guanine, respectively, where the adenine is associated with increased risk of melanoma).

Also featured are methods for determining risk of melanoma and/or identifying a subject at risk of melanoma by contacting a NRP1, NID2 or ENDO180 polypeptide or protein from a subject with an antibody that specifically binds to an epitope associated with increased risk of melanoma in the polypeptide. In an embodiment, the antibody specifically binds to an epitope that comprises a serine at amino acid position 656 in an NID2 polypeptide.

Applications of Genomic Information to Pharmacogenomics

Pharmacogenomics is a discipline that involves tailoring a treatment for a subject according to the subject's genotype as a particular treatment regimen may exert a differential effect depending upon the subject's genotype. Based upon the outcome of a prognostic test described herein, a clinician or physician may target pertinent information and preventative or therapeutic treatments to a subject who would be benefited by the information or treatment and avoid directing such information and treatments to a subject who would not be benefited (e.g., the treatment has no therapeutic effect and/or the subject experiences adverse side effects).

For example, where a candidate therapeutic exhibits a significant interaction with a major allele and a comparatively weak interaction with a minor allele (e.g., an order of magnitude or greater difference in the interaction), such a therapeutic typically would not be administered to a subject genotyped as being homozygous for the minor allele, and sometimes not administered to a subject genotyped as being heterozygous for the minor allele. In another example, where a candidate therapeutic is not significantly toxic when administered to subjects who are homozygous for a major allele but is comparatively toxic when administered to subjects heterozygous or homozygous for a minor allele, the candidate therapeutic is not typically administered to subjects who are genotyped as being heterozygous or homozygous with respect to the minor allele.

The prognostic methods described herein are applicable to general pharmacogenomic approaches towards addressing melanoma. For example, a nucleic acid sample from an individual may be subjected to a prognostic test described herein. Where one or more polymorphic variations associated with increased risk of melanoma are identified in a subject, one or more melanoma treatments or prophylactic regimens may be prescribed to that subject. Subjects genotyped as having one or more of the alleles described herein that are associated with increased risk of melanoma often are prescribed a prophylactic regimen designed to minimize the occurance of melanoma. An example of a prophylactic regimen often prescribed is directed towards minimizing ultraviolet (UV) light exposure. Such a regimen may include, for example, prescription of a lotion applied to the skin that minimizes UV penetration and/or counseling individuals of other practices for reducing UV exposure, such as by wearing protective clothing and minimizing sun exposure.

In certain embodiments, a treatment regimen is specifically prescribed and/or administered to individuals who will most benefit from it based upon their risk of developing melanoma assessed by the prognostic methods described herein. Thus, provided are methods for identifying a subject predisposed to melanoma and then prescribing a therapeutic or preventative regimen to individuals identified as having a predisposition. Thus, certain embodiments are directed to a method for reducing melanoma in a subject, which comprises: detecting the presence or absence of a polymorphic variant associated with melanoma in a nucleotide sequence set forth in FIG. 1 in a nucleic acid sample from a subject, where the nucleotide sequence comprises a polynucleotide sequence selected from the group consisting of: (a) a nucleotide sequence set forth in SEQ ID NO: 1, 2 or 3; (b) a nucleotide sequence which encodes a polypeptide consisting of an amino acid sequence encoded by a nucleotide sequence of SEQ ID NO: 1, 2 or 3; (c) a nucleotide sequence which encodes a polypeptide that is 90% or more identical to an amino acid sequence encoded by a nucleotide sequence of SEQ ID NO: 1, 2 or 3 or a nucleotide sequence about 90% or more identical to the nucleotide sequence in SEQ ID NO: 1, 2 or 3; and (d) a fragment of a polynucleotide sequence of (a), (b), or (c); and prescribing or administering a treatment regimen to a subject from whom the sample originated where the presence of a polymorphic variation associated with melanoma is detected in the nucleotide sequence. In these methods, predisposition results may be utilized in combination with other test results to diagnose melanoma.

The treatment sometimes is preventative (e.g., is prescribed or administered to reduce the probability that a melanoma associated condition arises or progresses), sometimes is therapeutic, and sometimes delays, alleviates or halts the progression of a melanoma associated condition. Any known preventative or therapeutic treatment for alleviating or preventing the occurrence of a melanoma associated disorder is prescribed and/or administered. For example, the treatment sometimes is or includes a drug that reduces melanoma, including, for example, cisplatin, carmustine (BCNU), vinblastine, vincristine, and bleomycin, and/or a molecule that interacts with a nucleic acid or polypeptide described hereafter. In another example, the melanoma treatment is surgery. Surgery to remove (excise) a melanoma is the standard treatment for this disease. It is necessary to remove not only the tumor but also some normal tissue around it in order to minimize the chance that any cancer will be left in the area. It is common for lymph nodes near the tumor to be removed during surgery because cancer can spread through the lymphatic system. Surgery is generally not effective in controlling melanoma that is known to have spread to other parts of the body. In such cases, doctors may use other methods of treatment, such as chemotherapy, biological therapy, radiation therapy, or a combination of these methods.

As therapeutic approaches for melanoma continue to evolve and improve, the goal of treatments for melanoma related disorders is to intervene even before clinical signs (e.g., identification of irregular nevi based on A—asymmetry, B—border irregularity, C—color variation, D—diameter of >6 mm as described by Friedman R J, et al. in CA Cancer J Clin. 1985 May-June;35(3):130-51) first manifest. Thus, genetic markers associated with susceptibility to melanoma prove useful for early diagnosis, prevention and treatment of melanoma.

As melanoma preventative and treatment information can be specifically targeted to subjects in need thereof (e.g., those at risk of developing melanoma or those that have early signs of melanoma), provided herein is a method for preventing or reducing the risk of developing melanoma in a subject, which comprises: (a) detecting the presence or absence of a polymorphic variation associated with melanoma at a polymorphic site in a nucleotide sequence in a nucleic acid sample from a subject; (b) identifying a subject with a predisposition to melanoma, whereby the presence of the polymorphic variation is indicative of a predisposition to melanoma in the subject; and (c) if such a predisposition is identified, providing the subject with information about methods or products to prevent or reduce melanoma or to delay the onset of melanoma. Also provided is a method of targeting information or advertising to a subpopulation of a human population based on the subpopulation being genetically predisposed to a disease or condition, which comprises: (a) detecting the presence or absence of a polymorphic variation associated with melanoma at a polymorphic site in a nucleotide sequence in a nucleic acid sample from a subject; (b) identifying the subpopulation of subjects in which the polymorphic variation is associated with melanoma; and (c) providing information only to the subpopulation of subjects about a particular product which may be obtained and consumed or applied by the subject to help prevent or delay onset of the disease or condition.

Pharmacogenomics methods also may be used to analyze and predict a response to a melanoma treatment or a drug. For example, if pharmacogenomics analysis indicates a likelihood that an individual will respond positively to a melanoma treatment with a particular drug, the drug may be administered to the individual. Conversely, if the analysis indicates that an individual is likely to respond negatively to treatment with a particular drug, an alternative course of treatment may be prescribed. A negative response may be defined as either the absence of an efficacious response or the presence of toxic side effects. The response to a therapeutic treatment can be predicted in a background study in which subjects in any of the following populations are genotyped: a population that responds favorably to a treatment regimen, a population that does not respond significantly to a treatment regimen, and a population that responds adversely to a treatment regiment (e.g., exhibits one or more side effects). These populations are provided as examples and other populations and subpopulations may be analyzed. Based upon the results of these analyses, a subject is genotyped to predict whether he or she will respond favorably to a treatment regimen, not respond significantly to a treatment regimen, or respond adversely to a treatment regimen.

The prognostic tests described herein also are applicable to clinical drug trials. One or more polymorphic variants indicative of response to an agent for treating melanoma or to side effects to an agent for treating melanoma may be identified using the methods described herein. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

Thus, another embodiment is a method of selecting an individual for inclusion in a clinical trial of a treatment or drug comprising the steps of: (a) obtaining a nucleic acid sample from an individual; (b) determining the identity of a polymorphic variation which is associated with a positive response to the treatment or the drug, or at least one polymorphic variation which is associated with a negative response to the treatment or the drug in the nucleic acid sample, and (c) including the individual in the clinical trial if the nucleic acid sample contains said polymorphic variation associated with a positive response to the treatment or the drug or if the nucleic acid sample lacks said polymorphic variation associated with a negative response to the treatment or the drug. In addition, the methods for selecting an individual for inclusion in a clinical trial of a treatment or drug encompass methods with any further limitation described in this disclosure, or those following, specified alone or in any combination. The polymorphic variation may be in a sequence selected individually or in any combination from the group consisting of (i) a polynucleotide sequence set forth in SEQ ID NO: 1, 2 or 3; (ii) a polynucleotide sequence that is 90% or more identical to an amino acid sequence encoded by a nucleotide sequence of SEQ ID NO: 1, 2 or 3; (iii) a polynucleotide sequence that encodes a polypeptide having an amino acid sequence identical to or 90% or more identical to an amino acid sequence encoded by a nucleotide sequence of SEQ ID NO: 1, 2 or 3; and (iv) a fragment of a polynucleotide sequence of (i), (ii), or (iii) comprising the polymorphic site. The including step (c) optionally comprises administering the drug or the treatment to the individual if the nucleic acid sample contains the polymorphic variation associated with a positive response to the treatment or the drug and the nucleic acid sample lacks said biallelic marker associated with a negative response to the treatment or the drug.

Also provided herein is a method of partnering between a diagnostic/prognostic testing provider and a provider of a consumable product, which comprises: (a) the diagnostic/prognostic testing provider detects the presence or absence of a polymorphic variation associated with melanoma at a polymorphic site in a nucleotide sequence in a nucleic acid sample from a subject; (b) the diagnostic/prognostic testing provider identifies the subpopulation of subjects in which the polymorphic variation is associated with melanoma; (c) the diagnostic/prognostic testing provider forwards information to the subpopulation of subjects about a particular product which may be obtained and consumed or applied by the subject to help prevent or delay onset of the disease or condition; and (d) the provider of a consumable product forwards to the diagnostic test provider a fee every time the diagnostic/prognostic test provider forwards information to the subject as set forth in step (c) above.

Compositions Comprising a NRP1, NID2 or ENDO180 Directed Molecule

Featured herein is a composition comprising a melanoma cell and one or more NRP1, NID2 or ENDO180 directed molecules. NRP1, NID2 or ENDO180 directed molecules include, but are not limited to, a compound that binds to a NRP1, NID2 or ENDO180 nucleic acid or polypeptide; an RNAi or siRNA molecule having a strand complementary to a NRP1, NID2 or ENDO180 DNA sequence; an antisense nucleic acid complementary to an RNA encoded by a NRP1, NID2 or ENDO180 DNA sequence; a ribozyme that hybridizes to a NRP1, NID2 or ENDO180 nucleotide sequence; an NRP1, NID2 or ENDO180 polypeptide, protein or fragment thereof, or a nucleic acid that encodes the foregoing; a nucleic acid aptamer that specifically binds a NRP1, NID2 or ENDO180 polypeptide, protein, nucleic acid or variant thereof; and an antibody or fragment thereof that specifically binds to a NRP1, NID2 or ENDO180 polypeptide, protein, nucleic acid or variant thereof. Compositions comprising an anitbody often include an adjuvant known in the art. The melanoma cell may be in a group of melanoma cells and/or other types of cells cultured in vitro or in a tissue having melanoma cells (e.g., a melanocytic lesion) maintained in vitro or present in an animal in vivo (e.g., a rat, mouse, ape or human). In certain embodiments, a composition comprises a component from a melanoma cell or from a subject having a melanoma cell instead of the melanoma cell or in addition to the melanoma cell, where the component sometimes is a nucleic acid molecule (e.g., genomic DNA), a protein mixture or isolated protein, for example. The aforementioned compositions have utility in diagnostic, prognostic and pharmacogenomic methods described previously and in melanoma therapeutics described hereafter. Certain NRP1, NID2 or ENDO180 directed molecules are described in greater detail below.

Compounds

Compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al., J. Med. Chem.37: 2678-85 (1994)); spatially addressable parallel solid phase or solution phase libraries; synthetic library methods requiring deconvolution; “one-bead one-compound” library methods; and synthetic library methods using affinity chromatography selection. Biological library and peptoid library approaches are typically limited to peptide libraries, while the other approaches are applicable to peptide, non-peptide oligomer or small molecule libraries of compounds (Lam, Anticancer Drug Des. 12: 145, (1997)). Examples of methods for synthesizing molecular libraries are described, for example, in DeWitt et al., Proc. Natl. Acad. Sci. U.S.A. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37: 1233 (1994).

Libraries of compounds may be presented in solution (e.g., Houghten, Biotechniques 13: 412-421 (1992)), or on beads (Lam, Nature 354: 82-84 (1991)), chips (Fodor, Nature 364: 555-556 (1993)), bacteria or spores (Ladner, U.S. Pat. No. 5,223,409), plasmids (Cull et al., Proc. Natl. Acad. Sci. USA 89: 1865-1869 (1992)) or on phage (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991); Ladner supra.).

A compound may alter expression or activity of NRP1, NID2 or ENDO180 polypeptides and may be a small molecule. Small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Antisense Nucleic Acid Molecules, Ribozymes, RNAi, siRNA and Modified NRP1, NID2 or ENDO180 Nucleic Acid Molecules

An “antisense” nucleic acid refers to a nucleotide sequence complementary to a “sense” nucleic acid encoding a polypeptide, e.g., complementary to the coding strand of a double-stranded cDNA molecule or complementary to an mRNA sequence. The antisense nucleic acid can be complementary to an entire NRP1, NID2 or ENDO180 coding strand, or to only a portion thereof (e.g., the coding region of human NRP1, NID2 or ENDO180 in SEQ ID NO: 1, 2 or 3). In another embodiment, the antisense nucleic acid molecule is antisense to a “noncoding region” of the coding strand of a nucleotide sequence encoding NRP1, NID2 or ENDO180 (e.g., 5′ and 3′ untranslated regions).

An antisense nucleic acid can be designed such that it is complementary to the entire coding region of NRP1, NID2 or ENDO180 mRNA, and often the antisense nucleic acid is an oligonucleotide antisense to only a portion of a coding or noncoding region of NRP1, NID2 or ENDO180 mRNA. For example, the antisense oligonucleotide can be complementary to the region surrounding the translation start site of NRP1, NID2 or ENDO180 mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length. The antisense nucleic acids, which include the ribozymes described hereafter, can be designed to target NRP1, NID2 or ENDO180 nucleic acid or NRP1, NID2 or ENDO180 nucleic acid variants. Among the variants, minor alleles and major alleles can be targeted, and those associated with a higher risk of melanoma are often designed, tested, and administered to subjects.

An antisense nucleic acid can be constructed using chemical synthesis and enzymatic ligation reactions using standard procedures. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When utilized as therapeutics, antisense nucleic acids typically are administered to a subject (e.g., by direct injection at a tissue site) or generated in situ such that they hybridize with or bind to cellular mRNA and/or genomic DNA encoding a NRP1, NID2 or ENDO180 polypeptide and thereby inhibit expression of the polypeptide, for example, by inhibiting transcription and/or translation. Alternatively, antisense nucleic acid molecules can be modified to target selected cells and then are administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a pol II or pol III promoter, in the vector construct.

Antisense nucleic acid molecules sometimes are α-anomeric nucleic acid molecules. An α-anomeric nucleic acid molecule forms specific double-stranded hybrids with complementary RNA in which, contrary to the usual β-units, the strands run parallel to each other (Gaultier et al., Nucleic Acids. Res. 15: 6625-6641 (1987)). Antisense nucleic acid molecules can also comprise a 2′-o-methylribonucleotide (Inoue et al., Nucleic Acids Res. 15: 6131-6148 (1987)) or a chimeric RNA-DNA analogue (Inoue et al., FEBS Lett. 215: 327-330 (1987)). Antisense nucleic acids sometimes are composed of DNA or PNA or any other nucleic acid derivatives described previously.

In another embodiment, an antisense nucleic acid is a ribozyme. A ribozyme having specificity for a NRP1, NID2 or ENDO180 encoding nucleic acid can include one or more sequences complementary to the nucleotide sequence of a NRP1, NID2 or ENDO180 DNA sequence disclosed herein (e.g., SEQ ID NO: 1, 2 or 3), and a sequence having a known catalytic region responsible for mRNA cleavage (see e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in a NRP1, NID2 or ENDO180 encoding mRNA (see e.g., Cech et al. U.S. Pat. No. 4,987,071; and Cech et al. U.S. Pat. No.5,116,742). Also, NRP1, NID2 or ENDO180 mRNA can be used to select a catalytic RNA having a specific ribonuclease activity from a pool of RNA molecules (see e.g., Bartel & Szostak, Science 261: 1411-1418 (1993)).

NRP1, NID2 or ENDO180 directed molecules include in certain embodiments nucleic acids that can form triple helix structures with a NRP1, NID2 or ENDO180 nucleotide sequence, especially one that includes a regulatory region that controls NRP1, NID2 or ENDO180 expression. NRP1, NID2 or ENDO180 gene expression can be inhibited by targeting nucleotide sequences complementary to the regulatory region of the NRP1, NID2 or ENDO180 (e.g., NRP1, NID2 or ENDO180 promoter and/or enhancers) to form triple helical structures that prevent transcription of the NRP1, NID2 or ENDO180 gene in target cells (see e.g., Helene, Anticancer Drug Des. 6(6): 569-84 (1991); Helene et al., Ann. N.Y. Acad. Sci. 660: 27-36 (1992); and Maher, Bioassays 14(12): 807-15 (1992). Potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

NRP1, NID2 or ENDO180 directed molecules include RNAi and siRNA nucleic acids. Gene expression may be inhibited by the introduction of double-stranded RNA (dsRNA), which induces potent and specific gene silencing, a phenomenon called RNA interference or RNAi. See, e.g., Fire et al., U.S. Pat. No. 6,506,559; Tuschl et al. PCT International Publication No. WO 01/75164; Kay et al. PCT International Publication No. WO 03/010180A1; or Bosher J M, Labouesse, Nat Cell Biol 2000 February;2(2):E31-6. This process has been improved by decreasing the size of the double-stranded RNA to 20-24 base pairs (to create small-interfering RNAs or siRNAs) that “switched off” genes in mammalian cells without initiating an acute phase response, i.e., a host defense mechanism that often results in cell death (see, e.g., Caplen et al. Proc Natl Acad Sci U S A. Aug. 14, 2001;98(17):9742-7 and Elbashir et al. Methods 2002 February;26(2): 199-213). There is increasing evidence of post-transcriptional gene silencing by RNA interference (RNAi) for inhibiting targeted expression in mammalian cells at the mRNA level, in human cells. There is additional evidence of effective methods for inhibiting the proliferation and migration of tumor cells in human patients, and for inhibiting metastatic cancer development (see, e.g., U.S. patent application No. US2001000993183; Caplen et al. Proc Natl Acad Sci U S A; and Abderrahmani et al. Mol Cell Biol Nov. 12, 2001 (21):7256-67).

An “siRNA” or “RNAi” refers to a nucleic acid that forms a double stranded RNA and has the ability to reduce or inhibit expression of a gene or target gene when the siRNA is delivered to or expressed in the same cell as the gene or target gene. “siRNA” refers to short double-stranded RNA formed by the complementary strands. Complementary portions of the siRNA that hybridize to form the double stranded molecule often have substantial or complete identity to the target molecule sequence. In one embodiment, an siRNA refers to a nucleic acid that has substantial or complete identity to a target gene and forms a double stranded siRNA, such as a nucleotide sequence set forth in SEQ ID NO: 1, 2 or 3, for example.

When designing the siRNA molecules, the targeted region often is selected from a given DNA sequence beginning 50 to 100 nucleotides downstream of the start codon. See, e.g., Elbashir et al,. Methods 26:199-213 (2002). Initially, 5′ or 3′ UTRs and regions nearby the start codon were avoided assuming that UTR-binding proteins and/or translation initiation complexes may interfere with binding of the siRNP or RISC endonuclease complex. Sometimes regions of the target 23 nucleotides in length conforming to the sequence motif AA(N19)TT (N, an nucleotide), and regions with approximately 30% to 70% G/C-content (often about 50% G/C-content) often are selected. If no suitable sequences are found, the search often is extended using the motif NA(N21). The sequence of the sense siRNA sometimes corresponds to (N19) TT or N21 (position 3 to 23 of the 23-nt motif), respectively. In the latter case, the 3′ end of the sense siRNA often is converted to TT. The rationale for this sequence conversion is to generate a symmetric duplex with respect to the sequence composition of the sense and antisense 3′ overhangs. The antisense siRNA is synthesized as the complement to position 1 to 21 of the 23-nt motif. Because position 1 of the 23-nt motif is not recognized sequence-specifically by the antisense siRNA, the 3′-most nucleotide residue of the antisense siRNA can be chosen deliberately. However, the penultimate nucleotide of the antisense siRNA (complementary to position 2 of the 23-nt motif) often is complementary to the targeted sequence. For simplifying chemical synthesis, TT often is pyrimidine (C,U), often are selected. Respective 21 nucleotide sense and antisense siRNAs often begin with a purine nucleotide and can also be expressed from pol Ill expression vectors without a change in targeting site. Expression of RNAs from pol III promoters often is efficient when the first transcribed nucleotide is a purine.

The sequence of the siRNA can correspond to the full length target gene, or a subsequence thereof. Often, the siRNA is about 15 to about 50 nucleotides in length (e.g., each complementary sequence of the double stranded siRNA is 15-50 nucleotides in length, and the double stranded siRNA is about 15-50 base pairs in length, somtimes about 20-30 nucleotides in length or about 20-25 nucleotides in length, e.g., 20, 21, 22, 23, 24, 25, 26, 27, 28, 29, or 30 nucleotides in length. The siRNA sometimes is about 21 nucleotides in length. Methods of using siRNA are well known in the art, and specific siRNA molecules may be purchased from a number of companies including Dharmacon Research, Inc.

Antisense, ribozyme, RNAi and siRNA nucleic acids can be altered to form modified NRP1, NID2 or ENDO180 nucleic acid molecules. The nucleic acids can be altered at base moieties, sugar moieties or phosphate backbone moieties to improve stability, hybridization, or solubility of the molecule. For example, the deoxyribose phosphate backbone of nucleic acid molecules can be modified to generate peptide nucleic acids (see Hyrup et al., Bioorganic & Medicinal Chemistry 4 (1): 5-23 (1996)). As used herein, the terms “peptide nucleic acid” or “PNA” refers to a nucleic acid mimic such as a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. Synthesis of PNA oligomers can be performed using standard solid phase peptide synthesis protocols as described, for example, in Hyrup et al., (1996) supra and Perry-O'Keefe et al., Proc. Natl. Acad. Sci. 93: 14670-675 (1996).

PNAs of NRP1, NID2 or ENDO180 nucleic acids can be used in prognostic, diagnostic, and therapeutic applications. For example, PNAs can be used as antisense or antigene agents for sequence-specific modulation of gene expression by, for example, inducing transcription or translation arrest or inhibiting replication. PNAs of NRP1, NID2 or ENDO180 nucleic acid molecules can also be used in the analysis of single base pair mutations in a gene, (e.g., by PNA-directed PCR clamping); as “artificial restriction enzymes” when used in combination with other enzymes, (e.g., S1 nucleases (Hyrup (1996) supra)); or as probes or primers for DNA sequencing or hybridization (Hyrup et al., (1996) supra; Perry-O'Keefe supra).

In other embodiments, oligonucleotides may include other appended groups such as peptides (e.g., for targeting host cell receptors in vivo), or agents facilitating transport across cell membranes (see e.g., Letsinger et al., Proc. Natl. Acad. Sci. USA 86: 6553-6556 (1989); Lemaitre et al., Proc. Natl. Acad. Sci. USA 84: 648-652 (1987); PCT Publication No. WO88/09810) or the blood-brain barrier (see, e.g., PCT Publication No. WO89/10134). In addition, oligonucleotides can be modified with hybridization-triggered cleavage agents (See, e.g., Krol et al., Bio-Techniques 6: 958-976 (1988)) or intercalating agents. (See, e.g., Zon, Pharm. Res. 5: 539-549 (1988) ). To this end, the oligonucleotide may be conjugated to another molecule, (e.g., a peptide, hybridization triggered cross-linking agent, transport agent, or hybridization-triggered cleavage agent).

Also included herein are molecular beacon oligonucleotide primer and probe molecules having one or more regions complementary to a NRP1, NID2 or ENDO180 nucleic acid, two complementary regions one having a fluorophore and one a quencher such that the molecular beacon is useful for quantifying the presence of the NRP1, NID2 or ENDO180 nucleic acid in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Anti-NRP1, NID2 or ENDO180 Antibodies

The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin. An antibody sometimes is a polyclonal, monoclonal, recombinant (e.g., a chimeric or humanized), fully human, non-human (e.g., murine), or a single chain antibody. An antibody may have effector function and can fix complement, and is sometimes coupled to a toxin or imaging agent.

A full-length NRP1, NID2 or ENDO180 polypeptide or antigenic peptide fragment can be used as an immunogen or can be used to identify anti-NRP1, NID2 or ENDO180 antibodies made with other immunogens, e.g., cells, membrane preparations, and the like. An antigenic peptide of NRP1, NID2 or ENDO180 often includes at least 8 amino acid residues of the amino acid sequences set forth in FIGS. 5A-5B, 6A-6B or 7A-7B and encompasses an epitope of NRP1, NID2 or ENDO180. Antigenic peptides sometimes include 10 or more amino acids, 15 or more amino acids, 20 or more amino acids, or 30 or more amino acids. Hydrophilic and hydrophobic fragments of NRP1, NID2 or ENDO180 polypeptides sometimes are used as immunogens.

Epitopes encompassed by the antigenic peptide are regions of NRP1, NID2 or ENDO180 located on the surface of the polypeptide (e.g., hydrophilic regions) as well as regions with high antigenicity. For example, an Emini surface probability analysis of the human NRP1, NID2 or ENDO180 polypeptide sequence can be used to indicate the regions that have a particularly high probability of being localized to the surface of the NRP1, NID2 or ENDO180 polypeptide and are thus likely to constitute surface residues useful for targeting antibody production. The antibody may bind an epitope on any domain or region on NRP1, NID2 or ENDO180 polypeptides described herein.

Also, chimeric, humanized, and completely human antibodies are useful for applications which include repeated administration to subjects. Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al International Application No. PCT/US86/02269; Akira, et al European Patent Application 184,187; Taniguchi, M., European Patent Application 171,496; Morrison et al European Patent Application 173,494; Neuberger et al PCT International Publication No. WO 86/01533; Cabilly et al U.S. Pat. No. 4,816,567; Cabilly et al European Patent Application 125,023; Better et al., Science 240: 1041-1043 (1988); Liu et al., Proc. Natl. Acad. Sci. USA 84: 3439-3443 (1987); Liu et al., J. Immunol. 139: 3521-3526 (1987); Sun et al., Proc. Natl. Acad. Sci. USA 84: 214-218 (1987); Nishimura et al., Canc. Res. 47: 999-1005 (1987); Wood et al., Nature 314: 446-449 (1985); and Shaw et al., J. Natl. Cancer Inst. 80: 1553-1559 (1988); Morrison, S. L., Science 229: 1202-1207 (1985); Oi et al., BioTechniques 4: 214 (1986); Winter U.S. Pat. No. 5,225,539; Jones et al., Nature 321: 552-525 (1986); Verhoeyan et al., Science 239: 1534; and Beidler et al., J. Immunol. 141: 4053-4060 (1988).

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar, Int. Rev. Immunol. 13: 65-93 (1995); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above. Completely human antibodies that recognize a selected epitope also can be generated using a technique referred to as “guided selection.” In this approach a selected non-human monoclonal antibody (e.g., a murine antibody) is used to guide the selection of a completely human antibody recognizing the same epitope. This technology is described for example by Jespers et al., Bio/Technology 12: 899-903 (1994).

An anti-NRP1, NID2 or ENDO180 antibody can be a single chain antibody. A single chain antibody (scFV) can be engineered (see, e.g., Colcher et al, Ann. N Y Acad. Sci. 880: 263-80 (1999); and Reiter, Clin. Cancer Res. 2: 245-52 (1996)). Single chain antibodies can be dimerized or multimerized to generate multivalent antibodies having specificities for different epitopes of the same target NRP1, NID2 or ENDO180 polypeptide.

Antibodies also may be selected or modified so that they exhibit reduced or no ability to bind an Fc receptor. For example, an antibody may be an isotype or subtype, fragment or other mutant, which does not support binding to an Fc receptor (e.g., it has a mutagenized or deleted Fc receptor binding region).

Also, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BCNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibody conjugates can be used for modifying a given biological response. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, γ-interferon, α-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Also, an antibody can be conjugated to a second antibody to form an antibody heteroconjugate as described by Segal in U.S. Pat. No. 4,676,980, for example.

An anti-NRP1, NID2 or ENDO180 antibody (e.g., monoclonal antibody) can be used to isolate NRP1, NID2 or ENDO180 polypeptides by standard techniques, such as affinity chromatography or immunoprecipitation. Moreover, an anti-NRP1, NID2 or ENDO180 antibody can be used to detect a NRP1, NID2 or ENDO180 polypeptide (e.g., in a cellular lysate or cell supernatant) in order to evaluate the abundance and pattern of expression of the polypeptide. Anti-NRP1, NID2 or ENDO180 antibodies can be used diagnostically to monitor polypeptide levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Also, an anti-NRP1, NID2 or ENDO180 antibody can be utilized as a test molecule for determining whether it can treat melanoma, and as a therapeutic for administration to a subject for treating melanoma.

An antibody can be made by immunizing with a purified NRP1, NID2 or ENDO180 antigen, or a fragment thereof, e.g., a fragment described herein, a membrane associated antigen, tissues, e.g., crude tissue preparations, whole cells, preferably living cells, lysed cells, or cell fractions.

Included herein are antibodies which bind only a native NRP1, NID2 or ENDO180 polypeptide, only denatured or otherwise non-native NRP1, NID2 or ENDO180 polypeptide, or which bind both, as well as those having linear or conformational epitopes. Conformational epitopes sometimes can be identified by selecting antibodies that bind to native but not denatured NRP1, NID2 or ENDO180 polypeptide. Also featured are antibodies that specifically bind to a NRP1, NID2 or ENDO180 protein or polypeptide variant associated with melanoma. For example, the antibody sometimes specifically binds to an epitope that comprises a serine at amino acid position 656 in a NID2 protein, polypeptide or peptide.

Screening Assays

Featured herein are methods for identifying a candidate therapeutic for treating melanoma and detecting occurance of melanoma. The methods comprise contacting a test molecule with a NRP1, NID2 or ENDO180 nucleic acid, substantially identical nucleic acid, polypeptide, substantially identical polypeptide, or fragment of the foregoing in a system. The nucleic acid often is a NRP1, NID2 or ENDO180 nucleotide sequence represented by SEQ ID NO: 1, 2 or 3, respectively; sometimes is a nucleotide sequence substantially identical to the nucleotide sequence of SEQ ID NO: 1, 2 or 3 or sometimes a fragment thereof, and the NRP1, NID2 or ENDO180 polypeptide or fragment thereof is a polypeptide encoded by any of these nucleic acids. The method also comprises determining the presence or absence of an interaction between the test molecule and the NRP1, NID2 or ENDO180 nucleic acid or polypeptide, where the presence of an interaction between the test molecule and the NRP1, NID2 or ENDO180 nucleic acid or polypeptide identifies the test molecule as a candidate melanoma therapeutic.

As used herein, the term “test molecule” and “candidate therapeutic” refers to modulators of regulation of transcription and translation of NRP1, NID2 or ENDO180 nucleic acids and modulations of expression and activity of NRP1, NID2 or ENDO180 polypeptides. The term “modulator” as used herein refers to a molecule which agonizes or antagonizes NRP1, NID2 or ENDO180 DNA replication and/or DNA processing (e.g., methylation), NRP1, NID2 or ENDO180 RNA transcription and/or RNA processing (e.g., removal of intronic sequences and/or translocation from the nucleus), NRP1, NID2 or ENDO180 polypeptide production (e.g., translation of the polypeptide from mRNA, and/or post-translational modification such as glycosylation, phosphorylation, and proteolysis of pro-polypeptides), and/or NRP1, NID2 or ENDO180 function (e.g., conformational changes, binding of nucleotides or nucleotide analogs, interaction with binding partners, effect on phosphorylation, and/or effect on occurrence of melanoma). Test molecules and candidate therapeutics include, but are not limited to, compounds, RNAi or siRNA molecules, antisense nucleic acids, ribozymes, NRP1, NID2 or ENDO180 polypeptides or fragments thereof, and immunotherapeutics (e.g., antibodies and HLA-presented polypeptide fragments).

As used herein, the term “system” refers to a cell free in vitro environment and a cell-based environment such as a collection of cells, a tissue, an organ, or an organism. A system is “contacted” with a test molecule in a variety of manners, including adding molecules in solution and allowing them to interact with one another by diffusion, cell injection, and any administration routes in an animal. As used herein, the term “interaction” refers to an effect of a test molecule on a NRP1, NID2 or ENDO180 nucleic acid, polypeptide, or variant thereof (collectively referred to as a “NRP1, NID2 or ENDO180 molecule”), where the effect is sometimes binding between the test molecule and the nucleic acid or polypeptide, and is often an observable change in cells, tissue, or organism.

There are many standard methods for detecting the presence or absence of interaction between a test molecule and a NRP1, NID2 or ENDO180 nucleic acid or polypeptide. For example, titrametric, acidimetric, radiometric, NMR, monolayer, polarographic, spectrophotometric, fluorescent, and ESR assays probative of NRP1, NID2 or ENDO180 function may be utilized.

NRP1, NID2 or ENDO180 activity and/or NRP1, NID2 or ENDO180 interactions can be detected and quantified using assays known in the art and described in Examples hereafter.

An interaction can be determined by labeling the test molecule and/or the NRP1, NID2 or ENDO180 molecule, where the label is covalently or non-covalently attached to the test molecule or NRP1, NID2 or ENDO180 molecule. The label is sometimes a radioactive molecule such as ¹²⁵I, ¹³¹I, ³⁵S or ³H, which can be detected by direct counting of radioemission or by scintillation counting. Also, enzymatic labels such as horseradish peroxidase, alkaline phosphatase, or luciferase may be utilized where the enzymatic label can be detected by determining conversion of an appropriate substrate to product. Also, presence or absence of an interaction can be determined without labeling. For example, a microphysiometer (e.g., Cytosensor) is an analytical instrument that measures the rate at which a cell acidifies its environment using a light-addressable potentiometric sensor (LAPS). Changes in this acidification rate can be used as an indication of an interaction between a test molecule and NRP1, NID2 or ENDO180 (McConnell, H. M. et al., Science 257: 1906-1912 (1992)).

In cell-based systems, cells typically include a NRP1, NID2 or ENDO180 nucleic acid or polypeptide or variants thereof and are often of mammalian origin, although the cell can be of any origin. Whole cells, cell homogenates, and cell fractions (e.g., cell membrane fractions) can be subjected to analysis. Where interactions between a test molecule with a NRP1, NID2 or ENDO180 polypeptide or variant thereof are monitored, soluble and/or membrane bound forms of the polypeptide or variant may be utilized. Where membrane-bound forms of the polypeptide are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

An interaction between two molecules can also be detected by monitoring fluorescence energy transfer (FET) (see, for example, Lakowicz et al., U.S. Pat. No. 5,631,169; Stavrianopoulos et al. U.S. Pat. No. 4,868,103). A fluorophore label on a first, “donor” molecule is selected such that its emitted fluorescent energy will be absorbed by a fluorescent label on a second, “acceptor” molecule, which in turn is able to fluoresce due to the absorbed energy. Alternately, the “donor” polypeptide molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. An FET binding event can be conveniently measured through standard fluorometric detection means well known in the art (e.g., using a fluorimeter).

In another embodiment, determining the presence or absence of an interaction between a test molecule and a NRP1, NID2 or ENDO180 molecule can be effected by using real-time Biomolecular Interaction Analysis (BIA) (see, e.g., Sjolander & Urbaniczk, Anal. Chem. 63: 2338-2345 (1991) and Szabo et al., Curr. Opin. Struct. Biol. 5: 699-705 (1995)). “Surface plasmon resonance” or “B1A” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In another embodiment, the NRP1, NID2 or ENDO180 molecule or test molecules are anchored to a solid phase. The NRP1, NID2 or ENDO180 molecule/test molecule complexes anchored to the solid phase can be detected at the end of the reaction. The target NRP1, NID2 or ENDO180 molecule is often anchored to a solid surface, and the test molecule, which is not anchored, can be labeled, either directly or indirectly, with detectable labels discussed herein.

It may be desirable to immobilize a NRP1, NID2 or ENDO180 molecule, an anti-NRP1, NID2 or ENDO180 antibody, or test molecules to facilitate separation of complexed from uncomplexed forms of NRP1, NID2 or ENDO180 molecules and test molecules, as well as to accommodate automation of the assay. Binding of a test molecule to a NRP1, NID2 or ENDO180 molecule can be accomplished in any vessel suitable for containing the reactants. Examples of such vessels include microtitre plates, test tubes, and micro-centrifuge tubes. In one embodiment, a fusion polypeptide can be provided which adds a domain that allows a NRP1, NID2 or ENDO180 molecule to be bound to a matrix. For example, glutathione-S-transferase/NRP1, NID2 or ENDO180 fusion polypeptides or glutathione-S-transferase/target fusion polypeptides can be adsorbed onto glutathione sepharose beads (Sigma Chemical, St. Louis, Mo.) or glutathione derivatized microtitre plates, which are then combined with the test compound or the test compound and either the non-adsorbed target polypeptide or NRP1, NID2 or ENDO180 polypeptide, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of NRP1, NID2 or ENDO180 binding or activity determined using standard techniques.

Other techniques for immobilizing a NRP1, NID2 or ENDO180 molecule on matrices include using biotin and streptavidin. For example, biotinylated NRP1, NID2 or ENDO180 polypeptide or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with NRP1, NID2 or ENDO180 polypeptide or test molecules but which do not interfere with binding of the NRP1, NID2 or ENDO180 polypeptide to its test molecule. Such antibodies can be derivatized to the wells of the plate, and unbound target or NRP1, NID2 or ENDO180 polypeptide trapped in the wells by antibody conjugation. Methods for detecting such complexes, in addition to those described above for the GST-immobilized complexes, include immunodetection of complexes using antibodies reactive with the NRP1, NID2 or ENDO180 polypeptide or target molecule, as well as enzyme-linked assays which rely on detecting an enzymatic activity associated with the NRP1, NID2 or ENDO180 polypeptide or test molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci August;18(8): 284-7 (1993)); chromatography (gel filtration chromatography, ion-exchange chromatography); electrophoresis (see, e.g., Ausubel et al., eds. Current Protocols in Molecular Biology, J. Wiley: New York (1999)); and immunoprecipitation (see, for example, Ausubel, F. et al., eds. Current Protocols in Molecular Biology, J. Wiley: New York (1999)). Such resins and chromatographic techniques are known to one skilled in the art (see, e.g., Heegaard, J Mol. Recognit. Winter; 11 (1-6): 141-8 (1998); Hage & Tweed, J. Chromatogr. B Biomed. Sci. Appl. October 10; 699 (1-2): 499-525 (1997)). Further, fluorescence energy transfer may also be conveniently utilized, as described herein, to detect binding without further purification of the complex from solution.

In another embodiment, modulators of NRP1, NID2 or ENDO180 expression are identified. For example, a cell or cell free mixture is contacted with a candidate compound and the expression of NRP1, NID2 or ENDO180 mRNA or polypeptide evaluated relative to the level of expression of NRP1, NID2 or ENDO180 mRNA or polypeptide in the absence of the candidate compound. When expression of NRP1, NID2 or ENDO180 mRNA or polypeptide is greater in the presence of the candidate compound than in its absence, the candidate compound is identified as a stimulator of NRP1, NID2 or ENDO180 mRNA or polypeptide expression. Alternatively, when expression of NRP1, NID2 or ENDO180 mRNA or polypeptide is less (statistically significantly less) in the presence of the candidate compound than in its absence, the candidate compound is identified as an inhibitor of NRP1, NID2 or ENDO180 mRNA or polypeptide expression. The level of NRP1, NID2 or ENDO180 mRNA or polypeptide expression can be determined by methods described herein for detecting NRP1, NID2 or ENDO180 mRNA or polypeptide.

In an embodiment, binding partners that interact with a NRP1, NID2 or ENDO180 molecule are detected. The NRP1, NID2 or ENDO180 molecules can interact with one or more cellular or extracellular macromolecules, such as polypeptides, in vivo, and these molecules that interact with NRP1, NID2 or ENDO180 molecules are referred to herein as “binding partners.” Molecules that disrupt such interactions can be useful in regulating the activity of the target gene product. Such molecules can include, but are not limited to molecules such as antibodies, peptides, and small molecules (e.g., siRNA). The preferred target genes/products for use in this embodiment are the NRP1, NID2 or ENDO180 genes herein identified. In an alternative embodiment, provided are methods for determining the ability of the test compound to modulate the activity of a NRP1, NID2 or ENDO180 polypeptide through modulation of the activity of a downstream effector of a NRP1, NID2 or ENDO180 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), e.g., a substrate, a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtitre plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

Also, binding partners of NRP1, NID2 or ENDO180 molecules can be identified in a two-hybrid assay or three-hybrid assay (see, e.g., U.S. Pat. No. 5,283,317; Zervos et al., Cell 72:223-232 (1993); Madura et al., J. Biol. Chem. 268: 12046-12054 (1993); Bartel et al., Biotechniques 14: 920-924 (1993); Iwabuchi et al., Oncogene 8: 1693-1696 (1993); and Brent WO94/10300), to identify other polypeptides, which bind to or interact with NRP1, NID2 or ENDO180 (“NRP1, NID2 or ENDO180-binding polypeptides” or “NRP1, NID2 or ENDO180-bp”) and are involved in NRP1, NID2 or ENDO180 activity. Such NRP1, NID2 or ENDO180-bps can be activators or inhibitors of signals by the NRP1, NID2 or ENDO180 polypeptides or NRP1, NID2 or ENDO180 targets as, for example, downstream elements of a NRP1, NID2 or ENDO180-mediated signaling pathway.

A two-hybrid system is based on the modular nature of most transcription factors, which consist of separable DNA-binding and activation domains. Briefly, the assay utilizes two different DNA constructs. In one construct, the gene that codes for a NRP1, NID2 or ENDO180 polypeptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. (Alternatively the: NRP1, NID2 or ENDO180 polypeptide can be the fused to the activator domain.) If the “bait” and the “prey” polypeptides are able to interact, in vivo, forming a NRP1, NID2 or ENDO180-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with the NRP1, NID2 or ENDO180 polypeptide.

Candidate therapeutics for treating melanoma are identified from a group of test molecules that interact with a NRP1, NID2 or ENDO180 nucleic acid or polypeptide. Test molecules often are ranked according to the degree with which they interact or modulate (e.g., agonize or antagonize) DNA replication and/or processing, RNA transcription and/or processing, polypeptide production and/or processing, and/or function of NRP1, NID2 or ENDO180 molecules, for example, and then top ranking modulators are selected. Also, pharmacogenomic information described herein can determine the rank of a modulator. Candidate therapeutics typically are formulated for administration to a subject.

Therapeutic Treatments

Formulations or pharmaceutical compositions typically include in combination with a pharmaceutically acceptable carrier a compound, an antisense nucleic acid, a ribozyme, an antibody, a binding partner that interacts with a NRP1, NID2 or ENDO180 polypeptide, a NRP1, NID2 or ENDO180 nucleic acid, or a fragment thereof. The formulated molecule may be one that is identified by a screening method described above. Also, formulations may comprise a NRP1, NID2 or ENDO180 polypeptide or fragment thereof and a pharmaceutically acceptable carrier. As used herein, the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation often utilized are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams (e.g., sunscreen) as generally known in the art. Molecules can also be prepared in the form of suppositories (e.g., with conventional suppository bases such as cocoa butter and other glycerides) or retention enemas for rectal delivery.

In one embodiment, active molecules are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Molecules which exhibit high therapeutic indices often are utilized. While molecules that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such molecules lies preferably within a range of circulating concentrations that include the ED₅₀ with little or no toxicity. The dosage may vary within this range depending upon the dosage form employed and the route of administration utilized. For any molecules used in the method, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, sometimes about 0.01 to 25 mg/kg body weight, often about 0.1 to 20 mg/kg body weight, and more often about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, sometimes between 2 to 8 weeks, often between about 3 to 7 weeks, and more often for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

With regard to polypeptide formulations, featured herein is a method for treating melanoma in a subject, which comprises contacting one or more cells in the subject with a first NRP1, NID2 or ENDO180 polypeptide, where genomic DNA in the subject comprises a second NRP1, NID2 or ENDO180 nucleic acid having one or more polymorphic variations associated with melanoma. The first NRP1, NID2 or ENDO180 polypeptide comprises fewer polymorphic variations associated with melanoma than the second NRP1, NID2 or ENDO180 polypeptide. The first and second NRP1, NID2 or ENDO180 polypeptides are encoded by a nucleic acid which comprises a nucleotide sequence selected from the group consisting of the nucleotide sequence of SEQ ID NO: 1, 2 or 3; a nucleotide sequence which encodes a polypeptide consisting of an amino acid sequence set forth in FIGS. 5A-5B, 6A-6B and 7A-7B; and a nucleotide sequence which encodes a polypeptide that is 90% or more identical to an amino acid sequence set forth in FIGS. 5A-5B, 6A-6B and 7A-7B. The second NRP1, NID2 or ENDO180 polypeptide also may be encoded by a fragment of the foregoing nucleic acids comprising the one or more polymorphic variations. The subject is often a human.

For antibodies, a dosage of 0.1 mg/kg of body weight (generally 10 mg/kg to 20 mg/kg) is often utilized. If the antibody is to act in the brain, a dosage of 50 mg/kg to 100 mg/kg is often appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., J. Acquired Immune Deficiency Syndromes and Human Retrovirology 14:193 (1997).

Antibody conjugates can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1” ), interleukin-2 (“IL-2”), interleukin-6 (“IL-6”), granulocyte macrophage colony stimulating factor (“GM-CSF”), granulocyte colony stimulating factor (“G-CSF”), or other growth factors. Alternatively, an antibody can be conjugated to a second antibody to form an antibody heteroconjugate as described by Segal in U.S. Pat. No.4,676,980.

For compounds, exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

NRP1, NID2 or ENDO180 nucleic acid molecules can be inserted into vectors and used in gene therapy methods for treating melanoma. Featured herein is a method for treating melanoma in a subject, which comprises contacting one or more cells in the subject with a first NRP1, NID2 or ENDO180 nucleic acid. Genomic DNA in the subject comprises a second NRP1, NID2 or ENDO180 nucleic acid comprising one or more polymorphic variations associated with melanoma, and the first NRP1, NID2 or ENDO180 nucleic acid comprises fewer polymorphic variations associated with melanoma. The first and second NRP1, NID2 or ENDO180 nucleic acids typically comprise a nucleotide sequence selected from the group consisting of the nucleotide sequence of SEQ ID NO: 1, 2 or 3; a nucleotide sequence which encodes a polypeptide consisting of an amino acid sequence set forth in FIGS. 5A-5B, 6A-6B and 7A-7B; and a nucleotide sequence which encodes a polypeptide that is 90% or more identical to an amino acid sequence set forth in FIGS. 5A-5B, 6A-6B and 7A-7B. The second NRP1, NID2 or ENDO180 nucleic acid may also be a fragment of the foregoing comprising one or more polymorphic variations. The subject is often a human.

Gene therapy vectors can be delivered to a subject by, for example, intravenous injection, local administration (see U.S. Pat. No. 5,328,470) or by stereotactic injection (see e.g., Chen et al., (1994) Proc. Natl. Acad. Sci. USA 91:3054-3057). Pharmaceutical preparations of gene therapy vectors can include a gene therapy vector in an acceptable diluent, or can comprise a slow release matrix in which the gene delivery vehicle is imbedded. Alternatively, where the complete gene delivery vector can be produced intact from recombinant cells (e.g., retroviral vectors) the pharmaceutical preparation can include one or more cells which produce the gene delivery system. Examples of gene delivery vectors are described herein.

Pharmaceutical compositions can be included in a container, pack, or dispenser together with instructions for administration.

Pharmaceutical compositions of active ingredients can be administered by any of the paths described herein for therapeutic and prophylactic methods for treating melanoma. With regard to both prophylactic and therapeutic methods of treatment, such treatments may be specifically tailored or modified, based on knowledge obtained from pharmacogenomic analyses described herein. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

Administration of a prophylactic agent can occur prior to the manifestation of symptoms characteristic of the NRP1, NID2 or ENDO180 aberrance, such that a disease or disorder is prevented or, alternatively, delayed in its progression. Depending on the type of NRP1, NID2 or ENDO180 aberrance, for example, a NRP1, NID2 or ENDO180 molecule, NRP1, NID2 or ENDO180 agonist, or NRP1, NID2 or ENDO180 antagonist agent can be used for treating the subject. The appropriate agent can be determined based on screening assays described herein.

As discussed, successful treatment of NRP1, NID2 or ENDO180 disorders can be brought about by techniques that serve to inhibit the expression or activity of target gene products. For example, compounds (e.g., an agent identified using an assays described above) that exhibit negative modulatory activity can be used to prevent and/or treat melanoma. Such molecules can include, but are not limited to peptides, phosphopeptides, small organic or inorganic molecules, or antibodies (including, for example, polyclonal, monoclonal, humanized, anti-idiotypic, chimeric or single chain antibodies, and FAb, F(ab′)₂ and FAb expression library fragments, scFV molecules, and epitope-binding fragments thereof).

Further, antisense and ribozyme molecules that inhibit expression of the target gene can also be used to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular polypeptide, it can be preferable to co-administer normal target gene polypeptide into the cell or tissue in order to maintain the requisite level of cellular or tissue target gene activity.

Another method by which nucleic acid molecules may be utilized in treating or preventing a disease characterized by NRP1, NID2 or ENDO180 expression is through the use of aptamer molecules specific for NRP1, NID2 or ENDO180 polypeptide. Aptamers are nucleic acid molecules having a tertiary structure which permits them to specifically bind to polypeptide ligands (see, e.g., Osborne, et al., Curr. Opin. Chem. Biol.1(1): 5-9 (1997); and Patel, D. J., Curr. Opin. Chem. Biol. June;1(1): 32-46 (1997)). Since nucleic acid molecules may in many cases be more conveniently introduced into target cells than therapeutic polypeptide molecules may be, aptamers offer a method by which NRP1, NID2 or ENDO180 polypeptide activity may be specifically decreased without the introduction of drugs or other molecules which may have pluripotent effects.

Antibodies can be generated that are both specific for target gene product and that reduce target gene product activity. Such antibodies may, therefore, by administered in instances whereby negative modulatory techniques are appropriate for the treatment of NRP1, NID2 or ENDO180 disorders. For a description of antibodies, see the Antibody section above.

In circumstances where injection of an animal or a human subject with a NRP1, NID2 or ENDO180 polypeptide or epitope for stimulating antibody production is harmful to the subject, it is possible to generate an immune response against NRP1, NID2 or ENDO180 through the use of anti-idiotypic antibodies (see, for example, Herlyn, D., Ann. Med.;31 (1): 66-78 (1999); and Bhattacharya-Chatterjee & Foon, Cancer Treat. Res.; 94: 51-68 (1998)). If an anti-idiotypic antibody is introduced into a mammal or human subject, it should stimulate the production of anti-anti-idiotypic antibodies, which should be specific to the NRP1, NID2 or ENDO180 polypeptide. Vaccines directed to a disease characterized by NRP1, NID2 or ENDO180 expression may also be generated in this fashion.

In instances where the target antigen is intracellular and whole antibodies are used, internalizing antibodies often are utilized. Lipofectin or liposomes can be used to deliver the antibody or a fragment of the Fab region that binds to the target antigen into cells. Where fragments of the antibody are used, the smallest inhibitory fragment that binds to the target antigen often are utilized. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al., Proc. Natl. Acad. Sci. USA 90: 7889-7893 (1993)).

NRP1, NID2 or ENDO180 molecules and compounds that inhibit target gene expression, synthesis and/or activity can be administered to a patient at therapeutically effective doses to prevent, treat or ameliorate NRP1, NID2 or ENDO180 disorders. A therapeutically effective dose refers to that amount of the compound sufficient to result in amelioration of symptoms of the disorders.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices often are utilized. While compounds that exhibit toxic side effects can be used, care should be taken to design a delivery system that targets such compounds to the site of affected tissue in order to minimize potential damage to uninfected cells and, thereby, reduce side effects.

Data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of effective dose determination for an individual is the ability to directly assay levels of “free” and “bound” compound in the serum of the test subject. Such assays may utilize antibody mimics and/or “biosensors” that have been created through molecular imprinting techniques. The compound which is able to modulate NRP1, NID2 or ENDO180 activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al., Current Opinion in Biotechnology 7: 89-94 (1996) and in Shea, Trends in Polymer Science 2: 166-173 (1994). Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, et al., Nature 361: 645-647 (1993). Through the use of isotope-labeling, the “free” concentration of compound which modulates the expression or activity of NRP1, NID2 or ENDO180 can be readily monitored and used in calculations of IC₅₀. Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. A rudimentary example of such a “biosensor” is discussed in Kriz et al., Analytical Chemistry 67: 2142-2144 (1995).

Provided herein are methods of modulating NRP1, NID2 or ENDO180 expression or activity for therapeutic purposes. Accordingly, in an exemplary embodiment, the modulatory method involves contacting a cell with NRP1, NID2 or ENDO180 or an agent that modulates one or more activities of NRP1, NID2 or ENDO180 polypeptide activity associated with the cell. An agent that modulates NRP1, NID2 or ENDO180 polypeptide activity can be an agent as described herein, such as a nucleic acid or a polypeptide, a naturally-occurring target molecule of a NRP1, NID2 or ENDO180 polypeptide (e.g., a NRP1, NID2 or ENDO180 substrate or receptor), a NRP1, NID2 or ENDO180 antibody, a NRP1, NID2 or ENDO180 agonist or antagonist, a peptidomimetic of a NRP1, NID2 or ENDO180 agonist or antagonist, or other small molecule.

In one embodiment, the agent stimulates one or more NRP1, NID2 or ENDO180 activities. Examples of such stimulatory agents include active NRP1, NID2 or ENDO180 polypeptide and a nucleic acid molecule encoding NRP1, NID2 or ENDO180. In another embodiment, the agent inhibits one or more NRP1, NID2 or ENDO180 activities. Examples of such inhibitory agents include antisense NRP1, NID2 or ENDO180 nucleic acid molecules, anti-NRP1, NID2 or ENDO180 antibodies, and NRP1, NID2 or ENDO180 inhibitors. These modulatory methods can be performed in vitro (e.g., by culturing the cell with the agent) or, alternatively, in vivo (e.g., by administering the agent to a subject). As such, provided are methods of treating an individual afflicted with a disease or disorder characterized by aberrant or unwanted expression or activity of a NRP1, NID2 or ENDO180 polypeptide or nucleic acid molecule. In one embodiment, the method involves administering an agent (e.g., an agent identified by a screening assay described herein), or combination of agents that modulates (e.g., upregulates or downregulates) NRP1, NID2 or ENDO180 expression or activity. In another embodiment, the method involves administering a NRP1, NID2 or ENDO180 polypeptide or nucleic acid molecule as therapy to compensate for reduced, aberrant, or unwanted NRP1, NID2 or ENDO180 expression or activity.

Stimulation of NRP1, NID2 or ENDO180 activity is desirable in situations in which NRP1, NID2 or ENDO180 is abnormally downregulated and/or in which increased NRP1, NID2 or ENDO180 activity is likely to have a beneficial effect. For example, stimulation of NRP1, NID2 or ENDO180 activity is desirable in situations in which a NRP1, NID2 or ENDO180 is downregulated and/or in which increased NRP1, NID2 or ENDO180 activity is likely to have a beneficial effect. Likewise, inhibition of NRP1, NID2 or ENDO180 activity is desirable in situations in which NRP1, NID2 or ENDO180 is abnormally upregulated and/or in which decreased NRP1, NID2 or ENDO180 activity is likely to have a beneficial effect.

The examples set forth below are intended to illustrate but not limit the invention.

### EXAMPLES

In the following studies a group of subjects were selected according to specific parameters relating to melanoma. Nucleic acid samples obtained from individuals in the study group were subjected to genetic analysis, which identified associations between melanoma and certain polymorphic regions in the NRP1, NID2 or ENDO180 genes. Methods are described for producing NRP1, NID2 or ENDO180 polypeptides and polypeptide variants in vitro or in vivo. NRP1, NID2 or ENDO180 nucleic acids or polypeptides and variants thereof are utilized for screening test molecules for those that interact with NRP1, NID2 or ENDO180 molecules. Test molecules identified as interactors with NRP1, NID2 or ENDO180 molecules and variants are further screened in vivo to determine whether they treat melanoma.

### Example 1

### Samples and Pooling Strategies

Sample Selection

Blood samples were collected from individuals diagnosed with melanoma, which were referred to as case samples. Also, blood samples were collected from individuals not diagnosed with melanoma or a history of melanoma; these samples served as gender and age-matched controls. A database was created that listed all phenotypic trait information gathered from individuals for each case and control sample. Genomic DNA was extracted from each of the blood samples for genetic analyses.

DNA Extraction from Blood Samples

Six to ten milliliters of whole blood was transferred to a 50 ml tube containing 27 ml of red cell lysis solution (RCL). The tube was inverted until the contents were mixed. Each tube was incubated for 10 minutes at room temperature and inverted once during the incubation. The tubes were then centrifuged for 20 minutes at 3000×g and the supernatant was carefully poured off. 100-200 μl of residual liquid was left in the tube and was pipetted repeatedly to resuspend the pellet in the residual supernatant. White cell lysis solution (WCL) was added to the tube and pipetted repeatedly until completely mixed. While no incubation was normally required, the solution was incubated at 37° C. or room temperature if cell clumps were visible after mixing until the solution was homogeneous. Two ml of protein precipitation was added to the cell lysate. The mixtures were vortexed vigorously at high speed for 20 sec to mix the protein precipitation solution uniformly with the cell lysate, and then centrifuged for 10 minutes at 3000×g. The supernatant containing the DNA was then poured into a clean 15 ml tube, which contained 7 ml of 100% isopropanol. The samples were mixed by inverting the tubes gently until white threads of DNA were visible. Samples were centrifuged for 3 minutes at 2000×g and the DNA was visible as a small white pellet. The supernatant was decanted and 5 ml of 70% ethanol was added to each tube. Each tube was inverted several times to wash the DNA pellet, and then centrifuged for 1 minute at 2000×g. The ethanol was decanted and each tube was drained on clean absorbent paper. The DNA was dried in the tube by inversion for 10 minutes, and then 1000 μl of 1×TE was added. The size of each sample was estimated, and less TE buffer was added during the following DNA hydration step if the sample was smaller. The DNA was allowed to rehydrate overnight at room temperature, and DNA samples were stored at 2-8° C.

DNA was quantified by placing samples on a hematology mixer for at least 1 hour. DNA was serially diluted (typically 1:80, 1:160, 1:320, and 1:640 dilutions) so that it would be within the measurable range of standards. 125 μl of diluted DNA was transferred to a clear U-bottom microtitre plate, and 125 μl of 1×TE buffer was transferred into each well using a multichannel pipette. The DNA and 1×TE were mixed by repeated pipetting at least 15 times, and then the plates were sealed. 50 μl of diluted DNA was added to wells A5-H12 of a black flat bottom microtitre plate. Standards were inverted six times to mix them, and then 50 μl of 1×TE buffer was pipetted into well A1, 1000 ng/ml of standard was pipetted into well A2, 500 ng/ml of standard was pipetted into well A3, and 250 ng/ml of standard was pipetted into well A4. PicoGreen (Molecular Probes, Eugene, Oreg.) was thawed and freshly diluted 1:200 according to the number of plates that were being measured. PicoGreen was vortexed and then 50 μl was pipetted into all wells of the black plate with the diluted DNA. DNA and PicoGreen were mixed by pipetting repeatedly at least 10 times with the multichannel pipette. The plate was placed into a Fluoroskan Ascent Machine (microplate fluorometer produced by Labsystems) and the samples were allowed to incubate for 3 minutes before the machine was run using filter pairs 485 nm excitation and 538 nm emission wavelengths. Samples having measured DNA concentrations of greater than 450 ng/μl were re-measured for conformation. Samples having measured DNA concentrations of 20 ng/μl or less were re-measured for confirmation.

Pooling Strategies

Samples were placed into one of four groups, based on gender and disease status. The four groups were male case samples, male control samples, female case samples, and female control samples. A select set of samples from each group were utilized to generate pools, and one pool was created for each group. Each individual sample in a pool was represented by an equal amount of genomic DNA. For example, where 25 ng of genomic DNA was utilized in each PCR reaction and there were 200 individuals in each pool, each individual would provide 125 pg of genomic DNA. Inclusion or exclusion of samples for a pool was based upon the following criteria: the sample was derived from an individual characterized as Caucasian; the sample was derived from an individual of German paternal and maternal descent; the database included relevant phenotype information for the individual; case samples were derived from individuals diagnosed with melanoma; control samples were derived from individuals free of cancer; and sufficient genomic DNA was extracted from each blood sample for all allelotyping and genotyping reactions performed during the study. Phenotype information included sex of the individual, number of nevi (few, moderate, numerous), hair color (black, brown, blond, red), diagnosed with melanoma (tumor thickness, date of primary diagnosis, age of individual as of primary diagnosis, post-operative tumor classification, presence of nodes, occurrence of metastases, subtype, location), country or origin of mother and father, presence of certain conditions for each individual (coronary heart disease, cardiomyopathy, arteriosclerosis, abnormal blood clotting/thrombosis, emphysema, asthma, diabetes type 1, diabetes type 2, Alzheimer's disease, epilepsy, schizophrenia, manic depression/bipolar disorder, autoimmune disease, thyroid disorder, and hypertension), presence of cancer in the donor individual or blood relative (melanoma, basaliom/spinaliom/lentigo malignant/mycosis fungoides, breast cancer, colon cancer, rectum cancer, lung cancer, lung cancer, bronchus cancer, prostate cancer, stomach cancer, leukemia, lymphoma, or other cancer in donor, donor parent, donor aunt or uncle, donor offspring or donor grandparent. Samples that met these criteria were added to appropriate pools based on gender and disease status.

The selection process yielded the pools set forth in Table 1, which were used in the studies that follow:

TABLE 1

| | Male control | Male case | Female control | Female case |
|---|---|---|---|---|
| Pool size | 217 | 236 | 233 | 266 |
| (Number) | | | | |
| Pool Criteria | control | case | control | case |
| (ex: case/ | | | | |
| control) | | | | |
| Mean Age | 48 | 51 | 47 | 49 |
| (ex: years) | | | | |

### Example 2

### Association of NRP1 Polymorphic Variants with Melanoma

A whole-genome screen was performed to identify particular SNPs associated with occurrence of melanoma. As described in Example 1, two sets of samples were utilized: female individuals having melanoma (female cases) and samples from female individuals not having melanoma or any history of melanoma (female controls), and male individuals having melanoma (male cases) and samples from male individuals not having melanoma or any history of melanoma (male controls). The initial screen of each pool was performed in an allelotyping study, in which certain samples in each group were pooled. By pooling DNA from each group, an allele frequency for each SNP in each group was calculated. These allele frequencies were then compared to one another. Particular SNPs were considered as being associated with melanoma when allele frequency differences calculated between case and control pools were statistically significant. SNP disease association results obtained from the allelotyping study were then validated by genotyping each associated SNP across all samples from each pool. The results of the genotyping were then analyzed, allele frequencies for each group were calculated from the individual genotyping results, and a p-value was calculated to determine whether the case and control groups had statistically significantly differences in allele frequencies for a particular SNP. When the genotyping results agreed with the original allelotyping results, the SNP disease association was considered validated at the genetic level.

SNP Panel Used for Genetic Analyses

A whole-genome SNP screen began with an initial screen of approximately 25,000 SNPs over each set of disease and control samples using a pooling approach. The pools studied in the screen are described in Example 1. The SNPs analyzed in this study were part of a set of 25,488 SNPs confirmed as being statistically polymorphic as each is characterized as having a minor allele frequency of greater than 10%. The SNPs in the set reside in genes or in close proximity to genes, and many reside in gene exons. Specifically, SNPs in the set are located in exons, introns, and within 5,000 base-pairs upstream of a transcription start site of a gene. In addition, SNPs were selected according to the following criteria: they are located in ESTs; they are located in Locuslink or Ensembl genes; and they are located in Genomatix promoter predictions. SNPs in the set were also selected on the basis of even spacing across the genome, as depicted in Table 2.

TABLE 2

| General Statistics | | Spacing Statistics | |
|---|---|---|---|
| Total # of SNPs | 25,488 | Median | 37,058 bp |
| # of Exonic SNPs | >4,335 (17%) | Minimum* | 1,000 bp |
| # SNPs with refSNP ID | 20,776 (81%) | Maximum* | 3,000,000 bp |
| Gene Coverage | >10,000 | Mean | 122,412 bp |
| Chromosome Coverage | All | Std Deviation | 373,325 bp |

*Excludes outliers

Genotyping Results

The genetic studies summarized above and described in more detail below identified allelic variants associated with melanoma. The allelic variants identified from the SNP panel described in Table 2 are summarized below in Table 3.

TABLE 3

| | | | | | | | Melanoma |
|---|---|---|---|---|---|---|---|
| SNP | Chromosome | Contig | Contig | Sequence | Sequence | Allelic | Associated |
| Reference | Position | Identification | Position | Identification | Position | Variability | Allele |
| rs1360457 | 33767168 | NT_008705 | 15592254 | NM_003873 | intragenic | T/C | T |
| rs753050 | 50495413 | NT_026437 | 32425413 | NM_007361 | intronic | A/G | A |
| rs729647 | 61089738 | NT_010783 | 16040858 | NM_006039 | intronic | A/G | G |

Table 3 includes information pertaining to the incident polymorphic variant associated with melanoma identified herein. Public information pertaining to the polymorphism and the genomic sequence that includes the polymorphism are indicated. The genomic sequence identified in Table 3 may be accessed at the http address www.ncbi.nih.gov/entrez/query.fcgi, for example, by using the publicly available SNP reference number (e.g., rs1360457). The chromosome position refers to the position of the SNP within NCBI's Genome Build 33, which may be accessed at the following http address: www.ncbi.nlm.nih.gov/mapview/map_search.cgi?chr=hum_chr.inf&query=. The “Contig Position” provided in Table 3 corresponds to a nucleotide position set forth in the contig sequence, and designates the polymorphic site corresponding to the SNP reference number. The sequence containing the polymorphisms also may be referenced by the “Sequence Identification” set forth in Table 3. The “Sequence Identification” corresponds to cDNA sequence that encodes associated target polypeptides (e.g., NRP1) of the invention. The position of the SNP within the cDNA sequence is provided in the “Sequence Position” column of Table 3. Also, the allelic variation at the polymorphic site and the allelic variant identified as associated with melanoma is specified in Table 3. All nucleotide sequences referenced and accessed by the parameters set forth in Table 3 are incorporated herein by reference.

Assay for Verifying, Allelotyping, and Genotyping SNPs

A MassARRAY™ system (Sequenom, Inc.) was utilized to perform SNP genotyping in a high-throughput fashion. This genotyping platform was complemented by a homogeneous, single-tube assay method (hME™ or homogeneous MassEXTEND™ (Sequenom, Inc.)) in which two genotyping primers anneal to and amplify a genomic target surrounding a polymorphic site of interest. A third primer (the MassEXTEND™ primer), which is complementary to the amplified target up to but not including the polymorphism, was then enzymatically extended one or a few bases through the polymorphic site and then terminated.

For each polymorphism, SpectroDESIGNER™ software (Sequenom, Inc.) was used to generate a set of PCR primers and a MassEXTEND™ primer which where used to genotype the polymorphism. Other primer design software could be used or one of ordinary skill in the art could manually design primers based on his or her knowledge of the relevant factors and considerations in designing such primers. Table 4 shows PCR primers and Table 5 shows extension primers used for analyzing the polymorphism set forth in Table 3. The initial PCR amplification reaction was performed in a 5 μl total volume containing 1× PCR buffer with 1.5 mM MgCl₂ (Qiagen), 200 μM each of dATP, dGTP, dCTP, dTTP (Gibco-BRL), 2.5 ng of genomic DNA, 0.1 units of HotStar DNA polymerase (Qiagen), and 200 nM each of forward and reverse PCR primers specific for the polymorphic region of interest.

TABLE 4
PCR Primers

| SNP | | |
|---|---|---|
| Reference | Forward PCR primer | Reverse PCR primer |
| rs1360457 | TTGTCCCAACTGAGGCTTTG | AACAGCCTTTCAGCTTTGGC |
| rs753050 | ACGTTGGATGCCTCTGTTTCCAACTCAAGG | ACGTTGGATGTTCACAGAGGTTACTAAGGG |
| rs729647 | AACCAACCCACTGGGTTGAC | TGATCTGGGAGAGTTTGGAG |

Samples were incubated at 95° C. for 15 minutes, followed by 45 cycles of 95° C. for 20 seconds, 56° C. for 30 seconds, and 72° C. for 1 minute, finishing with a 3 minute final extension at 72° C. Following amplification, shrimp alkaline phosphatase (SAP) (0.3 units in a 2 μl volume) (Amersham Pharmacia) was added to each reaction (total reaction volume was 7 μl) to remove any residual dNTPs that were not consumed in the PCR step. Samples were incubated for 20 minutes at 37° C., followed by 5 minutes at 85° C. to denature the SAP.

Once the SAP reaction was complete, a primer extension reaction was initiated by adding a polymorphism-specific MassEXTEND™ primer cocktail to each sample. Each MassEXTEND™ cocktail included a specific combination of dideoxynucleotides (ddNTPs) and deoxynucleotides (dNTPs) used to distinguish polymorphic alleles from one another. Methods for verifying, allelotyping and genotyping SNPs are disclosed, for example, in U.S. Pat. No.6,258,538, the content of which is hereby incorporated by reference. In Table 5, ddNTPs are shown and the fourth nucleotide not shown is the dNTP.

TABLE 5
Extension Primers

| SNP | | |
|---|---|---|
| Reference | Extend Probe | Termination Mix |
| rs1360457 | CAGCTTTGGCCAGGAGATG | ACT |
| rs753050 | AGCACATAACACAGCATGGC | ACT |
| rs729647 | GGTTGACGTCAACACAGGC | ACT |

The MassEXTEND™ reaction was performed in a total volume of 9 μl, with the addition of 1× ThermoSequenase buffer, 0.576 units of ThermoSequenase (Amersham Pharmacia), 600 nM MassEXTEND™ primer, 2 mM of ddATP and/or ddCTP and/or ddGTP and/or ddTTP, and 2 mM of dATP or dCTP or dGTP or dTTP. The deoxy nucleotide (dNTP) used in the assay normally was complementary to the nucleotide at the polymorphic site in the amplicon. Samples were incubated at 94° C. for 2 minutes, followed by 55 cycles of 5 seconds at 94° C., 5 seconds at 52° C., and 5 seconds at 72° C.

Following incubation, samples were desalted by adding 16 μl of water (total reaction volume was 25 μl), 3 mg of SpectroCLEAN™ sample cleaning beads (Sequenom, Inc.) and allowed to incubate for 3 minutes with rotation. Samples were then robotically dispensed using a piezoelectric dispensing device (SpectroJET™ (Sequenom, Inc.)) onto either 96-spot or 384-spot silicon chips containing a matrix that crystallized each sample (SpectroCHIP™ (Sequenom, Inc.)). Subsequently, MALDI-TOF mass spectrometry (Biflex and Autoflex MALDI-TOF mass spectrometers (Bruker Daltonics) can be used) and SpectroTYPER RT™ software (Sequenom, Inc.) were used to analyze and interpret the SNP genotype for each sample.

Genetic Analysis

The minor allelic frequencies for the polymorphisms set forth in Table 3 were verified as being 10% or greater using the extension assay described above in a group of samples isolated from 92 individuals originating from the state of Utah in the United States, Venezuela and France (Coriell cell repositories).

Genotyping results for the allelic variant set forth in Table 3 are shown for female pools in Table 6 and for male pools in Table 8. In Table 6, “F case” and “F control” refer to female case and female control groups, and in Table 7, “M case” and “M control” refer to male case and male control groups.

TABLE 6
Female Genotyping Results

| SNP Reference | F case | F control | p-value | Odd Ratio |
|---|---|---|---|---|
| rs1360457 | C = 0.869 | C = 0.903 | 0.089 | 0.71 |
| | T = 0.131 | T = 0.097 | | |
| rs753050 | A = 0.270 | A = 0.210 | 0.021 | 0.71 |
| | G = 0.730 | G = 0.790 | | |
| rs729647 | A = 0.578 | A = 0.620 | 0.187 | 1.19 |
| | G = 0.422 | G = 0.380 | | |

TABLE 7
Male Genotyping Results

| SNP Reference | M case | M control | p-value | Odd Ratio |
|---|---|---|---|---|
| rs1360457 | C = 0.876 | C = 0.917 | 0.042 | 0.66 |
| | T = 0.124 | T = 0.083 | | |
| rs753050 | A = 0.270 | A = 0.190 | 0.009 | 0.66 |
| | G = 0.730 | G = 0.810 | | |
| rs729647 | A = 0.543 | A = 0.640 | 0.004 | 1.48 |
| | G = 0.457 | G = 0.360 | | |

The single marker alleles set forth in Table 3 were considered validated, since the genotyping data for the females, males or both pools were significantly associated with melanoma, and because the genotyping results agreed with the original allelotyping results. Particularly significant associations with melanoma are indicated by a calculated p-value of less than 0.05 for genotype results, which are set forth in bold text.

The odds ratios were calculated for the alleles at each SNP and the results are shown in Tables 6 and 7. An odds ratio is an unbiased estimate of relative risk which can be obtained from most case-control studies. Relative risk (RR) is an estimate of the likelihood of disease in the exposed group (susceptibility allele or genotype carriers) compared to the unexposed group (not carriers). It can be calculated by the following equation:
RR=I_{A}/Iₐ
I_{A} is the incidence of disease in the A carriers and Iₐ is the incidence of disease in the non-carriers.
RR>1 indicates the A allele increases disease susceptibility.
RR<1 indicates the a allele increases disease susceptibility. For example, RR=1.5 indicates that carriers of the A allele have 1.5 times the risk of disease than non-carriers, i.e., 50% more likely to get the disease.

Case-control studies do not allow the direct estimation of I_{A} and Iₐ, therefore relative risk cannot be directly estimated. However, the odds ratio (OR) can be calculated using the following equation:
OR=(n_{DA}n_{da})/(n_{dA}n_{Da})=p_{DA}(1−p_{dA})/p_{dA}(1−p_{DA}), or
OR=((case f)/(1−case f))/((control f)/(1−control f)), where f=susceptibility allele frequency.

An odds ratio can be interpreted in the same way a relative risk is interpreted and can be directly estimated using the data from case-control studies, i.e., case and control allele frequencies. The higher the odds ratio value, the larger the effect that particular allele has on the development of melanoma, thus possessing that particular allele translates to having a higher risk of developing melanoma.

NID2 Non-synonomous SNP

In addition, a non-synonomous, coding SNP (rs3742536) located at amino acid position 656 (P656S) of NID2 was genotyped and found to be significantly associated with the occurrence of melanoma. This SNP (rs3742536) was genotyped as described in Examples 1 and 2 herein using the following PCR primers, extend primer and termination mix: forward primer—ACGTTGGATGAGACCAACATTCAAGGCCAG; reverse primer—ACGTTGGATGAGTGGTACAGCTCCTTGTAG; extend primer—GGCTGTGAAATTTGCTG; and termination mix-ACT. The genotyping results for female (F) and male (M) cases and controls are shown in Table 8.

TABLE 8
Genotpying Results

| | Chromo- | | | | | | |
|---|---|---|---|---|---|---|---|
| | some | | | | Control | p- | Odds |
| Rs number | Position | Sex | Alleles | Case AF | AF | Value | Ratio |
| rs3742536 | 50497467* | Male | C/T | T = 0.261 | T = 0.192 | 0.0144 | 0.67 |
| | | | | C = 0.739 | C = 0.808 | | |
| | | Female | | T = 0.257 | T = 0.205 | 0.0561 | 0.75 |
| | | | | C = 0.743 | C = 0.795 | | |

*based on NCBI's Build 31

### Example 3

### NRP1 Proximal SNPs

It has been discovered that a polymorphic variation in a gene encoding the receptor neuropilin 1 (NRP1 ) is associated with the occurrence of melanoma.

Seventy-six allelic variants located within or nearby the target gene were identified and subsequently alleotyped in melanoma case and control sample sets as described in Examples 1 and 2. The polymorphic variants are set forth in Table 9. The chromosome position provided in column four of Table 9 is based on Genome “Build 33” of NCBI's GenBank.

TABLE 9

| | | | | | Allele | |
|---|---|---|---|---|---|---|
| dbSNP | | Position in | Chromosome | Allele | Present in | IUPAC |
| rs# | Chromosome | | Position | Variants | | Code |
| 2776925 | 10 | 111 | 33718511 | C/T | G | R |
| 2776924 | 10 | 357 | 33718757 | G/C | G | S |
| 1952983 | 10 | 774 | 33719174 | A/C | G | K |
| 869636 | 10 | 3878 | 33722278 | C/T | C | Y |
| 869637 | 10 | 4044 | 33722444 | C/T | T | Y |
| 2804470 | 10 | 5469 | 33723869 | G/A | A | R |
| 2776928 | 10 | 6244 | 33724644 | T/C | C | Y |
| 999966 | 10 | 6524 | 33724924 | C/T | C | Y |
| 2804475 | 10 | 6526 | 33724926 | C/G | G | S |
| 4934858 | 10 | 10127 | 33728527 | C/T | C | Y |
| 4934863 | 10 | 10917 | 33729317 | C/T | T | Y |
| 4934864 | 10 | 10927 | 33729327 | C/T | T | Y |
| 1331326 | 10 | 11729 | 33730129 | G/A | C | Y |
| 1331325 | 10 | 12008 | 33730408 | A/G | T | Y |
| 4934871 | 10 | 14928 | 33733328 | G/A | G | R |
| 4277057 | 10 | 18529 | 33736929 | G/T | G | K |
| 2776930 | 10 | 19720 | 33738120 | C/T | C | Y |
| 1331324 | 10 | 21304 | 33739704 | G/C | C | S |
| 2776932 | 10 | 24245 | 33742645 | C/T | T | Y |
| 4934896 | 10 | 25448 | 33743848 | A/G | A | R |
| 2776933 | 10 | 26684 | 33745084 | T/C | T | Y |
| 4934901 | 10 | 27885 | 33746285 | A/T | A | W |
| 1015300 | 10 | 29083 | 33747483 | G/C | C | S |
| 2776934 | 10 | 31681 | 33750081 | G/C | C | S |
| 2776935 | 10 | 32137 | 33750537 | G/A | G | R |
| 2776936 | 10 | 32720 | 33751120 | C/T | C | Y |
| 2804491 | 10 | 32726 | 33751126 | A/C | A | M |
| 2804492 | 10 | 33885 | 33752285 | T/C | T | Y |
| 2804493 | 10 | 37515 | 33755915 | A/G | A | R |
| 2776937 | 10 | 38028 | 33756428 | A/G | A | R |
| 2804494 | 10 | 41255 | 33759655 | G/C | G | S |
| 4934914 | 10 | 42818 | 33761218 | G/A | G | R |
| 2776922 | 10 | 43721 | 33762121 | T/C | A | R |
| 2804495 | 10 | 44339 | 33762739 | G/T | G | K |
| 2804496 | 10 | 45640 | 33764040 | T/C | T | Y |
| 4934597 | 10 | 46560 | 33764960 | T/C | C | Y |
| 2804497 | 10 | 47876 | 33766276 | G/T | G | K |
| 1360457 | 10 | 48768 | 33767168 | T/C | A | R |
| 2804498 | 10 | 52546 | 33770946 | T/C | C | Y |
| 2026319 | 10 | 53341 | 33771741 | G/C | C | S |
| 1888684 | 10 | 54816 | 33773216 | T/C | A | R |
| 3750733 | 10 | 55090 | 33773490 | A/G | G | R |
| 4934927 | 10 | 59901 | 33778301 | G/C | G | S |
| 2776946 | 10 | 64404 | 33782804 | T/C | A | R |
| 4934599 | 10 | 65801 | 33784201 | T/C | C | Y |
| 4934934 | 10 | 66740 | 33785140 | A/G | A | R |
| 2776944 | 10 | 66911 | 33785311 | T/G | A | M |
| 2776943 | 10 | 68053 | 33786453 | C/T | G | R |
| 2804886 | 10 | 68779 | 33787179 | G/A | A | R |
| 2804900 | 10 | 69241 | 33787641 | G/A | G | R |
| 2804902 | 10 | 69272 | 33787672 | T/C | C | Y |
| 2768397 | 10 | 70324 | 33788724 | G/C | C | S |
| 2768399 | 10 | 70511 | 33788911 | A/G | A | R |
| 2776941 | 10 | 71786 | 33790186 | C/G | C | S |
| 2804888 | 10 | 73346 | 33791746 | G/A | A | R |
| 2804499 | 10 | 73360 | 33791760 | C/G | G | S |
| 2804889 | 10 | 73703 | 33792103 | G/A | A | R |
| 2804891 | 10 | 74247 | 33792647 | G/A | G | R |
| 2776940 | 10 | 75828 | 33794228 | A/T | T | W |
| 2804897 | 10 | 76381 | 33794781 | C/T | A | R |
| 2804896 | 10 | 76859 | 33795259 | T/C | A | R |
| 2776939 | 10 | 76924 | 33795324 | C/T | G | R |
| 2804894 | 10 | 78930 | 33797330 | T/C | G | R |
| 2804501 | 10 | 80162 | 33798562 | A/T | A | W |
| 2183715 | 10 | 80602 | 33799002 | A/C | T | K |
| 2254822 | 10 | 80671 | 33799071 | G/A | G | R |
| 2150686 | 10 | 80862 | 33799262 | G/A | T | Y |
| 2804893 | 10 | 81378 | 33799778 | T/C | A | R |
| 2804502 | 10 | 81699 | 33800099 | T/C | C | Y |
| 2804892 | 10 | 81918 | 33800318 | T/C | G | R |
| 2183714 | 10 | 84909 | 33803309 | G/A | C | Y |
| 2804503 | 10 | 86555 | 33804955 | C/T | C | Y |
| 1320485 | 10 | 87874 | 33806274 | T/C | A | R |
| 4934603 | 10 | 90482 | 33808882 | A/G | G | R |
| 2804890 | 10 | 90522 | 33808922 | C/T | A | R |
| 1854145 | 10 | 90860 | 33809260 | A/G | C | Y |

Assay for Verifying and Allelotyping SNPs

The methods used to verify and allelotype the seventy-six proximal SNPs of Table 9 are the same methods described in Examples 1 and 2 herein. The primers used in these assays are provided in Table 10 and Table 11, respectively.

TABLE 10

| dbSNP | Forward | Reverse |
|---|---|---|
| rs# | PCR Primer | PCR Primer |
| 2776925 | ACGTTGGATGAGAAATCGAGGTGTTGGCTG | ACGTTGGATGCTGCAAGAACCTGAACTCTG |
| 2776924 | ACGTTGGATGCAAACACGAATCTAGGAGCC | ACGTTGGATGCTCCCTTTCTTGAGGTTGAC |
| 1952983 | GGCGCACGCCTCCACGTGTCTCTAGACGTGTGTTGG | GGCGCACGCCATGGATTGAGCCACACTCTC |
| 869636 | ACGTTGGATGGGTTGCGGTTATCTTAGCAG | ACGTTGGATGGCAGTGGGCACTAACCTAAG |
| 869637 | ACGTTGGATGAACGGACAAGTACTCTAAGC | ACGTTGGATGCCTCTTCTGTAATCTCTAGTG |
| 2804470 | ACGTTGGATGCTCCCTTTTTCTTATTAGTC | ACGTTGGATGGAAAAGAACATTATTTGCAGC |
| 2776928 | ACGTTGGATGGACATCTCATTTTCTAGGTG | ACGTTGGATGGAAATCCTGTGGTAGGAATC |
| 999966 | ACGTTGGATGAGTGGCTCAATCTTGGCTCC | ACGTTGGATGATGCCTGAAATCCCAGCTAC |
| 2804475 | ACGTTGGATGAGTGGCTCAATCTTGGCTCC | ACGTTGGATGATGCCTGAAATCCCAGCTAC |
| 4934858 | ACGTTGGATGCACCTAGGGATTTATAGACG | ACGTTGGATGAGTCTCACCAAGACCTTCTG |
| 4934863 | ACGTTGGATGTGCACATATAGACGTATCAC | ACGTTGGATGGGAATATAAGTCCTGCAGCC |
| 4934864 | ACGTTGGATGGCATACACAAATATATGCAC | ACGTTGGATGTAAGTCCTGCAGCCACTTTG |
| 1331326 | ACGTTGGATGCTACAGTGGCAAAGAACACG | ACGTTGGATGTTTAAACATGGTCTCGTGGC |
| 1331325 | ACGTTGGATGTCTGGGCAGTAAGTTTCAGG | ACGTTGGATGGGATCCTAATTTGCGCTGAC |
| 4934871 | ACGTTGGATGCCAAATAACATGCTGATCGC | ACGTTGGATGCACACTCACTGAAGCGTTTG |
| 4277057 | ACGTTGGATGCCTACTTTTCCTTCAAACAC | ACGTTGGATGTCCTTCCTCTCCTCCTTCTC |
| 2776930 | ACGTTGGATGCCCAGCACAAATAAGTGCTC | ACGTTGGATGAATACTCTCACCACCACCAC |
| 1331324 | ACGTTGGATGTATTTTCCTTTCCCACCAGC | ACGTTGGATGCAGGATTATCTGACAGGAGG |
| 2776932 | ACGTTGGATGTGTTTACCGATGCTCGTACC | ACGTTGGATGCAATCCCAGGGGATTATAGC |
| 4934896 | ACGTTGGATGGAGGAGTCGACTGTTGAATG | ACGTTGGATGGGATGAAATGAGAATGACCC |
| 2776933 | ACGTTGGATGCTCCTGGGTTCAAGAGATTC | ACGTTGGATGATACAAAAATTAGCCGGGCG |
| 4934901 | ACGTTGGATGAGCAGATTTACTCCCTTCCC | ACGTTGGATGGAATTGGGAGGCATGCTTTG |
| 1015300 | ACGTTGGATGCCTGTACGTGTACAGGTTTG | ACGTTGGATGGTGCTTATTACCCGAGTGAC |
| 2776934 | ACGTTGGATGGGGAAGAAAAGACGACCTAC | ACGTTGGATGTGGTTTATGGTCTTGGCGTC |
| 2776935 | ACGTTGGATGTAGAGCATGATCTAGGTCCC | ACGTTGGATGTTTCCAGCTTGCAGAACCTC |
| 2776936 | ACGTTGGATGTCAGCTAATTCCCAGCATTC | ACGTTGGATGGGAGTTGACAGTTCAAATCCT |
| 2804491 | ACGTTGGATGGGAGTTGACAGTTCAAATCCT | ACGTTGGATGTCAGCTAATTCCCAGCATTC |
| 2804492 | ACGTTGGATGTCAGAAGACCACATTCTCAG | ACGTTGGATGCCTAGACCTTAGTCTTACCC |
| 2804493 | ACGTTGGATGTTCTTGGCCCTGATTTCTCC | ACGTTGGATGAAAGAAGAAGGCCCCAACAC |
| 2776937 | ACGTTGGATGGAAAGAGCTTGGGATCATGG | ACGTTGGATGCATAAGGCCATTCATGAGGG |
| 2804494 | ACGTTGGATGGTGGCCAGATTCTCTGAATG | ACGTTGGATGAGCACTTAAGACAGCCACTC |
| 4934914 | ACGTTGGATGCCTTTAACCCTTACAGAGCC | ACGTTGGATGAAGTGTTCTTCTCCCACAGC |
| 2776922 | ACGTTGGATGCATTTGACAGCTGAGATCCC | ACGTTGGATGGACAGAAAGATTTGTGACGC |
| 2804495 | ACGTTGGATGGACACTACTAACTGCCCTAG | ACGTTGGATGAGTTCGTTGCACCTTGAGAG |
| 2804496 | ACGTTGGATGATGTTGGCCAGGCTGGTTTC | ACGTTGGATGTGGCTCACTCGCAAACATTG |
| 4934597 | ACGTTGGATGGTTACAAGTATGCGATCTTCC | ACGTTGGATGCTCTCCTCTGTGTAACCAAC |
| 2804497 | ACGTTGGATGGCAAATGTGCTAAGGCATCG | ACGTTGGATGACACCCTGCATTTTCTCTTG |
| 1360457 | ACGTTGGATGTTGTCCCAACTGAGGCTTTG | ACGTTGGATGAACAGCCTTTCAGCTTTGGC |
| 2804498 | ACGTTGGATGCTGGGCTGCCTATATTGTTC | ACGTTGGATGAATGTGGCAGCATTTCAGGC |
| 2026319 | ACGTTGGATGGAGAGATTGAACAGAGCCAG | ACGTTGGATGCACACCAGAGCTCTTTAATC |
| 1888684 | GGCGCACGCCTCCACGGGGCTTTTCCTTACCTCTGT | GGCGCACGCCAATTGGCATGCTGATCTGGG |
| 3750733 | ACGTTGGATGGCTCCTCTGCGCCGTGCTC | ACGTTGGATGCTGCCTGTCACTTACCGTTG |
| 4934927 | ACGTTGGATGTGGTGGTGGATAAGCCAATG | ACGTTGGATGTACATGAAGGCAATCGCACC |
| 2776946 | ACGTTGGATGAAATGAACTCCTCTTGGCCG | ACGTTGGATGAACTCCTGACCTCAAGTGAC |
| 4934599 | ACGTTGGATGATTACCCAAGTAGGCTTCCC | ACGTTGGATGACAGATATGCTGCAGTGACC |
| 4934934 | ACGTTGGATGAGGAACAGCCACATTTTGAC | ACGTTGGATGGGAAGATCTTTGAGAGTTAGC |
| 2776944 | ACGTTGGATGCATCATTCCCCCAGTTCTAG | ACGTTGGATGTGTGGCATCATTTGTCTTCC |
| 2776943 | ACGTTGGATGAATCTGTTTGCTTACTTTTC | ACGTTGGATGGTCCTCTCTCTGTTTTAGGC |
| 2804886 | ACGTTGGATGAGGTCCTGACCATGCATGAG | ACGTTGGATGACACCCAACTCTCTATGAGC |
| 2804900 | ACGTTGGATGTGCACACATGTAGTCCCAGC | ACGTTGGATGCTCGGCTTACTGTAATCTCC |
| 2804902 | ACGTTGGATGGAGGCGGAGATTACAGTAAG | ACGTTGGATGTTTTCGAGACGGAGTCTTGC |
| 2768397 | ACGTTGGATGGTTCTGTTGCCTTCATGCTC | ACGTTGGATGTTGAAATCCTTTCCACTGGC |
| 2768399 | ACGTTGGATGAGGTCTTCAAGACCTGTTCC | ACGTTGGATGCCCCAAGAATCTTGCACTTC |
| 2776941 | ACGTTGGATGTTTGTCCCCTATCCTCTCTC | ACGTTGGATGCCCATAAAGGAAGCAGAAGC |
| 2804888 | ACGTTGGATGGCACTCTCTGGGATAATTTC | ACGTTGGATGGTGAACAAAAGTAGACTCCC |
| 2804499 | ACGTTGGATGCCCATGAAAGACTTAACCTTC | ACGTTGGATGTAATTTCCTCCATCCCTCCC |
| 2804889 | ACGTTGGATGCAAATAGAACTGACATCTGC | ACGTTGGATGATCTGGTCAGGTTTGGTCTC |
| 2804891 | ACGTTGGATGCCACCCATCATTACAATCGC | ACGTTGGATGGATCATTGGTGGAAACAGGG |
| 2776940 | ACGTTGGATGCTCAGGGTGTATTTTACACG | ACGTTGGATGAACTCCCTCCTTCCCTTAAC |
| 2804897 | ACGTTGGATGCAGTGCTCACTCAGTTAGTC | ACGTTGGATGGCTTCTATTAGGGTGTTGGC |
| 2804896 | ACGTTGGATGAATTAACCCTCGGCCAGGTG | ACGTTGGATGTTCTTGACCTCGTGATCCAC |
| 2776939 | ACGTTGGATGTGGCCGAGGGTTAATTTTTG | ACGTTGGATGGGTGCAGCTAAAGAGAAACG |
| 2804894 | ACGTTGGATGCATGAATCCCTCTGCAACTC | ACGTTGGATGAAGTTTGAGAACCACTGGGC |
| 2804501 | ACGTTGGATGACTTTGCTTCATACCATGGA | ACGTTGGATGGCACTAGTCCAGCATTTTCC |
| 2183715 | ACGTTGGATGTGGACTGGTTTCTTCCTCAC | ACGTTGGATGCAAGATGAGATTTGGGTGGG |
| 2254822 | ACGTTGGATGGGAAGAAACCAGTCCAAGAG | ACGTTGGATGCCTCTTCCAAAAGTAAGCCC |
| 2150686 | ACGTTGGATGAGAGGTGCAATAACTGGTGG | ACGTTGGATGTTCAGTGAAAGTTGCCTGTG |
| 2804893 | ACGTTGGATGTTAGCCAGGTATGGTGATGC | ACGTTGGATGCCAGGTTCAAGCAATTCTCC |
| 2804502 | ACGTTGGATGAGGGGTACACAATTCAATCC | ACGTTGGATGCATTAATATGTGGCCATGGG |
| 2804892 | ACGTTGGATGTGCTTTCTCTCTCTTCTCCC | ACGTTGGATGAAGACCCTCTTCCTGGTTTG |
| 2183714 | ACGTTGGATGTGAAGGATCTTTACTCAGGC | ACGTTGGATGACCACTCATGTCTGCCTGCT |
| 2804503 | ACGTTGGATGGGATGGTTTCACTACTGTATG | ACGTTGGATGTAACATGTCCACAGCCTTCC |
| 1320485 | ACGTTGGATGAAAACTGCCATTCCTCCCAC | ACGTTGGATGCCTTCTTCATTCATCAGGGC |
| 4934603 | ACGTTGGATGATAAAGTGCAGTACCTCCCC | ACGTTGGATGTTTATTCATGGCAGAAGGCG |
| 2804890 | ACGTTGGATGTTCACATGGTGAAAGCAGGG | ACGTTGGATGCTAATTTCACCTGTAGGATG |
| 1854145 | ACGTTGGATGAGTTCCACATAGCTGGAGAG | ACGTTGGATGGCTGGCACTCATTTTCTCTC |

TABLE 11

| dbSNP | Extend | Term |
|---|---|---|
| rs# | Primer | Mix |
| 2776925 | CCAGTGGTGTGAGCTTC | ACG |
| 2776924 | GAGGTTGACTCATGATTT | ACT |
| 1952983 | CCTGATTGAAGCAAAACCAGAA | ACT |
| 869636 | TGTTCAACAGACCCTTC | ACG |
| 869637 | GTAATCTCTAGTGTTTATTGAAAG | ACG |
| 2804470 | TTGCAGCATTTCCCTAA | ACG |
| 2776928 | GTGGATCCTAATGATCAC | ACT |
| 999966 | GGAGAACTGCTTGAACC | ACG |
| 2804475 | AGAACTGCTTGAACCCG | ACT |
| 4934858 | TGGGATTTCGTTACCTAA | ACG |
| 4934863 | CAACCTGAGGATGTATAGAA | ACG |
| 4934864 | GGATGTATAGAATGCCTATATG | ACG |
| 1331326 | TTCCTTGATCTCTTTCTTC | ACG |
| 1331325 | TTGCGCTGACAAAAACC | ACT |
| 4934871 | GAGATATGTAGGACCCC | ACG |
| 4277057 | AGAGGGTAGTAAATCCTG | CGT |
| 2776930 | TGCTATTACTACTTCTACCTAC | ACG |
| 1331324 | GTGTGTGCACATCTCAA | ACT |
| 2776932 | CGTAGTTCTTCGCCCCT | ACG |
| 4934896 | ACCCCATCTAAACAAAAG | ACT |
| 2776933 | TAGCCGGGCGTGGTGGT | ACT |
| 4934901 | TGGGATCATGAATCCTAG | CGT |
| 1015300 | CTAAACCCTTGTGACATG | ACT |
| 2776934 | TTTTACTGACTCTTCAAGTG | ACT |
| 2776935 | TATCTTCATCTGCAGCC | ACG |
| 2776936 | GACAGTTCAAATCCTTTTTTT | ACG |
| 2804491 | TGTCTAGTATTTCTAAACACTG | ACT |
| 2804492 | CTTAGTCTTACCCATGAAC | ACT |
| 2804493 | GGAGTTGCCAATTATGTATAAG | ACT |
| 2776937 | GGTGGGGATAAGGAGCT | ACT |
| 2804494 | GGGTGTTGGAGTGGGTG | ACT |
| 4934914 | CCCTCATTCATACAGTTT | ACG |
| 2776922 | TTTGTGACGCAGCAACT | ACT |
| 2804495 | GGTTGATTGGCCTGTAT | CGT |
| 2804496 | AGGCAGGCATCACTTGA | ACT |
| 4934597 | TTTTTCCTAACTCAGTGC | ACT |
| 2804497 | GTATAGTCAACTTCCACC | CGT |
| 1360457 | CAGCTTTGGCCAGGAGATG | ACT |
| 2804498 | TCCATCATAAAATGTGACAAT | ACT |
| 2026319 | TGACTTCTGAGAAAGTGA | ACT |
| 1888684 | ATGCTGATCTGGGAAGGTAG | ACT |
| 3750733 | TTACCGTTGCGAAAAGC | ACT |
| 4934927 | TGAATCTTTGGTAACACC | ACT |
| 2776946 | GTGCTAGGATTACAGGC | ACT |
| 4934599 | CACAGAGCTCACCCACC | ACT |
| 4934934 | AGGAAAAAGGCTTATCAAG | ACT |
| 2776944 | ACTTGACCTAGCACCAG | ACT |
| 2776943 | GGCTAAAAAGTGTGTCAA | ACG |
| 2804886 | GATGTGCTCAGCTCTTA | ACG |
| 2804900 | CTGTAATCTCCGCCTCC | ACG |
| 2804902 | GGCTGGAGTGCAGTGGC | ACT |
| 2768397 | CTGGCTAGCCTTTTAGAA | ACT |
| 2768399 | ACAAAAACCACCATCAAA | ACT |
| 2776941 | ATGCCAACTGAGAAGGA | ACT |
| 2804888 | CTCCCTTCTGGGAGGAT | ACG |
| 2804499 | CCCAATCCTCCCAGAAG | ACT |
| 2804889 | GGTCTCCAAATGAGTGG | ACG |
| 2804891 | GAAACAGGGATGGAGATA | ACG |
| 2776940 | CAGCAACAGGTCTCACC | CGT |
| 2804897 | GAGAGGAATGGAGAAAAA | ACG |
| 2804896 | GTACTGGGATTACAGGC | ACT |
| 2776939 | AAACGTCAGAGCCAGCC | ACG |
| 2804894 | TCTCACTGGGGACCCAC | ACT |
| 2804501 | GCATTTTCCTCTCTCTG | CGT |
| 2183715 | GATAGAGGTCAAGGAGAG | ACT |
| 2254822 | GTAAGCCCTACCTATGC | ACG |
| 2150686 | GTTGCCTGTGTTCTGGA | ACG |
| 2804893 | GCAATTCTCCTGCCTCA | ACT |
| 2804502 | GTGAATATGGAAAAAACCAA | ACT |
| 2804892 | TGCCATCTTCTCATTGT | ACT |
| 2183714 | TTGTGTAGAATAGAGCCC | ACG |
| 2804503 | TTGTCCTCTGTGCTAGA | ACG |
| 1320485 | TGCTGGTGAGCAGGGAG | ACT |
| 4934603 | GCAGAAGGCGAAGTGGT | ACT |
| 2804890 | GTGCAGTACCTCCCCCC | ACG |
| 1854145 | CTCATTTTCTCTCTATTCCC | ACT |

Genetic Analysis

Allelotyping results are shown for female (F) and male (M) cases and controls in Table 12 and Table 13, respectively. Allele frequency is noted in the fourth and fifth columns for melanoma pools and control pools, respectively.

TABLE 12
Females

| | | | | Female | | | Melanoma |
|---|---|---|---|---|---|---|---|
| dbSNP | Chromsome | | Female | Control | F p- | Odds | Associated |
| rs# | Position | Alleles | Case AF | AF | Value | Ratio | Allele |
| 869636 | 33722278 | C/T | C = 0.442 | C = 0.444 | 0.950 | 1.009 | T |
| | | | T = 0.558 | T = 0.556 | | | |
| 869637 | 33722444 | C/T | C = 0.888 | C = 0.914 | 0.299 | 1.339 | T |
| | | | T = 0.112 | T = 0.086 | | | |
| 999966 | 33724924 | C/T | C = 0.828 | C = 0.828 | 0.995 | 0.999 | C |
| | | | T = 0.172 | T = 0.172 | | | |
| 1015300 | 33747483 | G/C | G = 0.948 | G = 0.960 | 0.682 | 1.307 | C |
| | | | C = 0.052 | C = 0.040 | | | |
| 1320485 | 33806274 | T/C | T = 0.404 | T = 0.393 | 0.766 | 0.957 | T |
| | | | C = 0.596 | C = 0.607 | | | |
| 1331324 | 33739704 | G/C | G = 0.621 | G = 0.612 | 0.786 | 0.961 | G |
| | | | C = 0.379 | C = 0.388 | | | |
| 1331325 | 33730408 | A/G | A = 0.261 | A = 0.189 | 0.019 | 0.661 | A |
| | | | G = 0.739 | G = 0.811 | | | |
| 1331326 | 33730129 | G/A | G = 0.438 | G = 0.466 | 0.462 | 1.119 | A |
| | | | A = 0.562 | A = 0.534 | | | |
| 1360457 | 33767168 | T/C | T = 0.278 | T = 0.211 | 0.036 | 0.693 | T |
| | | | C = 0.722 | C = 0.789 | | | |
| 1854145 | 33809260 | A/G | A = 0.038 | A = 0.023 | 0.571 | 0.598 | A |
| | | | G = 0.962 | G = 0.977 | | | |
| 1888684 | 33773216 | T/C | T = 0.296 | T = 0.229 | 0.070 | 0.707 | T |
| | | | C = 0.704 | C = 0.771 | | | |
| 1952983 | 33719174 | A/C | A = 0.616 | A = 0.553 | 0.103 | 0.771 | A |
| | | | C = 0.384 | C = 0.447 | | | |
| 2026319 | 33771741 | G/C | G = 0.902 | G = 0.909 | 0.778 | 1.084 | C |
| | | | C = 0.098 | C = 0.091 | | | |
| 2150686 | 33799262 | G/A | G = 0.116 | G = 0.127 | 0.674 | 1.107 | A |
| | | | A = 0.884 | A = 0.873 | | | |
| 2183714 | 33803309 | G/A | G = 0.267 | G = 0.186 | 0.021 | 0.627 | G |
| | | | A = 0.733 | A = 0.814 | | | |
| 2183715 | 33799002 | A/C | A = 0.905 | A = 0.908 | 0.899 | 1.038 | C |
| | | | C = 0.095 | C = 0.092 | | | |
| 2254822 | 33799071 | G/A | G = 0.465 | G = | | | |
| | | | A = 0.535 | A = | | | |
| 2768397 | 33788724 | G/C | G = 0.904 | G = 0.935 | 0.254 | 1.514 | C |
| | | | C = 0.096 | C = 0.065 | | | |
| 2768399 | 33788911 | A/G | A = 0.245 | A = 0.203 | 0.211 | 0.782 | A |
| | | | G = 0.755 | G = 0.797 | | | |
| 2776922 | 33762121 | T/C | T = 0.294 | T = 0.205 | 0.008 | 0.621 | T |
| | | | C = 0.706 | C = 0.795 | | | |
| 2776924 | 33718757 | G/C | G = 0.755 | G = 0.724 | 0.349 | 0.850 | G |
| | | | C = 0.245 | C = 0.276 | | | |
| 2776925 | 33718511 | C/T | C = 0.436 | C = 0.453 | 0.632 | 1.072 | T |
| | | | T = 0.564 | T = 0.547 | | | |
| 2776928 | 33724644 | T/C | T = 0.894 | T = 0.921 | 0.253 | 1.387 | C |
| | | | C = 0.106 | C = 0.079 | | | |
| 2776930 | 33738120 | C/T | C = 0.211 | C = 0.159 | 0.076 | 0.707 | C |
| | | | T = 0.789 | T = 0.841 | | | |
| 2776932 | 33742645 | C/T | C = 0.189 | C = 0.228 | 0.217 | 1.264 | T |
| | | | T = 0.811 | T = 0.772 | | | |
| 2776933 | 33745084 | T/C | T = | T = 0.535 | | | |
| | | | C = | C = 0.465 | | | |
| 2776934 | 33750081 | G/C | G = 0.256 | G = 0.293 | 0.262 | 1.204 | C |
| | | | C = 0.744 | C = 0.707 | | | |
| 2776935 | 33750537 | G/A | G = 0.460 | G = 0.380 | 0.022 | 0.719 | G |
| | | | A = 0.540 | A = 0.620 | | | |
| 2776936 | 33751120 | C/T | C = 0.294 | C = 0.214 | 0.012 | 0.653 | C |
| | | | T = 0.706 | T = 0.786 | | | |
| 2776937 | 33756428 | A/G | A = 0.643 | A = 0.599 | 0.289 | 0.829 | A |
| | | | G = 0.357 | G = 0.401 | | | |
| 2776939 | 33795324 | C/T | C = 0.400 | C = 0.360 | 0.252 | 0.842 | C |
| | | | T = 0.600 | T = 0.640 | | | |
| 2776940 | 33794228 | A/T | A = 0.274 | A = 0.198 | 0.017 | 0.654 | A |
| | | | T = 0.726 | T = 0.802 | | | |
| 2776941 | 33790186 | C/G | C = 0.697 | C = 0.732 | 0.310 | 1.182 | G |
| | | | G = 0.303 | G = 0.268 | | | |
| 2776943 | 33786453 | C/T | C = 0.314 | C = 0.260 | 0.104 | 0.766 | C |
| | | | T = 0.686 | T = 0.740 | | | |
| 2776944 | 33785311 | T/G | T = 0.550 | T = 0.520 | 0.474 | 0.887 | T |
| | | | G = 0.450 | G = 0.480 | | | |
| 2776946 | 33782804 | T/C | T = 0.375 | T = 0.329 | 0.219 | 0.818 | T |
| | | | C = 0.625 | C = 0.671 | | | |
| 2804470 | 33723869 | G/A | G = 0.182 | G = 0.240 | 0.097 | 1.420 | A |
| | | | A = 0.818 | A = 0.760 | | | |
| 2804475 | 33724926 | C/G | C = 0.024 | C = 0.059 | 0.178 | 2.554 | G |
| | | | G = 0.976 | G = 0.941 | | | |
| 2804491 | 33751126 | A/C | A = 0.236 | A = 0.178 | 0.056 | 0.698 | A |
| | | | C = 0.764 | C = 0.822 | | | |
| 2804492 | 33752285 | T/C | T = 0.603 | T = 0.628 | 0.520 | 1.108 | C |
| | | | C = 0.397 | C = 0.372 | | | |
| 2804493 | 33755915 | A/G | A = 0.234 | A = 0.210 | 0.447 | 0.872 | A |
| | | | G = 0.766 | G = 0.790 | | | |
| 2804494 | 33759655 | G/C | G = 0.382 | G = 0.311 | 0.061 | 0.733 | G |
| | | | C = 0.618 | C = 0.689 | | | |
| 2804495 | 33762739 | G/T | G = 0.400 | G = 0.323 | 0.043 | 0.716 | G |
| | | | T = 0.600 | T = 0.677 | | | |
| 2804496 | 33764040 | T/C | T = 0.365 | T = 0.251 | 0.002 | 0.583 | T |
| | | | C = 0.635 | C = 0.749 | | | |
| 2804497 | 33766276 | G/T | G = 0.968 | G = 0.972 | 0.862 | 1.122 | T |
| | | | T = 0.032 | T = 0.028 | | | |
| 2804498 | 33770946 | T/C | T = 0.758 | T = 0.771 | 0.673 | 1.078 | C |
| | | | C = 0.242 | C = 0.229 | | | |
| 2804499 | 33791760 | C/G | C = 0.796 | C = 0.836 | 0.222 | 1.301 | G |
| | | | G = 0.204 | G = 0.164 | | | |
| 2804501 | 33798562 | A/T | A = 0.381 | A = 0.374 | 0.846 | 0.972 | A |
| | | | T = 0.619 | T = 0.626 | | | |
| 2804502 | 33800099 | T/C | T = 0.766 | T = 0.815 | 0.104 | 1.346 | C |
| | | | C = 0.234 | C = 0.185 | | | |
| 2804503 | 33804955 | C/T | C = 0.416 | C = 0.376 | 0.272 | 0.845 | C |
| | | | T = 0.584 | T = 0.624 | | | |
| 2804886 | 33787179 | G/A | G = 0.870 | G = 0.909 | 0.129 | 1.491 | A |
| | | | A = 0.130 | A = 0.091 | | | |
| 2804888 | 33791746 | G/A | G = 0.053 | G = 0.073 | 0.388 | 1.395 | A |
| | | | A = 0.947 | A = 0.927 | | | |
| 2804889 | 33792103 | G/A | G = 0.815 | G = 0.859 | 0.124 | 1.381 | A |
| | | | A = 0.185 | A = 0.141 | | | |
| 2804890 | 33808922 | C/T | C = | C = 0.559 | | | |
| | | | T = | T = 0.441 | | | |
| 2804891 | 33792647 | G/A | G = 0.304 | G = 0.233 | 0.027 | 0.696 | G |
| | | | A = 0.696 | A = 0.767 | | | |
| 2804892 | 33800318 | T/C | T = 0.871 | T = 0.911 | 0.113 | 1.517 | C |
| | | | C = 0.129 | C = 0.089 | | | |
| 2804893 | 33799778 | T/C | T = 0.173 | T = 0.132 | 0.144 | 0.729 | T |
| | | | C = 0.827 | C = 0.868 | | | |
| 2804894 | 33797330 | T/C | T = 0.748 | T = 0.797 | 0.109 | 1.328 | C |
| | | | C = 0.252 | C = 0.203 | | | |
| 2804896 | 33795259 | T/C | T = 0.540 | T = 0.496 | 0.241 | 0.837 | T |
| | | | C = 0.460 | C = 0.504 | | | |
| 2804897 | 33794781 | C/T | C = 0.843 | C = 0.900 | 0.028 | 1.691 | T |
| | | | T = 0.157 | T = 0.100 | | | |
| 2804900 | 33787641 | G/A | G = 0.754 | G = 0.739 | 0.644 | 0.925 | G |
| | | | A = 0.246 | A = 0.261 | | | |
| 2804902 | 33787672 | T/C | T = 0.693 | T = 0.681 | 0.721 | 0.946 | T |
| | | | C = 0.307 | C = 0.319 | | | |
| 3750733 | 33773490 | A/G | A = 0.177 | A = | | | |
| | | | G = 0.823 | G = | | | |
| 4277057 | 33736929 | G/T | G = 0.868 | G = 0.861 | 0.784 | 0.939 | G |
| | | | T = 0.132 | T = 0.139 | | | |
| 4934597 | 33764960 | T/C | T = 0.224 | T = 0.197 | 0.386 | 0.849 | T |
| | | | C = 0.776 | C = 0.803 | | | |
| 4934599 | 33784201 | T/C | T = 0.088 | T = 0.075 | 0.563 | 0.837 | T |
| | | | C = 0.912 | C = 0.925 | | | |
| 4934603 | 33808882 | A/G | A = 0.586 | A = 0.643 | 0.141 | 1.273 | G |
| | | | G = 0.414 | G = 0.357 | | | |
| 4934858 | 33728527 | C/T | C = 0.506 | C = 0.526 | 0.578 | 1.085 | T |
| | | | T = 0.494 | T = 0.474 | | | |
| 4934863 | 33729317 | C/T | C = 0.191 | C = 0.189 | 0.970 | 0.992 | C |
| | | | T = 0.809 | T = 0.811 | | | |
| 4934864 | 33729327 | C/T | C = 0.536 | C = 0.508 | 0.457 | 0.893 | C |
| | | | T = 0.464 | T = 0.492 | | | |
| 4934871 | 33733328 | G/A | G = 0.769 | G = 0.710 | 0.061 | 0.735 | G |
| | | | A = 0.231 | A = 0.290 | | | |
| 4934896 | 33743848 | A/G | A = 0.861 | A = 0.830 | 0.269 | 0.787 | A |
| | | | G = 0.139 | G = 0.170 | | | |
| 4934901 | 33746285 | A/T | A = 0.837 | A = 0.804 | 0.246 | 0.798 | A |
| | | | T = 0.163 | T = 0.196 | | | |
| 4934914 | 33761218 | G/A | G = 0.920 | G = 0.927 | 0.771 | 1.097 | A |
| | | | A = 0.080 | A = 0.073 | | | |
| 4934927 | 33778301 | G/C | G = 0.896 | G = 0.907 | 0.664 | 1.127 | C |
| | | | C = 0.104 | C = 0.093 | | | |
| 4934934 | 33785140 | A/G | A = 0.918 | A = 0.915 | 0.913 | 0.968 | A |
| | | | G = 0.082 | G = 0.085 | | | |

TABLE 13
Males

| | | | | Male | | | Melanoma |
|---|---|---|---|---|---|---|---|
| dbSNP | Chromsome | | Male | Control | M p- | Odds | Associated |
| rs# | Position | Alleles | Case AF | AF | Value | Ratio | Allele |
| 869636 | 33722278 | C/T | C = 0.428 | C = 0.441 | 0.738 | 1.053 | T |
| | | | T = 0.572 | T = 0.559 | | | |
| 869637 | 33722444 | C/T | C = 0.948 | C = 0.942 | 0.807 | 0.886 | C |
| | | | T = 0.052 | T = 0.058 | | | |
| 999966 | 33724924 | C/T | C = 0.822 | C = 0.828 | 0.855 | 1.040 | T |
| | | | T = 0.178 | T = 0.172 | | | |
| 1015300 | 33747483 | G/C | G = 0.972 | G = 0.937 | 0.170 | 0.426 | G |
| | | | C = 0.028 | C = 0.063 | | | |
| 1320485 | 33806274 | T/C | T = 0.412 | T = 0.352 | 0.144 | 0.775 | T |
| | | | C = 0.588 | C = 0.648 | | | |
| 1331324 | 33739704 | G/C | G = 0.632 | G = 0.573 | 0.111 | 0.782 | G |
| | | | C = 0.368 | C = 0.427 | | | |
| 1331325 | 33730408 | A/G | A = 0.206 | A = 0.149 | 0.062 | 0.679 | A |
| | | | G = 0.794 | G = 0.851 | | | |
| 1331326 | 33730129 | G/A | G = 0.442 | G = 0.420 | 0.606 | 0.916 | G |
| | | | A = 0.558 | A = 0.580 | | | |
| 1360457 | 33767168 | T/C | T = 0.264 | T = 0.205 | 0.110 | 0.717 | T |
| | | | C = 0.736 | C = 0.795 | | | |
| 1854145 | 33809260 | A/G | A = | A = 0.067 | | | |
| | | | G = | G = 0.933 | | | |
| 1888684 | 33773216 | T/C | T = 0.246 | T = 0.161 | 0.053 | 0.590 | T |
| | | | C = 0.754 | C = 0.839 | | | |
| 1952983 | 33719174 | A/C | A = 0.614 | A = 0.589 | 0.516 | 0.902 | A |
| | | | C = 0.386 | C = 0.411 | | | |
| 2026319 | 33771741 | G/C | G = 0.920 | G = 0.952 | 0.344 | 1.717 | C |
| | | | C = 0.080 | C = 0.048 | | | |
| 2150686 | 33799262 | G/A | G = 0.106 | G = 0.112 | 0.829 | 1.060 | A |
| | | | A = 0.894 | A = 0.888 | | | |
| 2183714 | 33803309 | G/A | G = 0.219 | G = 0.188 | 0.344 | 0.826 | G |
| | | | A = 0.781 | A = 0.812 | | | |
| 2183715 | 33799002 | A/C | A = 0.922 | A = 0.926 | 0.866 | 1.058 | C |
| | | | C = 0.078 | C = 0.074 | | | |
| 2254822 | 33799071 | G/A | G = 0.421 | G = 0.420 | 0.980 | 0.996 | G |
| | | | A = 0.579 | A = 0.580 | | | |
| 2768397 | 33788724 | G/C | G = 0.951 | G = 0.946 | 0.798 | 0.891 | G |
| | | | C = 0.049 | C = 0.054 | | | |
| 2768399 | 33788911 | A/G | A = 0.200 | A = 0.155 | 0.150 | 0.730 | A |
| | | | G = 0.800 | G = 0.845 | | | |
| 2776922 | 33762121 | T/C | T = 0.265 | T = 0.223 | 0.203 | 0.796 | T |
| | | | C = 0.735 | C = 0.777 | | | |
| 2776924 | 33718757 | G/C | G = 0.751 | G = 0.727 | 0.503 | 0.881 | G |
| | | | C = 0.249 | C = 0.273 | | | |
| 2776925 | 33718511 | C/T | C = 0.440 | C = 0.459 | 0.604 | 1.080 | T |
| | | | T = 0.560 | T = 0.541 | | | |
| 2776928 | 33724644 | T/C | T = 0.919 | T = 0.936 | 0.445 | 1.298 | C |
| | | | C = 0.081 | C = 0.064 | | | |
| 2776930 | 33738120 | C/T | C = 0.226 | C = 0.170 | 0.081 | 0.702 | C |
| | | | T = 0.774 | T = 0.830 | | | |
| 2776932 | 33742645 | C/T | C = 0.207 | C = 0.175 | 0.369 | 0.816 | C |
| | | | T = 0.793 | T = 0.825 | | | |
| 2776933 | 33745084 | T/C | T = | T = 0.495 | | | |
| | | | C = | C = 0.505 | | | |
| 2776934 | 33750081 | G/C | G = 0.256 | G = 0.219 | 0.262 | 0.817 | G |
| | | | C = 0.744 | C = 0.781 | | | |
| 2776935 | 33750537 | G/A | G = 0.482 | G = 0.388 | 0.012 | 0.680 | G |
| | | | A = 0.518 | A = 0.612 | | | |
| 2776936 | 33751120 | C/T | C = 0.284 | C = | | | |
| | | | T = 0.716 | T = | | | |
| 2776937 | 33756428 | A/G | A = 0.622 | A = 0.582 | 0.341 | 0.848 | A |
| | | | G = 0.378 | G = 0.418 | | | |
| 2776939 | 33795324 | C/T | C = 0.370 | C = 0.350 | 0.566 | 0.914 | C |
| | | | T = 0.630 | T = 0.650 | | | |
| 2776940 | 33794228 | A/T | A = 0.235 | A = 0.196 | 0.210 | 0.793 | A |
| | | | T = 0.765 | T = 0.804 | | | |
| 2776941 | 33790186 | C/G | C = 0.739 | C = 0.732 | 0.850 | 0.964 | C |
| | | | G = 0.261 | G = 0.268 | | | |
| 2776943 | 33786453 | C/T | C = 0.266 | C = 0.236 | 0.354 | 0.850 | C |
| | | | T = 0.734 | T = 0.764 | | | |
| 2776944 | 33785311 | T/G | T = 0.542 | T = | | | |
| | | | G = 0.458 | G = | | | |
| 2776946 | 33782804 | T/C | T = 0.352 | T = 0.316 | 0.361 | 0.853 | T |
| | | | C = 0.648 | C = 0.684 | | | |
| 2804470 | 33723869 | G/A | G = 0.189 | G = 0.167 | 0.470 | 0.859 | G |
| | | | A = 0.811 | A = 0.833 | | | |
| 2804475 | 33724926 | C/G | C = 0.053 | C = | | | |
| | | | G = 0.947 | G = | | | |
| 2804491 | 33751126 | A/C | A = 0.225 | A = 0.168 | 0.073 | 0.695 | A |
| | | | C = 0.775 | C = 0.832 | | | |
| 2804492 | 33752285 | T/C | T = 0.635 | T = 0.587 | 0.218 | 0.818 | T |
| | | | C = 0.365 | C = 0.413 | | | |
| 2804493 | 33755915 | A/G | A = 0.212 | A = 0.210 | 0.944 | 0.986 | A |
| | | | G = 0.788 | G = 0.790 | | | |
| 2804494 | 33759655 | G/C | G = 0.364 | G = 0.315 | 0.176 | 0.803 | G |
| | | | C = 0.636 | C = 0.685 | | | |
| 2804495 | 33762739 | G/T | G = 0.351 | G = 0.323 | 0.426 | 0.882 | G |
| | | | T = 0.649 | T = 0.677 | | | |
| 2804496 | 33764040 | T/C | T = 0.332 | T = 0.212 | 0.002 | 0.541 | T |
| | | | C = 0.668 | C = 0.788 | | | |
| 2804497 | 33766276 | G/T | G = 0.977 | G = | | | |
| | | | T = 0.023 | T = | | | |
| 2804498 | 33770946 | T/C | T = 0.768 | T = 0.777 | 0.770 | 1.055 | C |
| | | | C = 0.232 | C = 0.223 | | | |
| 2804499 | 33791760 | C/G | C = 0.856 | C = 0.856 | 1.000 | 1.000 | C |
| | | | G = 0.144 | G = 0.144 | | | |
| 2804501 | 33798562 | A/T | A = 0.358 | A = 0.374 | 0.653 | 1.071 | T |
| | | | T = 0.642 | T = 0.626 | | | |
| 2804502 | 33800099 | T/C | T = 0.799 | T = 0.811 | 0.711 | 1.078 | C |
| | | | C = 0.201 | C = 0.189 | | | |
| 2804503 | 33804955 | C/T | C = 0.386 | C = 0.380 | 0.883 | 0.978 | C |
| | | | T = 0.614 | T = 0.620 | | | |
| 2804886 | 33787179 | G/A | G = 0.905 | G = 0.934 | 0.251 | 1.486 | A |
| | | | A = 0.095 | A = 0.066 | | | |
| 2804888 | 33791746 | G/A | G = 0.031 | G = 0.075 | 0.074 | 2.530 | A |
| | | | A = 0.969 | A = 0.925 | | | |
| 2804889 | 33792103 | G/A | G = 0.855 | G = 0.879 | 0.382 | 1.237 | A |
| | | | A = 0.145 | A = 0.121 | | | |
| 2804890 | 33808922 | C/T | C = 0.476 | C = | | | |
| | | | T = 0.524 | T = | | | |
| 2804891 | 33792647 | G/A | G = 0.283 | G = 0.241 | 0.203 | 0.804 | G |
| | | | A = 0.717 | A = 0.759 | | | |
| 2804892 | 33800318 | T/C | T = 0.904 | T = 0.932 | 0.255 | 1.464 | C |
| | | | C = 0.096 | C = 0.068 | | | |
| 2804893 | 33799778 | T/C | T = 0.145 | T = 0.127 | 0.515 | 0.857 | T |
| | | | C = 0.855 | C = 0.873 | | | |
| 2804894 | 33797330 | T/C | T = 0.779 | T = 0.816 | 0.247 | 1.258 | C |
| | | | C = 0.221 | C = 0.184 | | | |
| 2804896 | 33795259 | T/C | T = 0.454 | T = 0.525 | 0.073 | 1.333 | C |
| | | | C = 0.546 | C = 0.475 | | | |
| 2804897 | 33794781 | C/T | C = 0.893 | C = 0.924 | 0.233 | 1.464 | T |
| | | | T = 0.107 | T = 0.076 | | | |
| 2804900 | 33787641 | G/A | G = 0.746 | G = 0.738 | 0.810 | 0.957 | G |
| | | | A = 0.254 | A = 0.262 | | | |
| 2804902 | 33787672 | T/C | T = 0.710 | T = 0.678 | 0.368 | 0.859 | T |
| | | | C = 0.290 | C = 0.322 | | | |
| 3750733 | 33773490 | A/G | A = | A = | | | |
| | | | G = | G = | | | |
| 4277057 | 33736929 | G/T | G = 0.843 | G = 0.856 | 0.666 | 1.104 | T |
| | | | T = 0.157 | T = 0.144 | | | |
| 4934597 | 33764960 | T/C | T = 0.192 | T = 0.161 | 0.293 | 0.803 | T |
| | | | C = 0.808 | C = 0.839 | | | |
| 4934599 | 33784201 | T/C | T = 0.084 | T = 0.061 | 0.317 | 0.708 | T |
| | | | C = 0.916 | C = 0.939 | | | |
| 4934603 | 33808882 | A/G | A = 0.591 | A = 0.620 | 0.453 | 1.130 | G |
| | | | G = 0.409 | G = 0.380 | | | |
| 4934858 | 33728527 | C/T | C = 0.492 | C = | | | |
| | | | T = 0.508 | T = | | | |
| 4934863 | 33729317 | C/T | C = 0.204 | C = 0.236 | 0.344 | 1.208 | T |
| | | | T = 0.796 | T = 0.764 | | | |
| 4934864 | 33729327 | C/T | C = 0.573 | C = 0.520 | 0.193 | 0.810 | C |
| | | | T = 0.427 | T = 0.480 | | | |
| 4934871 | 33733328 | G/A | G = 0.751 | G = 0.755 | 0.928 | 1.018 | A |
| | | | A = 0.249 | A = 0.245 | | | |
| 4934896 | 33743848 | A/G | A = 0.842 | A = 0.827 | 0.619 | 0.896 | A |
| | | | G = 0.158 | G = 0.173 | | | |
| 4934901 | 33746285 | A/T | A = 0.812 | A = 0.827 | 0.703 | 1.106 | T |
| | | | T = 0.188 | T = 0.173 | | | |
| 4934914 | 33761218 | G/A | G = 0.930 | G = 0.950 | 0.379 | 1.410 | A |
| | | | A = 0.070 | A = 0.050 | | | |
| 4934927 | 33778301 | G/C | G = 0.908 | G = 0.936 | 0.262 | 1.479 | C |
| | | | C = 0.092 | C = 0.064 | | | |
| 4934934 | 33785140 | A/G | A = 0.924 | A = 0.935 | 0.623 | 1.191 | G |
| | | | G = 0.076 | G = 0.065 | | | |

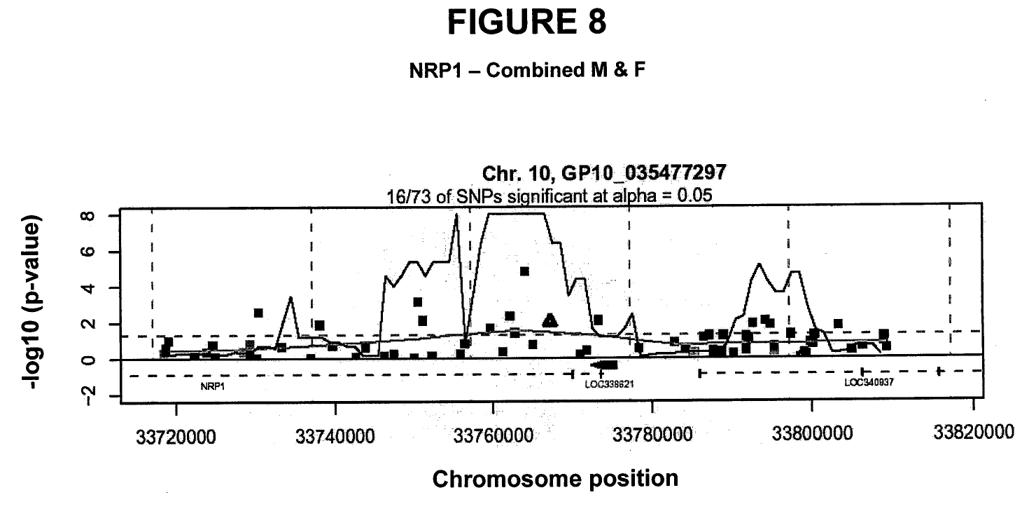

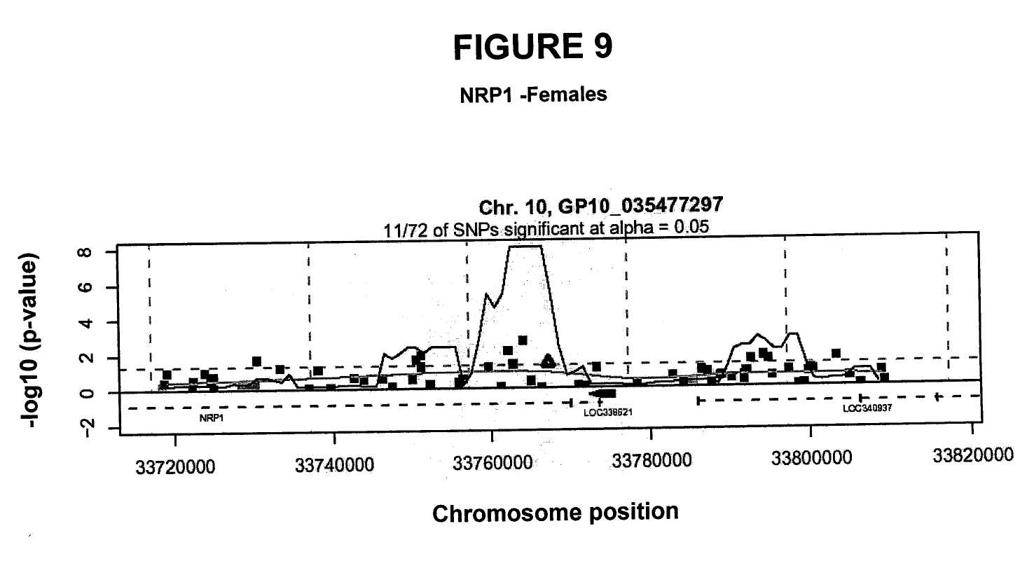

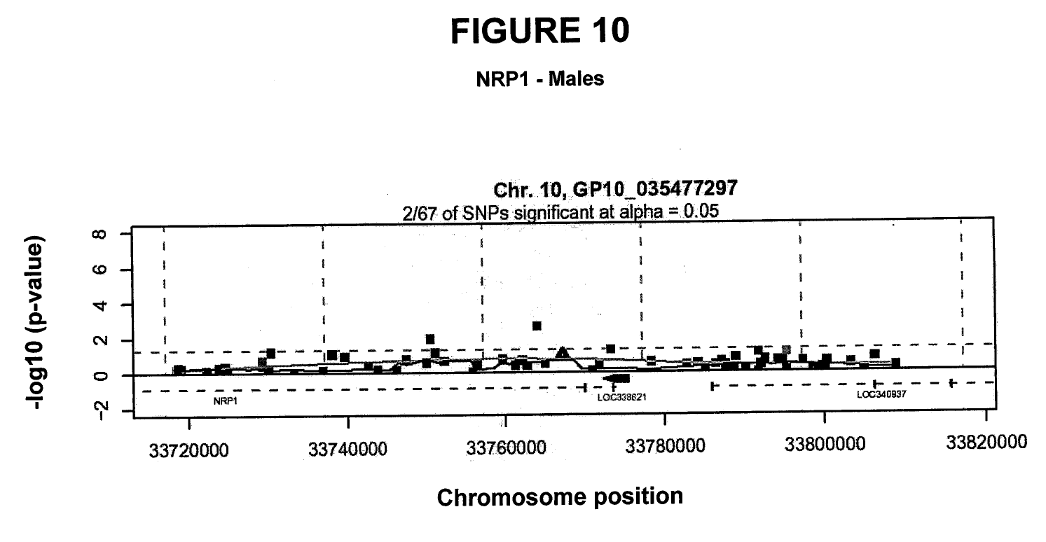

Allelotyping results were considered particularly significant with a calculated p-value of less than or equal to 0.05 for allelotype results. These values are indicated in bold. The assay failed for those SNPs in which the allele frequency is blank. The combined allelotyping p-values for males and females were plotted in FIG. 8 and separately for females and males in FIGS. 9 and 10, respectively. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIGS. 8,9 and 10 can be determined by consulting Table 12 or 13. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line (or generally top-most curve, e.g., see peak in left-most graph just to the left of position 92150000) provides a local test for excess statistical significance to identify regions of association. This was created by use of a 10 kb sliding window with 11 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is placed at the 3′ end of each gene to show the direction of transcription.

### Example 4

### Inhibition of NRP1 Gene Expression by Transfection of Specific siRNAs

RNAi-based gene inhibition was selected as an effective way to inhibit expression of NRP1 in cultured cells. Algorithms useful for designing siRNA molecules specific for the NRP1 targets are disclosed at the http address www.dhramacon.com. siRNAs were selected from this list for use in RNAi experiments following a filtering protocol that involved the removal of any siRNA with complementarity to common motifs or domains present in any target as well as siRNAs complementary to sequences containing SNPs. From this filtered set of siRNAs, four were selected that showed no off-target homology following BLAST analysis against various Genbank nucleotide databases. Table 14 summarize the features of the duplexes that were ordered from Dharmacon Research, Inc., and subsequently used as a cocktail in the assays described herein to inhibit expression of NRP1, NID2 and ENDO180, respectively. A non-homologous siRNA reagent was used as a negative control.

TABLE 14
NRP1 siRNAs used for cell transfection

| | | | |
|---|---|---|---|
| | siRNA | Sequence | SEQ ID |
| siRNA | Target | Specificity | NO: |

| | | |
|---|---|---|
| NRP1_425 | NRP1 | AGAGAGGTCCTGAATGTTC |
| NRP1_1282 | NRP1 | GATTATCCTTGCTCTGGAA |
| NRP1_1910 | NRP1 | GCACCATACAATCAGAGTT |
| NRP1_2538 | NRP1 | GCCAGGCAATGTGTTGAAG |

Two melanoma cell lines (M14 and A375) and a breast cancer cell line (MCF7) were selected for RNAi experiments. On day 1, cells were transfected with siRNA cocktails (18.75 nM) using LIPOFECTAMINE 2000 (LF2000™) Reagent. On days 1, 3 and 6, cellular proliferation was measured using the WST-1 assay (Roche, catalog #1 644 807). Briefly, the WST-1 assay is a colorimetric assay used to determine cellular proliferation by measuring the cleavage of WST-1 by mitochondrial dehydrogenases in living cells. By measuring absorbance, a highly accurate measure of cellular proliferation is obtained. On day 1, WST-1 reagent was added to each well and allowed to incubate for 3-4 h. Subsequently, absorbance was measured at 450 nm and 620 nm using a Tecan Ultra plate reader. This process was repeated on day's 3 and 6. The extent of proliferation on days 3 and 6 were calculated relative to day 1, based on absorbance readings for each sample on each day. From the triplicate repeats of each time point, means and standard deviations were calculated and the effect of siRNA inhibition of each target on cellular proliferation was assessed and compared to cells transfected with a positive control siRNA (siRAD21_{—}1175) and a negative control siRNA (siLuciferase GL2). All experiments were performed in duplicate.

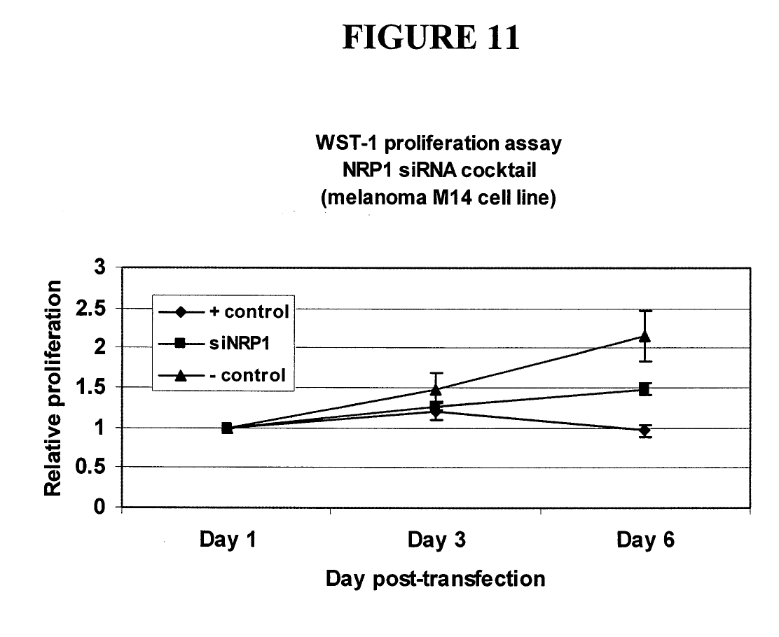

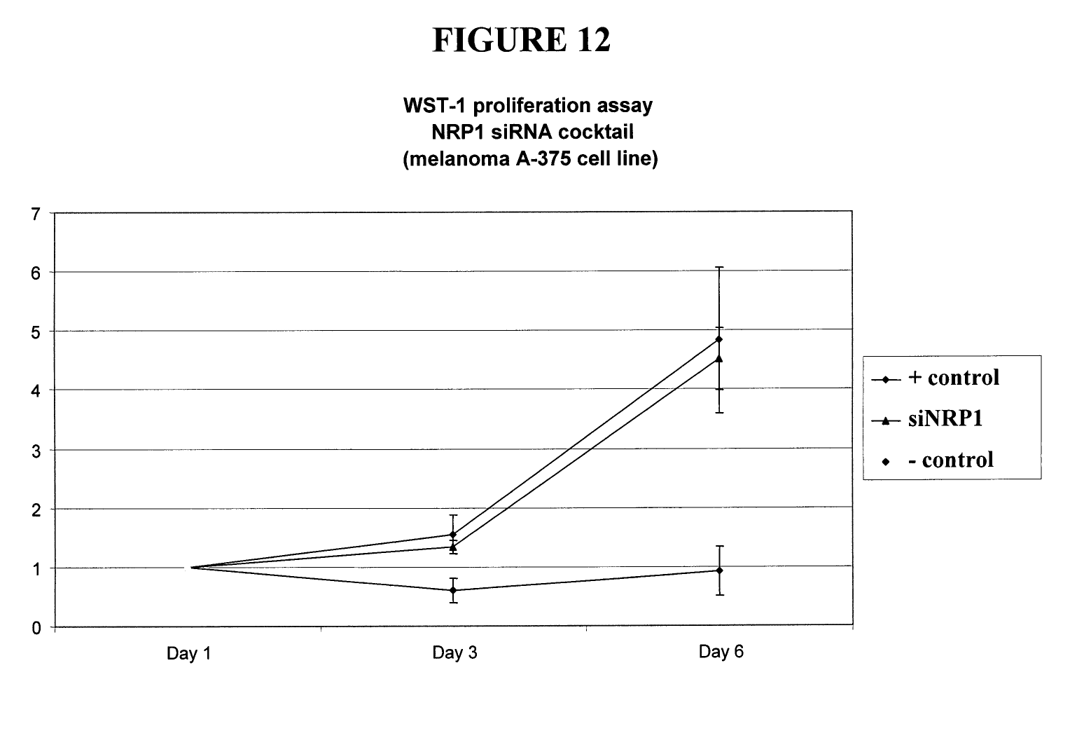

The difference in absorbance between these 2 wavelengths is an indication of the metabolic activity in each well that was measured. Metabolic activity is directly proportional to the number of cells in each well. Suppression of target mRNA levels correlated with decreased cell proliferation. The NRP1 siRNA suppressed proliferation of melanoma M14 cells and A375 cell (see FIGS. 11 and 12 respectively).

### Example 5

### Screening Assay to Detect Modulators of NRP1

The following is an exemplary assay for finding modulators of NRP1 . Plasma membranes are prepared from cells expressing NRP1 by standard methods. The membranes are immobilized on wheat germ agglutinin-coated scintillation proximity beads (Amersham). Test samples and radiolabeled ligand, which is ¹²⁵I-semaphorin-3, are added, and bead-bound radioactivity is measured. The ability of a given sample to modulate binding activity is determined by comparison of bead-bound radioactivity in the presence of the compound with values obtained in solvent controls. The test compound optimally produces a result that differs by at least 3 standard deviations from a set of at least 30 replicate control samples.

### Example 6

### Screening Assay to Detect Modulators of NID2

The following is an exemplary assay for finding modulators of NID2. Plasma membranes as prepared are prepared from cells expressing integrin alpha-3-beta-1 or integrin alpha-6-beta-1 by standard methods. The membranes are immobilized on wheat germ agglutinin-coated scintillation proximity beads (Amersham). Test sample and radiolabeled ligand, which is ¹²⁵I-NID2, are added, and bead-bound radioactivity is measured. The ability of a given sample to modulate binding activity is determined by comparison of bead-bound radioactivity in the presence of the compound with values obtained in solvent controls. The test compound optimally produces a result that differs by at least 3 standard deviations from a set of at least 30 replicate control samples.

### Example 7

### Screening Assay to Detect Modulators of ENDO180

The following is an exemplary assay for finding modulators of ENDO180. Plasma membranes are prepared from cells expressing ENDO180 by standard methods. The membranes are immobilized on wheat germ agglutinin-coated scintillation proximity beads (Amersham). Test sample and radiolabeled ligand, which is ³H-mannose are added and bead-bound radioactivity is measured. The ability of a given sample to modulate binding activity is determined by comparison of bead-bound radioactivity in the presence of the compound with values obtained in solvent controls. The test compound optimally produces a result that differs by at least 3 standard deviations from a set of at least 30 replicate control samples.

### Example 8

### In Vitro Production of Target Polypeptides

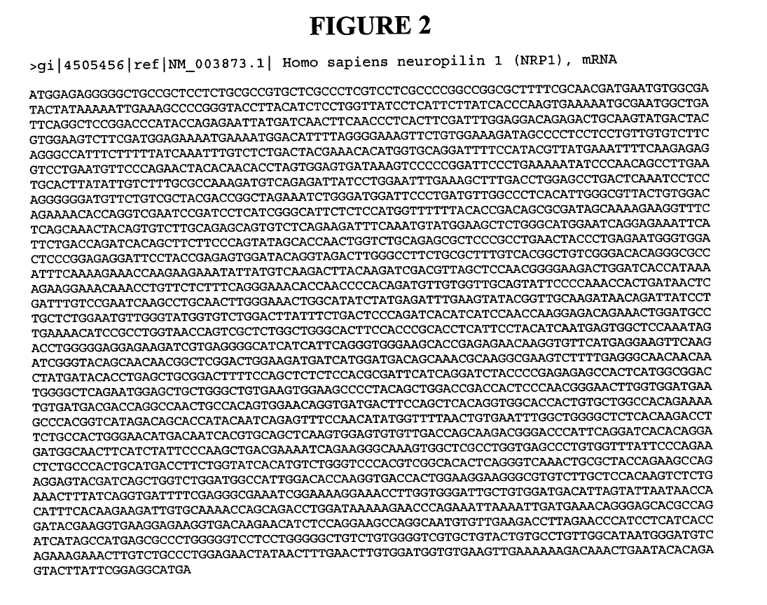

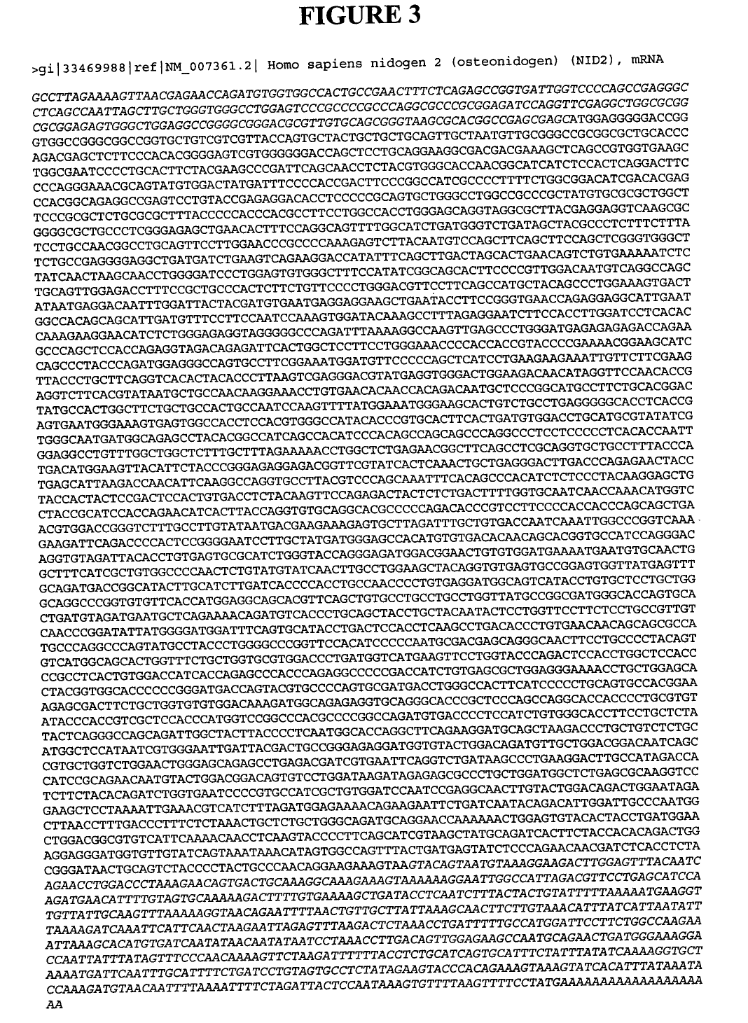

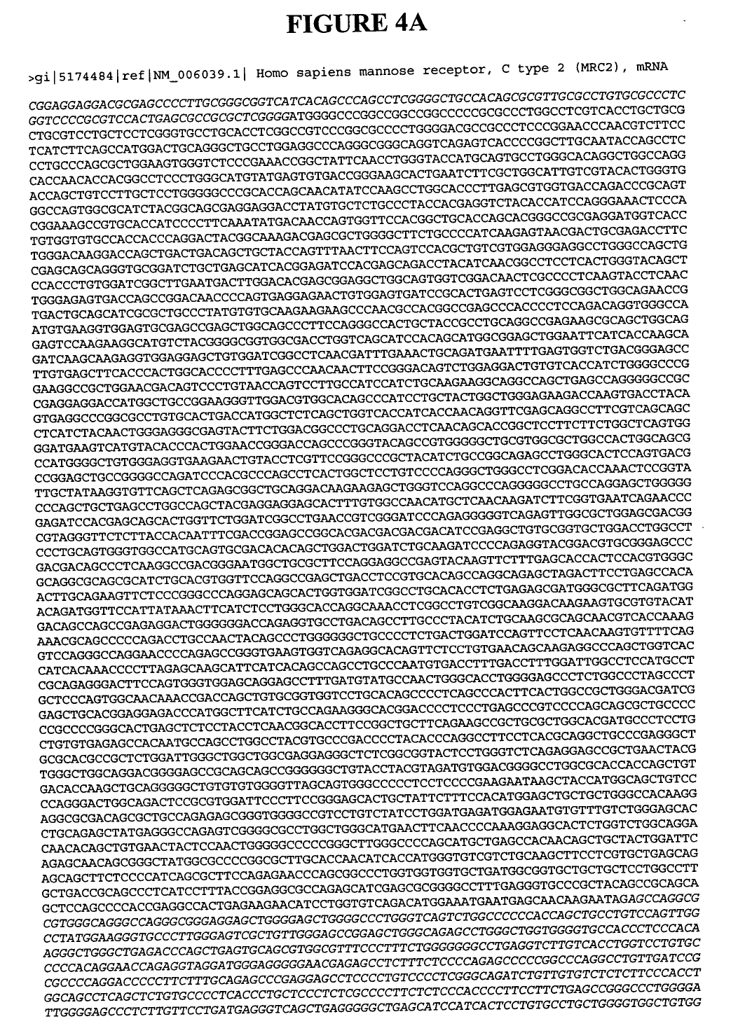

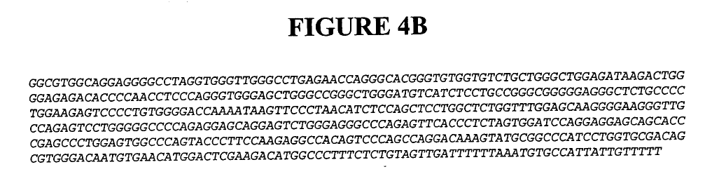

Target polypeptides encoded by the polynucleotides provided in FIGS. 1, 2, 3 or 4 may be produced by the methods described herein. cDNA is cloned into a pIVEX 2.3-MCS vector (Roche Biochem) using a directional cloning method. A cDNA insert is prepared using PCR with forward and reverse primers having 5′ restriction site tags (in frame) and 5-6 additional nucleotides in addition to 3′ gene-specific portions, the latter of which is typically about twenty to about twenty-five base pairs in length. A Sal I restriction site is introduced by the forward primer and a Sma I restriction site is introduced by the reverse primer. The ends of PCR products are cut with the corresponding restriction enzymes (i.e., Sal I and Sma I) and the products are gel-purified. The pIVEX 2.3-MCS vector is linearized using the same restriction enzymes, and the fragment with the correct sized fragment is isolated by gel-purification. Purified PCR product is ligated into the linearized pIVEX 2.3-MCS vector and E. coli cells transformed for plasmid amplification. The newly constructed expression vector is verified by restriction mapping and used for protein production.

E. coli lysate is reconstituted with 0.25 ml of Reconstitution Buffer, the Reaction Mix is reconstituted with 0.8 ml of Reconstitution Buffer; the Feeding Mix is reconstituted with 10.5 ml of Reconstitution Buffer; and the Energy Mix is reconstituted with 0.6 ml of Reconstitution Buffer. 0.5 ml of the Energy Mix was added to the Feeding Mix to obtain the Feeding Solution. 0.75 ml of Reaction Mix, 50 μl of Energy Mix, and 10 μg of the template DNA is added to the E. coli lysate.

Using the reaction device (Roche Biochem), 1 ml of the Reaction Solution is loaded into the reaction compartment. The reaction device is turned upside-down and 10 ml of the Feeding Solution is loaded into the feeding compartment. All lids are closed and the reaction device is loaded into the RTS500 instrument. The instrument is run at 30° C. for 24 hours with a stir bar speed of 150 rpm. The pIVEX 2.3 MCS vector includes a nucleotide sequence that encodes six consecutive histidine amino acids on the C-terminal end of the target polypeptide for the purpose of protein purification. target polypeptide is purified by contacting the contents of reaction device with resin modified with Ni²⁺ ions. target polypeptide is eluted from the resin with a solution containing free Ni²⁺ ions.

### Example 9

### Cellular Production of Target Polypeptides

Nucleic acids are cloned into DNA plasmids having phage recombination cites and target polypeptides are expressed therefrom in a variety of host cells. Alpha phage genomic DNA contains short sequences known as attP sites, and E. coli genomic DNA contains unique, short sequences known as attB sites. These regions share homology, allowing for integration of phage DNA into E. coli via directional, site-specific recombination using the phage protein Int and the E. coli protein IHF. Integration produces two new att sites, L and R, which flank the inserted prophage DNA. Phage excision from E. coli genomic DNA can also be accomplished using these two proteins with the addition of a second phage protein, Xis. DNA vectors have been produced where the integration/excision process is modified to allow for the directional integration or excision of a target DNA fragment into a backbone vector in a rapid in vitro reaction (Gateway™ Technology (Invitrogen, Inc.)).

A first step is to transfer the nucleic acid insert into a shuttle vector that contains attL sites surrounding the negative selection gene, ccdB (e.g. pENTER vector, Invitrogen, Inc.). This transfer process is accomplished by digesting the nucleic acid from a DNA vector used for sequencing, and to ligate it into the multicloning site of the shuttle vector, which will place it between the two attL sites while removing the negative selection gene ccdB. A second method is to amplify the nucleic acid by the polymerase chain reaction (PCR) with primers containing attB sites. The amplified fragment then is integrated into the shuttle vector using Int and IHF. A third method is to utilize a topoisomerase-mediated process, in which the nucleic acid is amplified via PCR using gene-specific primers with the 5′ upstream primer containing an additional CACC sequence (e.g., TOPO® expression kit (Invitrogen, Inc.)). In conjunction with Topoisomerase I, the PCR amplified fragment can be cloned into the shuttle vector via the attL sites in the correct orientation.

Once the nucleic acid is transferred into the shuttle vector, it can be cloned into an expression vector having attR sites. Several vectors containing attR sites for expression of target polypeptide as a native polypeptide, N-fusion polypeptide, and C-fusion polypeptides are commercially available (e.g., pDEST (Invitrogen, Inc.)), and any vector can be converted into an expression vector for receiving a nucleic acid from the shuttle vector by introducing an insert having an attR site flanked by an antibiotic resistant gene for selection using the standard methods described above. Transfer of the nucleic acid from the shuttle vector is accomplished by directional recombination using Int, IHF, and Xis (LR clonase). Then the desired sequence can be transferred to an expression vector by carrying out a one hour incubation at room temperature with Int, IHF, and Xis, a ten minute incubation at 37° C. with proteinase K, transforming bacteria and allowing expression for one hour, and then plating on selective media. Generally, 90% cloning efficiency is achieved by this method. Examples of expression vectors are pDEST 14 bacterial expression vector with att7 promoter, pDEST 15 bacterial expression vector with a T7 promoter and a N-terminal GST tag, pDEST 17 bacterial vector with a T7 promoter and a N-terminal polyhistidine affinity tag, and pDEST 12.2 mammalian expression vector with a CMV promoter and neo resistance gene. These expression vectors or others like them are transformed or transfected into cells for expression of the target polypeptide or polypeptide variants. These expression vectors are often transfected, for example, into murine-transformed cell lines (e.g., adipocyte cell line 3T3-L1 (ATCC), human embryonic kidney cell line 293, and rat cardiomyocyte cell line H9C2).

Modifications may be made to the foregoing without departing from the basic aspects of the invention. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. Also, citation of the above publications or documents is not intended as an admission that any of the foregoing is pertinent prior art, nor does it constitute any admission as to the contents or date of these publications or documents. U.S. patents and other publications and documents referenced are incorporated herein by reference.

1. A method for identifying a subject at risk of melanoma, which comprises detecting the presence or absence of one or more polymorphic variations associated with melanoma in a nucleic acid sample from a subject, wherein the polymorphic variation is detected in a nucleotide sequence selected from the group consisting of
(a) the nucleotide sequence of SEQ ID NO: 1, 2 or 3;
(b) a nucleotide sequence which encodes a polypeptide consisting of the amino acid sequence encoded by the nucleotide sequence of SEQ ID NO: 1, 2 or 3;
(c) a nucleotide sequence which encodes a polypeptide that is 90% or more identical to the amino acid sequence encoded by the nucleotide sequence of SEQ ID NO: 1, 2 or 3; and
(d) a fragment of a nucleotide sequence of (a), (b), or (c);
whereby the presence of the polymorphic variation is indicative of the subject being at risk of melanoma.

2. The method of claim 1, which further comprises obtaining the nucleic acid sample from the subject.

3. The method of claim 1, wherein the one or more polymorphic variations are detected at chromosome positions selected from the group consisting of 33767168, 50495413 and 61089738.

4. The method of claim 1, wherein a polymorphic variation is detected at chromosome position 50497467.

5. The method of claim 1, wherein a polymorphic variation is detected at one or more positions in SEQ ID NO: 1 selected from the group consisting of 12008, 32137, 32720, 43721, 44339, 45640, 48768, 74247, 75828, 76381, and 84909.

6. The method of claim 5, wherein a polymorphic variation is detected at position 32137 in SEQ ID NO: 1.

7. The method of claim 5, wherein a polymorphic variation is detected at position 32720 in SEQ ID NO: 1.

8. The method of claim 5, wherein a polymorphic variation is detected at position 43721 in SEQ ID NO: 1.

9. The method of claim 5, wherein a polymorphic variation is detected at position 45640 in SEQ ID NO: 1.

10. The method of claim 3, wherein one or more polymorphic variations are detected at one or more positions in linkage disequilibrium with one or more chromosome positions selected from the group consisting of 33767168, 50495413 and 61089738.

11. The method of claim 4, wherein the one or more polymorphic variations are detected at one or more positions in linkage disequilibrium with chromosome position 50497467.

12. The method of claim 5, wherein one or more polymorphic variations are detected at one or more positions in linkage disequilibrium with one or more positions in SEQ ID NO: 1 selected from the group consisting of 12008, 32137, 32720, 43721, 44339, 45640, 48768, 74247, 75828, 76381, and 84909.

13. The method of claim 1, wherein detecting the presence or absence of the one or more polymorphic variations comprises:
hybridizing an oligonucleotide to the nucleic acid sample, wherein the oligonucleotide is complementary to a nucleotide sequence in the nucleic acid and hybridizes to a region adjacent to the polymorphic variation;
extending the oligonucleotide in the presence of one or more nucleotides, yielding extension products; and
detecting the presence or absence of a polymorphic variation in the extension products.

14. The method of claim 1, wherein the subject is a human.

15. A method for identifying a polymorphic variation associated with melanoma proximal to an incident polymorphic variation associated with melanoma, which comprises:
identifying a polymorphic variation proximal to the incident polymorphic variation associated with melanoma, wherein the polymorphic variation is detected in a nucleotide sequence selected from the group consisting of:
(a) the nucleotide sequence of SEQ ID NO: 1, 2 or 3;
(b) a nucleotide sequence which encodes a polypeptide consisting of the amino acid sequence encoded by the nucleotide sequence of SEQ ID NO: 1, 2 or 3;
(c) a nucleotide sequence which encodes a polypeptide that is 90% or more identical to the amino acid sequence encoded by the nucleotide sequence of SEQ ID NO: 1, 2 or 3; and
(d) a fragment of a nucleotide sequence of (a), (b), or (c) comprising the polymorphic variation;
determining the presence or absence of an association of the proximal polymorphic variant with melanoma.

16. The method of claim 15, wherein the incident polymorphic variation is at a chromosome position selected from the group consisting of 33767168, 50495413 and 61089738.

17. The method of claim 15, wherein the incident polymorphic variation is at chromosome position 50497467.

18. The method of claim 15, wherein the incident polymorphic variation is at a position in SEQ ID NO: 1 selected from the group consisting of 12008, 32137, 32720, 43721, 44339, 45640, 48768, 74247, 75828, 76381, and 84909.

19. The method of claim 15, wherein the proximal polymorphic variation is within a region between about 5 kb 5′ of the incident polymorphic variation and about 5 kb 3′ of the incident polymorphic variation.

20. The method of claim 15, which further comprises determining whether the proximal polymorphic variation is in linkage disequilibrium with the incident polymorphic variation.

21. The method of claim 15, which further comprises identifying a second polymorphic variation proximal to the identified proximal polymorphic variation associated with melanoma and determining if the second proximal polymorphic variation is associated with melanoma.

22. The method of claim 21, wherein the second proximal polymorphic variant is within a region between about 5 kb 5′ of the incident polymorphic variation and about 5 kb 3′ of the proximal polymorphic variation associated with melanoma.

23. An isolated nucleic acid comprising a nucleotide sequence selected from the group consisting of:
(a) the nucleotide sequence of SEQ ID NO: 1, 2 or 3;
(b) a nucleotide sequence which encodes a polypeptide consisting of the amino acid sequence encoded by the nucleotide sequence of SEQ ID NO: 1, 2 or 3;
(c) a nucleotide sequence which encodes a polypeptide that is 90% or more identical to the amino acid sequence encoded by the nucleotide sequence of SEQ ID NO: 1, 2 or 3; and
(d) a fragment of a nucleotide sequence of (a), (b), or (c); and
(e) a nucleotide sequence complementary to the nucleotide sequences of (a), (b), (c), or (d);
wherein the nucleotide sequence comprises one or more nucleotides selected from the group consisting of a thymine at chromosome position 33767168, an adenine at chromosome position 50495413, a guanine at chromosome position 61089738, a thymine at chromosome position 50497467, an adenine at position 12008 in SEQ ID NO: 1, a guanine at position 32137 in SEQ ID NO: 1, a cytosine at position 32720 in SEQ ID NO: 1, a thymine at position 43721 in SEQ ID NO: 1, a guanine at position 44339 in SEQ ID NO: 1, a thymine at position 45640 in SEQ ID NO: 1, a thymine at position 48768 in SEQ ID NO: 1, a guanine at position 74247 in SEQ ID NO: 1, an adenine at position 75828 in SEQ ID NO: 1, a thymine at position 76381 in SEQ ID NO: 1, and a guanine at position 84909 in SEQ ID NO: 1.

24. An oligonucleotide comprising a nucleotide sequence complementary to a portion of the nucleotide sequence of (a), (b), (c), or (d) in claim 23, wherein the 3′ end of the oligonucleotide is adjacent to a polymorphic variation associated with melanoma.

25. A microarray comprising an isolated nucleic acid of claim 23 linked to a solid support.

26. An isolated polypeptide encoded by the isolated nucleic acid sequence of claim 23.

27. A method for identifying a candidate molecule that modulates cell proliferation, which comprises:
(a) introducing a test molecule to a system which comprises a nucleic acid comprising a nucleotide sequence selected from the group consisting of:
(i) the nucleotide sequence of SEQ ID NO: 1, 2 or 3;
(ii) a nucleotide sequence which encodes a polypeptide consisting of the amino acid sequence encoded by the nucleotide sequence of SEQ ID NO: 1, 2 or 3;
(iii) a nucleotide sequence which encodes a polypeptide that is 90% or more identical to the amino acid sequence encoded by the nucleotide sequence of SEQ ID NO: 1, 2 or 3; and
(iv) a fragment of a nucleotide sequence of (i), (ii), or (iii); or
introducing a test molecule to a system which comprises a protein encoded by a nucleotide sequence of (i), (ii), (iii), or (iv); and
(b) determining the presence or absence of an interaction between the test molecule and the nucleic acid or protein,
whereby the presence of an interaction between the test molecule and the nucleic acid or protein identifies the test molecule as a candidate molecule that modulates cell proliferation.

28. The method of claim 27, wherein the system is an animal.

29. The method of claim 27, wherein the system is a cell.

30. The method of claim 27, wherein the nucleotide sequence comprises one or more polymorphic variations associated with melanoma.

31. The method of claim 30, wherein the nucleotide sequence comprises a polymorphic variation associated with melanoma at one or more chromosome positions selected from the group consisting of 33767168, 50495413 and 61089738.

32. The method of claim 30, wherein the nucleotide sequence comprises a polymorphic variation associated with melanoma at chromosome position 50497467.

33. The method of claim 30, wherein the nucleotide sequence comprises a polymorphic variation associated with melanoma at one or more positions in SEQ ID NO: 1 selected from the group consisting of 12008, 32137, 32720, 43721, 44339, 45640, 48768, 74247, 75828, 76381, and 84909.

34. A method for treating melanoma in a subject, which comprises administering a candidate molecule identified by the method of claim 27 to a subject in need thereof, whereby the candidate molecule treats melanoma in the subject.

35. A method for identifying a candidate therapeutic for treating melanoma, which comprises:
(a) introducing a test molecule to a system which comprises a nucleic acid comprising a nucleotide sequence selected from the group consisting of:
(i) the nucleotide sequence of SEQ ID NO: 1, 2 or 3;
(ii) a nucleotide sequence which encodes a polypeptide consisting of the amino acid sequence encoded by the nucleotide sequence of SEQ ID NO: 1, 2 or 3;
(iii) a nucleotide sequence which encodes a polypeptide that is 90% or more identical to the amino acid sequence encoded by the nucleotide sequence of SEQ ID NO: 1, 2 or 3; and
(iv) a fragment of a nucleotide sequence of (i), (ii), or (iii); or
introducing a test molecule to a system which comprises a protein encoded by a nucleotide sequence of (i), (ii), (iii), or (iv); and
(b) determining the presence or absence of an interaction between the test molecule and the nucleic acid or protein,
whereby the presence of an interaction between the test molecule and the nucleic acid or protein identifies the test molecule as a candidate therapeutic for treating melanoma.

36. A method for treating melanoma in a subject, which comprises contacting one or more cells of a subject in need thereof with a nucleic acid, wherein the nucleic acid comprises a nucleotide sequence selected from the group consisting of:
(a) the nucleotide sequence of SEQ ID NO: 1, 2 or 3;
(b) a nucleotide sequence which encodes a polypeptide consisting of the amino acid sequence encoded by the nucleotide sequence of SEQ ID NO: 1, 2 or 3;
(c) a nucleotide sequence which encodes a polypeptide that is 90% or more identical to the amino acid sequence encoded by the nucleotide sequence of SEQ ID NO: 1, 2 or 3; and
(d) a fragment of a nucleotide sequence of (a), (b), or (c); and
(e) a nucleotide sequence complementary to the nucleotide sequences of (a), (b), (c), or (d);
whereby contacting the one or more cells of the subject with the nucleic acid treats melanoma in the subject.

37. The method of claim 36, wherein the nucleic acid is duplex RNA.

38. The method of claim 37, wherein the duplex RNA comprises a strand comprising the nucleotide sequence AGAGAGGTCCTGAATGTTC (SEQ ID NO: 250); GATTATCCTTGCTCTGGAA (SEQ ID NO: 251; GCACCATACAATCAGAGTT (SEQ ID NO: 252); or GCCAGGCAATGTGTTGAAG (SEQ ID NO: 253).

39. A method for treating melanoma in a subject, which comprises contacting one or more cells of a subject in need thereof with a protein, wherein the protein is encoded by a nucleotide sequence which comprises a polynucleotide sequence selected from the group consisting of:
(a) the nucleotide sequence of SEQ ID NO: 1, 2 or 3;
(b) a nucleotide sequence which encodes a polypeptide consisting of the amino acid sequence encoded by the nucleotide sequence of SEQ ID NO: 1, 2 or 3;
(c) a nucleotide sequence which encodes a polypeptide that is 90% or more identical to the amino acid sequence encoded by the nucleotide sequence of SEQ ID NO: 1, 2 or 3; and
(d) a fragment of a nucleotide sequence of (a), (b), or (c);
whereby contacting the one or more cells of the subject with the protein treats melanoma in the subject.

40. A method for treating melanoma in a subject, which comprises:
detecting the presence or absence of one or more polymorphic variations associated with melanoma in a nucleic acid sample from a subject, wherein the polymorphic variation is detected in a nucleotide sequence selected from the group consisting of:
(a) the nucleotide sequence of SEQ ID NO: 1, 2 or 3;
(b) a nucleotide sequence which encodes a polypeptide consisting of the amino acid sequence encoded by the nucleotide sequence of SEQ ID NO: 1, 2 or 3;
(c) a nucleotide sequence which encodes a polypeptide that is 90% or more identical to the amino acid sequence encoded by the nucleotide sequence of SEQ ID NO: 1, 2 or 3; and
(d) a fragment of a nucleotide sequence of (a), (b), or (c) comprising the polymorphic variation; and
administering a melanoma treatment to a subject in need thereof based upon the presence or absence of the one or more polymorphic variations in the nucleic acid sample.

41. The method of claim 40, wherein the one or more polymorphic variations are detected at one or more chromosome positions selected from the group consisting of 33767168, 50495413 and 61089738.

42. The method of claim 40, wherein a polymorphic variation is detected at chromosome position 50497467.

43. The method of claim 40, wherein the one or more polymorphic variations are detected at one or more positions in SEQ ID NO: 1 selected from the group consisting of 12008, 32137, 32720, 43721, 44339, 45640, 48768, 74247, 75828, 76381, and 84909.

44. The method of claim 40, which further comprises extracting and analyzing a tissue biopsy sample from the subject.

45. The method of claim 40, wherein the treatment is one or more selected from the group consisting of administering cisplatin, administering carmustine, administering vinblastine, administering vincristine, administering bleomycin, administering a combination of the foregoing, and surgery.

46. A method for preventing melanoma in a subject, which comprises:
detecting the presence or absence of one or more polymorphic variations associated with melanoma in a nucleic acid sample from a subject, wherein the polymorphic variation is detected in a nucleotide sequence selected from the group consisting of:
(a) the nucleotide sequence of SEQ ID NO: 1, 2 or 3;
(b) a nucleotide sequence which encodes a polypeptide consisting of the amino acid sequence encoded by the nucleotide sequence of SEQ ID NO: 1, 2 or 3;
(c) a nucleotide sequence which encodes a polypeptide that is 90% or more identical to the amino acid sequence encoded by the nucleotide sequence of SEQ ID NO: 1, 2 or 3,; and
(d) a fragment of a nucleotide sequence of (a), (b), or (c) comprising the polymorphic variation; and
administering a melanoma preventative to a subject in need thereof based upon the presence or absence of the one or more polymorphic variations in the nucleic acid sample.

47. The method of claim 46, wherein the one or more polymorphic variations are detected at one or more chromosome positions selected from the group consisting of 33767168, 50495413 and 61089738.

48. The method of claim 46, wherein a polymorphic variation is detected at chromosome position 50497467.

49. The method of claim 46, wherein the one or more polymorphic variations are detected at one or more positions in SEQ ID NO: 1 selected from the group consisting of 12008, 32137, 32720, 43721, 44339, 45640, 48768, 74247, 75828, 76381, and 84909.

50. The method of claim 46, wherein the preventative reduces ultraviolet (UV) light exposure to the subject.

51. A method of targeting information for preventing or treating melanoma to a subject in need thereof, which comprises:
detecting the presence or absence of one or more polymorphic variations associated with melanoma in a nucleic acid sample from a subject, wherein the polymorphic variation is detected in a nucleotide sequence selected from the group consisting of:
(a) the nucleotide sequence of SEQ ID NO: 1, 2 or 3;
(b) a nucleotide sequence which encodes a polypeptide consisting of the amino acid sequence encoded by the nucleotide sequence of SEQ ID NO: 1, 2 or 3;
(c) a nucleotide sequence which encodes a polypeptide that is 90% or more identical to the amino acid sequence encoded by the nucleotide sequence of SEQ ID NO: 1,2 or 3; and
(d) a fragment of a nucleotide sequence of (a), (b), or (c) comprising the polymorphic variation comprising the polymorphic variation; and
directing information for preventing or treating melanoma to a subject in need thereof based upon the presence or absence of the one or more polymorphic variations in the nucleic acid sample.

52. The method of claim 51, wherein the one or more polymorphic variations are detected at one or more chromosome positions selected from the group consisting of 33767168, 50495413 and 61089738.

53. The method of claim 51, wherein a polymorphic variation is detected at chromosome position 50497467.

54. The method of claim 51, wherein the one or more polymorphic variations are detected at one or more positions in SEQ ID NO: 1 selected from the group consisting of 12008, 32137, 32720, 43721,44339, 45640, 48768, 74247, 75828, 76381, and 84909.

55. The method of claim 51, wherein the information comprises a description of methods for reducing ultraviolet (UV) light exposure to the subject.

56. The method of claim 51, wherein the information comprises a description of chemotherapeutic treatments and surgical treatments of melanoma.

57. A composition comprising a melanoma cell and an antibody that specifically binds to a protein, polypeptide or peptide encoded by a nucleotide sequence 90% or more identical to the nucleotide sequence of SEQ ID NO: 1, 2 or 3.

58. The composition of claim 57, wherein the antibody specifically binds to an epitope comprising a serine at amino acid 656 in a NID2 protein, polypeptide or peptide.

59. A composition comprising a melanoma cell and a RNA, DNA, PNA or ribozyme molecule comprising a nucleotide sequence identical to or 90% or more identical to a portion of a nucleotide sequence of SEQ ID NO: 1, 2 or 3.

60. The composition of claim 59, wherein the RNA molecule is a short inhibitory RNA molecule.


SEQUENCE LISTING (255 sequences)

SEQ ID NO: 1 - DNA, 91000 bp, Homo sapiens
caggtcagaagtctgggatgaccttgctggattctctgctcagggcatcacggggcagaaatcgaggtgttggctgggctgcagtctcatccgaagctggggatcctcctrgaagctcacaccactggcagagttcaggttcttgcagctgcagggctgaagcctctgccctctttctggctgccagctgggggtgctcccagatcctctccatcttcagagccagcaattgataatctccctagcatcacattcttcccacatttccacaggcttctggaaaagctcagtcccttcaccagatgaagtcatctcacccaggataatctccctttcttgaggttgactcatgatttsgtaccttaattaaatctgccaaagccttttgcagcggctcctagattcgtgtttgattgaataactgggagacagtgtgtggatatgaggggctgcggatcttggggaccctcttggaattctgcctaccactaagaccacgctctgctaggacacgtggagattgaaagctgtgataggtactgaagggaggagggatcatctgctccatcttctgatacagcaaagggtagtcgggtccagggtaaagccaagggcaggactgggaagcaagtgatggcagaggctatttcaatgtcctctcatcatagtgactgtcggtttgggagctctgagcctataggcattgttcacaatgaataggaaagcaggataatggattgagccacactctcctgattgaagcaaaaccagaakctccaagattgcccaacacacgtctagagacagaaaagtgataacagcttaactcaaaaataaatgtatctaagaattcaagacatatggtgttaaatattgatcaaaatatacaatgttaatggaaaaatatttggaatctaaatttcttgtttctgaggagtttcatttcataattaagatttattgtccaggaagattttgttttttaaaaaaccacccttcacatgggaaaatatttctctgatttagctcagattataaaacccaaggattcattttaaacggaagggaaggaacgcctcccttaaaacaactgtgttccagaagtgatggaatcttctagtgtcagacttgcaaagttcagtatttgtagaaaattcacatgcctttaattacaataaactcaattcccatgtaaaactgtgccatctggaagatattaacatcatttataacttgttcacctccctatttcatagaaaatttttactccttgccaactttccactgttcttcagggaaaaaaaaatacaattactttcatggattgtctctagaagagtttatatagcaagtccagccatcaagaagtgataaaggaggcattttgtacatatttacttccttcttattgctttaagtccttatatccttactttgaaggttttttccctataaagaatattctccaacaggcgctgtggctcacacctgtaatcccagcactttgggatgtcgaggtgggcagactgcttgagttcaggagtttgagaccagcctgagcaatgtggtgaaagcccgtctctaccaaaaatacaaaaattagttgggcatggtggcacacgcctgtagtcctagctacttgcggggctgaggcaggaggatcacttgaacctgggaggttgaaccgagatggcaccactgaacttcagcctgggtgacaaagtgagaccttgtctcaaaagaaaaaaaaaaaagacaagaaaagaaaaagaaataaagaatattctcctctctctgctccactccctgcagccttccaaatttttttttttttgagaccaagtctcactttgtcacccaggctggagtgcagtggcacaatcttggcttactgcaacctctgcctacaaggttcaagcgattctcctgcctcagcctcccaagtagctgggattacaggcaagcaccaccacacccagctaatttttgtacttttagtagagacagggcttcaccacattggccaggctggtcttgtactcttggcttcaagcaatccacccatctcggcctcccaaagtgctgagattacaggcgtgagccaccactcccagcctccaaagtaattttttttaagtgaactgactttttttttatcacagtcatgcagagagaaggttgaggccataagaaatggattctgttctttgttcccaggctcaggagccactcctgcacctttgatccaaaaggcggctcctccattaagaaggatcaccatctttgacacctcacaaagtgtcacaggggacaatgaggaaaggaggtgacatctacctgacatctggcagtgaagtcagccaaaactggatgactgtgatctcagtcaatctgtcctcacactgccactgacttgtgccctggataggtcactaactgacttctgtgagtctcagtggctcgtctgtaaaatggataaaatgatgattaccttgcagaatcccagggagaaataaacgaaatcatacatgtcatgacgcttgtaggtgttcgatggtccctttccaagtgaggttgggatatggagagaaggagagagacaaatacatagagaaccaaatggagaagagctttgttttccacagtgtatactattgggtcaaaacaggctatagtgtaggtagtccaggaaatttagctattgttatctcccaggggactctgtatcacttagaagataaccaactctcagaatatgaccatagcctgatattctgaaaaatactgtttggaatgacagtcagttttgctctagcattgctctcaaagctcaatcaattaaaaccattgtcagtaatggagttaaataaagcaatttatatataccatagtatgttttttttttttaaatcatgaaatccgttaagtgtttccattgatgtacccaaccgatttaatgaagtgcagttggattagtgctattagcgtgcagcactgaatttgctcaaaaagcccccttaattggtatggtggaggcactagggtgcaatttccagttctggaaaggcagtgcgtccggcttactgcatcacaaaatcatgatcctcaaattttctttttaattctttggaaacaagaaatgaagcaataattctgttttatattataggacctaatgtgctaatttaaagatatttttaaaatcctcctgattaattagtaattattcagtggggatctaggtagtttgctaatcaaagctacaggaattaaagttggcaaattcttattttttaaagttttttcaccccccagtaactaccatagaaccttttcagcatcaactaagggagattatttattctggagcagggaagtcctttaccttaaaatgaccgtcatcttttcctaaaaaaagcaaaaaatagttgaagaaaaatgggtctactgacaccagttaaatgcttcttccttttagtggcactaacattaaaagaaaggttgagctgaacttttcttttttttttttgaaggcagagtcttgctctgtcacccagggtggaatgcagtggcacgatcttggctcactgcgacctctatctcttgggttaaagcaattctcctgcctcagcctcctgagtagctgggaccacaggtgcccaccaccacacccagctgagttttgtattttcagtagagacagggtttcactatgttggccaggctgatcttgaactcctgacctcatgatccacctgtctcagcctcccaaagtgctgggattacaggcgtgagccaccgcacctgaacttgaagaagatgggcagtgggcactaacctaagtaaaaattgcacaattttgttcaacagacccttcyagtttccttgaagtacaaactgctaagataaccgcaaccaagaaatatgaatcttttagagatgtacctaataatatttatgatgctagaatgaaatgtacatgagaaaaacagatcatgtattagagagtttcctcttctgtaatctctagtgtttattgaaagyagcatactgtttgctataaatcatttttagattctgatacattcatgcttagagtacttgtccgttttctttttttcattttttgaaattctattaagtcagaaggtgggtgtatcagctccatactactgaacttctagtagtcttactattctgccaaatatcctctatatgcccaaaatcaatctgtaatgcagtgaataatttatataatgagaaatatatctctggatgtacaataatacttcagatatcaactagagcttttttgtatctgacaacatgtgaaaataaaagattcagagaatcattgtttattgtctagccagtagttttacatgattttgtttcttttcactgatttgccttatagactaatgaattggtgtacagcaaacctttaactatctctatattaggaacaaactcatggagagaagaaaggattgtaatgatctaggtaacctcatgatttatatggaccttaaggagtaattgtgtgtttagaagaaaggaaagcaatggccataaacattacctaagagcaggagcaccatctcagctaagtttaaccatatgaccacacaaaggtgcagcttgctgaaatgggctctgtggttgaacacatttcaaactaaacaaggggaacatacaacccccatcctaccatctaaagtgctcctcaatcccaagataaccttatggcaatcctaacaggaaaaaatggaatagggtcaagttcactaaagtaaaacgtggcataaagcaaacatgtccaaatgtactcttctcttaacacaatagaagctagagaaaggtataaaaaattccccctgataatacttaagtctcagctgtgacaacaagacaggtaggcaaagggagagagaatgttgttctttaccttctttgacagattattattccagcaaaaaggggatttgtttttactaaaaaccagaatatgtaaaataacttagctttttattatatttaagcagcttaatgtctttgaatacatttaatgtagacagctgcagttgactgccctttatgcataaaatttattaagaggattgatagtgtagataaagtcacaggaagagataggaacaagcttacttttcagcattttagttcttgaattatttgggggcatataagtgctcatttaaaaatgtacaataaaacaggccttttggaaatccttccagttgaaacttattaaaaacattacaggttcctgaaattgtcatgtttaaaaataccccatatttcagatatttcattaattcttttttttccctttacggcaaattccctagatcatattattgttttcatcactttctaatctattgtgtttactccctttttcttattagtcttataaaatgcttcaagagaaattttttttgatagtarttagggaaatgctgcaaataatgttcttttcatgaatattcacaatttatatcagtaaattaaaggctctgagagttatgaaaccctgaattaacttggttaaacccaacatacgccaatctcattgatcacatgacccttttatgacataatacctatggaatcagtgccgtcatgaagggcttgctatgtgctattttgaaaagtcatttactttgcacaagcacagatcagaagcaataaaaaaattcagggagagaatcaactttgtagatgaaaacatacatttgagaagacagagataaatttggcaatctagttctttctcctattctgccttcaacattaaggatttaatttgactcagtgctttgaaagactgttttagcaactgtttctggctaaacaagtacgatgctgatacctaagactgtgcctatacttagtaaaaagagctcaaccaaggctgacaaatgcacttagcttgttttcctctagtttgtctccctgagagcaggaatcaagaatgccctggttttactacttagagttaaggtgaccgccatgaatagaactaagcaatattttacagtgggaagatataatagaccaaggttgcattttatagatggaaaaactggagcccagaacactcaatggagtggaacaatatttcagatccagttataatagaagaattagaacttaccacaatattctgatgaaagacaaatcatgacatctcattttctaggtgatgatgagaaattttttygtgatcattaggatccacttctaatttaagattcctaccacaggatttctcatccttgctcaaaaccctacaggaagggggctgggtgcagtgttgtaatcccagcactttgggagctgaggtgagaggatcacctgaggtcaggagtttgagaccagactggccaacatggtgaaaccgcgtatctactaaaaatacaaaattaccagccatggtggtgcatgcctgaaatcccagctactctactctggaggctgaggcaggagaactgcttgaaccygsgaggctgaggctgcagggagccaagattgagccactgcactccagcctgggggagggagcgagactccatctcaaaaacaaaacaaaacaaaacaaaacaaaacaaacaacaacaacaaagaaaacctacaagaaggggaaattccgaatccagacaggaagctcatcccatctttgacccccgggtgagtattccaagtcttccttactttgcctccaatctgttccgtcaactattgtttcttcagtatttaatgtcattcccacccattgatcctaagactgcatttaattcctcttctgcatgacagccttacagttatctgcaccgtgatttcacttccctatgatgagtttctaattttatggtattatgagaagacaaaaatttaaagaactcatttattagactgacacctaagaaacaggcccaaacacagataagcaaagaaacggcttctctaattgcataaaggttgttgagatgttattactttagcaagacagggatggcaagtatgcatgtaagtatttcacaggctctgcaaaacgtgtgggaagcctgtgtcacagagggaaggatggatgactagacttcccctctgtgttcttatcccttgaatgccttgcttttccctccattcctacatcctcctcctacatctgcccttcctgctaatctgggaccatgcaatctagaggtagagtcttgtgatgagaatgaagataatcccatcattctttggagcaaggatccaggaaatggagagaggtccagggcagaatgaagggctgatccaggtctcccatagatggccccaagcaccagagagccaaagaaatgcctggatgaccatttaacacaagcattttccacatctattttgagggttggctttctgatgccacagaacaaacttaacttattctgctgacttttcttttacaaactgggcatgccagccaagaactccaaattctggacagagaacatgactatttttatagctgtttagttgctatgctgaattttctctccgcccccccacccaagcaactaggcaatttcatgccaaatcttatagtccatttaacatttttgcgtgtgtgtaatataataagattattgctgcactgtaaaaatgtcattgattcaggagacctttcttaatgactgagattgcaataatcccccttcccagacataggcaaaatacccattttcactaattaaacacacacataactttcataaattttaaatttaaaataggcttttgtaaatttcaaagtcaaatggtctttatatcaattacttactaaggaattcctccctaattattcaccaacgaacatacactcaaaaacacaggttaacagctcaaatactactggagcagaagtaacattgaatttggcagtcattaaaaataataacttcaacttgatgtttttctaagttttcttctttaaatacaatgtcttcaaaaatactttctcattaatgtgtgacaggcacgttaaacacaggctgtgcaaatatttgaacagtaataatattgatttcttccatggcaattttccatacaaatggtagaagactactagcagaagctctgatttctactctcatgatgctctgcaattaaacaggaaaggattcttagcatgtacgacatacataatttttggtggaaaaagtagtaatagcttatatttacatagcccatttttctgaaaagctgaaagaggttttatgcttgtcattattttagaagcttggagagaaagaatgaacaggtggtgtcctgttacacatgagaatatctttactgcagctatatccccatcactattgagatagctagttgacttgataagaccatgggtttcacaggcaattctttagcctgactagtgagtctactgaattaactcctgcattcttcacccagaggcccatagaggtgtctggagtggaaggaatggcatagacagagtcaatgcctcatccttgggtccggaccttgctgcctgtgactaggatatcaatcaatagcatgggtgctgtggagaaggtaagagaaatggaagctgtgtctgctgaagatcatttgaggacatttccatagcagcttgtctgccatggtctctcgagcatgcttagcccattcacaacgtcagagctgtctcaagatagagtgtgagcccgagcttttctgggttgcaaacaacacggtgcctttccttctaaccctaacccaaacttggtaaagaatggtatctaaatttggttaaagtgaactttgatgacagtcctaaaaaataaaatatatatcctagtagataaatgtcagggtaaaatgaaaattagaacggttgatttggaggtcaaatgtatgtccaggtattggccacatgattttcatttttaaagctcaaatagaaagaggcatgccttggctaatcctgtgggtatgtttctgccccaagtccctaccagttactattcttgggatagtgttagttcttttttcatttcttagagatggagtctcactgtgttgccttggctggagtgcagtggctattcataggcacaatcccactactgatcaccatgggagttttgagctgctttgtttccaacctgggctggttcacccctcgttaggcaagctgttggtcccctgttccaaggaggtcaccattatcgatgctgaacttaatgtggacatctcatcagcacagtgcactacagcccagaacgcctaggctcaagtgatcctctcacctcagcctcctgagtagctgggactacaggcacaccacctcacctggcatgttagttcttttgaaggacaaaggcaagagcctgatgtcgcttatctaaagataactagagaatttttctggcatccctttctagatgtttcttttttcttattgctgctgattcaaatctcactctttcaaagcccacccaaggttcatttattctgtgaagcttttcttttactcaataacctgtaaatgttcctctccttgaatgtataaaacccttaattagggagtaggagccaaggaataaaagtttgccaaatgttgcactacaactgtttttaggggggtcttgtgtgccttatttacttccctgataaaatgtatacttttaggtaggaaatatcttcctacttaccctaaatttttctgagaatcaaaaacagtattgtttccctgggattaagttcatgctactgagaaaatgaacctgcaagagaaactaaaacaaccaccgcatagcccaggtagtcaaactattttatattcatacttttaacaacacttaaacagaatcttctaaatcgtttaggaactagctctggaagagaacaccttaaactttcagacatttttttctacaaatcacataaatcacttctgggaaaactcaaagcatggcggggtgtactgcttttgttccaattacattaaaccttataaaaatagccataatgacaacaacatctcttcagaagggcatgccaaatacttagggggcttagtctcaccaagaccttctgggatttcgttacctaaygcctattagtgataataggtattacgtctataaatccctaggtgcttatatcaatcttatctgttttagtttccacaggtggagtgataactctaatgaggtccacgtcatcatgactcctgtttagagaaataaaatgtttagatctcaactttcctgccattccctagtggtgttataaataagggtgacattgacgcatttcaaattgttcctcgaagagcggcaattcattatcttttaaagaaatttgctccacatcaagtctcaatccaactgacattagcgtgtgttagatacacatacgaatggtaagttttgcctgatgaagctgttaattattctcctggctatgttcactccttactttaccactctcctctatttccttcaattaggcaccaagcatcaaaaggtttgctacgttttatcgttatcactaatctgagtgcacgacacacaattcattctcttttctttagtgcctgaaaataaaaatccgctaccttttgtacggaaggtcagtatctgcggaggccagcattttagacagactacttgttccttcatttgaatttcaaattcttatctataaatgcttttcacccccactttaaatccaggggctgaggagcattgtgcgccctgctttaataatcttggtctcttgcatagcttggatatgtaaattacaggttttgttcttttatttctcccttttagtatctaacttccattgaaatggaatataagtcctgcagccactttgcaacctgaggatgtatagaaygcctatatgygatacgtctatatgtgcatatatttgtgtatgcttgagcataaatataaaatattacaatcttatctgtctacagaagattaatttacaaaataaaaagtttccaaatctgtgctagtttagtttgtccttcagaggacaaaatgctgtcctttgaagctcattttgtgtgtctttgttaccaagaggtttttttttttttttttttaattcataggggccaccaaggcatgaggggatgaccgaccctggttaatccaaaccacaggacagacaaaagattaattccagtcgccagatgattaactctggtcattgggtcacaaccttgcctgtcagtaacttactcctaaactccttcaggaaaataaggaaacttgaacacgccacttttaaagtcaaactcagtgccattatgaaagaccctgtattctgtgcaaggtttcttggaggaatctgatttccagcttttgttcaattagtttcgtatcagtcaatccttgtttaaaaacatgtcataagtctctgtggtttgtgcacgcttcaattacctccgattattacatcaagattcacaaacgagataccagagtcactcctatttactgggaaatgctattgaagcaaactgaaattcaatttcagaaaagattgcaataacccgtcaaaacatccaaggagagaggaagttgggcctcaaggtgcgtggggccattgttaaatgcaagacggcatctcacctaataatttaaacatggtctcgtggcctgacatttttaatgcttccttgatctctttcttcyagccctgcgtgttctttgccactgtagggggcagtgcatagcgggttgtggattaaataccaagcctcattctgccaatagcacttttaggaggggaaaaatcattgggtgcttttataaatagtcacgccgtaggtgtccacagaaagcaacctttattcagggcttttaaaagctgcctgctttttagatacaaaccaatccataaaactaccaagttcaaattgtcagctattatctgggcagtaagtttcaggcacctctgcaggctgatttgayggtttttgtcagcgcaaattaggatcctaggtttaagtaacgcaatctggctctaaaaattaatttaacttgcccaatccccacaatacacagcccggagttcacatttactttgtgactcattgcatttgaattaaactagagggctaagaattttatgcctgaagcagctctgactctgaagaacagaaacaaacacatatttctcccatcatttgctgcttcagggcaagaaaaagaaagccactgtaatttggagacaagacttttccagacaactatgaacaacagcaaccacagaccagtactaaacagaaatgtgttgtggaaacctggacttcagtaagaaattagtaggagcatatcaaacagatgcaaatatacaatttttgatagatatggtgttggcatgaattctagacgtaaactcaaagggattggaaacttgtttgtaaatgaacatttaaaacacagattgcgaagtgttttttggaagcagagaatcaattaaatgaaggaaaatttagaagagtcatagacctgaaaataaactagaaaattgtctgtttcaatatatccttattttcaaatatggaagctaaggctctaagagattgggttggccaagatcacagttaggtttttttgtctgttttgttttgtttttcattttgttttttgaggcagggcctcaatctgtcacccaggctggagtgcagtggtgcaatctcggctcactgtagcctcgacatcctgggctcaggtgatcctcccatcttagcctccagagtagctacgactacaggtgcatgccatcacacgtggctaatattttgtagagacagggttttgccatgttgtccatactggttttgaactctctggctcaagtgatccacccacctcagcctctcaaagtgctgggattacaggcatgagtcacggtgcccagcccacagatttagttgtgaagtcagaaaacccatgtcttctgactctcatcttaaacttttccataaacttctcaaacaggttttcccacttgtatcgattaagaatgcaaatcctctctgctaatttacttctttcccaattgttcttggaaagcaaaattctacaatatcaaactcttgattgtaggccctcttatctttccagcatatttttttacataaacaaagtggtttttaaaatcttcattcaaaaaaatatatatgcattttgaagtgttctgatggtcttggttcatgtttaattccaaatataagtagagaggaaagtaagttgtctatgtatagaccacatgctgaccttgacactgaagcccagcaggtgaaagagggcatccgagaattttggtggcaatcttaacgtagggcttgaaaggccctgaagagttcatttgtccaacctaatccagttccagcagataagcagatttgtatctgtcctgtttgtaaaacctgccaacgacggtggctcatgcctgtaatcccagcactttgggaggccgaggtgggtgggttacctgaggtcagaagttcgagaccagcctggccaacatggtgaaaccctgtctctactaaaaatacaaaaattagctgggtgtggtggcacacacctgtaatctcacctactcagggctgagcctggagaattgcctgaactcgggaggcagaggttgcagtgagccgagatcgtgccattgcactctggcctgggcaacagagtgagactccatctcaaaaaaaaaaaaagcccgccaatgaaagtaattccacagcttccctatgacctcatttctgtagtccataacccctaccttcaagaaaattttagaagacaggcttccgatttgtcttgtttcagtttgagatcatgaatactaaaataataaaatctacttttatctggaatacttaaattccttattttttcagatgaggtaagaaacatttataaaatattaacttgaacacaatggtccagacaggagcttttttcccaggcttttccttccctggaatatttttggcctgacaaactttaacttctttagtacatatcttggcagccagtgcagaggaaaacagaaacaggggcctccttagaattttacaacctgtttggactgttgcatctttataatggggatggtaactctcttttcatgaattcagtcacatacaattaatttcattgtgccagaaacaaggaaaactgtcacggaccggaattacagaggagaatttggagggcatcttctctgacccctaaatgctaccgataaggaaacagagccccccgtaagttatgtgggctgctaaaggtcacacgaccaaccgtccacagcaaagtcagcactggattctggattctctgctttttctattaaaacacactcactcagcattcagcaaagaggggagagaaaacagaaaacatgttcgttatttttgaaggaagaatgcctgccatctgctaaaaacgtgctcgcgtgcatgcacacacacacgtgctcaaaacccaataaaattctttaaaacttagctgatttgcataagaaagatgtggcactattaccacacgatggattttatcaatttctgaatatccatgtgaatgaatggatttcactcatccatactcaacgtgaattcttttgtctgtcacagtcatagcacggataatgtgctttcttcagaattaacctcaggacaacccaaagaggtcgcaaataccaactaaaatacactcttcattttacattactattgaacaatgctggtgtgtgagcaaatggatctaagtagaatctgtgggctttgcaacctcgagtcaaaatcagtgtgattccttccaaataacatgctgatcgcttagtaattttcaagtgcatgtgcagttttatgtcatggtrggggtcctacatatctcaaacgcttcagtgagtgtgggatttggggatgagttcttagaatgtgagaatatagccagagtgtgacctggcatttagcttcctgcaaatgtgcatcactgttcttcatggagttgatgtctttgcagagcaatgctttatcaaaatctatgtcctggccaggcacagtggctcacgcctgtaatcccagcactttgggaggccgaagagggcggatctcctgaggtcgggagttcgagaccagcctgaccaacatggagaaacctcatctctactaaaaatacaaaattagccggtcattggtggcacacgcctgtaatcccagctactcgggaggctgaggcaggagaatcactcgaacccgggaggcagagattgtggtgagttgacatccgccactgcagttcagcctggacaacaagagtgaaactccgtctcaaaaaaaaaaaaaaaaaatctatgtcctaataactgtgctaagagagccaaggggaccagaagagtaagtatctcaaagttaatgaggaagataagaaggaaaatgccatttttgtgaaaccctgaagggttcaccaggcgtaagggaacctgggacagaacttcacctctctgggccttcgtttcctcatctgcaacctaaggggattaccctagaagatctctaaggcccttttgattaacaacaaacacatcatgattcaaaaacagttatgctgtgtttgggtttcatcatagcccagggtctctgacttgtcctgatgccacctatatactacatcaactttgggcaatgggtctcaagcaaagcattcttcaagtgccccacagttatcaaataatttaaaaatcttagattttcatttttgtgattatcttctaaaaatggcagttatacccattgtggagcacctagacaggtaccatataaggagtactgccatgtctttttgtagcaacacatatgtttgtgttgatgattgaatgactgccaagtgcggtgataggaaagcatgaatccctatatagccaaggttttccagttgttcaaaaataaaacagtggtgggagaggtgagccatcacttgggaacacaggagtaacatggatctgaactcgaaagaccccgcatcctggcctttggcagcatgttcaagtctgtggacaattgataagcaacagttcattcgtggtcagtctagacataaataggcagtgaagaactttccattagaagattttcttcccagaccaggtgcagtggctcacgcctgtaatctcagcattttgggaggctgaggtgggaggactgcttgagtacaggagttccagaccagcctgggcaaaagagccagatcctgtctctacaaaacaatttttaagttagtcaggcatggtggtgcacacctgtagtcccagctacttgggaggctgaggtgtgaggatcacttgagcccaggaggttgaggctgcagtgagccatgatcatatcactgcactcagtgtgggcaataaagtgaaacccagtctcaaaaaaacaaaagatttttctccctgtattatattttgggggtacaccattagatcatttggcctgttagtagataaaacaaattaaaagataaaaagcaaatacataaaaataaacagcaaaggcaataatgatggctaaaagggctgcctattatgctaaaactcaaggaaattaagttaataacaagcaaagcctaaagtgcaagtacgttctactagtatattacccaataacaggaattttgcttctacaaaatgccatcagtcatcacaccaaattaatgttgtcatctgagtgtttgggttttgtagttatgttgggattagtgtgtaaagttgcttggactcatttgttgtatgggaatactaagggtaaaaaaatgtgtgagtgaattttttgtaggtatataataagattagaataaaagagtgatcttcaacaccaaagatccttggaaatgtttccctttaaaagagggtcatacattccttaagttggggaaatactgtgttttctttcagctccatcctcgggcctttcagtgaggacaggaacccacggccagacaggaaggcctgtttgaagccaggttgcctccctagtaagtttgttctggagccttctggtctccttttcttttagttactttaagtggttttaggaaaaagtactcttttcatcttaatcctgctccttacacccaagccagcccagagccatctggaatcctcagaacagacctattctttgaagagaagaaggacaagatgtttttggacccttgcaggaggtctggatacagacggggccctggggagggttgatccccagcagtaactgctcagctggaggcatgaggggtcccttctgacctccaagcttcagaggggatgggaaaaggtaatttggcagctttgtgttccaaggcttcacccctagattggtttaggcaagagtcagtgagtccctgggaaggcactgggccagcagagcccagacatcaaggctagcatttcagtcttaataatccaatttaggctgggcgtggttgctcatgcctgtaatcccagcaatttgggaggctgaggcagatggatcatctgaggccaggagttcaagaccagtatggccaacatggcgaaaccccgcctctactaaaaatacaaaaattagccgagcatggtggtgcatgcctgtagtcccagctacttgagaggcttaagcaggagaatcacttgaacccaggaggcggagtttgcagtcagccaagatcgtgccaaaagaaaaaaaaaagaataaaaaaccaatttaaacaaagaaaaacctttcattgagctctttaaatatcctaagaattgattataaatcacaaattggaagccaacataagctgtaatgcaaagcaagcactgaacatctctctacctgatgccacttaagttatggtaaatttagtcaaggctgtgggttggggttgtgtgccttattgcaggaagcagcacacttctgagggataaatattgcatttggaacctatgagttggtagtttttctccaaggatgttcttaaaagccatttgcaatgaatttgtcagtttctttagatttggatggaatataacgattctggaacattactggggcctctgaaaattgtatttcctaccttatatcttacgtggctaaagtacatggctgggagcggtggctcacgcctctcatcccagcaatttgggaagccaaggtgggtggatcacctgaagtcaggagtccaagaccagcctggccaacatggtgaaaccctgtccctactaaaaatacaaaaattagctgggtttggtggcatgtgcctgtagtcccagtctacttggaaggctgaggcaggagaattgcttgaacccaggaggcagaggttgcagtgagctgagactgtgccactgcactccagtctgggagacagagcgagactttgtctcaaaaaaaaaaaaaaaaaaaaagtacatcttgttccaagaaacctacttttccttcaaacactttgaggtttaaagkcaggatttactaccctctgtcctgacggcagagggagaaggaggagaggaaggaacagaggctgtcaccccttgatgattttctgggatcctacagttagatgggggtatggtctcctgccctatgaaaaccagattctaggatgctattctaagaaaaaaaaaatcccctggaatcatattatcttctgaaaactagtgccacattttcctgttgatactatactcatcaacttcagttcacagtcaaaattcagttatcctgttaatcttgctcattttggcaggcacgaaaatacacatgtttgtatggagccaagcaagtttttgagatcttgggatcctctggaaatattttttcagttgttgaaaaggaacttgcaagaaagcatatattttcaacactcgtcccaggaacagtgtgtgaaagtgacattaaacttatttctacactgggtgactagtgactccctgctgggtaatcaggtagacctctgtcatctctgcttcactggggtttactgccagggcgtagagagtctcatctcatggatcggaagaatgggctgtacacacagacagctctcaccctcctgttctggcgctgggctattcttcacggccactctttacactgagaaacacctcatgaattttcagcctggaagacgatgggcccatttaatgttctaaccatatagagtgcctgaattcccaagatctccaactgcttttcacttactaccctggaaagaaccccagtagcatccgttatgaagacaagaactccgcaaatctcctccttgctggctggggagcaggaggtctcataattctgtatctggccaggcatcgtccttggctgcatgttggttttggcatcagaggcagctgcctctaagcccttgttctgccatttcaagttatgtattcttaggcagatcttttcatctttttatacttagtttcctcctttgtaagatgagaaagatagtatcttgcagtgtacttgtggcgattaaagattatatttacacgcataccaccaggcacaccgtagacaccacacgaattctagctgtcatattccaggcctaggctagaatgttgtcagaataaaagtttcatattcttcccactaatcaaaatactctcaccaccaccactactgctattactacttctacctacyatgcattgagcacttatttgtgctgggaacttcactaggtactttgctgcataagcttggtttgttttatactttaaggaccccacaaggtaggcactatttttagccccacttggtcactgagataaatgaggcatgatgaggttacattgctcaaggtcacagaggaagtgtctaaccctggctctgtctgacccaaagttactgatctgaaaacgatgctacgccttttctagaaaatgagccaaacctgcaccccaccctacaaagcagtccctgcccccctgaccctgccccaatccttagaactacctatcttctttgtattcatagctcttccaagcaggagtccatcagcactgcaggctgagcacaacataccagcttttgggtcagacaccagctgctgaaacaggcccttcaatctgttgtactccccagaattgcctctgatccttgcctgacaaccatcccaggatttcagatcaagatgtctgaggcaggcccacttctaggtattttttgaaaaaatttgttacaggtgacagggcgtggtggctcacacctgtaatgccagcactttgagaggctgaggtgggaggattgcttgcggccaggaggtcaagaccagcctgggcaacatagtgagaccccagtctctacaaacaaataaaatttaaaaatcggctggttttagtggcacgcacttgtggtcccagctactgaagagtctgaagcaggaggctcccttgagcccaggagtttgaggctgcagtgagctatgattgcatcactgctctccagactgcatgacagagagagaccccgtctctaaaaaaaataaaccaattgctacaggtaattgattctgatgacgatgcttgcttgagaatcctggagagatcctcgctgccttgatagaagctggggactttagggggaccctgaactccatctgaactcttggttattgactgctctctacttctctaaagagacggctgtaaaaaattgacttagaaagccactcgaaaaagttcacactctgaactgcctgctaactcccatttcctaccaaatcaagatgaacactttgcttgactttcacaatgtctagcatcttcacagtcccctgtattcctgtacaacctgctccttctcttttctttgctatcacggggacaccctgtgtacatttaggcctagagttttcttctaaccttggtgtcaaagtgtttccaatcaccaacgagctttcaaggggacaagaaagttccttctctcctattttcattggtcttctaacagcaataaaatttctacacacttcaagagtgtccacctggaaggagaaacatagtgaaggatgcaaatgtccatcaacaaagtgcatctgtgtcagaaagaactcaggtacaaatctgtcactgcatcaggtgggagctctaaggctccactttggacagcggccaggagaggatttccaaagagttcgagttcctcccgcccggcctggacagaatcaggcaggattatctgacaggaggatgctgcagtggttcctgagtgtgtgcacatctcaasccccaatgctggtgggaaaggaaaataaatcttggggcgcccaaatcactaagctaaagggaaaggtgaagctgggaactgctcagggcaaacctgcctcccattgtattcaaagtcacccctctgctcacagagataaatgcttatttgattgcctcctttggagaggctagtcagaaactcaaaagaatgcaaccatttgtctcttatctacctttgacctggaagcccctcccctgctttgagttgtcccgcctttacttcgagttctcctgccctttccagactgaaccaatgttcatcttacatatgttgattgatgtctcatgtctccctaaaatgtattaaagcaagctgtgccctgaccaccttgggcacatgtcatcaggacctgtgtcatgggcatgcatcctcaacattggcgaaataaactttctaaattaactgagacctgtctcagatttttggggttcatactggctgcacccctttccatggggttgctgccccattctctcctccatgggagccgaacaaagtccaaatcaggacgttgccacacaaaacaacacaaatgttgtattattcaaataggagagaggctcggtgctgtcctcatttgggttatgcctgcagtccagaggagcagagtttggtgcatggggagaactgaatgtcgagggcttgacagcctgttcgcttggctcctctgtcctgagattacattcaggattttctctttggcctgaagttagggaggtaggtggaacaagggtgacctttggagtggaattttcataaaataaataaactggatgacagcgtataaaagttacaaacctctttagaatgccatatgtgagcaacttgtaaacagtccataaatatcacctgcagggtggaagtcaaggaggctgatgcagaattctgtaggaacacattttctgcaacactgaaccatctggtaacgacaatttattctctggttggcataggagtacaatgataaaacatgttactactgacttctgtctggaaaaagacgatcacggtccactgtgttgtgtggacatggggccagtgcgcgtgactctcagccactcatatgccctgtggtggtgtcaggagaggaattaaaaaattaaaggggtgttttttggacatccctactccacaagcaggatttcaacagcaaacaacaccaagaaaaaaaagtttttggagaaaaattgttcctttcataataatgccactttttgagcgattcgttttttaaaaagttcacaaacatctgtgtcattcttggggaataagtgtagaaagaacatatggatagcaactttaccctttataaacattgcttattttcaatatcccaataattagttctagaataatgaattctgcaaaggataacaaggtctttcctaggttctatttcacatagagaatttataaaaagaaagaaaaagagagcaagagacagagagagagagaaaggagttttgattttcctccaaatagactacttagtacagagtctctgtataataaaaaaaaatttccaatttctccaaagtgaagaataatgtaacaacggaaacgcaactgaaggagatgggctaatggctttattataacaaacacacatacaaactagacgcattttaatgtcattatcccctaccccacccaacaccaaccttctaactgtgcttttaatcaagaattccttcttatgttgttacttcctgcccttctgggttcctatgatatgcagtttccatcctggtctcatcctgtttggcccctttgcgtaaaagctaaggatggctggcccacagcctaccagggctttcaaagccccagaagtctgtaaacaggaaagatttcctctgaggttgtgggctcctccttgatcaaaagatggctattggataagaacttcccctaaattgccagccactcttattttccagatgagatcctctgttggagggtacattactaggtatctatggcagaggtgacaggcaatttgccaggactccatgaagttgcaggagaagtaagttgcagctgctctgctgttaatgaggaggtagggcttggagaaacacaaagattcctctgagcaatggccctggcaggggtcccctcctggttagcagccagggagggtgagcgaagcaaacatcagttcagaatatgaaagagggacagaaggactgcatgaagagccccagcaaccttgagggtctcagaggttggaaataagaataatgacttccaccaaaaaagtttagttttatccccatggttggtaagtggcctgtttttgagaaatgggaggatagtcaatgacttcctagttggagacatcattccatgtaaaaggactgcttaggtctgtttttagggtcaactttagagtcacagctcataatttcaaagttgggtacatctttgtccaaggcagcgaatctttcccccgtgatttcattatgagctaaccagttcaggcaaaaagctgtccctcaagagtcactcaagaattttgcatttatttctagttaatcacacctgtctctgaaccttgccatggaagagtgaagtgagggagagaacaaagcttttcctctaccttctttctaaactgggtgagtagatcaaagtgtaccaaaacaaacaaacaaacattttcgaaagaccttctttcctgaatgcatcttatcttggaacacatccttctttgttaattacctgacaagaagaaaggatgctttgaattcttatcgtagatttctctgcaccactcagagggcacttaataaattatcttcttccatttactatctcattatttggataaacttcatcttttgaaatagattataaacttttcaatcccaggggattatagcgtagttcttcgcccctyccttccatactttttaggtacgagcatcggtaaacagtttcgtatttaaactctactttatttcatttctaattgacaatgacctgggccttacttcattgtaataaggaaacaaaatgatgaagccagaacttgcagatggctgttctgtctgtttcagtgtctcgaagtcagaggttggtggttaatgataagaaagtaaatgttaaatgtgcttttgcacatttagcaaacagactgagaaccaacttagaaaacattttatttacattttagttatagcattcaccttcttaagatataacatatctgtttgcaagtcctgactccccacttgaccgtgaccccttcaaggacaataaacaagtcattgtccatctttctatacccgtgtagtactcactatgatactggcatatgatatacactgatataagctccagtgttttctctgattaacatcaaataatattgtcatgacaatgagaaataagaaatgtaggttctgattttttcccaaatcttgagaatctgtttttccttcaagctataaattggggattcaaccaaatcacatgaagcagcaagtatttcctgtgtggcacttagctgttctgggaaataactcaatgtcatttggttaacacacacacccaggccaaatagttgaaaataacggaaagacatcatggtaattaggttaggagttgacccaagacatgttctcattaaaattctttctatttctggtctccattccgtcttctttcactgattgacttttctttctttgaatgaaattccctcccccagatggactatttcaccacagcaaaaaattgagaaagctaagaagacaatggggaggaaaaccaatactaccaaaaggaaacctcggagagttttgctttctgtgagttctctgtcagtcaaccctgtcctgttctataagacccttctcatagacccctctttcaagggttttcaaaaaggaaagcctatttatttacttattaccaattatgacctcactggtcttcccactctttagcactagggcaccagaaccccagtgtatgatgatggtcccctagcagccattccaatgacattattcaactcaaacagagctttgcaataagctgtgtctttccttatgttctctgggggatgaaatgagaatgaccccatctaaacaaaagrccacatggttggcattcaacagtcgactcctccccatttatttgtggatccaattcagtctaggtctgaggctttacagaaatgcatggcactcctcacagatggcatctggccaccccttgaaagcttctggtggaaggggagctcgctgcttttcgaggcagtctgtcacacgagtagaaatagcgtagaaacccatatgtcttgagtctaagcaatgacattcaaaaagcagaagcatccatgcaaccagattgacttaaagtaatgtaaacagtccaatcgattctttgaaattgaagaatgacttgctgtagagcattgtgctccatgatggtgtgttctgccagcctaggcatgagcactttgtggtagtacaataaactactccatagcacatggggaggagaatgtgaatcaatgatttagtgtgatctcattggtaactcagtgctgaagatctgctatcatatatttaggtgaataactattccaatgtgctggatggcactttacaaacatatataatctacaaaaatgatttaaaaaatttttgtgcacctttcaagtcaaaatagctcaagatataatgtggacatcctaggaaaataataattgatgcatgttggaaaccttttcatggtatattagggctataatttctacccattggtccctgttctgcccttaaggatgacacagcataagttataaacccctgaaggtctagctgcaaaatgggtgcatgcactcccaaacaccagatcgtgattatgcattacctggaaaagtaagaagtagggaaagcaatatggctgaagacaagtgtgttcagtgaggggatcgttgtctcgggaatcctgggaaccaagttctcactgtggacccactcactgggcaagtcacttcctgcctaaactccactttctctacccatcaactagagaggtcagattctccgttagctgccaaacagtacatgggtaaaattctataggtttaaaaaatcatataccatgactaaaagtctatgataacgggaacacaattttgggatctctttgtaaaaaaaaaaaaaaatctgttctttttttcttttttctttgagatagagtctcactgtgttgcccaggctggagtgcaatggcatgatctcagctcactgcaacttccgcctcctgggttcaagagattcttctgcctcagcctcccgagtagctgggactacaggcacayaccaccacgcccggctaatttttgtatttttagtagagatggggtttcaccatgttggccaggctggtctcgaactcctcacctcgtgatctgcctgcctcagcctcccaaagtgctgggattacaggcgtgccaccgtgccaggccagtaaaaaatatttctaaccaaaattttcactccagctgtcatacatgtagggaagtgattgtttatattaatttgcaaaggctaaatccatttttccccccaactctttgtgacttgcttgggctagcctgcaaaaacacttctgttcatggcagagagctgtctgcatccagctgacattgtttctctggcttaaataaaagaggcaggacaatgagtgttaaaccactgccggactctgatgtagtgtttacgaaggcagctgcttttgcattctgcgggtcagaagggtgtgtgtgtgtgtgtgtgtgtgtgtgtgtgcacgctcctgcatctatgtaagatctgagaatcactgaaagcagcttccttcatcctctattttgacataaagattctgaattcaaagcaccttcagaagcaacaagagattaagacaggccaggcaacgacttaatatggtaaaacatatcttatccctgtggtcagtttacaatttccttgacagataactgctaattaagacatattatttcagaaattatggttgatttcctttgttatttacacttttgttaaaggtagtcaatgatgtcttttagctgctcaataaagggtcacctaaggaaaagcaactgtatgcctaacatctgggaaaaatattcccaatggggtaggtttcatgagaacaaagttatttctcagatgactctttccttgcagcttcaagctttgttttcctaggaattgtgtagaaatgctggaattaatgagtgatagagactttgattgaatcaatattgtgtgaaattaacttgcaaacatggttgtgattacttttgtgggagttggactgaatttctcaagaaagtacacaaagaaaagactcagggcagagctacagctgcctgacagaggcaaacagaagcctaactttatggacttttccatactctgcatatgttgaaatgacaggctgattcatagcttccttgcttagtttccaaatcgctttgcaacaaaagcagatttactcccttcccaagactatawctaggattcatgatcccataggctcaaatcttcataaagggaattttgcaaagcatgcctcccaattcttgacagtcacaattcaaatattttcacaaaacaacaggactctcaattaagacagaggtcagcaaactatggcctgtagatcaaacctatctgactgcttgtttttgtaaataaagttttattggaacacaaccatgctcattcatttgcctattgtctatggctactttcacagtatgacggacgagctgaacagttgagattgaggctgtcttgcatgcaaagcctaaaatatttcctatctggccgttttcagaaagtgtgccaattcctgatttaaaatatcaatttgtgccataaaaaaaaatcaatttctttgaaaacatagatatagttttaaagtaaacctccccaacctcccctacctccatgaattatcttctaaagcaaattggagtatatatcaagagtcttaatactggttttattctttagttcagcaaccacacttctagaaatctcttctaaagaaatccctaagcatacatcaagatttatgcatacagatgttcattccatcataatttataattatttataatcttgaaaaattgaaaatgtcctaaatattctaggccatttagataaaaattattttagaaagatgtgatttaccaaatgcttgtttttaaaaatatttaaagaattaccatttgacccagtaatctcattactgggtatatacccaaaggaatataaatcattctaccataaagacacatgcacacatatgttctttatagcactattagcaatagcaaatacatggaatcaatctacaagcccatcaatggcagactgaataaagaaaatgtggtgcatgtacaccacggaatactacgcagccataaaaaagaatgagatcatgtccttttcaggaacatggacgcagctggaggccatcatcctaaatgaactagtgcacgaacagaaaataaaatagtgcatgttctcatttataagtgggagctaaacattgaatacatatgggcgcaaagaacaacagatagcagggtctacttgaggatggagggtgggaggagggaaaggatcaaaaaactacctattgtgtactgtgcttattacccgagtgacaaaataatctgtacactaaacccttgtgacatgsaatttaccaatataacaaacctgtacacgtacaggtttatatataaaacaaaagttttatatatatacatatatatgtatataatatatatatttaagagcctaatataatgttttacatgtaatttcaagagaaaatggtaggataaaagctgtctatagaaaataatctcattattctttttttttgagatggagtctcactctgtcgcccacgctggagtttagtggcgcgatctcggctcactgcagcctctgcctcccgggttcaagtgagtctcctgcctcagcctctggagtagctgggattatgggcatgcatcactatgtccagctaatttttttgtacttttagtagagatggggtgtcactatgttggcctggctggtcttgaactcctgacctcaaatgatctgcctgacttggcctcccaaagtgctgggattacaggtgtgagccaccatgcctggcctcaatttttttaaaaaatgcatatttctaaatgcaaagaaaataagcagaaagaatatatgccaaaaatatatattttaaaattcatttcagataattttgtgctcaagaaaaataaatatacatgcctttttgtttaatttatgaaaatgttagtcccaaataaagttcttcttaatttatagaaatgttagccccaaataaagttagtacccatttaaaaatgagtattaacctatttccaaaacagtccactttttaatgtaataaaaattaaatattttctaaaatattatttttatgaatcacattatcttttcatagatggagcagagatggcagttttggggcacttgtgctgccactttttcctcttgcccacactgaacactgcaacctattaagccatttctttctaagaagtttctcaaatgtactacagatggccactactaatcagtcagaagtattaagtgatatcaaacatatttgccctctgtgagagggctacataagttttccaagttaattccacccttcctcttaagactacatggtgaaatacatttttatcaccgtagatcttgatcatcttgcttcgaatgtcttagaatgttctgcgtgtatttctttcaaatccaaagaaatgcgcggtgtctcttcatatgcattcaatgctgaatgtctgtgttaggatgtactgccccaagggctgggtgaagctgctgcctcaagggtcctggtgagcctagttactgtatcgcccatgtccacaatttcctgtatttctttctagcacctgacactgtgaacaatgcttccgtttttgaaatgctcacatcagcagctttctgtagcaagacactgttgtaactggtacctctctcataacgccttcccattgtccttggaacaccctctaaccttaatccatcccatcaagggggctttttctcgagacatttatttcttttctttttctccttgcccttctacgaaaagtcaatttctacagctttagccaacaagtgattcccacatttctatgtccagctttgcccagtttcccgtttctcaagttcttttttgcacatctctacctgcaatgtttaaaagcctctcaaaatgttcgaaataggacctactctcttgctgtatttgcctttttttttttttttgagacggagtctcgctctgtcgcccaggctggagtgcagtggcgcgatctccgctcactgcaacctccgcctcccgggttcacgccattctcctgcctcagcctcctgagtatctgggactacagacgcccgccaccacgcccggctaattttttttgtatttttagtagagacggggtttcaccgtgttaaccaggatggtcttgatctcctgacctcatgatctgcccgcctcggcctccgaaagtgctgggattacaggcgtgagccactgcgcccggcctttttttttttttttttttcgagacagagtcttgctctgtcgcccaggctagtgtgcaatggcaccatctcggctcactgcaacctccgcctcccaggttcaagcgattctcctgcctcagcctcccgagtagctgggattacaggtgtgcaccaccacgcctagctaatttttgcatttttagtagagacggggttttaccatgatggccaggctgattttgaactcctgacctcaagtgattcactcgacttggcctcccaaagtgctaggattacaggagtgagccaccgcgcccagctgctttatttactcttagtgttactagtaccactgttctttcggacccaaagatggaaacatttgtatcatcattaacatcctggcctccatctcctctcaaattctatcactggtggagtcctttccatgtttccttcaggacgtacctttctttctaattttactgccgccagtcaccttcagagagtggctggggttctttcgtgggaagaaataagcgtgaaatggaaagacaaagcattctactaatgtatttagtttttaacatttattggataattattatttttagaccccggcttgggatgtggggaagaaaagacgacctacagtttctatttattgtttctgttcaatttgtgscacttgaagagtcagtaaaatgacgccaagaccataaaccaattccaaaaagtcagagctgacttggttcagtcccctttgaagagataaggcctgtcattccagagtatgaggcacagcaggctggtgagtgtaaaggggttgattgggacctagaattataggcaaagaagacaaacatctaagttagagctagcagagggtgtctaacatggagaatacgcatatgctagtcaagtgtccagggtcgggggattgagtgaccttgggaaacaagagtattccagctgatggctgaatctcagagggtctaggaccccagcagagggttacaagtagagagcaccagcaaccaggaacaaacctgatcctcagccaacaattgcctctgccagatgttttttatatgcgtcttgtttagagcatgatctaggtcccaggtgggcctgccttggrggctgcagatgaagataatgaggttctgcaagctggaaaggcatggaggccactcggagaacagcccctgagtagcaggtctcttctacttgtcagtgtctttctagagaataaatatcacaagggcagagaatgatgcctgttttgttcactgatctatcactgtctctatcactatcgctatcagctgtcacagtgatcggcacatactggttggtcaataaatatttattgaatgaatgaatgaatgtggtagtaagcaacaaatgaagttttcagaagaagaattctggtgtcccaagaaacaatgcttgggaaagtgcctgaatctccctcaatttctgagtttgtaagttctgaatttattaccacttatatatatttttaagcacatgttgtgatgaatgtttaaccatgacctcatgttcaacatcaatctcaggacttgtataatattccacatataaaccaaaccctaaaacaacattgataagaaacaggaacagtttaagtcgcaaggccagtctcaggaaaggtgtaatgacaaaaataatgggagttgacagttcaaatcctttttttytcatamcagtgtttagaaatactagacattttccaatgaatgctgggaattagctgaataactttagaagttagtcctctgttgagcacttgctttaacaaaaatatggctcaggaggaccaaatggaggagaaaagtgcaattaatatattcactcattttaggtcctgtttacatttgcatcttgaaggaattgcatttattattttaattattttttaaatcaagtttcctgtctgtaattgaatcattaatagggctctttagtctttattggtgacaaacacatctatgtttaaattgctcagaattttctagtcagatgtgctaaggacacattcagaaagaaaacaagtttaaaacacatctgtgtccttgtttagcctttgaaaagctactttatcattatacattttgggcatttcctttaaaaatgggtctggtacaactaaaccatttcccaaggatgtgtgaattaaccagtggttacaaagagcttcgaaatgatgatggactatcagtgctaagtcaaattatgaaattgcacctttgcaaaaatgctattgtaggaataaatgaatctgaacgtggtatttacagtgtgatttgcttctgaaatagaatatttctaatgggcctctgcaggcaacaacaaagtgccatcttatctcctgttgatagaccacaatttcagaaaattatttttacaatggcatctgaataagctccttaatttgagactgttttccaaatagatacatagcttattactttgcttacgctgcctttgagacacaaagagtgaggtgggattcattaacaaaatcgctgttgtcattcctttgtggtttttttttcaaagtaatttctcttctttacaagctaatgttaatgagctatgttgaataaaaagatcaggtaaaagctacttttgtgttagtgatcaaatccgagtttgggttaatactcaggttatcaattgcacttaaagcacacctcaaacttggtttgcggctcctgtagtttgaagaccaaaccaatcactcatgtatggaatttctactgactgtgaatgttatattcaacaacagcagaacaatacatgtgtatttcagaagaccacattctcagttcttctaaataactgattagataaaatgcatcygttcatgggtaagactaaggtctaggtgaaaaagtaggcaattccttagaataaattttgccaaaacaaggtctaaaacgtctgataccacttaggggttgaaaactgacataaaacatgtgtctcagtgaaatatgagtaggaaggaagagaaggctgtgttgcaaaagtcaaggagggtctgggctgggctggaacagcagcttggatctagggaagaagaaagcaaaaagtagggagggtcttgcaaagaccaaagaaagccccagggatcaggaccagcctcagctgtcagaaaagacatctctgattattacacattgtaggcctgtatcaaaatagctcatgtaccccagtaatgtatacatctgctacatacccaaaaattaaaagttaaaaaaaaaagacatctttgtaatatgctctatggcaggaggattttttctggagaattagaatcctgttactttagagacaaaagtgcttaaagagaagagcttcattcctctcatttcacagatgaagagacaaaggcccagaaagtctcaatacttgagctcacagctaattagagctcaagtgattcctagaactcaggatcacaatgagtacctgcgggcatcacacatgaagaccgcaccgttaactgagcaaatttcttgagagagctacggacatggtccacacaggacgggcaccaaggagacaaagctgagggcacaatcaagagaagcttctgcctctgccccagacagtcttttgtctttcatcccatggaaatggaaatgcaaacgtttattagtttattacgtaagttccttccttccttccttccttccctccctctctccctcctttctctctttccctccctccctcctttcgttttccttccttccttctctctctctctttctctctctctttctttctttctattcctccctccctccctccctccctcctctctctctctctattttcagaggtagactgttgcactgtggcccaggctagagtgcagtggtgtgatcatggctcactgcagccttgaactcctggggttaccgtgatccttcctccacagcctcccgagtaactaagactacgggcgcatggcaccatgcttggctaattttttaaaagtttttatagagacggtattgcccaggctgttctcaaactcctggcctcacgccatcctcccatcttggtctcccaaagtattgggattacaggcatgagccactgtgtccagcgtcatgtaaatcattcttttctatggttgcagacaaactagagaggtgagaaggagggagtgctttctgtttgtttctaaaatagacagataacagaaacatttggaaatattgcggaaaactaagtgtaagagagtaacattgaagagctagtaagaaagataaaaccagctgtggaggctgggtttgcctgttgtaggtgtcatcatacatctactactttgcctcttcctcttgggctggcatgaacacattgtgccacacgccttaagaaacacttaggaaacactggtatggagggagaggggagagcccaggcagcagtggaatcctctcattgccccctccagaaacaagcaggcctgggctagttcctccaagtagcatctttgtctaggacaggaggcttgttatttttctggtggtatggactgacatatttacaccattcttcaaggaacgcatcccatgttaacttctctaatgaagaaattcactttctgcctcttccctggcaagcctgctgtgaagttaacatgtgcatttactggggttgccagctggaacggaagcaggctctaaattatttgtgtttaatttatgtaaaggtggtttggacctgagcccagatgtgaatatacaatagaccaagtttacagaattgtgacacaaagtcccaagagttgtggggatgtcagaaaacactcagcaatcaggacgtatgcctacaacattaatacattttatgcgttcaactccaaaacagattatggagacgactgtaagacagaaagaaaaactttcagccaagcactatctctgcagattttttgagatgtagctagagctactcaaatgtaaaaattcaagcttttaaaaatagaggtttttagggggaatatgtaagtctgactcactacccaggaaaatgaagggtttaccatgtaaatgagaagaataagtgagcattttacagtatgattaagttaagcctttgtccagagagctgacttggtcttcctgtgctggaggaacacactggagaagtctaattggatgtccattggctaactggagaagactcattggttgtcaattggccaattggagaagactcactggatgtccattggccaattcaatttggcaaatacagcacctactatatgcaaaacactaaactcggctctgagcagtagaaaaacaatactagtctctgtaactactgtatgatcttgggagaatagtggaataatattaatgattacaacacaatacagaatagcataaggaacatgaatgaagtataaaaacggttctgagaagtgtcagaggacggagaaagaaagaccgcatttacttggtggctggttatcaaaaatggcttcatgaaggaagcatcatttcaggtagaactaggatgataagtaacattttgacatggagtctcaggtgtgtaaagggcatttacccccaccacacctgtgaaggaaacatcctatgcacaggcatggggctgggaaaaggaggagtgagcttcaagaactagaaagtgaccatttttggctagaatagataagggagccacaggggataaggccagagagaaggccttatctgcccttatctccttatttggcctcctgtggctcccctacttattctagttaaaaatggttacttccttagggaaagccaagaagcacagaccgaggcagggaggaaacggggaactctaaatgcttctgagcagagggcaagaaggaggaaaacctcttctggaggctgatgcaatcgctccagggataggttaaacagtgtttgcatttggcaaatagctgcagaaatgtaacagaagagataggagagacatcgtagatagataattgagaggcctttaaactgactagatttgggggacaaataggaatcaggagacattccagagtttcaaatttgggtaagtaggaaatgaaggaatcagtaaaacagagacaaggcagaagagaagtcatttgcataggaagctgcaaagtttgtttttgtttttaattttaggaaatggtcagttctccaggtggtcaacatcctccagaaagaatttgtaagtttgaggccaacgctcacgtcagattcaggaagctaggtattatctgagcctagtggctaagatcaaaaagcttattcagaggaaaaattcagggcaaaggcaaaaccttggccaatgcctacatttcagtgttggaggagaaatagaggcaaaaaagagaagaggtcagagaagtggggagaaaaccaagatggcacagcaccatgagatgcaggagagaagaaggccccaacactggggagttgccaattatgtataagrttgcacaaaagttggagaaatcagggccaagaaatggccaatgttttgaagcattagaagagccactggggtatttgatttgagcaaacattttcagtccactggagggaatgttacgctgaagttatgctgtcatgctgaagacagctaagtaagaaagtggagttctgaactgcagtcaccactttcaataccttgacagtaataagaaagagagagagagtgtgagacagagtgtgagagacagagatatatgaaatgtttgcttgcaaggtaacaaggtccaagaaggattttgctactcacaaagaggtagatcctacagaattcatttgacccatagaggcccataacagtcccacaaaaaagaaagccaatcccttctattgggtctcccagccacatctgtagccatttgaaatgaagaagcgttgagagtgcttttgaagagttgcagatatagtgtgtatcacataaggccattcatgagggtgaggtggggataaggagctrgaatagtaacctcttctctgccatgatcccaagctctttcctgcaaacatgctctttggtgttgccactaagaaccaactcccctccctactactggaaaatctgtcataaccagaaatgcaggaaggagatggcaaggtcaaaggacaggggatactagaaatagaggggaaatgatgtgaatggaccatcagtttaaaaacatttatttcccagctgcctttcgctatggatttatgctacaaaatccatatcattttctctttcttggaaaggaacataatgacactattttgttgaatggattgtcaaggactggcaaccctgtgtaataaccacaaagctattggcttgaatactgctatttatgtatgggttggcttatggtctgaatgcactaaacatatttacaattcctcaagcacacatgaaagaataatttttttaagtcattgatgctattatcatcaagttcgtgacactgaaatagcatcttctatgaggttttccagaaactccatccatctagatcttcagaaggctgccattataacaaacttaactggtcaacttactagaacactcagaaattcttattggctgaatgcacactgagcatagatactatctctgttgtgtgtatgtgcagtgccttggtgcttttgaaatatttataagtgtctcctcctaaggatgcagacgttactgtttacagtctgaacttatttaaggaaccaaacctgcggatgcacttcctgttatagtaatacctaggtcttattatttataaatatgtatgtgtctcttgtattcataagacacttccatctacattagctcatcaacactgtgttgctgagataggtagaggaaggaaagcacttttgtttcggaattaaagctgtgtctaggtggcattactcaggcagactctgatgtaggcaacctggtcactgtcacatcttcctgggctgtactggttaacactgtcaaatccacgatgttaatggtgccataatgagccattcacatatcgggacccagcccatatgacccatctcctgtctcacagacaagaatgaatggcagaggtgcaattataacgcacatccctaattcaccagaactctttttcacagggagctgctaacaggatcccctgacaatccaaaatccccagcttgcgtgaatgaactaacagacacatcagtttgctagaacagctgaagagggaaatggtgatgtctggaaggactgaacctatgtattctaatagttactcagtagtgtccccagacacgagcaaagaaattggcatggggagaaacgggaagaagaatgcaaagctgtttcctaaggaaaaaagaaatgacaattccaatcagtagtaaacacacaaacaaacaaaatccctcactgtttccttagcttagtgccaatcgtcattgaaaaataaaattatgggctgccctttgtattaggttggcgcaaaagttatcgtggttttggtggccaaaaccacgataacttttgcgccaacctaatagcattgaggaaggtgctagcacagctttggtaatataaggaactctaatagagccagggctgaaatatgttccaaacatctgcgtcaggcttggcagcctgttcccagtgtaactgatcatggaagagggtgagaggtgaagccaactggacttccggggtcgagtggggacttagagaacttttcttttctttcttttttttttttttttttttgagacggagtctcgctctgtcgcccaggctggagtgcagtggcacgatatcagctcactgcaagctctgcttcccgggttcacgccattctcctgcctcagcctccccagtagctgggactacaggcgcccgccaccatgccaggctaatttttttgtatttttagtagagacgggggtttcactgtgttagtcaggatggtctttatctcctgacctcgtgatccgcccgcctcagcctcccaaagtgctgggattacaggtgtgagtcaccgcgcccagaggacttagacaccttttctacaagaggtttgtaaaatgcaccaatcagtgctctgtaaaaatgcaccaatcagcgctctgtagctagctagaggtttgtaaaatggaccaatgaacaggacatgggtggggacaaacaagggaataaaaactggccccctaatctctgctcaggtccccttccaggctgtggaagctttgttctttccctcctcacaataaatcttgctggtgctcactctttgcggtggtgccatcttttttttttctttctttctttctttcttttttttttttttttgagacagtgtctccttctgttgcccaggctggagtgcattggcacgatctcggcttattgcaagctctgcctcgcgggttcatgccactttcctggctcagcctcccaagtagctgggactacaggtgccagccaccatgcctggctaattttttgtatttttagtggagacggggtttcaccatgttagccaggatggtctcgatttcctgacctcgtgattcacctgcctccgcctcccaaagtgctgggattgcaggcctgagccaccgcgcccggcctgggtcggtgccatctttaagagctgtaactctcactgcgaaagtccccagttccgttcttggtcagctagaccgcgaacccaccggaaggaataaattccggacacaagggggcgcgcggggcatgcgtaaaagcctgctccggggccgggcgtggtggctcacgcctgtaatcccagcactttgggaggccgagatgggcggatcacctgaggtcaggagtttgagaccagcctgaccaacatggggaaaccccatctctactaaaatacaaaaaattagctgggcgtggtggcgtgtgcctgtagtcccagctacttgggaggctgaggcaggaaaatcgctggaacccaggaggctgaggttgcagtgagccgactttgcgccactgcactccagcctggcgatggagcaatactccgtctttaaaaaaaaaaaaaaaagcctgcaccgggacccagtctctctccagacaatccgtagcccttaactgtgctgctgggaaccagaggccccaaaattcaaaatcaggtgcctctgattaagctagagggagtgaggaggagcggatgtggccagattctctgaatgtggacatgcagscacccactccaacacccagggagtggctgtcttaagtgctttctgaaaggttgtcatccagacaaaccacccacttcagcagttattgaaattgcccctttcactctctctttgctgaatttttgctgatctcttctccatatgacctactgtgggtcacagacgcttctcagggacacattgcggtttttcacctcaactcttcatatacctcattttgagttcaatacaagtgtcagctgtttaaactagtgtttacatgtgcaaagcattctgtgggacagcgaaaggtagaaatgtaagtaagacatggtttttgtcttgctggggtctgccacccaaacgagagatgaacacctgtacagttagaatgaggactctacggagatgggtgctggaaggcaaaatagagtgctgcagtcagactgtcacattaactggaatttgtcaacttgactggctgccatctttccaaaagagttgatctagaaatttacattttatttatttatttttgagacagagtctcgctgtgatgtcccggctagagtgcaatggcctgatcttggctcactgcaatttctgcttcccgggttcaagcgattctcctgcctcagcctcctgagtagctgggattacaggtgcgtgccaccacatctggctaatttttgcatttttagtagagaccgggtttcaccatattggccaggctggtctcgaactcctgacctcactgatccgcttgccttggcctcgcaaaatgctgggattataggtgagccaccgcgcctggctagaaatttacattttattttatttttaccatcttttctgtgaccaaatgtgttaacaaacaaatgcccactttttcactttattggagaataaaaaggacagacaactatgaactgagattttttttaaaagataaaacagtgatcttagttagccaggggttaccacatctaaaacagcctaatctcttatcagaccaagtcagccttagctcctccaggtttgtagagacttgtgtctgttttttaatctttaagtcaagagtgtgttccttttttctttaaatgtaagaatgtatgcctttaaatattttgaagtgatctatgtaagattatgattttgaccccgtcttaattctttccatgccacttcctgggttgcttgtatcttgcataatagctgccctgctggtagcgtccaacagttaagagagtcagacaaggacttggaatcgtgactatattgactggcctttgctatcttcggctattctggagaaatatgagaacttgaattgagagacagttagcatcctctttcagcccgggactggaagctctctccgtgacatagcagtctatcatagaagcagcaggccatcacctccaacctggctggctgcttgtacaaagtatccacccagataaaacgaaatacactggagcatctgtttcttctcagaaaactcttccatttctcctaggaacccagtgcctgagcagtctccctttaacccttacagagccaccgtggcaaatgcaaggatcraaactgtatgaatgagggcgctgtgggagaagaacacttccctgctatttacctgagattaagtagatgaccagcgcttggaagggaatgtggccttctcacctctgggcctgagaacaagcactggacacagtggtatctaatatgcccttttaccaattgaacagatcaaacccatgtgtggcctggttgtcaggctttaaataaacattgtatttatatgcgctttgtgcttttaaactccatcaacaaaaagcactagaatttagatatttactccccaggatttccattcctaagttaaggcaagtttttatttatttatttgtttttttaaaaactgaaggatagagtctcgctctattgccgaagctggagtgcagtggcaagattatagctcactgtcacctcaaactccaaactcctggcctaagcgatcttcctgccttggcccccaaaagcactgggattacaggtgtgcaccaccacaattcactaattttttattttttgtagagagagggtcttgctatgttgctcaggctagccttgaactcctggcctcaagcaatcctctcacctccgtctcccaaagtgctgggattacagacctgagccaccactctggccaagtttcaattgagtggaaaaccaccaattggttgaaagtttctatgcacagttttcacaagtgcttatttaatctgaacttaaagttcaaaaattaagtccttcctcagaattagttggtttatacttaaactttcctgagaacttgttaataaaagagttgcttgaagaaaaaataagctggtaacgtttcatgagaatttcctcattcaaaaagcacatccatgtgtgactttgatcacaaagacagaaagatttgtgacgcagcaactrtttatttgggttctgttcatatgacttttcccagcttcagggatctcagctgtcaaatgaggatgagagaaaagtgagtgctcatccccataatgggcacatattgaagactcaacagcaagagcccggcataggttggacatggtggctcaggcctgtaatctcattactttgggaagctgagacagagaatggcttacctcaggagttcgagaccagcctgggcaacatagtgagaagcccccccatccttctgtgttttaaataaataaataaatatgtttttcaaaaaaaaaccagtcataattattccttcccaaacactctatgattatttaagttaaagaagaaaaaaacaacatgttgaccaaagaagatctagcaataaagacctgggcttgatgaggaggaagaccacgttataaggcaaaagttctccatgaacttgaactctataaaggaattgagtagacggaagccaaaatattctgtttgtttttttttcttcctctgctcctctgcagaacttagtagagctctgaatgaatgtgcttcacttcctaagctatctatgaattgtttcaaccataagacactactaactgccctagataccakatacaggccaatcaaccagagaagagcaggagaaccctcctctcaaggtgcaacgaactgccaagccactgactcttacaatcaggatggatgacaaacacccaggcaagaggcatttggaacctgatccaaatgaatttttgtttcttgttgttctttaggcttaatgatcagtctagatgaatactaccactgcaattagttacgtttattttttagcttcctttacacacacatatgtgtttgtgtgtgcgcttctgtggagccatgtcagcaggcatgcaagtatcaagttttatgctggaaagaaaggagctgagaatgtgcatgcccgctgaaggcatatgtctcacttggatcagttaagtgcctgctccctaaagtgaaatctcgttggaaatgccatgcacatgttgattcagtctgataagatttcaaccaaccttctcctttagctcgcaaaagttgcttcctttaaattacagaaatgcctaacacaaaacaacctcgtaatactcaaaattcattatgatcaaatgtgattgcttatactaagggataataaagaaaattaattttattaaacacacgaggctctgttcaaattaaatttggtgctaatggggaaaaaaatcaaaaggaagaaagcagataattatctgcctgagtaataaccaaggatgctgcaagtggtagtaacaactttggccagcagttcttctgaggagggttcattcttccaccaatagttttttaattgaattgagcttggtggtggtgcctctcacattgagaagtcaccagagatcaaaaccagaaataaaaagatgaagaaaaatacggcaaaggagaatgtttctgaatttgcttgcacataaaatctgcataataaagcaggctccagattgaaaagtttgaaacaaagacaagatatttccatcagtagccactgaaaaaaattagaaaatgcatcagtaggattctgctttttgattagaaatagtacctattttttctatatccactccaaagactttcactgtctatgttcaaggatagtacccacaggcttcttttttttgagacggagttttgctcctgttgcccaggctggagtgcaatggtgcggatcttggctcactgcaacctccgcttcccaggttcaagccattctcttgcctcagcctcccgagtagctgggactacaggcgtgcaccaccttgcctggctaatttttgtatttttagtagagacggggtttcaccatgttggccaggctggtttcagtcgaactcctgacytcaagtgatgcctgcctctggctcccaatgtttgcgagtgagccaccgtgcctggcctgtacatacagacttctaagactctagttgagttttacctgattttgaaatgtataaaaatgaagtcacagaagttaggacagcagttgcccttgagggcaaaggttggtgactaaaactgacatcagagggcttctaggaagctggaaatgttggcctctagattggttttgtggatgtatccaccttgcaaaattttatcaaatcttatacttgggttgtgtatttttctaaatatatattatactttactaaaggtatatattcttctctatttcatatggaaaatagagatgtagtttggcttaaaaaactcagttttccataaaacaaggattttgttttagtctaagtgatgctctaaaatacaagactagagtgaggtacttcccttggatacaaaaagccaaaaactcagtgattaagataaatagtattttaatgtaacattttttaaaaatccaaattaatcctaaaaaatccatgctagtaataataaagaaagattaaaacagaaaatattactcatattactcaggaaaacagaaaatggaacttacactccaaaaaatgccaataagtgtaaatgtgcaggaataaatgtaaatgtgcgggaataatcagatttgagaaaccaagggcagattatttttcattccataagtttatattctctgaaaataaaagacattagcactgtttcaaagtttttaataatttaggtaatttaaaaactagcattaaaggaacaatatctgaattatcccaacaaaattctgaaacgaatatcatctctatttgcttataatttcactatgattaaacttcttacgaagttacaagtatgcgatcttcctttgaaygcactgagttaggaaaaacatgcctattgttggttacacagaggagagattacaaatctaaatcatagttgaaaaaagcaactgtgccatgatatgaaacagcactgtcaaatcagacactgaagtcggtaagtaagattttcttcttgtcagtaggaaaagcactactgggtttgagtcccccaatgctaccacctttttagacttttgttagagctttgcattcatttccttaatgtttttaacccaatgtctgtgaaatttttattgctccccggatggtgacatctgatctgaatgtaaccatacacctctttgctcagcagtatttgtcacaaaccattataaattatcatgctgattgtgtgcattgctgggaaactggagaagtttcataaattctaactaccatatcaatgacattcttgtttattatacagatataaacttttatgagattcaagatgtctgccttaccactgaagaaaactttagtggagtaagatctaactagttcgatgtacatactgaaaaccattttcaatgtgcaatgaacatgtgtttccgaaaactctccactggtcacttctttatttcttttctatttttaattatgctttgacacttcacaagtgtgcaggattttgctggcaaaaaagaaatcaacattccttcctctccctcaacaccttcttcggtcagagcattctctagaatcctctcaatctaataataaaatcatgcatatatatagctgggaaaaaatcctagttctgtgttagccctgcagacagacgctccactggtctgttgatcgactgatttacaccacatcttgttcaataaaagaattaaggggttatagtattatggttctgatgcttatttgtaatgaaacctcattcagtgctggcatagacacagaatgttgcatgttaaaactatggaggcacaacttacacatttaattttcctaacgaagtatccacctttgacagccactgcaaaaacttcaccattttgcaaagtatgatccagaattaactcatgaactgggtcagtcttggtgctattcacatgagaagttgtgactgtatactgtattgttaagattatgaaatgatgttttttacctaattatacataaaacaataaaaatctaaataaaaaatttttttttttctaccaggactatgtagctggtctaatgacttaaccatccctatactctacaaaagaaaactgtttacttgggtttcaaagatgtttcacaataagcaaatgtgctaaggcatcgatttcttaagctttctcakggtggaagttgactatacaaatactgaccatttcaagagaaaatgcagggtgtatcatgtgcaaatcatctcattactgggtcaatagatttagttttgacgtatgtttagtttcatatttccaaatgttctaaaaatcaccaacttgttaaagatatcataataatgataacttattttgagggtcacttttaccttttaaaaaggctttcctatctactacttgatcctactagtaaccctatgacatcagcatagcaaatgttttttactgccaacagacatataatgaagctggaacaaggaaaaatattaaatgtcttaaataagatgaatagtttgttcttaacaagagtgataactaaaagctcaccagctttcagaatgtggttttaactgtgcctagttttgtgccacctccaatccatttttgtaaatagcctgatataaagtacaaaaaaaccctaataacaaaactatgtttgtaggaatctaatctaaactttgaagtctatgaatttgttcttcaaataatttctggtgttcccaggttttttttttccccctagggcattccaatcccctgtaaaatttaaatctgtggattcttctcaaagccttcacaaaaggaggtaagtattttgccacatttgtagaatctcaaatgcacccttcaacactgagcactagtcattcatatgaatcagcttagaaagatagtgaaaggaatgccttatgctggtcttcctatatctttctggagtttctcattgataaaaagctcacaatgcaacacttcaaaacacagcaggaagatattttccatggatctaatctccattgacttcatcaggctaaatataacagcctttcagctttggccaggagatgrcaccttcacagagagcaaagcctcagttgggacaattaagaaatcaccttactctgtagcattaaaaaaacaaacaaagtcaccattattaagattgttatgtaacatcttcttggccatgtcagtttctcaaatactttgctgatatcactaaaactgtcattttctgtgaattacaatcatcttttaatgtactttttaaaaagaaaactaaccaaaaaggtgctcctttaatcccattataccccacctgacaccatgatatgacaaatcattatttaaggtttactgtgagctagacaccattgtaatactgtgagatgctgttgtaataattagtatacgctgattatttggaaactgatcatcagcctatggatttatgctaattctctaggcaccaaaatgttgaatgaaccagatgaccttatttccctgaccatgaaaagctagtttactacaccaactcaacaggttgttgagcgctttctacaggtaggaagatttttatattactctgttctttcccagaagaaatgtatgtgcaaatagaattgtaagccctttacatatattaagtgattcaactggcttcccttcttaaatttctactccagttgtgtgctggatctggcgcccaccagctcataagaaccaactgtgcacatttctttgctagtctgggcacagtgacatcacatggttagtttgaaattggctatgatgagagtatttacaccacagaaattggcatgtgctacatacaaatgaggacttgactcgtcttctccaaagagccagctgttaaacatttaccaccacatcactgccaataactagtgattattctgaattctttaagtattatattacacgtattgatggtcatgatttccagattagaaattagtaaacgtcagtttattgaattctaacagcactttattttttggaataatgttattacttgagtatgttttaccagagtttctcctatagcagtttaaacatggtcaaaaagcaaatactaataactactgctactactaatgaagtaaatcaaatataaaagaaaacaagtctccagattgtctaggtgattgacactgtaaaaaccaccataaagaatattagcaaaaataaataaatcaatacaatgaactctttgtttccaagcagtcactggaacactattaatggtaacttccatacacaattaaaaagtcaatataatgccctgaggcacttctgaatcacaaagcaaagataaattggaattggtgtaattttgttgcactgtagtatacactgattatttggaaactggtcatcagcctgtggatttatgctaattctctagatactaaaatgttgaatgaaccaaatgacctcatctctctgaccatgaaaagctagtttactaaaccaactcaacaggttgtcgagcacttcctacaggtaggaagattttgatattactctgttctttcccaaaagaaatgtgtgttcaaatagactgtctttaacaatgaatatacttgcaaaataatatttaggtccagcggtagcagtagtctgtagatatcatatacggttacaccaaactgacccactttaaaacttgtgaaaacctctcagtccctgagaaacactttagaattgagatttaaatcgattatttttctttgataaaactatttatatatgtacaaacttaaaagatggatattcaggttcaagtctctggagactacaccactttcagtgggtcagaatgggataatgtggacaagacagagacagggggtctgttgggtgttaagagagacgagcaaacatgtatctgagtggactgctacgtgcaaggtaaaaacatactttcgatactgtggaaagcaatatccacataccagttctttcacataaagtgaggaaatataatttcgactgtctctatagtgagaatgtcttcataataacgaaattgggcataagttgatgtttagaaacttaacaatagaaagagatagcaagactaagcaggtggatgaaacactgatgtgtatttgagttcccattgtattcatcgtgtcttaacaattattttctagcattttccataaaagtattttaatcagtaatccaccatttacaaaacccctcatctggcttatcctgtaggccttattttattcaatttgctttttccatgtcaaacttacttagtaagatgaatgcagttcttaaatcataagatgtgccacagttcaatatatctcaaatagtgagatctccaatcacactatctgtggatgcgatgtatattagtgatccactgtatttcgtaaaaatacaagtgagatttttgagtttctagttcttcccctctccaggaaaaaaaaaaaaagaaagaaaaaagaaacatacatactgtatacatttcttaacaagagttcccaaaccaaaagtgctaacaggaaggagtataagagcttacacatttaactataatcttcctggtttggggaactcagaaatcacttaacgtacttcaatttccataccattgacatataatctggctgagattggaatccctcctggatccatttataatctacattgtacacaccgacttcccccccgtagacaggcgtgaccactagatggtgctgtggtgccctgttcaaaaacattcccacttacttgcagtctctgtcctccaaatcgaagtgagggttgaagttgatcataattctctggtatgggtccggagcctgaatcagccattcgcatttttcacttgggtgataagaatgaggataaccaggagatgtaaggtacccggggctttcaatttttatagtatcgccacatttatctgcaatgaaagtaagattttagcagatctttcctgacaacctgttaggtaatctagctttatatgtaaattactggttattaaacattccttaactttttaaggggaactctaaaaggtccccgtaagtccttcgtgtggctctgagtgcaacactccaggaaaaaacactctttgatctcaggaaggttgactcaggcagaaagccaactgagatctgagaggactcttccccccagccccgaagtttcttcctaaggaaaaggtcttctagcattaaaaacattaaatcccctcaattgatgttaacatggtcccctgggagcttctgctagacttaattatcccccaagaagtgtaatcacattaagtagcaagttctaagtggaaagtgactgaaagatgctgttcgaccccttgtgttgttttttgatgataagcctggtctatgtcattgatcagtggggattgtctttgattggcttaaagccaaaccttttgttgttgttgttttatctgcccataaatctaagtgattttgagttatctctgctcttgagatccaaatcaacagagaacaaagtatatttggtgaagcagaaataattctgctttacttctccaagtaaagttgttcaagcttggagatcggatcaaagcaacggggcagaaggaaaagtaaatagccacatggcattaattctaattacctaaatgttgggtgaaaaataacccaccacacaggtctgggtatcacatatggtaacccacattctcacattctgggaaaggtgagctaagtagttttgagccttcatttcaaaaatgctcaagtgaagccaaataaaccaagtaaaccagtctctgggctgcctatattgttcagcatagattctyattgtcacattttatgatggatttcatactggagcacctcagcctgaaatgctgccacatttattgatgcttaattttgagcaataaaaattcggtgcataaactatataagagaaacggtatgaaaacagtgaaatatataagcctatcaatcacaaattttcagtaatggtagatgtagattttaacactgcatctaatagattccctgtggaaagagaatggcattctttgctctcagcatgaatttataaatctacagaattttattacaggaaaaccaggtcaaatggaaatgaaagatgaagtgcacatcaaaaatgcataaagctacatgcattacagaaatcatttcccataaattaaaaaaagaagacaatataagttatattgtttttctcaatcgctaggtgcttctgactttgttccttagactgggggtagtcttatgctggtttcctttgcactggcttgtaaaagtgtaagctttgccaggaagaaatgatgttgcaactggagttcaaaatctcagctaatgatcatgttggactctgttatgtgattctgctgagactcttgcattattttgaatgtgcagaaaacttggaaagtgacctcgtctcattaagtttcctctagctctggacaaacatcatggtagtgtctggcatctgttacaggagtgaaataaaacagcactggttctagtcactcgacagagtcacttcagaagacatttaggtggacttggtaccacaccagagctctttaatctgtgacttctgagaaagtgasagtgtaataagataatgtggactgctagactccctggctctgttcaatctctctagcagttgattaagaaagagcagtttctaatgactctggaccattagtcctaaataaggttaatgaattaatttagcagacactggttactcagaggaggtggacatggctttgagaggtctgctgagttacagaggcattttctggctctggtcacttcaagcatagcgacatggcttttgctggtaaagctcaacaactaaagccagattttcagcattccccagatttctgctaagaaaactaagaagtgtgtgtgtgtgtgtgtgtactgcatatatatttgtgtagttcacaacctaaagtctgatagctctttgcattgagcttcaaatctatcttgtgaacacccccactatggatttagtctcaagagttgtggaggacagggttactataatttctagtccaaccatttgagctctttcagttggacgagaatgctatttcattccctgccagagtatagaaagatgctaccagctcaggttcattaataaattgaattctacattcataattttttgtgtcttgctttcctgccagattatagaaaaatgccatcaacttgggctcattaataagctgaattccatgttcataatttttttttttgcatgtgttttgctttaagcatctcttaaaatactgcttggtagctgctgagggaaatccattgctaaaatcacaaaaccttgaaaaagcatattagagagattaacttccatcataaagatgtgatttaaattcctgttggtatgaggccctccacttgcggtctcttattaaaacaatataccacagtaagtagtggatgttcttttcttctaattactgtgattgaaatctaagccagacaaaagtaaatatttgatcattagttaataacaattgctgacgacttactgtaaagataattgcatacccttgcctgcacttactttacagtgtgaagtcaaacatgcctacaaccagatcccattttaaaacacattatttaggttacatggtactgtttagaccccagtatagtttttcatttacttaacagtgagtgattttaaaagctaagtggaagctgcaactagcttagggattttcagatatcctggtgccttacactgtatatgtcagattcctgtattgatactatgtaactttcagtcatactccagacagattacggaaggtgtacaaaggcgggggagggagtttcatcaacccacaaaccgatatctctggccgtcacccaccagaggaaacggcttaggggagcagcaaaggaaccaggaagtaagatctcctggtaaagtgttcattaactttcaacagccatcagatttttgaaagggagtctcttctaattgcctttgggaggaaacattattaagataattggcatgctgatctgggaaggtagrcagctctgtcttgtaacagaggtaaggaaaagccccacagcccaaggatcaacgtcaggagggagcccaaagacctgaaatcctcctgctctaaaccgcctgatctcatttcttcttctctcctcccagataaaagtttccttcgcccgggagtcggttgttcccggctgatcccgggaagccccgcctgagccccggtccggggcgggcagctgggagccggggcgccgctgtcacccgcgcctctgcctgtcacttaccgttgcgaaaagcrccggccggggcgaggacgagggcgagcacggcgcagaggagcggcagccccctctccattctcccttctccgggtccgcaggcagacgcgggagaacgaggacgtggggggaaatgcagcaaagaggagaatctaagcgatccgaagagccccaactccgcctagagctgtacaatcctcagcccgtcttggagaaaagaaagcagcgaggcaatgcctggatccgagaggaacgcttctctttttgtgtctcaagtcgcctgcatcctgtcatttagctccggcttcctctcccttttcccacacttgttcctcttccaggagccactgcccgggccatgtctcaaaaaaaaaaaaaaaaaaaaaaaaggccgggggggggctgtgggtgggagggggaggagggaggtagggcaacacacaccaaagccaatttccaggaagaaaaaaaaaaaatccggcttgtttctggacccgttggagccgcggagctggcgcccaggggaggtccgggtgtctgcgggaaggaggggaacgagcaatgaaaagatgagcgggagacagaggagtttcaccaactgcacccccgcctcccctcctgcggcctccccccactccggagcccttccctcgcttcctcccgcccctctccccgcttgggctcgcctgtaattgcctgacaggagactgggaggaacaactttccctccgagtgctctccccgtccgtctgtctgtctttcccagagaccctgcagggatctccgcgggaaggaaggcgctgggagtctgcgccccggtcgcgtgggtgcgggcgtcggaggaaggggcgcggaggtcagtgccagccgcagggtctggccagcccacctcgcgtgctcgctctgcacccctagagggagagctgggcagctcctgcaggcgcagatcccgccgccgcgcagtgcggagcgcccgggaggtggcggctgcttctcgcaacttcaaatcccggggcccgcgccctcggcggcgaggaacgcagggtcaccccctgccactcctcatcgcgccatcctgagacctgtggcggggagcagcggggcacccctggacgcccctgcccagagctggagggcgaggaggggaaagccgggctggagtgggagccgcaccgcgaagccgggcgaggggcagcagtgtcgctgtggtgtgaagcgaagacagaccgcctgggcgggcgaggccgggctgcagccccacgcggcctccctggcccgcatcagggcgcgcggctgggtgccctctgcgcgccgcggaggcttcattgtttcccgagatttggtgcccgttcctctggggcagagggaacaccggagggtgggaggtgctgcgccttccaagcaggggggcaatcgccgcccgaccccgcccaaccttgtctgggagcccaggtgctagggaggggtgtggaaatcaggggtcgggcggcccccgtttctctgtgcatcccgagggagggaaagagtggcgggggaggggtgctcttcgctgcgccctctcagtgtccgctcccggccacagacaacgtgcacgccggccctggagctcctgccacccccacttcccacagcggctcgtgcccccagcccgctggatgccccccaggcccgtccccgagggttttccgagaagtgctgcaggaagcgtttgtagatgcgaaaagcaaggacatttctgggaagaaacaggttgcggtcaccgatcaggccccggagaagggtcagtgcccacggggcacctcccagggagtttgtgggtaaggtgaaaaggtcacacgcctcctggggattggcgtctacacacacagccaaagtacagcctgattcctacctggcacaaatagctggccaaagggaggtaggaatgctggcaaggagactctggtcaaaggttttggcggaagaaaaaggggcgggcagggagaaagcccaaggcgcgcgggccccaggccaggactgcgcgctcgcgactcgggctgggagcagtgacgtgcgcccctcggggtgcaggccgttcccgggccagtgggaggtgcacccacggagcccgcactcgccactctcttgccccgggtcaagcctgtcttctcctccccacgcgcctcgggattcccagtcgccaagttggaaactcggcccccccgagcgcctctttcctctgccagtttcctccttcttctcagctgtcggacccctgggaggcgcggggtgggaagtgggcaccgaagggctgggagttaaatgcctaagttgagcggaaggagcactgtcgaagccatgggcagccctgcgcccgggcaggcgggcaggcgggaggcgaggcgggggcgccgaccgcgcccagcgcagaccccgggggagtgagcgccgcagaggcagcctgggctctcggctactacccgcgcgcggtcgccgcctccccaggtgcagccccgccagtcccagcccccggctgcgccgggcgagcggcatcatcagatttagggtgacccggagcgacctcggtccaggctcggagggagaggcgcctctcaggacctggcaggttgacagggaaggggaaccgggagggagcggcggccggggcggcctggagtctcggaggcccgcctgcgtcctggggggacccgacctccgcgctcgctccggagcgcccgtttggatagctctgagcctcggcggggtgagcgagggggcacacgagggagacttgagctgtgttgtttttagaaaactgccgccaatctctcttcccctttccctgctgagtgtgcattttccagcctagacccgccaggccagcaaataaagccaccggatcgtgcactccggagcgactgcccccaccccgaccttgtctttcccgaagggaatggctcctccggcgcctcaacgcacagaaaacgcctgcctccagctgagcgcttttgccagctttctccaaccagtgaataagggatgcactatggactcagaaggaaatgtcatatttaagttctgcctctgatttctctgctcttagctgatctaaggtttttctagactccttagtttccactcacagtgctgggtaatggagccctaccgcactgattgagaattgccggccagtgggttggtacagagcaaattgtccttgctaacaacctttttcctttgctgttggattccactaagtggtgagaggggtcagttttagatttttgcaattccccagcgtggagacaggccggctctatacgagaataagtgataggcagaggaagaatttggcagtgttcccgggttgattttattttggtctcttgaacacattattcttcctgatagaacaaatccctggacttggtaggacccagttccagggaagggaagagtcctctaccgcacccccagaacgccctgcagaggctgtgagtgggcaaagcccagtttcctggactcccacttaggaggtgctgcagaaataaggccttcagctggggctccctgagtcggattggaaccgtggcccatcagtaagagtagactggatggtaactgctcgattcctttcatcaagctctcatccctttgtcagaaatagatgaaatcgattgctcccagaaggccatttgagccactaactccctcaaagtacttcagattcatgcaatatggagattcgggttggtagttcaaactgcatcatatctagtcaagttttttgaaaacatcaaacccataaatttcagaaaagcaatacaagctccatatttttagtaggagctaacctagttttgagatgtatgaatagatgaagtgaaactggaaaaacattaaaagttggaattttaattaactaatgtttaaaataagcctattagtggttattctgtcaagtgactgtgtcatgcaagaggtgtcatctgggcctcatttttggccatctccttgatttgtactgacagagaaggaaggaaataaacaaatgaatcttcactttctgattctacctttttagccttttaaaagttaccttgaaaattcctcaaatattggagaaacgactaccaactaaaacgttaacacaaagttacaatgtgaagtttttgtgatacagtatatttactaaccaacaagatgaagaaagataatgctaaattttgaatcacactagattcaaaacatttgctagagtagaggttacagccatatttgaaaactgtattatttttctgagataataatattaaatgattttgccttcattggattacttttaaattgggaaaattaaaataattataaccaacagaaacactcttcaaatcaatagagacatcattagagagaacatattcaaaatttgaaaggactgttttaaatagggcaatctttaaaatataatttgaacaaccagaatagaaaactggctgcctcttaatatttaatcatagttttatgtaaatagctaaaattttaaatcaataaaaaataatttttaaaaatatcaaacaatggccacaacagttaagaattgttgaatttattctcaaaggatgtaatttcattttgcatttttattagatgactctggcagttgcctgtagaatgggttatgggcggagcaagggaagaaatgaagagaccagttttgaagtaaccatagtagaatctaaaggagaacttcaacaaggatctggcggtggagattggaaaaggatgctttccagatgtattgagaaagtccaactggacagatggtggtggataagccaatgaaggagasggtgttaccaaagattcaggccctcatcactgatgggtgcgattgccttcatgtaagcaaagggacactagcaaaaggctaatttggaaggaagatcatgaatttgattggggttaggtacagtttctggtgtctttgaggcaatcaaatgagttaggaattcagaggagatgtttggattggagattttaagtttgtgagtcactgacatgtagatgatggcaatcaaagtggaaggaatggataaaattgctgaggaaatttcaggagacccaggataggaagttaaggaccctttaaagcctggattgtcacagtgagctaacaaaataccttgagaatgagcagctgggtggtaggtggaaaaccaggagagcctattgtcataaaagtcaaggagagggggaatttctaaaaggaagctctggacaagagtggaacaccatcaagagttccactcggatgaggactggaatgcaattggacttagtggtatcatggttactgagaattgactcttccaagagtcattcagtggagcgatggaggaacaggactagaggtcagattgcaatgggctgaagagtcaatagaaggtaaagaaagagagagaacacataaagataaaattctctctggaagtttctactaaaggggaggagacagacgatagctctggaaggaggagatgtggagggaggttgactttttattttattttatgtttgcttttaagatagggtacttgtgaacattcttgaatgccaatggatagaatccagtattgggggatatgttgagattaaactactgaaaagataatccgggtgttgattgcctgggaaggcaggaagggatacaatctacagcccagatggacagatttaccgtaagttggaagagggacagctcctctgttatagcagtggggaaagaggagctgatgggtgtgattacaagaaggtttgtcatttcagtgggtgggtgtggggggcaatttctctctactctggttttctctgtgaagtaagtcgtacctttgagtaagaatgacagtggaggagaggagccgaaagtgcctggagagcagaaaaaaattgacggggacgactaagctgaccagagaagctgttgttatcatgttattgttttgataatttcgtaagttaaaaatttgagctgggagcacattttctatggaaggcattctcctagaaaaattggctagtttttcatgaaactcaaaaaaatctgcttaactcacaaggaagtagtaatataatatgaatggtattctttcagctttagtgactcaaagtaggtgttctataaatgttcttttttaaaaagaaaattttccaaccattatttacagcacattaaggaatgtgtctattgatgggtaggaaaagtttccccccaaccaaataattctttgtaataatgtcttcacttcctccctgccctttgtgctttatcctcctagagatagccaaagaagaatttaaagttatcatttatgtcctgtaccagacctccacagactggcttatttggataattcaagtgaaatacaaccaagatgtttctttagttttgcgaagtctagactcaacatcaatattgcacacatttttcagatatatatgtgatgttttttgaattgttatacaatttcctgccttgatatttcagtcaaatgtattgggtaggttatcagagggcgttgttttcaaaaaaagcctttttccttcaatttattttgttaaagcatcagactgccaaagaatcttaacttctatgtaaggggtgatacattaatacctttttggttgattttcagctacagaatgcacagccttgatggacttgctttaatccacactaaagcttatatgacatgaggaagtcatgcagaacagaacaggcctttctggaacttttatattttaagaagtcttcagcctgttagtacaacaggttaccttttggcaggcacattttcctcctgctcgaatgctataaacaccccagcagggacgataaatagcaatgtagacagcaagggaaaatatttgctaaaagatactcctgcactttctctttttgatgtttcccttgtaagttttagttgaagaaactggaaaaaatagaaatgtaaaagttgaaacagcaaaattataaagtgacaggtgatacaacaaaacatatcagtttgtactcaaaatgtaaagcaataaagcaagcacacattgagactctctaattagtataatttgcattcacaaagttcaaagtggaacttgggtattgccattctcttgcttctctccctaacaagccacaggtagccgaaatgaggcacattgagagttcaaatttggcaaaactctgcagcaattcagtgacagaataaaacatgggaaatggatgttttgaaattgagtctgtatcttaaagaaagtgaaagtaaaactttataataaaataaggctaaatcatacacaattagtgtatccttttgaaaattagtttgtgttttataaggtcaaatggcttctttaaattcatactgtgctattgtatctagcctggcatgtttctttactgtagttctttttctgaatttttaaacccctttgtgatttcacagagttagtaattatgttgggatgttggggcgtggactggatgtagctaaacatactcagatttgactcacattcaaatgtactcaaacattcaaatttaaagttgtctgccaaagcaaacttctgtctcattgatttttaaaattctctggaccttggtttgaaaatgtgttctttgggtttcaaaacagttatctgtcttagatagatcaactctgacatctacatatttgacactagttctgattatgtcaagtgcatatcttttattatgtagataaaagacaacaacttgcatgtcacgtaaaagaggaagcatctcttcaggtcccgggtacatgagtgagtggtgatcctggtttttcagttgcatatcaagaaacacaacctagatgagcttaatgaaaaaggagattagtggtcataacagggaaactttggagtgaacttggcttcaggcatagctggaaccaacggttcagtggatgccaatcagcagggtgtcttctgtgttttcatctgagctggcttctttctcaagcaagaactccccaggcagtgtcaaagatagtaatgacagctccgtagttaaggggcccttcaaggtcatgatcactgagaaagtgttcaggcagagggtgctttctccctcagagtatgtaattatcattaataaaattctgattcactccagttggatgatgtacccatccctgggccagttagttgtcaagggagacgggttactcttaagcccaccctggctgtgggcatgagaacacatggtagacagaatgcagacaggccatctccaagaggaaaagggagtctctggattctgtgatacaaataaagggaaaaggacgccaggcagtcagcggaaacaggtgcccaccactcggcagcccattctcttggttatagttagttgttttcaagtgatagtacagttgactcttgaacaatatgggggctaggggtcccgacccctcccctgcacagttgaaaattcacatataatttttgactcccctaaaacttagctagtaatagctcactgttggtcagaagccttcccaataacatagtcaactaacccatatcgtgtacatgtattatatgctgtgttcttacaatgaagtaagctagagaaaagaaaatgttattaagaaaatcagaaggaggagaaagtacgtctactattcgtgaagtggaagtggattatcgtaaaggtcttcatcctcatcatctgcataaggagtagattgaggaaggaggaggaagaggaggggttggtcttgctgtctcaggagtggcagaggcagaagaaaatctgtaaagaagtgaacttgcagagttctaacctgtgttgttcaagggtcagctgtacatgttcttagtttttgttttggttggtgttagtgtcgtgtttgttttattttgctttgctttcagttttttatttgaatatccagctgtggcagacagcatgaatctatagattttacttcaaaatctgaggccagtactgtatgcagggccagctgtcagtaaaattataaagcaggaagagcttttgtttgttttttgagacagagtctcagtctgtccccaagctggagtggagtggtgcaatctcaagtcactgcaacctccacctcccgagttcaagtgattctcatgcctcagcctcccgagtagctgggattacaggttcccaccaccatgcccagctaatttttgtatttttagtagagatgggggtctcaccatgttggccaggctggtttcgaactcctgacctcaagtgacccgcctgcctcagcctcccagagtgctaggattacaggcrtgaaccgctgtatacggccaagaggagttcatttttaactccatctccttttgggtgtatgtccctaatttaattcgctcttccctgtcctctcacaaggacttccaaagaaatagagcagtggcagaacacaatacttaactcatagaatggtgagattgcaggcaccactgttagccaggtgaggaaggtttttggaggcaaggaaccttccaaaatatcttcataaggatgtaaggaacctatgcatacttggagttggagtgtactgctgtgttcagacatttaaagctgcctttgtaaaggaaatgagattatatttactctggggagattgccatgaagacagtgcttgagaagcacctttaaaatatgaagttttcctcaacaaatgcaacatttttaaggggctacgactggtggactacaggtgtgaaaagtgtgcagtataacagcaatagcatcataaatggaaaagcttggaacagactgttcttctgcagagcatatcttgtgtggtgtgtttttttcgaggtatgttaagagtcttggaacaaaagaattacttgactatattgagatgtatctggaacaggcccacacacgtctgtggtgccagtttattcttttacaaaatgttacctttaaaaaaaatttttaaatacacacttaaatggggatgggagcactgtcagggagagtctcacctatgaaagcaaaaagcacagcttaatttcagagcactgcactgggagtccagtctccgctctccacaactctcagagaaattctgaacacagcactttgtcatattttccactaatttgttcccaaggtctctctccggagagcgtctacaaactcaacccctgccctgaaggggctgtggaggaagtgggcaaggttgtctacagtgtgactttcttgggatacttcttcatcctgggggaaggcctaatgtctaagtgaccacctgtgagcaggtgtttctctcacagggatccgtttctacgagcagacacccttgtggctcttgtttgatctgtgttcagtttattcctaccaagatagctactctctaggaaagcctggatcggtttcctctctaggagaccccagaaggaggttgggttcaggtatgtcaggcaggggagacacaaaggaggcaactcaacaaagcacatgaaacagcagaggatgtgtattactcacaggtcccagagaggaagggagcaggactcatagggccagctggaagtggggaactgtctgagacacacacatgcaaccagggagtggggagcaagagagagagttgtaggggggacctgtgggccaaagcccttactagggtctagggcattacccaagtaggcttcccatgggaagttctaatyggtgggtgagctctgtggagactgcaactgagagggggtcactgcagcatatctgtgtgttctatgttggtgggggagttgttggggcagtcagtggggccagtcaagtaggttgtatccagctctcccttagggaggtggtcaccagcaagtggttgtcaaccaccttgagttactgggaggaagcagagagcagaagactatgtcaaagatgactaagccctgctccccgtatgagaaagtcaagcctagattcaggaccgatgccagagcaacataaaactatagaactcactacatgcttcatgcagctcggtttccagtgtttccatctgtaagagacaagtattgaattaagtgatgtatcaggaccttatcagttgccaaattgaatcattctactgtttttttcagcagctttgataaaacattagtgcaggcttgtcattacccaatactactggaggccttgaaagtgtggagtgctgtactaggatggcacatgacgtgacttatgctgacattgactcctatgctgacattgactcctttgcacagctgatatttgaaacgctcttggtatattttctttctgaattgtttgtgttcctaacatgggccatgaatctctgaagtggacatggataccatgtcctatgtctacctgtacatattgtcagcttgcaataagaaatatgataataaaatgaagttctatcagaggagtggcaggggatgatcaaaccacaaagaaaacaaagcaaggaaacagcacaacgcaaacacagacacccctcaccaaaactcttctctgcctgattgctctaaaacacaaggcaagtcaatattcctgttcagctgagaacaagagtgtttacttcatggaagatctttgagagttagctaaattaaaaatttaaggaaaaaggcttatcaagraaatatgagtcaaaatgtggctgttcctttttatgtttctccgatttcgtgctattttgctactggaaattttctgcctctctggatactcttgcaaatgggaacagatacgcctagcctgggttcctgctccatcattcccccagttctaggagcaggagcggagcaccmctggtgctaggtcaagtggaagacaaatgatgccacatttttgctgtggattttcttttatgtcgtctctagcttctggaccagcggtatattatttttcagagtctttttcagatcaattctgagcttaattgctgacttgcttgtgaggttggaagctctacctacagtgtaccatgcttttgatagagaaggtttctgggcagtttttggtttttcatagaggtattggataggctcatactgattaagctgtggctccagcaaacatgctcaatattatgaagtccaggaggtaggttttgttgctaacataaaaatattcttcagctccacgctggcccaaagcaggagggggaagttggagaggaacttcttcatccatcaaacaaagtgtatatttgttgccgttttgacaatcacatcccttagtaaaagatgtacttgaaaaacaatataaataaaataaagtttcataaaggctcccaaacattcttcctttccccctgaggctataatgactatcctggttttaaaatcccttttcttattccttctgagttttcccatttcgtgctccctgggagcatctctgtaaaataagcctgtggaaaatgggtaatgaggaaatcgttgccttaattgaaggtcgcatctacaaaggtccaggtaggtgcactttggctgatggaaaaatcccccagagtgacatcctgctgccctgaaatagaggctcggttgtcatttgttagaacaacggtggcgcctggttggcatctcgagtgttgtctttgttaatctcacaaccaggtgatttccattactgacgacaccccacatgggaagggggcaaggaagggcccccaaggactcccataattaagcacaaacacttttacagtattaagtcaaagccgaatgcctcccaaatcctcttggtgttgagttcgtgtgccctacacaatggtgacctttaatatgtccctttcagatccagctagattagtcctgtgctctggccttgatattttttgttttgttttctagtgctattcctttaaaagaagaactatgagagtgatttttttaatctgtttgcttacttttcttttctttttttggcttattgtcaacatgttgtrttgacacactttttagcctaaaacagagagaggaccttctaaatacaaaagataggatgctttcaacaactcgattccctaaagcactggagaattctgcgatctgttagacttcaatgtaagcattttatagaggttcccatgacaggattattagtcagcagtttcacttaaaggtcttttttatttttttggccagtagactttctccttctacagctgcaactgtattaaatatacatccccaataatttagctcctcttctttctctggtggaatattttcagttatgtaatttcagctcctgctgcttatttggggtgattattattatatgtggtccctagcctaggaatgaattctgctgtagataccagtttttaaattagttagaattctgcctggttccccacaggaaaaatgtcataaaagatgggccacaagacagtccaccaagccattttaaactcccacatagctgaactttcatcattggcagctctgagttgtgaggtctcttgtgacactggccctggttatttttaagaggggcaagaaaggtaaatcaagagggtagtgatagaagcaggctagaaaaagccaggctttggcagctgggctgctctcacgggattatgaggggccatattctaagtagcaggaattgaaagtggtgaatggattcaggtcctgaccatgcatgaggctcctgtcaggaggtgccrtaagagctgagcacatcaatttgaagaagaggctcatagagagttgggtgtccttcatgggaccaggatggcgttgaggggtaaaggtggacagcaattgcttcttatttgtacacagcttatttgggagttggagctcatcttattaatgaaggtaaaattatgacttgaaaaccaaccatatttcactactagagtatcattatttgatgctttcaaagtctccatgtgcttgcaccttattgatcttgtcaccagttctcctaaattccaatctggatgcataagaaatatcaaattgccgaggtgggcggatcacaaggtcaggagattgagaccatcctggctgatatggtgaaaccccatctttactgaaaatacaaaaaattagccaagcgtggtggtgcacacatgtagtcccagctacttgggaggctgaggcaggggaattgcttgaacccrggaggcggagattacagtaagccaagatcgygccactgcactccagcctggcaatagagcaagactccgtctcgaaaaaaagaaagaaatatcaaagtggttttggtataaagagctagccttcccgttggcctcaaggatacgttagagataacagcaagctgtctaaatatttgcagactacaacattcaaagataaggcaagaggaccaatcttaaagattttacaaaataattgcaacaagtgcatgcatctgttgtttctcaaatcatgttttaatgttttagctattaggtgaaaatggctatgtacaatgtgctatttttaaattgtcaagagtagatttggaaggttattatcaatatcctacacttttcctttcgtgactcaataataatttttgcactttcatttagccttaaaaaatagaacaataatgcatccaagtataaggtcaattgcaaagtgacccctgtagtttttgcttcaaaactattatattgtgctatttataatatcaaattgccactgtctagaggcaaaatgatcatttcaaaatctttgtaacaatctagagaaatatttacaaatattagccacaattctagtgtaatttttctgatgtgtttctattgctgacaattgttttgtaaaatcttgatgattggtcctcttgtctttgaatgtcgtaagtctggaactatttagactgcttgctgttattgctgatatatccttgagaccgtcagaaaacctagctctttttcccaacaccaatttgatatttcttatatatgcaggttggaatttagaagagctgggtgacatgatcaataagtgattttgagaattgctcagaacttcattttatcttttttttttttctcaaacaacctttaaaatcagaaactctggcatagcactctccaactgtgccacagaaggacataaggatgttggtgctttttcctgtctatttttgtcccctaccaaatcacagccctggaagggagctttagaagtcacaaatctgttctgttgccttcatgctcagtatcacagaagagggataatcaaccctsttctaaaaggctagccagtggaaaggatttcaaaatcagccaacaagtatttttgcttactgtgtacatggaacagtatggaaaaaagaatatggaaaactattagacattattcatacccccaagaatcttgcacttcatttggaaagaatagatacctatatgtacacaaaaaccaccatcaaarcttctgaatttggaacaggtcttgaagacctaactttctgactgttgtaaatatgcactcttgacatcctgccatttgatcctatcagttcatcactgggaaattttaattgcaaataaattctatccacttctaaattctaaaggctttaatgcatgacaggtctatagaagttaaatgaccgtagaaattgtcagcctggatagaaattttggctttaccaatcattaatagtgtggcctcaggcaagttacttaatgtttttgcctttcaatttcgtatcataaagtggggaaaaatactagtatcttcctcatagggttgggagagttaggtgttgaattttcagaatagcccctgatagagggtaagtccacatcacattaaatggtatttgtacaactgtcatacaaatatttgactcaacttttaatgttctgttttccctgctccagctacccgcttttttttttaaaggtaaaacctcttcatctgtgtgattaatgatagaactattccactacggtcacagaatatgatttataactgactaggtcctccttgttggtttttaatgccacccttactacaaatatggtatgaatagtaaaaaaaagtgggccaattattatgttctaaatagtatactactaacactgcatcccaagcttgcaaaaatgtttcaggaaaatttttcaggtttcaggaggccacctgagttagacaatggaggccttaagtctattttactgattgtgtttttcagaagcttgagtttgttctggactttagcttccaattgtgtgaaatgcttgaattgacccaagactatgttaccgtcattctgtcctgtgtgttgttctttctcaaatccatggatattggtgaagtcctagtgtttcatattaatttctttggcagatattcagtttaattctttgctgggatagtgtgcatgggtgtgcatgtgtgtgcatgcatgcacatgtgtacacgtgtgtacagtatcatgcttttgttccactcatagtgggataaatatattccttaataacctggtctgatattcctcctggagtcttactgttccagcctaccctggccacactcttgctttaggaactctcacatttatccttctaagttgcttacacaccaacctcctcatttttcaaagcccagctgaaatatcttctcttctgtgaagacttcctagatcccctggccaggatttgtcccctatcctctctctgctccccagcattttgttaattstccttctcagttggcattttggttattgatgtagcttctgcttcctttatgggactgtagaacactctagaagaagagctttatcttattcatctttgtatcctagggcctagcaagtggccatataaaatttgttcagctgaataaagaaataattttaaaaaaagaaataatgaaaaggttcacatgggggtgtattttctttctgatgttatcatcctaattattagaagacatggagggtctgggaggagaactgatttctaatgaacatccatttggtgccatatcctactcaatgtggtttataaattctctcatttgatctttgcagggactttataagattagtattattatccccacttatatatgagtaaaattaagttcagatagtttgaggcacaggcagagtctaatttataaattccaggtcttggaagtcaatgaaaactagaatgaggactcaaatttacttgatgttaccctaaagccatagacatatttaatgacaccgaagtacatgaagtggtttgacttctatggaaaaaagctgtataaatatgataatgccattttctcattcaaaaacccttcaagagcattgtttttgcaaaccaactaactacattccaatttttcagcctggcacttaagagccttgatattcgtttctgttcttcaatcccattgcttctaccttgatgctctgtccagttggactgaactctctggatatctgcagatttctacctttctacctttgctcactctctttctcccacctggaatgcactttgtcagggatatacttgcaatattaattagcttacttagccttatcataatcaaatacatggttctcttaagcttttggaaaaattaaattctaagtggaccaccattgcaaactctatggctctctggtggttcaggaatctcaggctagaaaaaattctcttaaaggaaaaaatattcatattggtaaatttttttaaaaaaatttaagtatagtacctttcattaattccttggaaacaatgctaaaacttaagaacagagaaagtttgttgttgttttgctttttttttttaataaaagaatctagcctaagacaccattctaaacaaatgtgtcagactcatcaacctcctaatcaaaactttcactcaactaattctttcacttacctcctgcccactgcagttatctacagccctggctttggcaaaatatcaggattgccctattcatctgcctattggatttgatccagaattagtcacaactttgtttaattctccagtcttcagtcaatctctggtccagcatgtataggacagtaattacacagtcacacaatgcagcataatgtatacttctcagcacattcaactctgtgttttttagggtagggggtgggagagagtatctctaaagatgactaaagcacagctgttgggtggatcctccctacaaatgtaaaccaaaggattggggctgacctcagagcgactcccgagccggcactctctgggataatttcctccatccctcccratcctcccagaagsgagtctacttttgttcacatatttttatagaaggttaagtctttcatgggttttgtataaaagttgaaactgggttttgattccttgagatgagcagtggtaaacatgtgtggcattcggagaaacagcaccttagaattacacctgttgtttgtgcggtgtggctctcttcctatggaaatgtgtgaactctcctaggagaaagaagagaaatatttaaaaaatcaaatgcctcagggcacagagggttattgtgctcacaatctcttatctgcgatcttaaaattcaaatagaactgaaaaattcaaatagaactgacatctgcatttatttatttattgrccactcatttggagaccaaacctgaccagatctaaacttccttggcaagatgacttgaccttacttattgcaagcctgtgaatagtctttccttatcccagttagtgtgaaaatccatggtgttgctacagaaatattcatgtgtttgattagaggatgctgccttgaatctcacaggggttatgtaacatagggcatgcatatcctatttttttgtgacattggaaaatttctgaatggtgaaagtgcctggtcaaagggattttgaataactgattatgacctataatgcaagagaaacaaaaatccagcaaaatcaaaatcagaatcttggttttcaacatccctctctctttataccctatcatattcccccaatcatatactctagtatgccacagcagcaattcttgtacattatcaagatctcaaatctaaagatcttgattgtccacactttcttccctccttattctccacccatcattacaatcgcttctttcatttgcttacccctcttattcttcattgcacactcctggcrtatctccatccctgtttccaccaatgatccgtctattccgtgctgtcgccgaagcagctgaacattgttagggggaagaatcacataattgtgctgactggtctcatttaaattcataaccacaactgaactggacactgttgtagtgcagccctactttatttcccaggttgttgcatgcttatgtgacaatgtcacaccttcttatgaccttttcaaacgtctctattgactgttctatcttgccccgctttttcaactcatacgtcattgaaaaaatataagcagaaagtgaaaatgaattaactactctttctttcaccaaatcaaccaacttaattgcatctgtacccatatagtattccttctctcctattttgatagacaaaatatccctggtcctagctaagccagcccctccacttttggcacctcctctgcctttctcaagaacttcagtcctacatttattctttctctctctctctttccttcccacatcatcagtttattcactgactcctttttatctgcacccaataagcagtagtggccaggtgcagtgagtcatacctgtaatttcagcacttcagaaggctgaggcaggaggattcccggaggcctggagtttgcgaccagcctgggcaacatagctcatctttaaaaacaaaataaaacaacgttagctgggcatagtggtgcacacctgtagtctcagctacacagaaggctgaggagaaaggatttcttgagcccaggagttcaaggctgccgtgagctgtgatggttccactgcactccagcctgggtgacagagtgagaccctgtctctaaaacaaataaacatgctgtagtattctctgtatcccctccagctcttgtgctcttctctggtttttatcacgatacaacttaggacataagttagcatcactccctccactttctctccttacccagttccatcaggtgatcactgccatcttttcactgatgttagtcttgttaaggtccccagagacctgcaggtggtcattggcagtggtcacttctctggcctcatccaagcctttcatttgtcccgcatttgacctctgtcttttagaaccattttcttctcttggcttctgtgacaacacaatctcctgatgttttttcttcctgaccttcttctcacatcgtttgctgcctgtccaacctctattagctggggtatcccaaagctcagtgctatacccctttctactccttatttcactaggtgatcttattcttttccatggctttttaagtaaggttccaaaagtacacccccagccttgggcttgagctcgggattcatatatctaattgccagtgtggattcatctatctaattgccagtgtaactctccacttgaaagactagtaggcacttcaaacttagcacgccaaacacagaactcttgacactcaccacctaaactgttccccctctagtaccgtccatcttagtgagcacagccatcaagtcactcaaggcagaatctaactccctccttcccttaactgccacatgtaatccagcaacaggtctcaccwttgccacgtgtaaaatacaccctgagtttattcctttctctctgttttcctagtttaagccaccagtatctcccacctgaactgttatggtaccttcctgcacgtcttcttctgtactctcttgattttttataattgactctacataacaacaaatactgtatttaatcttgcacatgtatttctatgtcccatttaacttaggataattttgaactctttgtgttggctcacagggctctcatgatccaaaccccatctccccatctccctttcctgccctccttggcatcctctctcccactacctcccagccatgctgccttcttcctcttctttataccagccaagcgcattgccacttcagggctttggaacttcctcttacctcccccctcgtagatctctacaagactcattcccctttggcactcaagtttcaaattaagtcacccctgcagtcactctctgtcacatccctctgtgtgacagcaattagagttcttgttggcacttctccctcagtgctcactcagttagtcaacaagttttrtttttctccattcctctcttttcctactgccaacaccctaatagaagccaccttcctcacttgtctggactgtagtagtagtagcccctcagccatctccccacatttgtacttattcccttctaacctgacacttggtaacacagctgccttacttagatgactgctgtgacttctcattgctcttaaggtgaatttttatttttatttttattttctgagacggagtctcactctgtcgccaggctggagtgcagtggcgtgaccttggctcactgcagcctcctgctcctggggtcaagtgattcttctgcttcagcctcccgagtagctggctctacaggcgtgtgccaccaagcccagctaatttttgtatttttagtagagacaggtttcaccatgttagccaggatggtctcgatttcttgacctcgtgatccacccgccttggcctcccaaagtactgggattacaggcrtgagccactgcacctggccgagggttaatttttgaatccttaacaggagtctaattctctgaatrggctggctctgacgtttctctttagctgcaccagaatatgtaagcattcgttcagttccttacatgattcacagcccatttcaacgaagcacatgctttttccttgactggtaataccactttacctgctcccttttgtcttcttaactcatgcccatccttcaagtctgagctaaggaatcattttctcaagaaaacccatcctgaccacctcaaaacatctggattttccccttctgccacataacatcatagcatcctgtactgctcattcacagtacatatcaggatggtatttaattcattcacctttaatgccatcttcctcgctagaatgtttcatcagctataatgtcatggtaaatggcgctggggccagattgcccaggttctaaaccctaattcctttttcttacatagctgtatgaccttatttaggctttcttgatgtaaaattaaggtaattatggtacccaccatgtagatttgttttgaggtttaaatgagttaataaacataaagcatctaatatagtgcctggcagataataaacactcaataaatcatcatgattgtcattactcttatgagctctgggattgcatctgtctcattcatcatggcacatccaacaagtatttgttgaatacataaataatatttttcatgagccaattaaagttagaagtaatattattaaatgcctagatgtacgctaggctaggggatatcaaaattaataaaacatcgttttcacaatcaaggagtttttaactcagtgaataagagtgagatacataaatgaggtctgctcaaatggtgtagttacacatctttgtgccctgggcattttcctaggagcctgatacggtttggctatgtccccacccaaatctcatcttgaattgtagctcccataatttccacgtgttgtgggagggacctggtgggatataattgaatcatcggggcagtttcctccatactgttctcatggtagagaataagtctcatgagatctgatggttttataaggggaaacctctttcacttggttctcattctgtcttccctgtcatcatgtaagaagtgagggtttgtttgtttttttttgacagagtcttactgtgtcacccaggctgaagtgcagtggcacaatctcggctcactgatccccagttcaagtgattctcatgcctcagcctcccgagtagctgggattataggtgcgcaccatcacatctggctaatttttttttgtatttttagtagagccagggtttcaccatgttgcccaggctggtctcgaactcctgagctcaggcaatccgcctgcctcaggctcccaaagtgctgggattacaggcatgagccaccgtgcccagtgatgtgactttttcttccttgccttctgccatgattgtgaggcctcccaagccacatggaactgtgagtccattaaacctctttttctttataaattatccagactttggtatgtctctatgagcagcgagaaaatggactaatacagagcctaccccatagcttgtgtacaatgtgtacacaccaaaccgtgacctgcctagctgtctcctagaggaaagaaggggcactctagagagaacagaaaacagcctgaggaagtggcaatgaattttgccttgaagggccggtaagatttcagcaggtacaattcaggaaatggagaggtaggaggcattccaggaaggacacaagtaaaggcaagaagccaggaaaatgaatggagagcatgaaccgtctgatggtggagcagtggttctcaaagcatggtcatgggcccagcagcatcagcatctggcctgatgattgttagagatgaaaatctttggacccctgaccagatttactgaatcataaaatcctagtaatctgtgtcttaacaatcctctggttgattctgttgctcaccgaagtttgagaaccactgggctagggcactgtgttctcactggggacccacraacctccagagagttgcagagggattcatgtggtgacaatgacagagcaagtcaagagtcaacccccccaccaccgcccctcacccaataggcaagggcaatcgtgtgtggggtcagagtcagtgctgttgtgacttggtggaataccactggtttcaaccttcatagaatttgggtggctttttgttgttgttttgttattgttgttgttgtggtttggtttggttttttgagacagggtttcactctgtcactcaggttggagtgcagtggcatgatcatggctcactgcagccatgggcccctgggctcaagcaatcctcccacctcagctttctctatagctgggactaccggtgagagccaccatgcctggctaacttttttccttattctatagaggcgggatctcaccatgttagccaggctggtctcagactcctggagtttttaattgattttttttcagctacatattggggctgaaaaggtggaatttttaaaaacacaatgtaaggaaaactagtgttagaatataagctgttgagaagcaaatgatagtaatatttctgcaaatttagctcaagataattttcacaagtcagatccacaagtcagatggcaaaattcttataaagcttatcaagatgtctcaaatgcttaaaaaataaacagaaaaaagagaaaaatatagtaataatcatatctagtttggctttcgtctaataaaaatgtgttatttggaaagtgcttgtaaatagcctaaaaatttctttcatatcatctttagaacttaacaattataaaaatgagccaattgatttttacattacaaaatgcagacattctaaatatcagctactaaagtagtcatatcttgatgatgtaacatgtaattaaattttaatctacatttttaatatacaatagtatattaaatcacatacagtgtcattttaaaatttataactcaaccatccttttttcttacataattaaagtaaatcaaacagtatgtgtcatataataaatgggaaaaagctatgatttttaacacatggaacagatattatagaaatttttattaaatgaattttgtaatatggtcttaaataatgcactgatctaaagaaatttaaaacccttttcaacttaaaattactttgcttcataccatggaaaattttaaaggaaattgttggtgaattattatgaaaagggttcatgagtcwcagagagaggaaaatgctggactagtgcactgggtacctggatctgggtaatgggggagtgtatcagtctgttttcacgctgctgataaagacatacctgtgactgggtaatttataaggaaaaagaggtttaatggactcacagttccacatggctggggaggcctctcaatcgtggcagaaggtgaaaggcatgtcttgtatggtggcagacaagagaagagagcttgtgcaggggaactcccctttataaaaccatcagatctcttgagacttattcactatcacgagaacagcatgggaaagacctgcccccatgattcaattatcttctaccaggtccctcccacagcatgtgggaattatggagctacagttcaagatgagatttgggtggggacacagtgaaaccatatcaggagatagaggtcaaggagagktactgcagaaaggtgaggaagaaaccagtccaagagatctccaggttgtgggaagttttgaaaccctargcataggtagggcttacttttggaagagggatcatttcctctgagctaaaggggaacgggtacaactttgaaacgtttcttaaaatactgtacgtgccagaagtcagaatgctcatgaagttctcaaagtccctatctagaaaattctggaacaattgctctattcagtgaaagttgcctgtgttctggayaccatcacctcaagtctcctctggtacgttgttccaccagttattgcacctctcctatatcttccatctttccttttccattcacctttcattagcaaatggatgaacacacattttaaacccgtattaaaatgggtttactcaggcaactgtaacaaaatatcataggctgggtggctgaaataacatacatttattttctctccattctgaggactagaaggctaagattaagggtttgtgtctagcaaggcctcacttgctgtcttgtagactgaagccttcttgctgagtcctcacatggcctcacatgtgagggtccagatgagagagatagccaatgtcctttcctcttcttataaggatatgttagggccccacccttaagacctcatttaatcttctttttttttttttgagtcggagtctctctctcgcccaggctggagtgcagtggcacaatctcggctcactgccacttccaccttccaggttcaagcaattctcctgcctcarcctacttagtagctgggattgtgggtgtgcatcaccatacctggctaatttttgtgtttttagtagagatggtgtttcatcatattggccaggctggtctggacctcctgaccttaggtgatctgcttgccttggcctcccaaagtgctgggataacaggtgtgagccactgggcccggcatcatttaaccttaattatctccctaaaggccctgtctccaaataaagtcgcatcatgggttagagcatcaacatgtgaatttgaggggtacacaattcaatccataacaaaactctttaatctgtgtatgtttccacttyttggttttttccatattcaccccatggccacatattaatgcatgtgtcttagtcagcttgtgttgatacaataaattccataggcagggtagcttaaacaacaaatacttatttttcacagttctggaggctggaactgtgagatcagggtggcagcatggttggggttcttgttgaagaccctcttcctggtttgcagactgccatcttctcattgtrtactttacccggtagagggagaagagagagaaagcaagctctttcttatctcttcttataaggtcactaatcccatcatgcagcctgcaccctgtgacctgattacctcccaaatgccccatctccaagtaccatcatatagataagtacatatagatgagactgacattattattgagagtaaattacatattaaatacacccagcttttagccgttaaataatgttgtgtttgtttgatcatttgtagatgaatcccattctgttttccacctttattacttttcttgtgtcgtcgcaagactggaagggcagacagtccaaatcttcatatcgaccccatggaaacttgctcaatgtatgaatagaaaggggaccatcagtcagactgtcagtctccaagcttcctctctatcttgtggactcagatgtcaaataatgtaactttcatattaggcaaatgcaaatatactaggttgtatgtatctgtatgtattttgatgactgacacactttggtatcggtcagcacacagaaaggaaaatgtacaagattgtactcctggaaagaaacaacttttagggttttcaaagattcccatacaaaaaactttaaagacaaaatgcaacacttaattgaaaattcagatttgtgaaaaagaaacaaagatatagatatattcgtattatatttttattatatattctagatgatatattaatatttgatatggtttggatctgtgtccctaccaaatctcatgttgaattgtaatccccaattttggaggtggggcctggtggggggtgattggatcatggtgctggttaattatgaatgggtcagcaccatcccctcagtgctcttctcatgataatgagtgagttttcatgtgacctggttgtttaaatgtgtgtagcacctcccttttctctaggcttcctgctccagccacgtaagacatgcctgcttccccttcaccatccgccatgattgtaagcttcctgaggcctccccaaaagcagaagctgctgtgcttcttgcacagtctgcagaaccatgagctaattaaacctcttttctttataaattaccccatctcgggtatttctttacagtgatgtgagaatggactaatacaatattataaacagcatctgtttgaatgttttcatattctttttcatatcaaatattgagtatactattttaaaatattttcacttttcacaaaagctaaattaggctgattaaatttaggcagggctttttgatttttacttatatgtgtctttcttgtttgaattattttaataagaagatacatggctttcagaaagaaagagagaagagagaagaaagaagagaagaaaagaaactggcagtgccggtgtatctgcatattttctattttgtcttgacaacgtatgcagattatagaacttcttaaaaggtcatttgataaagtgaaacaaatgacttcgaaaaaataatcctaagggaaaattgtttggaacaattaaattgtaataaaaataatttaaaccactttgcaattagtcttcttatgaacatctacaactccaaaatgattttgacaaaaatgaatcttggcaaccaattaatgctcagtctcatctaatttatttggttttcttgagagcagagaaagctgaagaagatttctagaaatgataacaatttaagacattcacacacaattgtattttttgaaaagcgtcttatgtgaatttttattacaaataaaaatattacaccttttgttgtcatctgaatacactgcaataactttgaagttgtctgaattgttatattcaaagcaaatctgcatggtgaagcttattattttcatttattaaaaaagaatataacaaactattttcagtaactgttactaactttatttagatatcaccatctgcattttaggattatctgtacaataacaattgcattgttacattaaatgcaagaacccctcttctgcttatcatttttaagagtaaagcaatagagatgaagttgcaacagtattggctattagtgatatttgccaagccaagtttctctctctctctttctctgtctctctgtatgtagtgactgtaaagctaaccatcagatttcaagtgtagattatggagtgtcttattatgtggaaataggcttctgtgagctcatacaagtatgtgtgatggttaggttcactatgaaataccttacagataaaatctcttttataatctttactatacattcatttatgattcatcaaaaacaacacaaagaatttaaaactaaatgtatgcccttgttatttgtttcatggaactcatttgaccaagatgactagatcgattaagaaaggatagacacacagaacttaattgctaagatcaaagtataattttagcaaatcaacaattgccacttatttcttcaatggtacctatgatcaagaggttaataatagaggtttatggataggactgtttccctgtagaaggcagaataccaaataccccaaattgcactgcccgtgtctttcaagtgcctatgattacaaactaaaataaaaacatatacaatgcaaacaaacgaaaagccctatggctacatttggacaatacagaaaacaatggaaagatttgtatctccctttttcctcccctgtctcctcacctcagtttttttttctttttagtagagagaggaaagaaaagaaaagtggcagaatccaaaaagagggaaggagcaagaagaggaaagagaaataggaggaattgggcaaggtgaaaatagacaaaagggttattttagttggcatttcacttgttcttccaataaaaggcatatcccatgttattttgctcattgcctttgtctgtctgctctctctgctcacccctgtgggcccatcaggccaccactcatgtctgcctgctgggggcttgtgtagaatagagcccyggcagagaagggcttgacaatcttttacataaagcctgagtaaagatccttcagatgtgtgtgatcttcaggaagccattagtttctttgagcctctgttttctctattctaaaataagaaaactgctgatgttacaaagtgctttggaggaacaacaaaaacaacagcacacatatccacacgtgcacacatacatacccggaactcacctcctgctccttcctttaagcctatgcatgcatggctttatctaatctataaaatgataataataactttgaaaaccaaaattatatatgttttttattatgcaagtgtaaacaggcagtctgtagaaaaaatacatttaaacaatataccactctcaaaataaacactaatggttttggtatattcttagagtttttcttcaatgaatatacatacatatatttttatcccaaaacacagttgtatatgtcattctgcaacttgctttttaaatgtgacagtgcaattcagacatctttttatatcaatacatatagattaatcttgctatttttaatagttgcatacttatatgcctataatttattttaactcatcctctttgtggacatttacataattttcctttttttggctaattcaagtaatactcagtgaatgtctttgcatatagattgtgttaatttttccgtaggataagttcttaaagtagaatgtttgtgtgaaaggtatgctccatttgaaattttgatggataacaccatattgccctcctaaaaagattacatcttatttgcaatatttttcatttcaattttccaagctagaaacttctgggtcatcaccaagcctctccctttcattttctagcttccttccaaattgtcaccatatcctgtgatcatactgcagaaaggatctcctcatttcatccccgcatcttcatccttccagccagtttctctttgctcatgtcttatagaagaaacttccaaaagttatccacaatccattgtccttgtcatcctgggcacttgagagaggacatggttttctaaggctccctggtatctacctggccatgccaatggaaagataaataaaatggcacatgccccttgcaggcttggcccctaggaacctgccattcacattcttcctgccatttcatcttctactggctgaatgttgccagttacaaggccctgtgggaggtggaactgcacatggcagagcctgggtctctgagtgaacatgccagaggacagctaccccacagtcctgagtgactgcccagagcaatgcctcttgtgttgagctatgacatggttgggtctatttgttacaacagttagacgtcagtagcagaagcagagaggccccatcattttttactcacagtatgagagcagccttcttgttggcctctctaactcctgctccttcccctttcagaagctctgctgccagaataatcccactttgaaccacagtgacctgtaactccttcctttaaagtcttcaggaactcattccctatagattcagatccagactctttggcctggaatggaagatcctttataacatgtccacagccttccatggggtattgggggcagagtcctcatctatcttgtcctctgtgctagayaatccaacatacagtagtgaaaccatcccccttccttctcccctgatgaaaactataaagtgtataaactgtagagttaatcaggggagaaggaagggggagaaataaaaataagccaagcttgaagcacactctgcattagtcatgaggtcagcctgcttcctcatagtggtttgctgcctgttgcctcagaatcatgtagaccctgttacaagataaaagttcccctttactgatctacagataacttgaacattacaaagaattgttttccctttgagatattctctcagttcctgcataccaataaaactactagtgactcagctggtctgaagaaccccacaggaggtgattcaccaaagaatacagtttccacatcctgatgatttcatcccccttaccctgaaaaatgaacaaccccaattttccagcccctcaccctccatgtctcccttaaaaactccagcccagaattcctaggggagatggttttgaaggggctcctcccatctcctcactctgctgccttgtgatcattgaactctttctctgctgcaagtcctgctgtctcattgtattggtctcttactgcacagtgggcatatgaacttgctgcttctctaacagtagcattcaatacatacttgcagaataaatcaattcatattgtttgctttagagacaaatataatgcaacatcaaggaggcagttttataaaaacaatttgctttgtagggttattaatttacgcttagcaattgctaaaaatactgatttcatggctcacctcttgagatagggtaaaacagcatgtttttgatattgctgatttaggtccataacattctaagcctaaaagggccttagagctcatctagtacaattttctcattttgcagataaaaacactgcattccagacagctgaagagactccaggctatgaaaccagtgggatacagtcccaagcaggccagcacccagttcataggtctgtctgccttctgtgacaccaggctgtccctaaggctaatggccctagacaacttgatctctgcagtctaatgtctgcttatattcaactggctttgtggctccatttaaggttttattttttggtgcctcatttgtcaatttggtttcccagtcttggtctgactgggaagaagcagagccagtgtaccatgcaggggacgggatgttagatctggcctcttgctctgcattcctctgtcacccatagaatttattgagcttcctcatgtacaggtccttcttcattcatcagggccagaggctgctggtgagcagggagrcattttttctaacatggtgggaggaatggcagttttataactataattataccctccagaaaataactgttacttaaaaggtgtatcgttctttgtacatggatctgaaggctcagcgatgggtacatttaaaaatcaataaagtagtcagccaaacattcagctcatgcatcacaagctcacagggattcagatttaacagaaagatggggtaggcactgcagtggtttgggccatgggcacctactgtgtaaaggaacagaaacattgtaagacatttcaaattgccctgcacttgttcaggttaggttcatcttgtcagatacctttcctattctctctggtacttctccatcacttaggatgaatggtgaagagttttctggcaaagtgcttgttgcactttggtgttaaccagctattgacagccacacactttgaagtgacttctcctacaggagtcactgatggacagcagagtgtagtgcaaatatcaggactgaggttgggaggtttaaattacaggtttcaaatgattttgggaggaaaacttccattttcaaacagggtgaattatgagaaacaacctgaaatgccacctttatgcctcttcttataacctaaaaatacagaatggtcctgagaaagtttgctttggctaagtagcatatttgtctgtaaactatgaaattgctcattcattcatccatacagtcattaagttgatacatatttattaagagtatcctcaacaccattgaatatatactcaatgagttgttctatgtggtggaattaacaataatgacaaaccaagcccctttactcatggagtttacatttcaatggaataagaataataatataacaataagaaaacataataagccatgagagctgcattggttatattttagtttgttgaaaaaacccctcaaactaagtatcttaaagcaagagttgttcatgatttcccactattttgtgggcaagagggtgggtcatttgctgccctcacctggactcacacttgtggctcctttgaaccggaaggtcagctggtggctgggcttggctgggcttggctgggcgcactgggtggccaggcccttctgaccacatcatgttttgtctctgggtggtcttagggttccatgggaacaagtaccaaagagcaagtgcttattgagtttctgtgtgtgtcatgtttgctaatgccccacaggccagagaaagtcacaaagctaggggcatggatatgagaatgcatgattcattaccgaccagtagagaaacaatctaacacattggtaataagaggcaggaagcaaactaaagaatgagggaatcaacggagagtgatggggcagttattttagataaggagatcaagatgactctctttgaggagagaacattgaaacagagacctgtgaagtagggtagggggtcaagtggaggtaccaagagagtgttccagccagagggttagcatgtgcaaatgccctgagtgggaacaacagggagccagtgtggctggagggcagtaagccaagggagaatgttatcatagtggtggaaccttgtaggctcttgcagaccacggtaaggacttcacaaggtgttctaagatggaaggaaacctgcacatgctcgttgtagaaaagggaagaatttgatttatgttttaaaaggatcactctggcttctgtgtagagaataaaccttataagagcaaaagtggaagcaggaaaagaagttaggagaaaagagagagtgaggtggcttggaatgcaattataacagcgcagatggtgagaaagagacaaactcagcatatgttttgaaatcagggctcacggtacttaaaatggattgaatgtgggttatgagagaaaggcatgaaggatgccttctaggttattgtctgaccaccttggtgagtggtgtttctattacgagagagatcgcattggctgggtcagggtagaatgcaacatagaagatagcggacatatttagaaaatgaaatcaatgacattgcctcacaaaattctaaagctgaaaatccagcaactgacataatcccatcattttatagacaaagaaattgggatctgggaaaatgaaggaatttctttttttaaaaaacactttctgtaatcccagcactttgggaggctgaggtgggcagatcatgaggtcaggagatcgagaccatcctggctaacacggtgaaaccgcgtctctactaaaattacaaaaaattagccaggcacagtggtgggcgcctgtagtctcagccactcgggaggctgaggcaggagaatggcatgaacctgggaggcggagcttgcagtgagctgagatagcactattgcactccagtctgggcaacagagtgggactccatctcaaaaaaaaaaaaaaaagacatttattaggccatttttgcattgctttaaaggaatactgggactggataatttataagaaaacaggcttaattgacttatggttctgcagactgtacaggaagcatggtgccagcatctgcttctggggaggcctcaagaaacttttattcatggcagaaggcgaagtggtrgcaggcagttcacatggtgaaagcaggggcggggccgggrggggggaggtactgcactttattagtttcatcctacaggtgaaattagtttgctgataaatgtcaaatgctttaagatttcggagaaagttatttagatactctgactctaatttttttgtggagggaggatcacattcgataaacactcaagagaataagatagaggaatgaggcaccggcagaagggttttgtattagtctgttctcaccctgttttgaataaatacctgagactgggtagtttataaagaaaagagtttaatttactcacagttccacatagctggagagcctcagggaacttacaattacggtgaaggcaccccttcacagggygggaatagagagaaaatgagtgccagcaggagaaatgccagatgcttataaaaccatcagatcttgtgagacttgctcattatcatgaaaacagcatgggggaaactgccctcatgattcagttacctccacctggtccc

SEQ ID NO: 2 - DNA, 63266 bp, Homo sapiens
ctttttacctgttgggcagtaggggtagactgcagttatcccgtagaggtgagatcgttgttctgggagatactcatcagtaaactggccactatgtttatttactgatacaacaccatccctggtaacagaaaggaagagtgaattcagggacagtcatttactagataaagaagtcagtcagccacagaaaatcagttgcaatagaggaaaatttctggcagccttctctgtcccagggctggtgctaaagccatactgaagtttgaagaccattgctctaaatccattgctcatctctagctgcatgttagaatcacatgatgagctatcaaatcagagttgtagggcatggtgtaaagggcttagactttaatgttccacagttcctcatttgagaactagtctaaatagtatttttcggtccctcagacaatatgtccacagtgtcaaaagccaacttaagccctgtaatatagctcatggtatcaagggtagtcttattctctaaagaacatatttagattaatatgctcccttttgagacattatccaatagctttgctactttttaagctatatgtgagcaataccattcgatttctgggtgaagtaaaaggatgatttcaaaaacatgaaaggatgaaaaacatccttgaaggatcttatctgccaatgaggaggtgatgaaacaaaagcctcagagggcttaatgagtcaagtcaggtactgacttttggtaaaacaagtggtgtgtcctctggaacagttggccttttcaggcttgtccagaatttgtggggctcacctcctccagtctgtgtggtagaagtgatctgcatagcttacgatgctgaaggggtacttgaggttgttttgaatgacacgccgtccagttccatcaggtagtgtacactccagttttttggttccttttaaaacaaagggggaaaatgaggtccttcagctgctttgccaaaaatgatagtgacatagtgatagtgacacagtgatagaatggggaaataggatattttacttccattacagtcatagattagcaactgtaaaattacctgtcctattactagtctccagtttttagtagagattcaaactttcaatccttttttggaagacaggattgcatttactgaaatctggtaagtttctgccaaagtagggcattaaacataattatttttataatttgtgtgatttcaagccttagcttataaattataacatctgggtaaatacttgccctttgtaagctatgtctataattactgaggcaaccttttaaccaaaatccttagtatttttaaaatattttctaggtaggaggaaaaaagtgactaaaatggtaacataactcatatttcaatgtaaaggaatcagtcttaaaaatttttacaatagctaaaccattcaagaatttccaagtttcacatttctcatggagccgatttaaatttcaaatccttttaagaaatgtaacttgttttaaacaactgggttataaaagtgacctatgtacattaggcccttttattagattacaaattcaagaacagtctttttcatattctttagtatagaactagacacacagcattttttcagttgtgctgataaaagcaaacaaatgtaggttgctaatacccatgttattactgaaggaagctaccctgctacaatgctttccaaagaatgtccttagcattataacagatgtttataggtaagttatagtgaccctctccccgcataagaataacttcacttaagtttgtcaattcaacaatggctaattcttttaactgttaaaaatatgccaggcactcatggtcaggcaagcagtacaaacttactgcttgatatatccttccctccacccaaaccccagaaagttcattttaagactcatttactagtacttaaatttactagtacttaaaagtttacagaccaaagaaaggagatctaagtgatgggatttcattttgtagtaaaatctgttagtgctcacattcactgtgatgagaggttatctatttgcattccatcagtagtattacctgcatctgcccagcagagcagtttagagaaagggtcaaaggttaagccattgggcaatccaatgtctgtattgatcagaattcttctgttttctccatctaaagatgacgtttcaattttaggagcttctctattccagtctgtccagtacaagttgctgtaagttaaaaatcaagattgtaaaagaatagccatgtagcctgtggtaggcagcatctaagcagcccccagtgatctccatctcctcatgtattatagccttaagcaatccccttgagtttgggctgcattagtagtgtcttgcttcttctagtgcatagagtatagcagaagtgataggctatcactgccgatattcggtctcaaaaaactggtttctgccttaggggctctctcttgctccctttgagggaagctggatgccatgttgcaagctaccctgtaaaggggaacatgtgccaaggaactgatgtccttgagggctgttggccagagacattagtcttgcctgtagcccatgtgagcatgcttgggctgtgtcttccccgagctgagccttgtgatcactgcagtttggccattacctgattgcagccttgtgagagactccatgcctgagggagctcggtgaagacacacttggattcctgactcacagaaactgtgagactaaatgttatcttacactgctgagggtttgggcaatttcttacacagcagtaggtaactaatacacagccatattccacttttacctgtatcacaaggttttctgattattgtcactatgtgggagaagtcattactaagttactatcagaatccctgcctccaagataccccttgactcaactgccccaaaaccctccgtttgttgaagtcttgaattggatcttgggttttagaatagaaacaaatacagctgttaggaatggcttcgatacaaactatggaacttgaagagatgtggaccagggatttgagaaggaaactgttctttcttctgccaaaaatacaatttggtacttgaaggggtacctggcacccagggatggaaacattttaagttaagcactttaccttttaaatgccacgttaaaaatgaaatatattcataacagatactaacgaggaaatatggtcctattaagtggaaataggttataaggaaacaaacaggtttatcgttagagcagaaataatgttgaaagcactttttataaaatactattttataaaagctatagaagctttgaataaatcagttgggctacagagaacctcagtgagaggagaggaagaaaccataggtttcccctgttgcgaatttcatcccttatgcccaacagatgaagcagagagaagctgagagcaaaagctagaaagcagtttccactttgcgctaaggtggaggtggccagagggtacagcatgcatatcattgcttcatcgcttctgatcagtctcctccccaaaatgatttaatatcatcaacaacaataagtctgaaacaagctcacacagatatttggtgtaaattatttggggattgccagaggaagaaagttgtaaaggtgaaatctatagaaagcaaatgagaaatatgcaggaaggcttgacaaccattctgttacgcagttgaaggagatgtaattacatgtgttatattgcaacctggttcaaggtgggttttctttttattaaaaattcaagtcctttagcatttatatttgctgtctggataagaaagttctactttcatatttaaactcatggctgtgatgtggatttcagcttaaaagtatgctttttccccagactccagagacatggccaaagtttcattgcctacaagatggcagctctccccagttttcaggtcaagcaccaagaaggaataaggggccgggcgcagtggctcatacctataatcccagcactttgggaggccaaggcaggtggatcacccagggtcaggagttggagaccaaaatggcgaaaccctgtctcgactaaaaatacaaaaattagctgggcatggtggcacatgcctgtagtcccagctacttgggaggctgaggcaggagaattgcttgaacccgggagatggaggttgtagtcagccgagactgtgccactgcaccccagcctcggcaacaaagcaagaccttgtctcaaaaaaaagaaaaaaaaaagaaaaaaggaccttccaaatgtccaaatgtcagaaaggcaagcctatgcaaagagtttttaagagggctattaattcctgttaagtcaactggaatagagaaggatacaggtacttttcctgtgtgtagaacccaaagtttgaaaccacctttgaggcactgcactaagtatttcatagtcaaaaagcacttacctgaaaattaaagctgaaggttttctttactgatttgaagggcatctgggaatttcatttcgtagaaactccacttaaacatggcaagagtttctcaagatataaaaattctccctgattgtaacaagtactgagacagtgaacaaatcctgtatgtcacatttgatgaccttagcaattatacagacagggaaaatgtttatcttgatttctgctacagcaacactgaagccagaggtgtctgagctttggctaatgtcacttgatgtaggtcagccctactgtgagcgtcaaccctggtttctaaatgtgctgctggaggaatcccacttcacttccagtttaagtgtcagctgaggcctcgcctgatcatttgattcagagtagccctagttctcacaacactatctgaattttctacatatcacatcacagactaatattgtttataccagtcttgtttcctcctaataaaatataagtgccatgaaagaacagaccttgtcttttcctagaacaggtcctgacacctcttagatcctcagtaaatctttgttacatgaatgcatttgagctttcactctcttgttctgaccttccccacattgtatttaaaacccaagggcttcacatcaggatctacaggtaagagaagaattagggaagcccagctgaccctcggattggatccacagcgatggcacggggattcaccagatctgtgtagaagaggaccttgcgctcagagccatccagcagggcgctctctatcttatccaggacactgtccgtccagtacattgttctgcggatgtggtctatggcaagtccttcagggcttatcagacctggaaataaagcaaagtcaggactggagtgtttgcaggatatggagggcaaacctgaagccctccaacggtcgggtgggcaggggggtcagcaggggaaagcaaagcccaagtgtctccaagtccccaagaaccaggttaatagagaaacaagtacttttgttacatattccactacctctagcttctagaccagcagacctaccagtttgatgacaaccggcatccacagtgccagagccaggctgaataaacgaaggcgtcaccctgaattcttaccatgaattcttttaagaagtgcgcacagtaggaatcacctatttcattccgtatgtatgaaggtgtttgcttacagagggggctagtcttatacagctcaataaaatgacttgcctagaacttaacctcccctaatggagaaaaatcgaggggagacttcggcgtaaagtagacaagtgactttgtagaatcaaatgaaatgcagactgctgagtgaagctgctgacctgaattcacgatcgtctcaggctctgctcccagttccagaccagcacggctgattgtccgtccagcaacatctgtccagtacaccatcctctcccggcagtcgtaatcaattcccacgattatggagccctttgtgcatcaaatcatagaattagaagaattaggttacatttcccctttgttcacactcagctgccttgctcatgcacagctgcagtgagcaacactgcactgtgatcctgcaggcaacagagcagagtagctggacatgtgggcactcgggtgaaatctaggctcagccacttagtagccatgtggctttgggcatttaacctctatgcgtttgtctcatttgcaaaatgaggccaatagtgtacctgcctcatatggttgctataaaaattaatactataagtgcttgtaaagtgtcctaatcagtaattggcatacagtaagcccttcaataggtgctattatgactgatgatttaaaaaaaaaaaaaaatcagccccttcagtaattagaggacatggccttaagcaaaataagtttttaatattttactgggtaaaaactgaatcttttcccaatgtcatcagcacttttcccagagccttaataaaatttgccactatatgccaacaaaatctactctcccctcctctgtgaatgagatctttcctctacctgaagtacaatcacctatactcctgcccctgactcttaggaggcaagatgttggttatatcctgtactaaagaaagtgtagtcaggcacagtgactcacgcctgtaatccctgtccgttgggaggccaaagcaggaggattgcttgagcccaggagttcgagactgagcaacatagtgagaccccgtctaaaaaacaaaaaataaatgaactgggcatggtggcacgcacctgtagtcccagctactcaggagaccgagttgggaggatcgcttgagcccaggaagtcaaggctgcaccaagccgagatggtgccactgcactgcagcctgggcaacacagagctacaccctgtctcaaaagaaggaaaaaatctaatttattgggttaaggaactagggagtagtggttatccaacctctggctagtgaggtctctcctcctcattgctaaataaatgacaagtcttttaaaaaaataataaaaataaaaaaaatgctgagtctgatatttttcctcaactccatgcagaacttgcttcattcagtttccaatccaggctcctgctctgagaagcccagcagatgtgcaaagccaacattcaggggttagctggctgtccctgaggtcatacaagttcctgccttgcctaacagcctcctggggcttggcttcacttacgccccctgggagcgtagtcagccaagaaagattgtacaaaacgggatttggcttcagtggaaaaacaggcagcttatatggggggcgggcctaaggtcaactacacttttatttgaaaattgaaaagtcgtttgtcccaggcaagagtacacattcaaatgagcttccattaacccgggaatcatgggattcataaacccatgtcataacctcaggttagagaaaaagtcccagtttaagtaaatttccttaaaatgtgcaaataacccacgtcccatttctttcagttccactaccactaccatcaccaccctgtcctggtcaggggcatgttcagaaaggcaatcaagctctatgacttgaataaaagagggttagcttaaagtgatgtcaacttcttagtctctacctcttccagagaaagttttacaaaacaatgtgagagttttcttgggggaggggacaggacccaacaaactcgaatcttaaaaagaacacatctcttcctaaacactggctgcctctaagcttttctggaaccaaataagtgtcgttcattaacagaagtaaatgtaaacttgaatgccgtactattctttctcagggatgagcaagtgcctggagtgtgaagttcaacaatggaatcagatgattttccactgtgtcgatgccattcgagacagccacccttttttttcctccatttttgagaaaacccgctccccaaaatggggtgccttgcattagggaggctgctggctcaggaagcatcaaacatcccctggggaaggctccatccactcctctcccagcatcacacactcctcaagttttcatctatggagtcagggaaggttaggaaagaagtgggtcaaagggacagaaccaaagtgaaagggaagggctgcaacggtgagacagtcattaactgacttccacttggagtccccaacaaaccgcagagatcacaccttgtgactagttgtcctgtcactttgatttccaggatttccatggagatgttatgacatacaaccctggggacttagggtaagagtcggtccattttcccttggatcaccatacagcctccatgtgctgacaactctaaagttccctttccatattatctgaaactccatgaacgaaccagagagcacagccttggtcagtgacaggtgactcccagcaagagagagccctggtcctatggtggtggcatcgggcccatgccgatgggctgcagctcagaaaccctccaggacttccctgcaccagtccacctcactgcaacagggcctgtgaggtggctcccactgggaaagccacgcagccctgccccctacaggagaaatccactttacatgcagagacagcagggtcttagctgcatccttctgaagcctggtgccattgaggggtaagtagccaatctgctggccctgagtatagagcaggaaggtgcccacagatggaggggtcacatctggccggggcgtgggccggaccatgggtggagcgacggtgggtatacctgtgggagagagggtgggagagcaggaaggggaacaagggcaggcagggagatgggaagaaaagttactttaccacataccagagcctccaaaaatatcaaaagttcttcgccctacgcagatgtgctccagatgttcttcaaaagaaattctaatttctagggtttttttaaggatcaattatgtaaatctgccatttactagggaagaagacagcaaaacacttccctcagaacaaagcagaacagatttttttttcaagagaaaaaaaaaacagagccagcattgtgtcagcttccttttaggactccagagtgacacgtgtcccctgtcaccctgggctggggagggggagtaactgcctgctgaaccgagttgcagatgttccaattatggaagctgctaatgacaaaaaccagacaccaaacgcaaattgggatcttgtgtggaccagattataaatcaacatagcacatagtgacttctttcattagacctggtgaccccacatgtccccccacttcaccacagcaggcaccatccctagcaaaggccttagatacagctgaggggagcgggggcggccaggtgcttacacgcaggggtggtgcctggctgggagcgggtgccctgcacctctctgccatctttgtccacacaccagcagaagtcgctctttccgtggcactgcagggggatgaagtggcccaggtcatcgcactggggcacgtactggtcatcccggggggtgccaccgtagtgctccagcaggttttccctccagcgctcacagatggtcgggggcctctgggtgggctctgagcagatggggaagagggaagaagaaaaacctttgattgtgagtcaaggacagaatgcaaaatgtagctcaagaccccatgcctggcctcctggccttccttctcctactgtccaggtctcagccaagcccgtggacaccaaacttgggaagcaaatgtgttctaagtacttctccaccatgggaatcagaaatagatcatgtgatgcttctttcttgccaggagggtctgtcatcaaggcccagccacatcaagaaagaacaagaggaggtaagatcagcagtgcagctcactcagcctgggaaggggcatgatctccttctggccaggagaggcccacaaggcccatcctgcctgtacctcgggcgcctctgctgacagagcacttgtgcaggtgatccttggggactcagtagccgaggtgatgcttaaagccaatttgtattcattccccacttgcttcaagcttcaagctcgaagctgctgaggagagggccatcttgcgactggccagtgtgtgtcttagaaagtattactgaaagcccagcgccacggtgccctctgtctacccactggaaaactacatggagtaaaggtaaaactgagtggaattagtagtaaagcatagcagaggtggtggcaaacagcttaaaaatggttcttccacagcccagtttccagaattgctttagccaccctgcgcctgggattatctcaaagctgaatcttttcctgcaggctctagcttcctgccgtgttgccctttccccccctttctccagcatcctctcctcttgtcccctccctcacagctcactccatggttgaagtcctgctggctttggttgcaaagcagtttcctcccaggctgcttccacagtgccattgaggctttgtccaaaatacaggttgttctgagaagactctgctgcagaaggttattgagataacttgttcatttatgttaggactacccaaaggaacttaaacaagaggaggtgcttctccttcctttttaattcctgcaacttaaagcacatggtctgcagaatcacatagccaataaccctacttgctaccaccatttagatgtataatagaaatcagaaactaatagaaactgagcccctgccttcaccatcaaaccagctgctcccctggtctttcccatatgagtaaatggcacctccatttttccactgctcagacctaattatccttgaccctcctttctctcaccatccacatttgatatgtcaaaaactctttttaggggtaacttcagaatccacttagaatatctttacttctcactactgcaccccctcaccctggtcaaaaagcctcatcctctccctgcaatttctgcaacagccttcccactggttttcctgcttccacccttctatgccctcactctctccaaaaccagtgtaaattcaattagcagcagccagaatggcccattttaaacataaaggcatttcatgtcactccgttcaaaaccctccagtgactctaaagaacaaagctgggagcattatgatcaggttccagcacctacaattcctcttggaacacaaaagactgcgaatgaagttgtcagagctcaaacccaactagctctagctcaactgcacgactcaccagaattgggggaagcgaatgccgttaagttgtcactgtagctcatgacccatgggagacaaagctctttttaagaccccgtggagaaggtaagactctgacttcttcagccaaatgtgtctcaaggaacacagagagggaaatgctacttcctcaaacactgcaggatttcctggagaagttcccaattccacccctgctccacattcagaactccaaggatccatttttgctttaagggaccaccctgtgctatgtgtctgcattccaaacaaagagacatgcctaactcctgggaaatgggtgtcccaaatcactcatacatgggcatccctttcattcgttactatttgctacttttcaacccaaccatgaatttctttctcttgtgcttctggttggcaagggtttaagtattttgaaattgtgggcacagagcgtccttaccctccctcaagctgaccccatcaaacatgagaccagtcagagatagcagttggggccaaaccagcgccgagagagtgtgggcaggaaaaagtccgcacaagagaacacgaccctattttcgtcattcacttctcagtctcttggttcccccccctttaaaatcttggttccccctttaaaatcattgtccttaacacatgcctgtcttaataaatttaatcgaattctgaaccattccccaaaggcacctcccaaccatttgatttcttcaaatcccagatggagttagaaccaagacattttcaaagagaaggcatgagggaaatagaatttctctgtaccttttttggacctttaaaatcttttcctaaaaatagaaacaaagagcgactatggatcaaaccattaccaatttttacctatctgagaaccgccaagacctgacctttttttaaaaaaacaaaaaaaacagagtctcactctgttgccaggctggagggcagtggcatgatcttggcctactgcaacctctgcctcctgggttcaagtgattctcctgcctcagcctcccgaggagctgggactacaggtgcgtgccaccacgcccagctaatttttgtatttttagtagagacggggtttcactatgttggccaggatggtctcaatctcttgacctcgtgatccacccgcctcggcctcccaaagtgctgggattacaggcatgagccaccatacccgggcaacagttaacaccattaaaagggaaaaaaactattggtaattctccagttcttcttactttcacacattaaaagccaagactagtatttatggaacccttctcaatagtaaaaatggatctttggatagcaagctgtgtcggaatcattttggctaaaacggtatcaagccaatcctatgctcttcattaaataaagttcttctgtaatgattaaagaaacaagttttacctatccctgaataattcaacaaggaacagaaagttgccttatctgcctgctgttggaatcgactgtgagcacttgatcaccatgccccacccagctggggaatgctctagatggtagcaggcatgttctcaggttgtatgggctaacagaaaccagatgtctgtgcctcctctgagatgttgggctgggttacagagggctgggattgtcagtttagagaaaactaactggctccactagcaggtgttatttttctaatttttcctattgatactaacagagcgttcatactgtttcctgccattcagaaggcagaggtaagtgagatagttacataggaaagcagttttccttctggtcaaaacgccagtctggccagatcttctaactgtcagatctccctaagctgctctataacaggtctagggtagtgttctcagtctgacctacatacacaatctcaactttcagtttgtactggacaagggtctaagaggcaaatgtgtaacagatcatatgtgtcatgtattgcatatctctatgtccttatgacacaaacatgcatatttgtacgcactattattcaagtcttctcaggaggaaagaggctatgaaggatggtgactggccccgtgcctgcgtggtctgggcaggaattatgatctacctaccaccagtgccattctgcttcttgagccccaggcctaagctcacctggtgatggtccacagtgaggcggggtggagccaggtggagtctgggtaccaggaacttcatgaccatcagggtccacgcaccagcagaaaccagtgctgccatgacactgtaggggcaggaagttgccctgctcgtcgcattgggggatgtggaaccgggccccagggtaggcatactgggcctgggcatggcgctgctgttgttcacagggtgtcaggcttgaggtggagtcttccaggatcaaggcagaggaagacacaaaagagaaatagaaggcattgcaacccatccacttcaccagcccacagcgtggcatggaaaagcgctgggattttggagcccgagattcttattttataataccttgccactatttacctcatttctctccagttcttctagttcatcctataggtccctagctggctctaagtgtaaagcagcccggcccctccctattgtcatctggggaaaatgagggatagcggtggcatctgcccaacatcccaagagactctggagaaaacaagccaaagctcattcatgccctgaggcccacaggttaagtctgagaagctgcaagccaacggcactcaccactcacgagactgcaccaagctagccaagctcctctaacacggtcgccacgggcttaaccctggctaccggcgcataaggctctgcttaataagcttgatctcaacacttcatccaaattgcactcatcaaggtcaagcagatcactagggcagttaaccagaggggctatgaggtaggacgatgggtgggtcgtctcccgcaggcctccagaaatgttacgaatcagtgaactatcagtaaactaggcagcaggaggtaaccaaggaaaaaaatccacagcttggaaaatccaccaaggctccagaccaggctaaatcaagagtgggctgtcatagaaaaatatccttgaaggagctaagagattcatgtgcctaatctggccctctgaaacttaccaggtatgcactgaaatccatccccataatatccgggttgacaacggcaggagaaggaaccaggagtattgtagcaggtagctgcagggtgacatctgttttctgagcattcatctacatctgcagaggtcagaaacagaagaaagaagcaaagaacactatgacttcactcacacaatctgtgcgactgcatgggctttggatatgtggatcaacacccagtgaggtgtcagcttcagaagacctttgagcatgagcagaaaaatgctgacacctaaaggtaagcttaatagaaaggaagaaaaaaaatcaaggaacatgtgagttaacacactagactaaacgtgttcagtgtagaaatcaaatgggtagtcgctcacagaaaaaaacaacacatcttacagcatgtttcattgacagtgaattcaaggaagttattacctttgaactaacctttagactataagtcattaacttcctttcagcaatgctgcaataaagacaaatatatccaacaatggtttttttttctttcgttgttggcaccatctccctgtaacattcgtctgtagatgcaaaggagccagatgaggcccaaatatttttcagctaagaagcacattctgtcttgactaattagaatcttactatctagaaccacacccccattaaccaggtattttctttactttccacttttagaagaaataagaaaaaaatattatctagggttttgaaataatacttacaaaatggaaatctaaatttgccaggcatatcagtacaaacaattcaggcccctacactctccacatagcaccatgaggattaattttcaccatcatatttctcaaaacaacaaaatcctgacacttcaaacaaaagccaacttacattccttttatttaatgaaatccgcatatgccgacaatagcaagtcacccacccctcccatccccagcagaaaaaaagcagctctgagaattgaatggttaaaactaacgcccaagggatgagataagctactttaagagacaggaagtctttcagagtaaaaggaggataaacaaagggtgtaggtccgattaaggcttggctgaaaataaaacaaagcagtagttccatccatagtaataaccaagtttctttaaaatgtgtgaatagaaaaaaaaaaaatgtgggatggtgtgatcagtgtgaaacctagaaaatgagaatgtgttgcatcaatacggtgcagcccatggtagagacacatcagcatgggaagacagatatttgaagccctaaatttgatgtgaatgggttgggaacagggctagctcctcacccaggctcccatgacccacatggaacctcttctagagttacctgcaccaccctttgtccagaccacactgtcactgggtcatcctctaataactaatgtgctttctcgaataccattaagtacacacactgggcattaagcgtaggtgttctgttccctagagaggaatctcttatgttataattagaaggtaagtattctgcttatcttctgtctctggggagtttagtcagcatatatcagttcctaggagtcctcccccaaaaattctgaactcaatgcaacccatagagtctccacccaggtgagtggttggcctccctggataccatcatttcgcctataaaggcaagaaagcagatatcaattcatctgcaagggtagaaacaaaccagattggctcctcagttaccctgaaatcaaccaatgttgtcataattatccctgagcttggctctgcctcttcaacaggacaattcaaatgcccagctgggcaaaactggtgaccatccaataccttggacctggaataggctgtaaaactggctgtaaaactaaacctgtaaaaattaacattcagccgaatgcaggaaattaaagctattcacaatacgcatgctaaaagtacttctaatggtaacaggaaccccaagcaatgacaggaagcaggaattcctgagacacagggcctatttctggagcccatggagcagtttcttattgttacttttgaactcaagagagaggggattccccagagctaccaaataaatcaacggtgtgcttagaaccttcagcctaagaattctgactcaatctgaaagaccagccttcctccccacagatactgttggcatcatcaaagatactaaagttctgggtagacagaaactattagaaatgaccaacaaggaggagaaagtccccaaagagactgaagatatcacaagtgagaaattcctgccccaaagattgagactccctgaattcgcagtcctgactcctccttggagggcctctcacaccatgatagagactagagtcccgggacctgggcatgcacaggacatcagatgtcaagagagaaaacatggaaagactttcagccccctggcaagggaggaactttattggagaaggagaaggaggccactgcttatgcttagtcattactagcaggggcctgtagggactgctataaagacaaaaacgttcaagaagtcgtagagcccctttatcactcaggtaatgagaggtagcttaatctcagtgcaatcctacactctattaagctagtaaagaacctgtttggaaaatgtgttaaaaatccttcgagttcatgctttcgtgcttcaacatccttctcactgcccaagtactgagcaaaaccagcaggaactgttggatgataaacctaacccctggcctcgagttcccccaggctcccgccccttcaccactgcccacttgcctttgagcccagtgaaatttcttggcatcaattaacctcaaacccatcccagatcccaataaaggattcaacccacaggtcccactctctctctactgcacaatctgttcaattgggcaccctgctgattgtacccctgtatggcatgtggtgaacccactccagacctgtaagtgtaataaactacttcctttcaagcttcatccttgcctcctcctataaccacactgattttaccataccatacccaacatctacctacattcttggaacaggaaacctggccaggcacataatggagatcatgctttcgttatcaaagaagagagaagctaggccgggtgtggtggctgacgcctgtaatcccagcactttgggaggctgaggggggtagatcgattgagctcaggagtttgagacaagcctgggcaacatgatgaaaccccatctctacaagagataccaaaattagctgggcatgctacttgggggggctgagatgtgaggatcacttgagcctggaaggctgaggctgcagtgagctgtaatcacaccactgccctccagcctgggtgataaagtaagaccctgcctaaaaaaataaataaataaataaaaattaaaaaaaaataagaaaagaaaaaacgccaggaaacccaagaaagaataatgtcagtatcttccctcaggcccctgagtaatgatggctctgggtaatgggccatctgtaacccagcttcctcaactcacacttgctcctcctgggaaggtcccaactcaagacaatctgcttttgcacctggccaagtcacaacaatccaaaaggacaaacatgtctgagaaatatccagctcacaccaacacaagtttgaaatcactccagattgagggaagctgcagaggatttaactctacttggataaaagcctcctggctagtgactcagaccagactgagaacccaaatccaagtgcagcctctccagggagccggtttttttaacaaactatggctggagaaaaactcccacacccccttattgttagtttgatggagtaaaagagcatgtgctttaacaatgcatagcacccacaaaatacagcctctgttctttggtcactgagacacatgtaccctatggaatgcctttaaatatgcacacatacacatatatttcaatatttattcttcaagttactttcatgattagcctagcctgatttatgcatgggtctttaggaaaatgaccattcattctctactactccacaatctgaaagtttagaagacaactacatggtaagtggctgtggtaggcagcctctaagatggcccccaacagtccctacctagtggtattcatggtcttgtgtaatccccaccctttcagtaacttgcttttaattaataaagcaacattgaagggatgtcatttttgtgattatgacttccatcttgctagcagactttctctggcattaataaagtaagctgtagtgttagagaggcccaagtggcaagaaactgaaggcagccaacaaccagcaaggaaccaaggctttcaatccaatggtcctcaaagaaccgaatcctgcccataaccacgagtggatctttccccagttaagccttcagatgagaccactgacccaccagacactttgactgtggccttgtgagagatcctaaagcacaggacccagctgagccattcccaggtccctggaccactgatattaaatgtctattgcttgaagctgctacattttagggtaatttattatacagcaatagataaccaatacactgggggaagcacctgatgggtttggcatatttttagtttgtaggctttggaaagaatcctaaaagcagagaaccagaaataaaatagggacaattgaaaatgacaaaaatagggcaaattaaaaaatgacaatacttaaattcccaatatggaaaagtagtcaatttcagagccctaacatgaccccagaaggtaatcaaagtcttgcaggacaagtagaagaaaaaaggagggagagaatcattgtagaagtgatcaaagcatgcagcatctaatctaaattaatattctgattaacactcatcatatctaggctgaaattaatttcaacttctttgctttaaaccagagagctaaggaactaaatattttaaaagcaccatctctgctgatctgagcagccagatgtttaaataaattctacagacaagatttttctcagtggattcacatttcttagagctggtactagggttaaaccaaacaaacgatgtgttccacacgaaggaaaagcatcattcccactgtcaggctttggcacctcaccctgaactccatacctgtagagggtatctgtaaagagtgtatctattatagatatactcaatgttttctcaccagtatatctatgacagatggagctcctggctggttaagtgggacaaacatctgtgaattacaaatctacctcattctaattgggaaatactctttatctagagctaggggtgtttattcaagaggtccatagtcttcttacattactgggcctaataccaagacacacaggaatcccaactgagggttaccatctgtattagtcaatttcctgttgtttataatagaatatctgaaactgagtcatttctaaaggaaaggaatttatttcttatagttctaaaggctgagaagtctaaggtcaagggttgaaatctggtgatggccttcctgctggttgggactctcagtagagggctgaggtggtgcaggacatcacatggtgagggggctgagtgtgctaacatgttattttaggcaggcctcccttcttatcaagcctctagttccattcccaagataacccattaatccattgataggggcagagctctcatgatccaatcaccttttaaaggccctacctctcaatactgccacagtggaatgtaagtttcaacatgagttttgaagaagacgaatattcaagccatagcttcaactttcaataccaccaccaaaagtatctatttagtgaaagaaaaaaataaaagggctgaactgagccacggctttgggttagttcagcataaattgttcgagaaaaccatgcagcagagcttctgtacagcacaagtggaaagagtaagaatgaggctcagaaaccaccatgtgcacttgccccatccagagagaagtggagggctgagctgctgtcacagtgacctttgttctgtcagtaacacatggcctcagcctgctgtccactcagcccatcctgctgaaaccaaaggggaaaaaggaatgggagaaggatgagaatcaaacatctggtgaggaggagggaggcccgtgctattcggatctggtataaagctcccaggaattgcaaagctgttcttagcacatcagctcggtgggcctgggcggatggaggcagatgcacacagcaacaccaggagcaggggaaagtctgccttttaaggttcatgttccccagcaggccctttcccattctaaacacgtttcccaccattctctcatccccagtgtgtcgttttaccctaactgtgtgtgtgtgactcacgtgaaactgagtttcaatctgatcacataccacagggtatgtttggaaaaccttttgtgatttaagatttatttaatgatttctattttaaagtactcctaatcctagtttcatggacatttatactcagagagttatgtcgagcctttaaataacaacctcacacattctttctaagttaaattccaatggctagccttgcaaatggtttggtgcttttataaaatactgcattaagtcttaaagatagatttctgggagaaaagttttgatatgtgtctttttatatatgtggagttctctcttttgaaagaggagaaaaattagaaattacatatgtgtttagagattgcttaaaatatcacttaaagcagaagagcacaaccataagtgttgaattaaggtggagaccgcctaacaggaaggtatgcaactttccctaaattcctccagcctgcgctggctccaaggaaccaaggttagaagctcccgagtgctacaggaacgtctccgccaaggaagccatatatgctcgcccatctagtccagaggaaatcacccttggctcccctttctaactcaaaggacgcttttttctcactattttggcagccacaaggctgttagagtcaaaggaagtcctttgtcttttctgggagagctccatctatgtggggatgtgcatccaaagcaactttccagtcattgaggctgacctgcagttactcaccagtgcactggtgcccatcgccggcataaccaggcaggcaggcacagctgaacgtgctgcctccatggtgaacacaccgggcctgcccagcaggagcacaggtatgactgccatcctcacaggggttggcaggtggggtgatcactgaaaagagaagaagataagggcatccagagtttaggcctgcaaaggatgagcctcactcctcatggcatgatccacagaccaacagcaacagaccagggaatgcaggttctcagaccctacccaaggtccagtgattcagaatctggattttaataagattcctagatggtttgtatgctattaaagttcaagtactggtatgaatttgctgttaaaatactaagcacaaaaacccatcacctaagcagtcctcaattaaagtttataacactccagagcaattgctacacacacacacacacacacacacacacacacacgcagtttctcctgtcacttttaccctcatcccactgaattaaagagtgcattttctttcttttttttttttttttgagatggagtctcactctgtcgccaggctggagtacagtggcgtgatctcggctcactgcaacctccaactccctggttcaagtgattctcctgcctcagcctcctgagtagctgggattacaggcgtgagccaccatgcctggctaaagagtgcattttctttactctcatccatctttatcaatgggatgaattgggatagttcttttgatccatttttaggtctgtagtattttctggaaatacagaagaggtaatttgttacagaaggagctctttagccagtcaagtaggacatgcatcagtgaattataaatccatctcttctaatttgaaaaatactttaggctctatctaaagagctggaggtgtttactagtgaagttcatcatcttgttacatgactgggccagaaatcccaactgaggtgggcatccttcaataactcaacaccaaaaatatctatttagggaaaaacagtgaaaataaacagggctgaaactgagccatagctttgagagattttgttgtttttttttttgtttttttgagacagagtctcactctattacctaggctggaatgcactagcatgctgtcagttcactgcaacctctgcctcctgcgctcaagcaatcctcctatctcagcctcctgagtagctgggactacaggcgtgtgccaccacacctggctaatgttttgtattgtttgtagagacggggttttgctatgttgcccaggctggtctcgaacttctgaactcaagcgatccacctgcctcagccttccaaagcgatgattacaggcgtgagccaccacatccagccagctttgggttattttgatgtaaactgtcttcttaagaaaaccatatagcagagcctctgaatagcagaagtcaaaacactgacagattaaagagcaagattaaagagcaccattttggccacacaggaaaatgattttggaactcacagatgcaagtatgccggtcatctgcaaactcataaccactccggcactcacacctgtagcttccaggcaagttgatacatacagagttggggccacagcgatgaaagccagttgcacattcattttcatctaagaagaaatgagaagagaaaaattctgcttaattcaagggtcccagaagtggagggtctaaaaactcaatgttttctcactagtatgatgcttcaatgggacaaatggttggcagatgtagaatttttaaaaatcagatatgaatgcatgttgcagataatcagaaatagtaaaaatttcatgccattcaagtaaacaccacaggaagataaatttttaagtgaatattctaagaattctttaactgttttaaaaagaatggatacttggaagaaaaccaacaattttttttaatatcaagaaatttctaccgttgccttttggggatataagacacacaatgtctgtctaaaatttaaagaaaatcagaaaatgtgctttttggtaagtctaagaactacttactacaaaagctcaaaattacaaaacttaactcaaaacatccaggaactgcaacagatgttttcttttcacagccccggtgcaaaatccactccactccaaaccatcttttcaaacataaacagctatgctttaatttttaaataactatattcagattaacagctgctaaaatcattgacaatggaggttgtaaaggtgacagatacttgtcttctacagacataaggctggggagggcagaggaaaaacaagggctacaagaaggagacacgagaacatggctcttacccacacagttccgtccatctccctggtacccagatgcgcactcacaggtgtaatctacacctgtccctggatggcaccgtgctgttgtgtcacacatgtggctcccatcatagcaaggattccccggagtggggtctgaatcctctgcatgagtagaggggaaataaaagcacaatctggctattggtaagcaaatgtcacggagatagaggagtcctcggaactgactgcatgtctgtgttttgtgacacaccggaagaccttactaaattatccatatctgaagctagggtggattttctgcacttcagttttaaactccagcatggcttcctattgcctaccctatagacaactagtacctttacttgctttcagtaggtttgcatccagggaataaaaacgcccaaaacatctgtttaaaagtgcttttgctctattatttcctgtgattcttgggatgattctatgtggtgagttgttttccccattatgtgtaattttcacagataaggaaatcaagtgagacatgggaaaaattgcccaagctcacaaagtcaggaagtggcacagcaaagactcagactccaacttcctggttctaaatctgttgatctcttactaaaattcccaatttttataggtaaatccaattaattttaaaaatcttgttttttaaagatttctaagactaaacaggaatgactttcccagtatttactcttcaggagtagccttaccaaggatccatctactcactaggtagcgattacttaacgactgaaatctcccctagagaaatctattttggctgagcatggtggctcatgcctgtaatcccagcactttgggaggctgaggtaggaggatagtttgaagccaggagttcaagaccagcctgggcaatatgccaagaccttatctcgagagaaagaaaagaaagaaagaaagaaagagaaagaaagaaagaaaagaaagaaagaaagaaagaaagaaagaaagaaagaaagaaagaaagaaaggaaggaagggaaagaaagaaagaaagaaagaaagaaagagaaagaaaaagaaagaaagagaaagaaagagaaagaaaaagagaaagaaagaaagaaaaagaaaagaaagaaaaggaaaagaaaagaaagaaaattagccgagcatggtggcccatgcttgtagtcccagctactccagagggtgaggcaggaggatgacttgagcccaggagcttaagcttgcagtgagccatgattgtgccactgcactccagcctaggtgacagaacaggaccatgtcttaaaatatatatatttatttctacataatagtcaatgctcattgagcatttatgtgtgaggcattgttctaaacagataggaggatgcctgattacatccttctaactacatggtgaggaaaatactatctccacttctgctgcatcacagagaggccaaataacctgcccagggttacacagtcagaagaggtggatggagctggaataaaagtctagcagtctggttccatagctcatgctttccaccgctttgctgcaaccctcactgaaggaacttcatgaagttatctagtctaagccaatggcacagaggtattggaaggagtgtcagatgctcaggtagaatgaatctatcaactctggcacttctgtgtacttctgagtctgccatgtaaaatggaaataggaacatgtacatctgccaatcctgctaaagaaaattaagaaaacccaacagaacatatttgaaaggcaattccccattggcaggtctacccagtctctactgactgcactccttctgccttctcagtggggccactgttcatcattatttgaggttctagctcaacctcagtccctagaattaactctaatcttagactcaaaagatttccagagccccagctaagcctgtcaggttctacattgagccatctctgctacaaagggagatccatctaagttgtaataactcaggcttctgcctcatacttctgtgcctgaggccctgtcttgatagccagcttagcatcctctgagtcccatcctgctcaccagttctcactactcacagacaccagcatcatggcatctgtaggcagacactggcagtaccacccattttaccacctcctcctggattctctatcatttcactctttctgacccgtgtctcccaccccactttgtacagaatcttctattaaaatggagaaataacaacaggagagaatcaggttcttgtcttatactgagccttcaatgaggacatgaccctgttgttctccattgtaacagatgctgactcaggaacctggtctatactctcaagttaacttggcaaccacaaccccactgctatggggggagacttgcccagagtgactcctaatctagatcccaagtgctgactgctgcagctgacctcatagactggactcgctcccaagtccttcagggtgtctggcattctcccccgtgcctgccctactgcccacgttatggctcttccagtgccctgtcatagcctgctgtgagctgggatgccttggctgagacagccttgttttctggtaaactctttccatcccactttataagaaacaccacagtgcattcttgtagtggaatattcccgtattattgattcccgtattattggctgaaaataaggagggctatgggtttatcctcagttcaccacaatgttgtcggctggagtaacagaatctttgccaaaacaaaggaccaataaataattccagcactttttaaacctgatgaactatgtcagcataagcctagctccctgaagcatgcagacatccttggaggtccgttcctctggatctcttggctgacaggaaagaggcaccgcactggggttgtggttgccgagttaacttgagcctagaccaggttcctgagtcagtcctcaataggttataactaaggacaaggtcatgtcttcatccattaggacccaatgtaagacaagaacgtgatcatctcccatcatttctccattttaggaagattctgtacaaaatgcctggcataccccaccaaagcaggaagtaattgtcaacatgttttgatttataaatactaatgcttagccttcttattcagtactttccagcatcaaaatttcaccatggcacattccagcaaagattagtgtgttcacatggacatcaagagcttaccaccctgctaggcattaaaagaaaaaaaaaaaatctcagctcaaaagttgacttgcaaacttggacaggatatattatcctacctaaaattgagaagaaagtgtccctaattactcaatactgatttaaaatgtatgtcactgacaggattagttccataatttgcaatccccagtgcaaaatgaaaatgtggagcccatggttcaaagcaaagcgttaaaccaagcatggggctcttctgagcacagggccccattcactgaacttggcttcccaaaacatttttaaaatacatttctaaaaccatttcacgactcattctctatgacttgtaagcttttctcctattatagtaagtaaagaacacagtatccttcttaagacattttctatttcgccctcaccctcttaaactgcctgactctcttaacagaggatctggaactaatttactcatttaatcaaggggaagagcggttttccttaaaggaacagtcccttcagcttcactgcacagccctctgcccagagcctcctcaactcgccaaaaggacccgtgaagtggaaggacaggaagcatttttggatcgtgttttatgaccccacaaaacaaccaaccaaccaacaacaacaaaaaacagctgactcacacttaaagcttaaagctcaaacaactggtggggggtggcactgctggttgagaagtcaccgagggacagaggctccaattactcactgggaaagacaccccagttctgcatcctcgccctgggaagtcgcaccacacccacatcaggtgtggacagctgcgctgtttcacatgaattccaacaactatcgttccctctggaaaaactgagcagttcttaggaagatactgctgtgtaccaagaggggaaagaaaaaacttttgggtgtttcttttttgtttttctttttttccctcctattcaggtagaatgaacagtactgctgtgtgtggatgaagtagggttcaaacacaaagggtttaaaagacacacattgcctctttcaaagtttggtctaaattatgaggcagtcatgaggccactgtggaacagcagagccatgaggggtaagggtgtgcgtactggagccagtgcctgagtttgagtcccagctcacccaggtcacttactatgaccaggcaaattacttcatttctatacctcagtctcatcatctgtaaaatgggataacaaataaccccacccccaccccaccattgggtagaggtcattgtggggattaaatgagttaattcatgtaaggtgcttagaacagtacttggcaccaagtaaaccatcctttgatattattatttgactggggtgggagggaaaatcataaaattctaaagaaaacaagcaacttggtacctgccagtttgctttctctgatcctttcttactctcatttggtatgtctctaggaaaaatatacccagagacacatctttttcctaaatggttccagaatctgaatgggactccatagcctgggaaatttcactaacagttgcttttacagatcagatgcctacacccaacacagggctgacctggacatttaaaggtaaagaccattctccccagtaggtttaaatctctctttgagaaatctactacagcaatctttccctttctaatttacatgttcctctaaaatcaactcacattttcatgtgttcatttgctaccttacaatccaatatctgggcacctaatgcctacattcccaaccaagtttcttaaaaccaaagcacctcgggattctcattacaagtcactttcgcatcctagaaaagagtctagtccttgtaccttaatcttgtcatattgctgaagcaacacttctttattgagcctgctgaggcttaaggagccctcagctccaataacaaagcacagaaccgcgtatcacttacagattcacctcactgaggagctaactctgggctcaacagtttcaatacagtggctgaattgaggaagatggtttttttctcctggtgtggctcttacattctaggggaaaacatgacctggttttcagaattaaaatgtccccttgatctgacaaagccacttcccatctgtagagttccctgacttcacaagctcatcctctggcttgcttgaaacattttttaataattaagaccttgctttttaggatggttttacatttagagaaaaatggagcagaaagtacgagctcccatatccccccacccaaaatgtgcagtttcccctattatcaacattgtgcattagtgtggtgcatttgttatccccaatcaatactgacacattattaactaatgcccatagtctgcagtagggctcactcttggtgttggacagttctatagactgacaaacgcataatggcctataaccaacattacaatatcatacagaatagtttcactgccctaaaaatcccatgttccacctatgcatccttctccttcttcctcctgaaccactggcaaccactgatctttttactgtctctatagtgtaaccttttccagaatgtcatggttagaatgattctgtatgtagccttttcagactagctgctttcacagagcaacatgcttttaagtttcctgcatgtctttctgtgacatgatatgcctagaacattttaaaatcagtcttcacaggtgaggctaatggtcattaaaatttgagaaccactgcttactttgagacggggtcttgttatgtggccaactctggcctcaaactcctgggctcaggtgatcctcccacctcagcctccagagtagctgggattacaggtgtaagccactgtacccgatgagaatggttttagagttttgtttctactactatttgcttccatttatttttattttttgagacggagtctcactctgttgcacaggctggaatgcgatggcacaatcttggctcactgcaacctccgcctcctgggttcaatcagttctcctgtttcagcctcctgagtagctgggactacagacacatgccaccacacctggctaaatttttttgtgtatatatatatatatatatatatgttttattttgtagagacagggtttcaccatgttggccaggctggtcttgaactcctgacctcaggtgatccacccacctcagcctcccaaagtgctgggattacaggcataagccagtatgcctggccttcagttgagaagtaattcacataccatacaattcatccgtttaaagtacataattaaacaatttcgtctattcagtgttatccaaccgtcaccacaatcaatttcagaacattttcatcacccccaaaattaaccctgtaccctttagctattaacgccctatgctctcattatctcttagccctgggcaaccatgaatatgctttctatgtatgtggatttgcctattttggatatttcacataattgtaaccattcagtatgctgtcctttgtcactgactaccttcacttaaggttttcagagttcatccatgttgcagcatgtatccgtacttcattcctttttatggttgaataatattccagtatatgaatatattttttggtgtccatcaactgatgggcatgtatgttgcttccaccttttggctattatgaatactgccactatgaatatacacgaataagtttttgtggggatctatgttttcatttcttttgggtatatagaaaggagtagaattgctccattaaatggtaactttgtgtttcactttgagaaaccactattttgtttttcacagtagccacatcattttataatcacaccagcaacgtatgagggttccgaattttccatgttctcgccaacacttgttattacccgttttttgattatactcgaccacatgatgtaaagtagtatctcattgtggttttgatttgcatttccctgatgactaatgatgctgagcatctttacacatgcccattgggtatctgtatatctttttggagagaggtccttgcagagcttttgcccatcttttagttagcttgtttttttattgagttgtaagatacttcgtatactctagaacaaatatcacatgatttaaaatactgtctcccattctatggctgtgttttcactttgttaataatgtcctctgaagcacaaactgttaaaattttgatgacacccagtttattttgctgttgcttgtgcttttagtgtcgtacctaagaaaccattagcaaattcaaagtcacaaaaatttacccatatgttttcttctaaaagttttaactcttacacttaggtctgttggcccattttgagttggatatgctgtgaggtaaaaggttcaacttcattcttttgtatgtggtcactcagtggtcccagcaccatagttaacaatagtttaaaagactattctttctccattgaattgttttggcactgtgttttttgttttgttttgtcttttaaccatcagaattctattattattatactttaagttttagggtacatgtttacaacgtgcaggtttgttacatatgtgtacatgtgccatgttggtgagctgcacccattaactcgtcatttagcattaggtatatctcctaatgctatccctccccactcccggcactgttgttgaaaaagcaattgacttttaatgtgagtatttctgggttcccacttctgttcctttgatctttacatgtatccttacgccaggaccacactgtcttgattactctagatttcgagtttgagaaatacaagtctgactttattcttttccaagattgttttggctattctgtgttctttgaatttccatataaattctagaatcagcttgccaatttcttcaaagagccagctgagattctgatagggattatgttgaattaatagatcaatttgggaagtattcacatcttaacagtattaagtcttccaatccatgaatgtggaacatcttcccatttaactgggtcttaaatttttttccaaaaatgttttgtgggatgcaatgtacaagtactgcacttctttttaaaaatttatctctttttgaaacaatttcttaatctttagtttgctcattgctactatagagaaatacaatttggtgtactgatcttgcatatcttataacattgcgaacttgttctttagttctaatagtgttttagtgaattcctgagattttctacacaaaatgtcatctgagactaattttactttgtccttttccaatttagatggcttttattttgtgttcttgcctaatcaccctggctagaaattctagtactgaattgaataaaagtggcaacagcagacatccttgtcttgttcctaagcttcagggaaaagcatccagtctttcaccattaagtataatgttctgggaatcatggagggggaggttatacatcaagttctatgatgtttggtttttccagatactagctctttgctccaccttagttttggatgaaattcttctcatctgttttagctaaaatgtcactttcttggggacatcttttctgaatcttcaaactagactcacagtgccctgctctcatcacacttgtaacaaatacttgtcagcatcactctccctcctaattccagtctgccttgttcacaggtatattcctgattggcacgcacagttgggtattgctataaaccagcttcacatactatgctctatttcctgactttggcagggacactgttccctgccctaaaactcttttctgcctctgtttccaactcaagggcaaggatctgctctattcatccttatacctagcacataacacagcatggcrctaggtaacccttagtaacctctgtgaatcattctaactctacttaaaaggatgtcatttttacccaacaacaaaaaaggaaaccttacctttgaccgggccaatttgattggtcacagcaaatctaagcactctttcttcgtcattatacaaggcaaagacccggtccacgttcagctgctgggtggtggggaaggacgggtgtctgggggcgtgcctgcacacctggtaagtgatgttctggtggatgcggtaggaccatgtttggttgattgcaccaaaagtcagagagtagtctctggaacttgtagaggtcacagctgcaacaaacacagtggtaattttttaaaaatattaaatacagagattttcatgtgaggtggatttttctgcagtttttttcatctaattcttggagtgtgttcccttgggaacctcctgagtttatatgaaaaaatgtgtaaggaaatcccaaaaacctgagtggatgcaaagccacagtgctgagagggagaaggcactacaattgtccatgttattatacacaaagaataagtgttactaagggccagccatgccaacagtgctctctacatactgcctcatttattcctcacaacagcctggtgaggtgggcactaaatcatgtgatcagaggctcacaaaggttaagtagcctaagatgcaatgctgccacatggtagagctggaactgaaactcacattggtttgactctacaccctaaattttaaaccaatccatcttctacatttcacagaagtgacgacttccaacacctcgcacttcagtaaatctgaaggattccagagaagggaaagggttgtctctcaccatctgagtctaggagtcaagctcaggtcagtgatccggacactgaccattttgttcacagcccaggtctctgcttccccttgtcttccctacatctcctgccaggctgccagctctcagcataggagcagcaggggtcagaggaaaggcacaactcaatccggattgctgtgctgcacaacagcagctgtgatggaggcagcatcctgtgcgcaggtagtactttcacagaacacatcacgcttcgcacacattgatcacataatatagaaaaagcatttttagttccatgtgaaagatgagaaaatacattctcagatattaagaggcctttccctatagatattaaatggcaagtccaggatataaattcttatcttttgactccaaatctagcattagttttataataaattatagcatctctgtgttttttggttttgtttttgagatggagtcttgctttgtcgcccaggctggagtacaatggcacaatctcggctcactgcaacctccacttcccaggttcagatgattctcctgcctcagcctcccaagtagctgggatcgcaggagcgtgccaccacaccctgctaatttttgtatttttagtagaaacaggttttcaccatgttggccaggctggtcttgaactcctgacctcaggtgatccacccaccttggcctcccaaagtggtgggattacaggcatgagccattgcacctggcctctttctgtgattttttttttttttaaagcagcctgaactcatcagctgaaacaaggttgagataaatataaattccaataatcactatttttccatgttgcagaaagtaggatggttaaaaactgtatataattataccatccctacccaccccatggacaatgttgctattgattttattatttgaaaatactatgtagggatatagatattcttatgtatagcagccattctgctcacttacacgatatatttcatggtaacaataagagtaataacataatagagctcattttatgttccatgcactgttctaaggacatgccatgtaagtcatttaagccttgtatctatcttgtgagatgggcataatccccattttacagatgtgaagactggttatacatactggagtcggagtagtggtacagctccttgtagggagagatgtgggctgtgaaatttgctgrgacgtaaggcacctggccttgaatgttggtcttaatgctcaggtagttctctgggtcaagtccctcagcagtttgagtgatacgaaccgtctcctctcccgggtagaatgtaacttccatgtcatgggtaaaggcagcacctggagatgaaaaacattcagcacagggatgaaaagaggtcttgcttaaacagttaccagtatatactgcccgctcccaatttctactgctgttggagatcgtgcctaaggagattatctgatatctgagagtaagcactatcagctgttgattccataagttcccaaacttttccattgaagaatatggtttcatcctgccttacctcacccagcttctctttgaacaagcttgtattctgtgagaaatactcttgggaacgtccttgacttttctctaaggttgctagacttagcaaaaaacaaacaaacaaataaacctaacaacaaaaaaacaaaaacaaataaactaccacctaagacattcagggttttgtttgaatttcagataagtatgtctcatgaaatatgtgggtaatacttatactagaaaattgtttatctgaaatacaaatttatccaggtgtgccattttttatctacttttgacccaagggctttattcaaattttgattaaagcaagaacctattaacagctaccgagctctgcagttaaagatggagaaagaacttctcacaaaactatttttaatgcaataaagataaaacctctagcaaacaagttttcaggttggaaggtgagaatccaaaaagtatcttctaaatccttgttgtcaacgggtggcctttccagcaacactggtacctcacctcctagcttggtagaaaagcaaaatcacggaccccaccccaggcctgctgaaccagaatttacattttgacaagacctccaagtgatgtgtgtgcaggttcaagtctggggtgcaccggcctatactattataggttgtggagctgggaggagaagagcaccccttccccatgaagtaaaatcatccccaggtgccccaagtaccatttttgcatttgacagatctgtgcataatttaaaaagcctgacaaatatgctgcaacaaattggggtaagaggacacaaatgttctgaatggaatcatcctgtttctcagaacagctgggagaacatttagacgaaatcactgcctttcaaaaatcagtgatgtgaggtcaaaccattcccaacaacctgtggccagaaaactacacacatgtggctctagtacattaaacaagtgattcctctgtcactgcgtaaaggcctaaagacagtgccccaaggagcaccactgccaaagcaatgctgactaccggccttctcagaggccctactcacctgcgaggctgaagccgttctcagagccaggtttttctaaagcaaagagccagccaaacaggcctccaattggtgtgagggggaggagggcctgggctgctggctgtgggatgtggctgatggccgtgtaggctctgccatcattgcccacgatatacgcatgcaggtccacatcagtgaagtgcacgggtgtatggcccacgtggaggtggccactcactttcccattcactcggtgaggtgcccctaaaagacagcaaatccagttaggcttggacgtcatcctggctagagatgggtgtacccaggtataattattccactgacaacttccaaaactcactctttagcaagtcactttaatgatttcctaaccaaccaattatccatctatgaaacagtcctgcctccagtctgttttctcccctcctccttctactccactacactttggctctaatgagctggcagaaggaagcatttctggataatcctcaaattgggaatcagcccaaaccagttcattcctcacacatttattgagcacctgcaagttgatagcactctgatttaagtagtggagatgtttggtccataaagcagagctcacagtggtagctggaaacaggtaagcagcaggaatagacacacaggagttcctggccccagtcaattaccttcaggcagacagtgcttcccatttccataaaacttggattggcagtggcagcagaagccagtggcatagtccgtgcagaaggcatgccgggagcattgtctgtggttgtgttcacaggtttccttgttggcagcattatacgtgaagactgaaaaataaaacaaacttgccttaaaaggactaaatgattccaatgtcactgctgcatcggggacacaacatctgctccatagaagcagtttagatcataacaaacagtttttgatgtttaagagaccggcataacattcaacccaagggaattaatctgctttgtctgctggcaagaagaattgcttactgctcccggctgggtcccattgcagagttggtgaactaagatgagactgcacccgtgggtttgagtgcagtaaaatgaaagcaaaccagtaagtacatgttaggttgaacttcatgaaaccactatttttgtgggtcaaaaagggctaaatatcagcacttttatatggttcaacctaaacacatcctgagaggaagacataatagagaagctaacaaatgtcttatgtctaaccatccttcttttagggaacactgaacttaaaaaagataatagctggtctcgctaccaagattgttttgagggaagggtcttaaagaacacacgcacctacctcaaagaaaaggcatggggagaaggggacagaggagggcaaaacaacagggattcaagctattaaaatgccctatgcccttggcagggtttaactatgatgggtatggtacaagcatgatttccacgcagtagcagacagcttgggccccactcgcagatcttccaagaaattatggaggactaggataaattcagtgctctggcagctgtttcttccatttcctacttattctgcccattaaaagaagagggccatctgtgctgcaaagaaatggggaaactggagcaaatgccatagttcataaaggaaatagccaagggggtagggtgtggcgggggttgtggatggacaggagggtgctttctccggggcactaggaaatatgccaatcccaagcatggccaccaggaggccaagcggctgcacacacctctgcctccacatccccaggaggtggagttggctggagactccaggagcatgctgttgagcagggatggcatggaattctagtgtgagacacggatgctggcatgaaggaggacacccaccctactcttaggaaaaaccaagtggtagttgtgttccaggtctatctggaaacgaggagacaggaggggaggaagaaatccaaagatcaatctttacccagtattcccagggagaacagaaagtaaaggagctaatgagacatagcattataggtacaatgtcaccaagataaagacctccatcagcaaataaaaatataacaaagttaacacttcaatcgcgcttaacatatacaagctttacatttaacaacctttttttttttttttttttttgaaacggagtctcgctctgtcgcccaggctggagtgcagtggcacgatcacagctcactgcaagctccacctcccaggttcacgccattctcttgcctcagcctcctgagtagctgggactacaggcacccaccaccacgcctgactaattttttgtattttttagtagagacagggtttcaccatgttagccaggatggtcctgatctcctgacctcgtgatccacctgcctcggcctcccaaagtgctgggataacaggcgtgagccaccccgcccggccaacaactcttaatttgcacaacaaccctaggtagcagggacttttttttgttgctgttgttgagacagggttttgctctgtcacccaagctggagagcagtggtgccatctcggctcactgaaagctcgacttcccaggctcaagtgatcctcctatttcagccttttgagtagccgcagccacaagcatgcatcacacctgcctactttattttttggtagggtgggttggggggaggggtctccctgcgttgcccaggttggtctcggtctcgaactcctgggctcaagtgatcttcccacctaggcctcccaaaatgctgggattacaggtatgagccaccactctggccagggacaattttttaacccgtttgcagataaagctgaggcacagtagactaaagcacctgaagtcgcataggcagtgagtggcaaaggtgggaaccgaacctaggacatctagctggagaatcttaactgctactctccacgaccccttcaatgggatccaagtacggagatcatgagcactagattaggggtcaccacatgtctggggttaatcagcacagacttcaagtataattcgagtcatactttataaactgggtttaatggtagaagcaagcagggaagattatcgagatgactattacagcatgttgaaaaacagcacaggtaagaaatgagagtagcagcagcggcattagcttttgaacagtactggagtgctgcctgcagatccttgggaaagcagcaagctgagggttggcaggacagacagtgtaggttcaagtggaaaatctgattagaacatagtaggaatgaagttatgactcaagatttagtatagggccttgatgacttggctaagaaattccagtttgataaaaatagaaaattaggcatgactcaactttatttaaacaagaaagtgacaagcttaacttaaacagaaaagtgatataaaactcaaggcagaaaaaaaaaaaaaaaaaacccaggatggatcatggtaaaatagatggctgtgaagaaaactgtatcactgtctattagtttaagaaagggaaaaaaacatttagagtaaaaggaaatgaatgtaacttggatgtgtcaaaatctcaggtttctccagctcagaaattcacaactaagtgtaaggaagcagctacaggagggactcacacactcaccagtgtgctcatggcttaagggccatcaggactttctcccagagctcaaggaatgaggggctgcagaaagcgccaagctctaggactcaacctcagccctccctcgggggctttgtggaaactatgcaaactgtctttcagcttcctcatctctaaaataagtagaacacctcttcctcacccctgcattgccagaagtcgcatttcttcttaatgtacttctgaacaccaagtaaaaaggcaatctgctataggtctaaagaacagccatttggctgggcccggcagctcacacctgtaatcccagcactttgggaggccgaggcaggcggatcacgaggtcaggagactgagaccatcctggctaacacggtgaaaccccgtctctactaaaaatacaaaaaattagccaggcgtggtggtgggcgcctgtagtcccagctactcgggaggctgaggcaggagaatggcgtgaatctggcaggcggagcttgcagtgagccgagatcgcaccactgcactccagcctgggcgacagagagagactccatctcaaaaaaaaaaaaaaaaaaaagccgttttcataatattcagataaaaatcaatccaaactttacaagctgttctgcccacaaattaaggaaataacaggcttggattgtatcttatttcagttattttgtgaagccttattctgcaacccaattcttattctgaggctgaattatgaaacaaggagagagaagatggaggagagacagtctagatatggagagttaaactaggtggagtcatagcttttatctaagtaaaaagaggcagggcgatgcagccagcaacttacaagtgcaactgtgcaaaggtttataaatagattggaagctgtactacatttttctattacagaaaaagaagttataacaatgagcaggctcccatgaaaggctattatgagacttaaactgtaattacaatgggctaactagttactgttaagctaacctgggagtctttttaaacagttttgtggaatattgtatttttaggatggtacttactgagccaagaacacacactgtctctttcagcttcataacagccacatgaaggaaagaagatggatcagcccctaatccgggggaaggcaagcctccatttataatccctcttccacgcctgccttcatacccaagtgcacacacaggtttgcacacttaccatttacaatggcagcttgttaggtgaaaggtaaatgcttcttacaatttttgttcattcattcactaattcaacaagtatttactgagcattcactatagacctgtacctagtctagaaactgaggacatggcagggagcaaaagagacaaaaattcacatcctgaagcttacattctccccagcagcagtcaataaacaagtaaattacttagtggggtggatgtgtaagagctgtgcaaagaaatacagcagagaaagacaagagggtgggagggggcaggtgtacatggctctttttaatagcatggtcagggaagacctcaggagaaggtgactgagcagaggccaggaggaggtgagggagtgaaccccacagatgagggcaggcgtggtgctgtgtcaggcaggaggggtgtgtgggcatgtgggagggatggtgagagaagaggtggcctgggtcattcatgcagggtctcagagatcacaggaaggatttgagctttcatctgagtgagatgggaagcactagagggtttcgagcagaagagtaacatgctccgactacagtaaatcagacaggagctggtcccttggtggtaagaagtgctggtacaggggataagaagggcccaggtctgggatattctgaaggtggaaccgagagggtttgctggtagactggatgtgggatgatgttcagtcctgatgtgggaaaccaaaagggtggagctgccagttactgagatagggagactgaggaggagcaggtttgtggggttcagttctgacaacagcatgaggcatcaaggaaattcaccaaggggtgaataacaacttacaccctcagcacagcaccatgcagtccttcacacggagagctgcatttctcccggtggattccagagaccactgcatcaggatcacctgagatttctgttaaaaatgcaaattctgagggcccaccttagacctaccagttccactctctgggtgcatcctagcaatctacactttagccaccccgccagtgattctcacaccaattggtgttgatagaaggcttgggatacaacaagccttggatttctgagctaggtgttgctcatcaatgtcgttaacaaatttaatctgaccaattggcagctcctggggtgggcagaatgttaggaaatttctcgtgctcatttctggccacctgagaagctgagctcagtgcttacggcccatttgggcatccatccacaagaatacaacgtgaagattctttagtgaaagtaatacagacgcacagcagccagatagcccttccctgaagaagggggagacctggagctagaacggtgctgagcccacgtgcagctatttatccagggcgagaaaagccagacctccggccaacgtgactgctcaaatttctatgggaattgcagtgaaattcaaagaggctgaacttcttgaaggctgggaatcaagatctggatcccagctagatgagatgtgcaataaacccacccgaggaaaagtggcactttttcttttcggtggtcattttcatgtccctatcatggctttgagtgtattttcatgcacttcttcctattctagttagaagatatctccattttataaatgagaaaattgaggcctacagagatgaaacaatcttctggctctaaatcccatcaaattgagggggcatcagaatcactgaagtgcagaggaatatggcagggtcagacaagagggtgagagagggcaggcacacctggctctttgaaatagtatggtcagggaagacctcgggagaaggagacatttgggcagaggcctggaggtgttgaaggagtgaggtggcttaaaaatgcagtttctggttcagaaggtagaggaggcgcttggaaatcagtgtttttaaccaggctcccagctgagtgagaggaaggtggatgagtgcccacactttgagaaatacagtttctagtccatggcctcctctgggacccacactatgccaggaagctcctgccacggagagtcagaccataaagaagtttgctgacacctcagggcacagcaggagggtggaggctggccaggtttgaatgaactcattttgtcatggatttgttaactgaggtttgatagttgctaaaggattcttaacatgaagattttttaaatctcttttttttctatttaatgttttttttgtttgtttttaacttttctcatgtccttaatagaaagaaaaatggggtggggagatatcagaggaaagaaagaaatgccctgccaaaggacttgagacttgtcctaactaggagttcgtaacaaaaatggaaaattctcctctgcacttgttctgagccatctttcaatcttcattttcccagaaacacttaagcacctgtctgcagcccttggccggcctgcccttcttcacagaaggccctgggccagacacaggggaggttccatggtgtcgactgggttcactgggccaacacagagggacacaggaggcttcgtgcacagggctgcttcaggacagctggagcaactactaacaatttttactgctacctcaaaaaaaaaaaaagtcattttcaccgcaggaaaaattcttctaagttttcccatctctcagaatgacagactagttagaaaaaccatatccaactttataacaaacagtggaatgctttttcgtccagttttcatgtttgtttaattttgatgtgtgtggcatgtaatgatgctggtggcagtgatcacgaccctgggggtggccttttacaactgctggcatgacactaacaggctctctccacttctggtgaagggagaggggccctaccatggaatctcctcatataggtgtcttcacaggcatcggggggcggcggctcctggtcacagaccctctgcaacctggaggtgcacccaggggatggtctttggcacctggagggttgcactggcagcagaacctgcttaaattcttctcagggtggaagttgagcttgcaacacactcctgcagcctttcacaaggccaaaagtggagtgggactacatgaggtttctgaaagccaggtgtccttcctcctaactcatcatgcagattgagattaaattaagctgcgtatagcactgcctcagcaggcttaagaataactaagacccctatcagtaaacctcattcctttctactcacaacacaggcagtcccatggtcccaggcagctgaagccctcactggcaactctctcaccccactgccttaatcaacctttgtctctgtctctctccctcctttcctccctcctcattcccatctctgcaactccgctcttgtcacttctctgttcataatgttttcacacattctccctctcagactttgaccccaggcctggctcaagcccctccttcctcacaaagacttccccaacagcagtgacactcctggacagtgcctttgactctctgtcaccctacagctttctctaatttcttcatgactttgttgcagtctttgcccttgctagacaagtcactaagtatttccaagctcagtttccatctctgtaaaatgggaaggatgattgtacctaccctacaggacaactttttttttcagacaatatttctctctgttgctcaggctatgcagtggtacaatcatggctcactgtagcctggaactcttgggctcaagtgatcctcccacctcagtctcccgagtagctaggaccacaggtgagcaccaccacacccggctaatttttaaaattgtttatagaggcaaggtctccctatgttgcccaggctggtctcgaactccccacagggcaattctgaggactaactgtgataatgcaatctaatgccaagttctcactttagagcagcaggctgcacttagcaaatgccagccaaaacttccactagcaccgagtcagagacaggtacacggcaggtgcttgggtaactttttgacttgacaacaaaacgaccagagctccctggaaacaagactcctcagagaaagcatgcttgcacatccatctactgctgttctgtggcctaggaagacagagctcgagtgtggccaggacattcctgcctggtgtagatatgatctgaaaatctaaatttctgaacaaaaagaaaggtctgtaaaatgagtccctaaggggaaaaaaatgtaaaaaggagtgatattttctgggcctttaactaaatcttaatttcttggtttagcaagatgactaccctaaagcttcaaagggaaactaaaatgtattgggcagggtgcttcccaagctcaatttcagttcttgcaagtatatgacgaggtcttcattaactctgttttagagctgaggagagccaagggtcaaacagactacatgtcttgccccaaaccacatagcagagctgggagtcaaggtcaagtccaaccagcaccaaaaccatgtgctttctattccatcagcagaaaaagccaggcagatgccacatggctgggtacttagtaccggacaatgttctgtgtctctgccactcccaccccacagcttcccggaggaagcccacacttaccagacagacctctgagcctccaatgttttatgtgaaagaaacagtcactgcatcccctcaaaagtatttaacaattgcatgcttctatacttcatacatccccgtaaattcttttaaaacaaaaactggattttttttgtctaagtttcaaacacttctagatttcttcctccaaaatgcgtattaaccagctttttcacaggcgttttttgccattaccaggatgtctatagtagactgcatccggcagctgataaatatcatagttcatgccccacaagggttatagctgtgcctctgttctactctatggctggttcctgaagcctcctgcactttactccagtgatctcaagctctcagagcccaaagcagggtgtgcagtcccttcttaaccctttaatacatgactccatagatctgctttttaatattcccacgctcttcccagggaaggaaatcacaacccacaaatgagaaggccattcaggaatgcgtctttacctcaaatccctttgtgaaaatctttgatacttggccttaaaatgtatttagaggaaactcatactgtaaattctggagattctcagaaggatctgaaggttctaaagacttccaataaaatttacatcttcaacttcatggaaatgtggtcccttaggcacaaattaatgtgaaactctttaataagcacattcttcattctctacccatcctccaccccattcccactgttttaaatcttctaaaattagtaacagcagccactatttattaagaaccagtgagtgcagacacgaagccaagaacttcacatacgctgtctcattgaatcctcccaagtaattcctgagaaatcagaggcttatgggttacataagttacttcaggtcacccagcaagcttggcagccaggattcaaactcgaagccgacaccaatgtactccctcgaatccttacacaatgccacattagtcagaaaatattttcttccctcctgaaattagtacctctccatgcaatagcctgcagataataccagagcagaacccagaaggccctctctgctaggtcagccatgtttttgccacaacagtgacaggaaacagaaaaggtggaaaaacatggagattcagaacaaagcagtcacctctgacctctttcttcctatcagaggtcagatgagcagtccctggaacaaacaatctggagtcagtttcccattcacatctttccatgtctgatggggaagaaacagctggcatgacaagtctcaacaaggactagaagactgacatagaccccatgtccatgataattaaccaccgcctgctttcgccatctccctcctcgcctctcagtccgatcagtgggtcatgaaatgaggacaaggctccagggaggttcatgccacagctgcatagccatgaaccagaaatgacaacagacacaggtgtttagtcattttctttccctgtgggctctcctctcccacacatcacaagcatatctccccaaccaagggaagcgctaggcttgtctcacaacagagtatgaatctggcatgttcatctgagccagcacaactcaccactttaaaaagcacaaacccgtccatcactggaatgtgtatgaagccgcaaaatgttttacctttgctgaactttcaaactgaaatagttcattacacttgagagcgtagctggcataacaaacccagttttcccccactcacttcatgaggcccatattttaaaaaaacattattaatgatcaagagattccgaacagaatctgcctgccaagatttatttggcaaaggaaatttactctatctcttgaaacacagggattaaaaattagatccaatcttctttttgctaaacttttcacctacccacttaagagaaaaatcacaacgctatgcaaaaccagcatggttaagatgaaaccttagcacccagttttctaatgcactcaccctcggtgttggaacctatgttgtcttccagtcccacctcatacgtccctcgacttaagggtgtagtgtgacctgaagcagggtaacttcgaagaacaatttcttcttcaggatgagctgggggaacatccatttccgaaggcactggccctccatctgggtagggctggatgcttccgttttcggggtacggtggtggggtttcccaggaaggagccagtgaatctctgtctacctctggtggagctgggcttctggtctctctctcatcccagggctcaacttggccttttaaatctgggccccctacctctcccagagatgttccttctttggtgtgaggatccaaggtggaagattctgtttcaggatagaatggccaataagtaggtgttggatgcaagggggaggacagatcagaatctggaggggagatcctaccctctaaaggctttgtatccactttggattggaaggaaacatcaatgctgctgtggccattcaatgcctcctctggttcacccggaaggtattcagcttcctcctcattcacatcatagtaatccaaattgtcctcattatagtcactttccagggctgtagcatggctgaaggaacgtcccaggggaacagaagagtgggcagcggaaaggtctccaactgcagctggcctgacattgtccaacggggaagtgctgccgatatggaaagcccacactccagggatccccaggttgcttagtctaaataaaaaggaaacagtcattgtaacaaatgtaacaaaagtgtccattcacccaccttcctagaaggtgaacaacttattttaaaacggaaagacagctgggcatggtggctcacacttataatcccagcactttgcaaggccaaagtaggaggatgacttaagcccaggagtttgagaccagtctaggcaacatagtgagaccctgcctctacaaaacccttttctaaaaaacttagccaggcatggtgccacatgcctgtgcttctagctactcagtaggctgaggcaggagggtcacttgagcccagcagttccaggttacagtgggctttggttgcaaccactgcaccccagcctgtgtggcagagtgagaccctgtctcaaaaaaacaaaaaacctgaaaggctattaagatcaggttcagttttcactggtatataaaaccaaataaaagcagacttacatggtaactaaagggatcatactggacttttgatgtgaactaccttttatttaacattatgtttcaaagctgtatgccacagaatgccagatcatcaaattatcaatctgccccataatccaaatggtttatctaaaatctggctatttgaaaataaacaaggaatgtaggaatgtttcatccttcctctacttgtctggaaacataacacattttaaaaatgtatgcttaaaactctaccaaatactgacacccaaatgactacgacatactttcatgaattggtgatgccccttccctgtgccaagtgtttcacattcttcaatacaatctgcaaatagaggtgatcatattcaattttatagaggggaaaataggctttaaaaggctaactgatcttctgaaggtcacataacaaacaattggatccgaattcagttaacaccaaagcccatgctctactcatctatgttatattatgacagaaaaccataccacacactgcagtcaataagcatctgaattatcattactctacagaatattaaagacatttcattgtttattcacttgaactgattaattccctaatccacaaagaatggtgagtcaatgtaaaaaaattcaaataaatacaaatgccaaaacctaaaaaatttgtttttgcactgaaaaagttaaggagcaaataagaaagtagcttagaagtcttttaaagacttctggaaacagctctctcgttttctccatgagaaaagaacatgaactccattgagtttccaaatatcccaataacccattattttagagtcaatggtataccctaaagagaacaactacatatgatttctaaaaatatgggaaaacttgagtgaacaagagtctggttaggttgaatatgccagtttgtatactcctctgatatttgtacaaaacactatgctcacattctcccaagcaatggaatttcctcaaaagtcagtcaatgaggaataacagtccatgcctaataagataataaaaacttcactaaattgtataagaacaaaagattttccttcatttgggtgggtactcatcatgttcatgtggatttctatagtaacaatatttttcagtacaatttgaaaatgtctttctcttgtgtgtgtttgtgtgtgtgtgtgtgtatgcatgtacatatatgtatatgctgtggttactgttgtggtaaatttagggaaaaaagcatagaaagaaaaactgattttgtgtgcaatatgctatacatatttgttaagttcataagaattaatgttggtgtttattggcaagcaaataaggaaaactaaagacagtaggagaaaatcctgaagttctgaggatattgagtttattatcacattagaggtggtgcaactatgaaatttcttcttgaagttagaaacagaaactataaaatcttcaaacagtggaaaaatacagattatggccaataaaagctaaataaaataaccttctaattatatataaatcactatctgggttttgtcattatgcataatggaaaagtaaccaatttgagtttattttaaaatccaaggtcatttggaaagcttagtaatagaaagaacttaatttatttctacaaaggaaaattgggttacaaaatgggatgacaatgccttcctccctgggttgttaggagaattaagtgagaaaaccacattaaaatgcctagcactgaccaggtacggtggttcatgcctgaaatcccagcactttgggaaatcaaggtgggcggatcacctgaggttaggagttcaagaccagcctggccaacatggtgaaaccccactataaatacaaaaaaaatagtcaggtgtgatggtgtgtgcctgtaatcccagctactctggaggctaaggcagaagaaaagcttgaacccaggaggcagaggttgcagtgaaccgaggtcatgccactgcactccagcctggatgacagaacgagactccatctcaaaaaaataaataaataaaatgcctatcactggccctaaacatcataaatacttattaactactgtcttgtcttccagaagatagaaaagtatgatttcaaaatatacacgtgtgaattcgtatttaaatttgcagaggaagagtatgacaaacattatgaactatataatttatgacctttgagctcacagttgtgagacaaatttgaggaagaaaacaattgactaggaaaataactttactcttaattacttatttatttagaggtagggtctccctctgtcacccaggctggagcatagtggtgtgattgatcatggctcactgtagcctcaaccccctgggctcaagtgatcctcccacttcagcctcccaaatagctgggtctacgggtgcacaccaccacacccagctaattttttaaatttttagtagagattaggtctcgctatgttgcccaggctggccttgaatgcctgagctcaagcaatcctcccaccttggcctcccaaagcgctgggattagaaccaccttgcccagcctaactttacccttaagagatgcatactaaggcatttagcaatgaactggcatgatgtttgtgacttactttaaaacatttaaacaggccgggtatggtggcttacacccataatccctgcactttgggaggctgaggcaggtggatcacctgaggtcaggagttttagaccagcctggccaacatgatgaaacccagtctctacgaaaaatacaaaaattagccgggtgtggtggtgggcgcctgtaatcccagctactgggaggctgaggcaggagaattgcttgaacccaggaggcagaggttgcaatgagcacaagatcacaccactacactccagcctgggcgacaagagcgaacctccgtctcaaaaaaaaaaaaaaaaaaaaaaaagaaaagaaaaaaatgtaaacaaacaaaaaaatgaatatggcaaaatgtttacaatgtataaatctaggtgatgggcatttgggtgttcatctagttattctctccacttttctgtatatttttgaaattttaaataatgaaaagtgaaatagaagattatattgaaggacatttctataaatttgatgtacagaagttcttcctaagaataacaacaagatccaaaatcagaaaggaaaagaccagtaactttgacaatctgaaatgtacaacttttaaatcatacaccctttctcagcaattccacttttaagaatttgttttagggagaaacgagaatgtgagcaatgtttattatgctgtttactgcaatgatatttataacaagaaaaatcagaaatacatgaaatgttcattaataagctgtctttatacatatatgacataacgttttgtagccttaaaatggtagtatagacagacatctgactaaaagacccctataagaagtgattaatgtgaaaacaccacttacatagcaaaagaaccattttggttttaaaagttttgtatatacctaaattctagaaggaccattaacacttgttatcacttgaagtagtgatcatggataacttttacttttatttcacattattctgcattgcttgtactttattatttgtattaagcatggattacaataaagaaaaatacgtgagtcatttggccccatatcttactctgctttcgccaagcaattcagagatttctgggaagtatcaaagataaaaatgagggaaggcatgaaagggggtaagtggaaatgctgtctgtgtgtttgtgtgtcacacgtgattgtggatgttagttccaagagacctttatggctctgctcagggaacacaactgctttttcccacaggtcttttggccccactgtctcctgtctcctgtgtagatgccagcacttacccagggctctgtaattgttgatctatttgtctatcaaccctaatagggtacaagctgctgatgtgcaggaccagtgtcttagaaatctttgtattccatgtgcttggccattgtgacctctctataataataaatctatggaataataaatctatgagctcccaagtagaagagagggtggaaccagaggagggagagtaaggaactgagtttgctccagagtccagactaagcggcagcctagatggagccctaaagatgcccaatgtggatcataaggatgggaaaggaaacaaggactcagcataagataaaataagtagttcagcactccagacttgagcttggattttggctttgccacttaactttgagatcataggcaatttacttaacttccctaagctcctctgtaaaactgggaaaatagtagtactaacctacaattcttgtgagttttaattaagataacatctggaaagtgtttagcatgatgcttggcaagtagtaagttttcaataaatgtcagctctgagtaagcaaacctggctatagtggtctctttctggtagccgagcagagtggatggcatgctaaaagtgtaaggtgtttctatacctacccaggattgatttaatgaagtcaagaagcagttaaaggaggccaggaagtaaccaatgtattctatctggcttctatattcaggaaacgaataaaagcgagggcacccctacctcccctcacacaccccccaaaaaaccctgaagtcaataaatatcctactggcactatctgtactggcaaaatgcagaagagcttattattgcactagctaagtaagccctctggatcattcattcattcacccatctatttattaaatgcccattatgggcagacactgatgagtctaaggaatgggttcaattcattttaggtaatgaagccagagagacatcctcagacttctggaaacctataaatcttagttatttttcctgcaaattacattgctcaatttaatgtttaacagtatctgtccaaaagactaaatcacaatgtttcctcctgtgaaatgattaatctggaaaacctcaaactgggcagtttatgactggatatttttatgtggttccaagagcaatgtttttcatagcaagcctcagtggctctacaaatacacacacatactgttctgtttccagtatctatacccaatagagcaggaaaaataaaggaccttttatgttttggtgaacttgatccttccagggttgaatctagttccttggttttgacttgatgccttacttagaagccaccaatatattatgtaatggtcttatggtcacttgtgttcaggtacttcctaaaagtccttatattcaaataggaaagggcttcagctcaatgttacttactgatagagatttttcacagactgttcagtgctagtcaagctgaaatatggtccttctgacttcagatcatcagcctcccctcggcagaagcccacccgagctggaagctgaagctggacattgtaagactctttggggcgggttccaaggaactgcaggccgttggcaggataaagaaagagggcgtagctatcagacccatcagatgccaaaactgcctggaaagtgttcagctgtgaggaaaaaaaaaaaaaaagagagagagagagagagaaacatcctgtaaaaaacaaataaaagaagaaagaaacagaatgcaagcaaagcatcataacgtgcaaagaacttcccctttttcaggattcctccaaattgtttttaaacataaccttctttaaaatttcctcttcaagttaacaatataatttcataagcaatattatttattattgcaaataccatttgtcaatttagattaatagttgaaatacgcattggtaaaacataggttaaaaaatatatactttgtaaggtttcaaacttctctcatgtaaactctttcagagctaagagcaatcagcaaaattaacttgtccatcctgcagtcactgccctagtttataaccctaggcactcagcatctctcatttgcatcattcattcattcattcattcatccatttattaaatgaccactatgggcaggcactgtgttaggcactagttggtcaaaaggcctccttactgtctcacagcatcactctctttcctcttccatctaccctttcatacgagcatcagggttatcctcatttcacagatgagaaaaagacacaggggctaacaggtcttcctcaggtcaggtggcaagtatgtggtggtgggccagccagctctagagcccacaccctaggacaccctaacccctcctcaacatggcattcaaggtgagttgtaacctggctgcatacgtccctcccaggctcagttccactgcatctttgtcagactatttaatagtccccatttgtaccatgtccttggatgcttctacactttgttcgtggttccctgtgcctagaaagcctcctaaccttcaaatgctgccactcagaggtgtttgtgattccctatagaataatttctgttctctgaacctctgtaacacctttatgggcatctctagtgagcattcacctcattatgcttggttagttgtttatatgtttgtaaacaccattcatttattcattcatacactaattcaaacagccaagtgtctacttaagcaccagacgttatgcttgatgctgaggatgtgaaggagcaaaagacaggctttgcccaggagagtctcacggtgtaggactgggaaacagccatggttgaaatggtaggagagtcactggagaaggtggtagaggccaagagccggggtgtgtgtccaggatctcagggtggaggttgggaggtgtcacagaggcagctggtgaatgttataaacactgcaggagccaacacagaccagagagggaccaggagcctgaatcagctgccaattcaacacccaggtccttataggatcactccttaacattctgtttaccctcatattttcaacaagatgagatttttgcctttaccttcattcagcgaccgctcctcctaaagatctgtgaacagtgcttgattctcaaaaagctgttatgtttaggcttctgaaactgtctctctctcactaaatcacactacttttacaatttagtaaggaactgggaaggtatttttaaaacacaacttacttcacaaataatgtgactcttagaaatattatttactggtaagtatcattacctttccattctgctagacagacaaactcgggcacagagaagaacatcacatttcactgaaggaaacagcaaggaaggaatgggctcttccctttgttctaagtctactcccacagttgttctagctcttttctaatgttaatttttcctttcaatgtttaatttcaaagtaaggttttgaggttttgctaataggataaagaaatatagatgtaacacacttttcaagagaaaataaaatacaatcagcagttcttttagttctctaacccagaaaaaacaacatcttgctttgcttttttcattcattcaattcacaaagtaacaggtgcctagtttatttgttattgttccagttgttgattgttcatcagcaaagcttgacttttccaaaaacaaaaagcaagaaatgtttttagagcttctcaatactaggaagccacaactactgccaaatataaacaaaggctgttctgcctcatgtctatccataagatagtcgacatcatcaactaatgtggactcaataacttcagagttatacattagtcagaataaagaaaaatcaactagggaaagaaaagtactagaggaaggagaaaaaaccccatttcaaaaagctaagctttcaaagctcctggcattgatctgccataataaatgtgagataggagagaatcctggttctaaatttagagaaattccataagataaggaggttgtagagactgtgaagaattgaaggggatggggaaggggggaagagagtagtaaacagagctgtgaagaggagagtctgactttttgctgtgaactctgggcatttagcactttattttattcattcattcattcaaccagccaacaaattcatttcaattaaaatttattcaacatcttctatatactgttcttgagatgagcactcattatgggtggcaagtatttaatttctttgtctccaccaaaataggcaaatatcagaggaaggaaacaacttccttttaagtagaatttatgatcccaaagtacacgccaaaagagtttggctgtttaaaagccacccacacactaaatgatcacttgtaaagttcttggtcagttctacaaaattctacatctaattgctaaagagcccacttcatgaagtcttgaagggctccccctctcaaaaaactccttgactggcttccaaacaggaaaggtcctcacattcctagatacaagcttttgcaagagccgcacaggctctgtctccaaagagaagccctcagatgggctgccaaacctgacaaacgagtcactggttcccagctcccccaggatcccagagaggagagaggggcctccacagggagaggagcagcacagagaggatcagggtgagtggctgcaggccctgttatcagaaagagagaaaagccagagccctctgcagaaaccagagcctgactctccctcccaggtacaggacatcccaatcaatgttgaagtgacactcatcagagccaggctgatgcatgcaacatacacccctctaagagtttctaattttttcaattcatttaattaactgcagctatctggacaaaaatcagaatagtgacatccttttcttcaaggggcttgcctatagaggcttatcaaaatgaaaatgaaaatggaaatgaagaattagggtgactcttataggggtcacaaagttctctttgtcatcttcctcaggaccacatgtcctctagagctctaggatcattgggtctggcctccagtgtttgctgtgaacactaacctccattccttagtgtagttcttcatctcccactgcctgtaggacactgtcacatgggggactgtccctcatttattttactgcactaaatcctacagatcctgccaaaaattcaaagacaaatgctgagtgctatcacatccctaatactatatccttcctgatactcttcagcaaaacgtcagcatttctaatttccatatatggttcagcattaaccaatttccatatatgtctctattctcttcatcttcacagtgatatctcagactcagtctgaactctcttggatgggctgtagaccacccctcttctaaaccttgtctccagaagtcaaaaagctacaataaagtcctgcttttacctctcattttctctgtgaaatccaaccacctccatcaatgactatcaacctttgaaccaagtcaacatgtctcagtctccacccagatggaggcctccattgggcagtgtcttaatttgtcttctgttgtttacaacaaaatacttgaaattgggtaattcacaaagaaaaggaatttacttcttacaattatggaggctgagaagtccaaagttgagaggtcacatctggtgagggccttctggctagtggtgactgcagagtgccagggtacttacggtatcacatggcgacaaagctgagtgtgctaatgtgctagctcaggtctcaattcctcttcttataaagcaaccaggcccactccatgataatccagtaatccatgaatgaataaattaatccattcctgagggcagagccctcatgacccagtcacctttcaaaggccccacctctcaatactgtcacattgaggattaagtttcaacatgattttcagaggggacaaatattcaaccatagcgggcaaccttgcactcatctgtcttatgttactgcctgtgactccttcatccatttctctggcccctttgctaataggttgctctctcaaccttctcctgggtcatgtttttgctcagaacaatctctcttgatacctcattctcaatatccattctttttagccctttcaacctgacttaaattccagataaacaaaacattccccacattacagagatctatataactcagctgtctctaaccccatccctaaaatgacaaaaacaaaacactcctctttccccacaaaaagcacagctaaacattagtcctctatgtctcccatcaataagtatttcaaaggtctacaccataacttttttgctctgtgaactacctatgagagttcaagaaatctcagattaactgaattgacaaacactatttgttcacatttttttttcagatacaattacaccttgacattcctgcaatatcaggtgcaacatctggtcacacctgacaggaatatccatggagaaaaagggcaccaactgcaattgtttttaatggcaaagatgccattgctttgaatcttccagtttccagtaactttgcaatataaagttagtaagccaaactttaagaacgccatgtagaactcagtccttcaagcaaattagattactgtttgccaaagtctatctccaaagaactattaaaacaagaactcaagtcccagagctctttaaaatattggaagaaacttcaatgtgggtaattgaaagaacatcccctatccaaacagaatcctcctttctttttttttttttttcccctttcttttttatttatttatttatttatttatttattttttattatactctaagttttagggtacatgtgcacattgtgcaggttagttacatatgtatacatgtgccatgctggtgcgctgcacccactaacgtgtcatctagcattaggtatatctcccaatgctatccctcccccctcccccaaccccaccacagtccccagagtgtgatattccccttcctgtgtccatgtgatctcattgttcaattcccacctatgagtgagaatatgcagtgtttggttttttgttcttgcgatagtttactgagaatgatggtttccaatttcatccatgtccctacaaaggacatgaactcatcattttttaaggctgcatagtattccatggtgtatatgtgccacattttcttaatccagtctatcattgttggacatttgggttggttccaagtctttgctattgtgaatagtgccgcaataaacatacgtgtgcatgtgtctttatagcagcatgatttatagtcctttgggtatatacccagtaatgggatggctgggtcaaatggtatttctagttttcttaatgaaacattctgaagccaactagcaaatggtaatgtctgcaggtggattttaattagaaaagtaaaagagagatggaatttacaaatctcttctaccctaaacctcccccaccccagaaaaggaccagacaaaagttttaattccaaaaaccttgcatttaaagagtccttatgcaacatagagagtaacaggatggttaccagaggctgggaagggtagtaggggtcaggggaaggtggacacagttaacgggtaccaaaaaaaaaaaaatagaaggaatgaataagacctaccatttgatagcacaacagggtgactataacctgtgataacaggttattatagtcaataataacttaattgtacatttttaaataactaagagtgtaattagattgtgtgtaatacaaaggataaatgcttggggggatggataccccattctccaagatgtgattattacgtattgcatgcctgtaccaaaacatcccatgtaccccataaatatatacacctactatgtaccctcaaaaattaaaaaataaagagtccccatacaaaagatataagtcacagcatccttacaagatagagatcagtgacatgggcaagcaacctcacctcttacagggggcatgtaccatccttctatagtgaagaagcatttccgtaagttaaaattgagatgaatcatatggtttcagcccctagagtatggtattttagattcaggtctaattccatagaaggaagcccttactttcttcaggaatgaggcagtttgccatttaccaagcacgctggccccactcatatccatttggaggacttcaaatgttgatgacaagactaaagaccaggagcctggaattttttactttccgcaggtagagactgtaaagcacattgacggcagactcctataaactaagaaacttgtaaactggtacctggaaggcaattaggcagaagttatcaagaactttatcaaaagtgcatcctctctgacctaccaattgcacttcctggaatgtcaactaagaacatgtgcaaagatttagctttttaaaaagtgattcacgctattatttataataatgaaaaaccagaaaaagccttaatggcccaacactggttgagcatactgtggtacacccactattatacaggataatagcaggatacagtagtatgcatcattataaatgatgtagaaatacatttattgacattgaaacatatttacaatacactattctgtgaaaaaagcaagttataaaacagtatgtgcagtgtaaactcatcattgttaatagacagcaataaagatatgcagaagaaaaaactctgaaaatgtacgcagaaaaatgtttatcagtggtgggaaaatgagtggttgttttcttttttaaaattatctgtattttctgattttttccccttcaatgaacatataattagaaatacttgtatacttagagatggttctaaaaataagcaaccaaacagtaaattgccttatgttcaaaggggctcccacatctgcactcccgctctcatatccgctaagcatttttttcctcaagggagtgaccacctgccagggcctcagaagatgcctagagacaacgtctttcctcactcccaagccactccagccctgctgcctccaaggtcaggttcagcgcaagagtaggctcagaaacaactccgagccagtccctgggtcgctgccccagaatttaagcccatccttcaatttcctcagcagtcaaatatccctggtcacttacctctcccgagggcagcgccccgcgcttgacctcctcgtaagcgcctacctgctcccaggtggccaggaaggcgtgggtgggggtaaagcgcgcagagcgcgggaagccagcgcgcacatagcgggcggccaggcccagcactgcgggggaggtgtcctctcggtacaggactcggcctctgccgtggctcgtgtcgatgtccgccagaaaaggggcgatggccgggaagtcggtggggaaatcatagtccacatactgcgtttccctggggaagtcctgagtggagatgatgccgttggtgcccacctgggagaggagagggacaaaaaggtgacagtcgctcaagcccaaggacgctaccctttcgcgcagggagggaagggaactgacttaggcaaaaagcctctctctccttccccactggccagggagtctggattcagggaaacaagcgcaacttttgcgactgtcccagctccgcgctgcagtaggtccaggggccctaaaactcgttcacctatcttctcactcccctcacaaccccggagccccatgtcctcggagaggaagcctccaggtctgaagagggaacaccccagctccgcagacgggcacagccttctcgcggtcctaactgcaaagcccgctggccgccctagcaacgccaccttttctgcctgggacgcgtacgtggctgcgaacaattcttgctctcccctccaggtcattcttgctcccctacagcagcggggcaggcagcggcatcgctctctggccgggtaccaccgcaagggtgctggggggctccaggagtggggtctgtttcctcccctctggaggcagggtttccgtgagaccgaccccagggaagaagctgcgggaaaagtgccaggagggggctgaacttacgtagaggttgctgaatcgggcttcgtagaagtgcaggggattcgccagcttcaccacggctgagctttcgtcgtcgccttcctgcaggagctggtccccccacgactccccgtgtgggaagagctcgtctgggtgcagcgccgcggcccgcaacattagcaacggcagcagcagtagcactggtaacgacgacagcaccggccgcccggccacccggtccccctccat

SEQ ID NO: 3 - DNA, 65793 bp, Homo sapiens
atggggcccggccggccggcccccgcgccctggcctcgtcacctgctgcgctgcgtcctgctcctcgggtgcctgcacctcggccgtcccggcgcccctggggacgccgccctcccgggtaaggcgctgccaacttggccaacttcagggcccgggcggggggagcgccgaggcgcgggccgctctcgacttttccctccttttcccccctcttttctccagaagcgtgtgtgcgtgtgtgtgtgactgggtttttaaaactcgtctccgcttttctgaaagcgggggaggagaggggaggaggagcgcgcctagaggtgggggaggacctgagtggaaccagatgtggcgggcggcgggggaggggagcccgggggactccagcagcctaggaggaagcgggtgggctcttgaggacccccgcgcaccgcgctcccggaccacccggcgccggcccgtcctcggcaggggacctgcagggcgcgcccggggagccagggctctctgtccgggagttcccgggcccagtctcctcgctctgccccaggactgatgcgttaggacggctttccaactatccgcgagggttcgtttcccccaccttccccccgccccctgcatctcgtgggcgttgatattttcttttccacacttagtcttcaaggaggcttgtggcgcgcggcatccgccccgcgtgagctcggcagccctgtgaggaggctcctgccactgggacgcagcaacaggctgggcgccgccgtgctgtgatgttgcgtagcgtgggcagctctgggcacactagtggccctccagaggggacagaccttgagcggcaaaagagctgctaaagattttaaaacccaacgaacaggaaacaaaacaaaaatcagaaaaccccaaaactgtgccattctcttcctgctccttattcaggggtaacagtgaagtgtggggacagacagatccctcgccaagaatctaagagttgaaaggaaagatccacttcctgggggagtgggggtgggggtgtctggagtgaccgggggaatcgcccggcccagcttggtcctgctggctgtttaattgggggatggggacagtccgtgcagtgtctgcagttttgtgcaaggaccagacagccccctggagagtctggggcctccagcctggcccgtgcttgctgggttggcctggcgtgacgtaggcacaagacacatatctgccagatgggaaaggtgggggccacggtgacccacattcacaccaaccggagacccccggggcaattctgggcccacctggcattgaatgtgggacagacacaaaccaggccgggtaaccccctaacccactgcagggcggcctcacaggcagttcccctacctcctcgcactcctgccttcctctgctcctctgcgcagggcctcaggaggcaggcggtgggaaggggtggggaggatggccattcccagagctccagagcacactttaaagccaggccctgggagagagttgtggccagagtgctgactcggcctttccccctggagcttttcatagagggggctggaagtgcctggtgagaggcccggtcagggaggacaaggacgcctggagtgtccctcctcctcctggtctctgtgcctggccctccctggggctgtgtccagggatgaatagcctcagagggtccccttcccgttggcactgcattgaccacaccctgaggaggcttggatctgggtggcaggcagtgtgaggtcttccagcagcagctgagcaggaggaggatggagctgctgcaggttccttccctcggaaccgtgccctctgcacagagtgcgcttgcccctgccctgccaaattccactgggccctaccctgggaagcctgttcagagcctctgggccgaattccttgcccgaagagctggcagcccctagtacctggtgaattacgttgttgttgggaggaattcaccagcaccagagaccaaatagcccaggaaatgcatgtgggaggtagggtgtggcaggccagtgctgctgggggcgaggcttatgccaggcagggcaggggctgcgtgtgggcagtttcctgaccaccatgggggagccctgtgtgcccagagcacggggcaggggcagcgaggcggcagtgccttcttggtgcgtgatggtggaatccatcttttctccaccttggtggagtggcccgcggagactgtcactggaaggagtcctctttgttagttgccatattaccgcatttctggagcatgcagtttgctcagccttgagccaagttcttggggaggagggaaggtagagaagagtgtaagatgcatcctctggaaaaagaaaatcgtgtgggagaatgaaacgtggaacagtgaactgcgtagggggcagaatgccattggtgggatgcctgcctgcccagacccacgctcaacccttcatgtgcaagttcccatttcattttgataacaacccaaggagatggttattgttggaccatttttgcagttcaggaaatgaaggctaagaggggtctgatcatttacattggtcacacccctagtaagtgaaaggctggagtgtggatgcttcccgtctggagattggaggcaggagagaccgtatgggccaaagggtggccaaggctgtctggagagaggcaggaaggggccaccaggctgggctcttctggggatgatgtggaagcttgctggctgggccggggagaaggttgagtgaaagaggtgcctgggaggccagaatggggatgggacctgcaagctggctgtggggaaccccaggggacccttggaggtttccgagcagaggaatgaggggatagttgctgggaaaggggcttctgggagctccagggatcaagtctcaaggccatggggtgaggagacctcaggaaatcttcctgtggggaaggtcggtgctgcctaaaggacagggagggtggcatcgggtacttggggctctgaccagagtcagccagaccagctccctacacttctggcccagcttctgctgagtcgtggggttcggaaacttcttactgttcccccacgcaaaactcagttttccttgctccaaccctctggctctctgtgagtcatcagggcccaggaggtgcccagccgagctgtggctagtgctgggatcctggctctcttctgggttcacctgggcaaggtgagggtacatggaggggaggaagaaccccgggttttggcccccatttttcgcaccagagaaatcctggctttctgatgcacgtagggcaccgtgacttggctcaatgagatgatttcggaagtgccatgcagttctcaggcagatattcatttgagttcctcatcagtttaagcatcgaggaggagtcctgttgcactgtccatacccccagagggatggagcgatggcacccacggctgcgctgtgcatgtggtgggcgccgtgctgggtcctgtgctggcagtagctcttcggcacctcccatctctgagcaggtgtcagagatttgcacacacctggcgtgaaaatgttaaagatgttaagggagtcactcaaggctgtgccaccacaccactagaaggtgttaccagtgagattcgaacccaggagtggttacggagcctgaacgctccagtaccatacagcgtttccatcgcatgaagcactgtcagtgtgtgtgtgttaggaaagcctgggaaattctgccttatgtgttctccccgcccccataactccaagccctctaggacacctctcaatgtgtcccaccgggaggactcctgaggaggagggtggagcgagcagccctctgtcacatgtcagcggtctgggcgcttgggtcagccacttccttgtgggtccaggaggcctggaccgagccgcctgcagtgtcctcaggatctctgagggaccccctccaagaaggcattgataagtgagtgactcaaggtgccccagcatggcactccagcttccctgaggtcaaggattcacatggttgttcattcattcattcattccttcagtgggtgtctgctgagctcctgcagaactgaagtagagatggccatggcctattctttatttgagacctccccaccgtgtctccagtgggggtccctggacttggaaggcctgagcactgctgataggaagcgtgtcttcctgttcaggcctacgtgagatgggagggggccgctttcccacgcctgggtcctgtgaatctgggtggggtgggccagtggggtggtgacgcgctcctggagctcaccctgggccctgcagatctgtcactcttcagctaggggttgtaccagtaaacggcagctggatgagggctgtccagtggcacctgtgacaccggggtctgccggggtccaccagctgcagccctttaggaaacagattgggtcgaccccctcaactgcacactgtggcccctcgtggaattccaccggttgtttcccttcttgcacttccccctggcccctcgtctctccttccgccgctgggcaccagcctcttggctctgcactctcacagcgcagacccttccacctgcgctctgctcccctcagcctcccactcctcctgatttctctttctctgactgtgcattctgccctccgcagctctggtttttgttgtagaagaaggaccaacttcttcctagtttccccggccccccatgcctctggtctcttgtcctgccccagctttctccacctccaggcttctgcccacgcacgatcactttgcctggtgtgcctcccctcccctcttctttcccaccttccagtgacccttccttcgagactcaaggatcctggggaggacgctgagggagcctggtccctgcctcttgggctgacctggggtgacgagagacaagcacacagacatggcccatggtgacaaatcccagattggatgggtggcctctctgtttatctccttcccagggatcacctcaggagggcacatgtagggaacactatagccagccttcctcagccttccccagccttccctgaaggcagccgggtgcgaggctgagcagtggccccttcctcctgtagatgaggcaggagtgtgggcatggctggcagtgtttccccccgtcagtcctctggaaatgtaaatagggacagagtcggtgggaccaggattgatggcactcaccagctatgtgacctcaggcagaggctcaagtgtcttcatttataagatgggaaaaatcaatacctcttgtagaggtattataagattacatacaaatgagatcatgaacgtatgagatatgtccctggtacactactgggcacagaataatttcttagcaaacaatactaaattattgctatttttattagaagagaaagggtgggtgaggtggctcatgcctgtaatcccagcactttgggaggccgaggcgggtggatcatgaagtcaggagatcgagatcatcctggctaacacggtgaaaccccatctctactaaaaatacaaaaaattagctgggtgtgatgtgggggcgcctgtagtcccagctactcaggaggctgaggcaggggaattgcttgaacccaggaggcggagattgcagtgagccaagatcatgccactgcactgcagcctgggcaacagagcgggactccgtctcaaaaaaaaaaaaaaaaaaaagagaaaacatgactatcaaggttcccaaatatgataaaacacagtcttcaatatacaaaatcctgctggacacggtggcccatgcctgtaatcctggcttgagcccaggagttcaagaccaacttgggcaacatgacgaaacccagtcactacagaaaaaaaaaaaaaaaaagctgggtgtcctggctcattcctgtagtcccagctactgggcaggctgagatgggaggattgcttgagcacaggaggttgaggctgtagtgagccacgattgcatcattgcattccagcctgagtgacagagtgagaccctgtctcaaaaaaaaaaaaaaaaaaaaaaaaaaaaaaaaaaaaaaaaaatcctaactacagctgtcaccagaaaggaggtccaatccagaccccaagagagggaactcacgcaagaaagaatttgaggcgaatccatagggaatagtgaaagcaggcttaataggaaagtaaaggaataaagaatggctactccataggcagaggagtagcactgacggctcagctgcttatacaattgttacttcttgatgatatgctaaacaaggggtggattattcatgagttttctgggaaaggggtgggcaattcccggaactgagggagtcttccctttttagaccctataaggtaatttcttgacgttgccatgacatttgtaaactgtcgtggcgctgataggagtatcttttagcatgccaatgcgttataattagtgtataaaaaaatgggcagtgagaatgaccacaagtcattttttttttttgagatggagtctcactctgttgcccaggctgaagtgcgatggcgtggtcttggctcactgcaacctctgcctcccaggttcaagcaattctcctgcctcagcctcccaagtagctgggactgtaggcatgtgccactacacgtggctaatgtttgtacttttagtagagatgggattttaccatgttgaccaggctggtcttgaactcctgacctcatgatctgcctgcctcagcctcccaaagtgctgggattacaggcgtgagccaccacgcctagcccagagatcacttttgtcaccttttagttttggtgggatttgggtggcttctatactgcgtgctgttttttcagcaaggtctttgtgacctgtgtcttgtgttgacctccggtgtcatcctgtggcttagaatgcctaacctcttgggaacgcagcccaggagttcccagcctcgttttacccagccccttttaaagatggagttgctctggtttgaacacctctgatacaacttttttttttttttttttaggacggagtctccctctgtcacccaggctggagtgcagtggcacaatcttggctcaccacaacttccaccttccgagttcaggcgattctcttgcctcagcttcccaagtagctgggactacaggcacgtgccaccacgcccagctaatttttgtagttttagtggagacggggttttaccatgttggccagacgggtcttgaactcctgacctcaggcgatccacccgccttggcctcccaaagtgctgggattacagaagtgcaccaccatgcccagccccattgttttttttttaaattgaagcaggtctgaacctgttacttcattgctatttttctctttttttttttttttttttttcagaaatagcctcaatcagaggcattacttcttcgtgacactttgctaactactgtaagtgccaaaaagtaatcatagtttttctgaaagtaaaagaaataccaaagaaaaaaaatcccaccctcaatggtcctaccccctaaagcccctcaatttatcaccatcctctttatcaccttcctcttcatccttacacttctctatttaaccttcatttagggaaggataaggagcttcctgggactctggaataaaaccaagacagactagggaacactgggatggttgggcatacctctcccctttttgcctctagttgcctttagcctactgtctgagggactgcctcccaagggtcatactgatgaggctccctaattactaaattaagtgacctttcttaggcctcaccttgcagcatgcatgctgttcattggcctcaattgtaagaatgggccaggcatggtggctcatgcctgttactccagtgcgtttggaggccacgggaggagagactgcttgaggccaggagtttgagaccagctggggcaacataacaagaccctgtgtctagaatttttttttttttttctgagacggagtctcgctctgttgcccaggctggagtgcagtggcacgatctccactcactgcaagctctgcctccccggttcacgcaattctcctgcctcagcctcccgagtagctgggactacaggcgcccgccaccacgcccggctaattttttgtgtttttagtagagacagggtttcactgtgttagccaggatggtctcaatctcctgacctcgtgatccacctgcctcggcctcccaaagtgctaggattacaggcgtgagccaccgcgcccggccaatttttttttttttaaattagctgggcacagtggtgcgtgcctgtagtctcagctactcaggaggctgaggcaggaggattacttgagcccaggagttggaggctgcagtgagctatgattgcaccactgcacaacctgggcaacagagtgagaccctgtcgcaaaaaaaaaaaaaagtaaaaatgcatgctcagtggaaaacattcaaaccatacacaagtaagtgcaaaaaaatgtgaagtccactcccttacactacactgattacacactagcacaggccacagtctaccactggtagattctgtgggtaatcttcccaaccaagattccccaatgaagattagtagtagttcattgggtaatcttctgatttttttttttttttttttttttttgagacggagtctcgctctgtcgcccaggctggagtgcagcggcgggatctcggctcactgcaagctccacctcccaggttcacgccattctcccacctcagcctcccgagtagctgggattacaggcactcaccaccacgcctggctaattttgtttttgaatttttagtagagacggggtttcaccgtgttagccaggatggtctcaacctcctgacctcgtgatccgcccgcctcagcctcccaagtgctgcgattacaggcgtgagtcacagcacctggcctgtcgttgttttttaactaaagacagagtctcactatgttgcccaggcctggctttgaacttctgggctcatgcgattcttctgccttggcctcccaaagtgctgggattacaggcacaaaccactgcacctggaccaatggacacttttgaaaactctcttcccccggcctttgtggtgtaatttacatccacagtttcctcccttagggtcttcccttccaccctcagtccagagctggcattgctgccaggttagcgctgctaaggggaagcttgaatcgatgtaaactttctgaatttagaatttctgctaggctcggtgactcatgcctgtaatcccagcactttgggaggccgaggcaggcggatcacctgaagtcaggagttcaagaccagcctggccaggtaaaacccccgtctctactaaaaatacaaaaatacaaaatagtgaaaccctgtctctactaaaaatacaaaaatcagccatgtgtgatggtgcatgcctgtaatcccagcactttgggaggctgaggcaggtggatcacttgaggtcaggggttcgagactagtctggccaacatggtgaaaccctgtctctcctgaaaatacaaaaattagctgggcatggtggcacatgcctgtaatcccagcagctcgggaggctgaggcaggagaatcacttgaaccctggaggtggaggttgcagtgagctgagatcacgccattgcactccagcctgggtaaaagaggaagactgtgtctcaaaaaaaaaaaaaaagaaaagaaaaagaaagaattcccggccttctcagtgcttgtgcaggtcaagctgagctccttaccgttcccatagctcccagtactgcccaacccggactctgctcaggctttttttccctgccaggagcaccagcttcccagggggctgtttctgagtcccaactcacacctgtgaccttctgattaccctcagctggaagaagcctttctctccacggctgctctagcattttactgctgtcgtccctcaaacacctccagagcaaggcctttttgtcactacacaggttccagcacagtaccttgtccacggtggcatttgtttttgtttgtttgtttgaaacagggtcttgctctgtcacccaggctggagtgcaatggcactcacgggagcctcgacctcctgggctcaggcgattcttctgccttagcttcccaagtagctggacgacaggcggcgccaccactctattttgttgctcctatttttagtagtgggagggtcttcctatgttgcccaagctggtctcgaactcctgggctcaagggctcctcctgccccagcctcccaaggtgttgggattacaggcgtgagcaactatgaggctggcatttggtattatttagtaaatatttgttgaaaatgagaaaagaaagacaagtgttccaaagaattttttttttaatagaaaggcaatgagagttacccttctagaattttacatgatgccacggtcatgaaaaacatcatatggttttaaattaaaataaacagaaaaaagtccaatgtactagaaaaaaattccagaagtggatttaaactgttataagaaaataatgtgagacacaccagaaaaaacacactaataggaaaaggaatttttacttttaaatgttatgaggatggccgggcgtggtggttcatgcctataatcccagccctttgggaggccaaggtgggcggatcacgaggtcaggagttcgagaccagcctggacaacatggtgaaaccctgtctctaccaaaaatataaaaattagccaggtgtggcgacgtgtgcctgtaatcccagctgcttaggagagtgaggtaggagtatcgcttgaacctgggaggcagaggttgcagtgagccgaggtggtgccattgcactccagcctgggtgacagagtgaaaccctgtctcaaaaaaaaaaaaaaaaaaaagaaagaaacaattagccaggcgtggtggtgggcgcctgtaatcccagctacttgggagattgaggctgctcttgaactcctaaccctggaggccgaggttgcagtgagctgagatcgtgccactgcactccagcctgggtgacaaagcgagactctgtctcaaaagcaaaataaaacaaaaataaatgttatgaggacacctggattataattagaaattttaatttcagtttatagttgatcacacttgaggaaattcagatagcaagctgtcacatagaaagtgcaaggattgggccgggcacagtggctcatgcctgtaatcccagcactttgggaggccaaggcaggcggatcacctgaggtcaggagttttgagaccatcctggccaacatggtgaaaccccatctctaccaaaaatacaaaaagtagctgggcgtggtggcacactcctgtaatcccagctactcagttggggagggggctgaggcaggagaatcacttgaacccaggaggcagaggttgtggtgagccgagattgtgccactgcactccagcctaggcaacagagtgagactttgtcttaaataaataaataaacaaacaaagtgcaaggatcactggaaatgaaggaaattagctttctttaactacttaaactagcaaaaacagaaaagctagtattggcagagctgtgaagaagccaatatatctgactgggggactgtaaattggttctgtttttcaggattcaatctttcggacaatatgtaagaaactggatatgtaagaattcctatcctttgatgtcataattttacttttggtaatttataagatattaatctcaaagataaaaaaaatgttcttagaagtgtttttaagagactgggtctcactctgttgcccgggctggagtgcagtggcacaatcatagttcactacagcctcaaactcctgggcttaagcaatgctcccgcctaagcctcctgagttgttgggactacaggcttgagccacagcctttagtagcaaaacaaattggaaaccacatgatggattatttacgtaactttaagtgacaaatatatggctaatgattctttttttttgagacagagttttgcacttgttgcccaggctggagtgcaatggctcgatcttggctcactgcaacctctgcctcctgggttgaagcaattcttctgcctcagcctcccgagtagctgggattacaggcacctgccaccatgcccagcttttttttttttttttttttttttgtatttttagtagagacgggggtttcttcatgttggtcaggctggtctcgaactcctgacctcaggtgatctgcctgcctcggcctcccaaagtgctgggattacaggtgtgagccaccacgcctggccagctgattcttacaaggaaagttctgaggactgtgagacagtcttacaacatttaatatgcacatactggaaacaagatgcatacctgacattcatctgtccgtttattcattcagttaactggtgtctttgttcattcattcagttaattggtgtctttgtttttgttttctttcctgaacattgaaaacgtaccaggcatggtatttggtgctgccaacaccgtggtgagcaggctaggcatgattcctgctttcctgaggcttgcgttttgaaagcagtcccttcccaatatagagtgccacaaataatctcaaacgtacactgatttcatttgcaggtccacttttttgttttgttttgttttgagacactgtctcgctctgttgcacaagctggaatgcaatggtgggattcactgcagcctcgacctcctgggctgaagtgatcttcccccctcagcctccgcagtagctgagaatacaagcacaccaccaccactggctaatatttaatttttttttttttttttgagacagagtctcgctctgtcgcccaggctggagtgcagtggcacgatctcggctcactgcaagctctgcctcccaggttcacgccattctcctgcctcagcctcctgagtagctgggactacaggtgcccgccaccatgcccggctaatttttttgttttttgttttttgtgttttttttagtagagacagggtttcactgtgttagccaggatggtctcgatctcctgacctcatgatccacccgcctcagcctcagcctcccaaagtgctgagattacaggcgtgagccactgtgcccggcctaatttttttttttttaaatagaaacaaggtcttgctatgttgcccaggctggtctcaaattcctggcctcaagtgatctacctcagcctctcaaagtgctggagttacaggcataagtcactgtgcctggcctgtaggtgcaatttttaagttcattaagagaaaaatcggctgggtgcgatggctcatgcccgtaatcccagcactttgggaggccgaggtgggtggatccggaaggtggaggttgcagtgagctgagatggcgccattgcactccagcctggccaacagaccgagactgtctcagaaaaaaaaaaaaaaaaagagaagaaaagaaaagaaagatggaggcaatcgactgctctaaaaccttagacctagaaacaggaacctgtctgacagtggggagaacgactcccagacaaactagcctggctgaataggaaggactcaacgggaagcagctttgcagtcatcttggtccccaaggaaaggggattctggtattaccccataaaaatttaacctcattcctgaaggatctaagtatgtaagggcattgatggtttgcaaagagcttggaagcactgtttatagtagcaaaaacaaaaatgtacttggaaagagatatggataaattatagaatagacacaatgaaatgtaacacagaggtggacgaatggactccagctatttgcaacaacatgggtgaatattagagacttaatgttgaatgggggaaaagatcccagaggactgcatatggtacaatactctttctcaaaccactggaaaaactgaaaaaataatacagtgggcacccatatactcttcacctagaagcaccagttgttaacattttgccacatttgcttatctcactctctgtgtgtgtgtgtgtgtgtgtgtggtgggtctgtttggtgaaactttgaaagttagttatagacatgacatttcacacctaaatacttcaacatgtaacaactaaaaacaaagacatgctactatgtaaccataatatctaatcacacccaagaaatagagcggatgcaattttttttctcatatgcagtccgtgttaggatttctccatttgtcccaataatgtcctttatagctgttttaaaattcagaattcaattaggtgtcatgtctctttaatctcctttaatctagaatagttgcttggccttgttgtcttttatcggcattgcatttttgaggatcccaggccagttgttttgtaaaacgtcccacagtctaaatttgtctgattgtttactcgttcctggattcatattaaacgtttttggaaagggaactcatgggtgatgttgtgtccttccattctgtcacaccaatgtcaatttgttcccttacgctaactttgaccatttgtttcagctggtgtctaccagatctctccactaaaaaggtcccttttccctttttctgagatgaagtctcactgtcacccaggctggagtgcagcagcacgatctcagctcactgcaacctccacctcccaggttcaagcaattattctgcctcatcctcccaagtagctgggattacaggtgcctgacaccatgcccggctaatttttgtatttttagtagagatggagtttcaccatgttggccaggctggttgtgaacccctgagctcaagtgatccatgtgcctcagcctcccaaagtgctgagattacatgtgtgaaccaccatggctggcctcccctttgtatttagtagataacctaaggaatttgaaccatagtcttaaaaaagcaaagttgttcatagtagcattattcataatagccaaaaactaaaaactagtgccaaactgaaaaccacccaaatgcccatcagcgatagaatggatgaattgtggcagcttcacaaaattgaatattagacagtgatgagaatgaacaagctccagttgcatgccacagcatggatggagttcacaaactgagccagacgctacttcataccatatgacccaatgtatagaaaacaccaaaatagccaggcgcggttgctctcgcctgtaatcccagcactttgggagtccaaggcaggtggatcacctgaggtcaggagttcgagaccagcctggccagcatgatgaaaccctgtctctactaaaaatacaaaaattagctgggcatggcggcaggtgcctgtaatctcagctactcaggaagctgaggcaagagaatcacttgaaaccgggaggtggaggttgcagtgagctgagattacaccactgcactccagcctgggtgacaaagagtgaaactccgtcaaaaaaaaaaaaaaaaaaaagaaaaagaaaagaaaaaagaaaataccaaaataaaagacacaactcatctgtgctgtttgcagtcaggagagtggttacctttagtgcgggagaagggtactcagcaaccagggagggtttcaaataaaatttagaaagaacaacaaacagggaagggatctgtaacaaacatggcaactagctcatataaaactatgagaaaaacgcaggaacctgcaatagttttaaggcaacaaagatcaaaatagataagactataaaagatctgaacggagttcaccaagaggaaatgccagtggctaataagccaatggaaaatgtttaaaatcactagcaaaacaatttcacctctcgcaacagtgccaggaaagattgatataataatatgttcaacgtaacattataattaggaaattctatttataagttaaagtaatagctaggaaatgatgaaatgaaacgtgatattatatcatggaataatatacagccattaagatgatttttacgaatatttttaaggacacagaaaaatccctttgctgaaatgttaagtgaaaaaagatccaaaaatgtttgcgtgtggatatgtgcatgtatatttatgtatatatatgtgtattacatgtgtgtgtatatatatgaatagaaatattttggatgtctagataaaattggtagaaaaaccacagtaaaactaaaagactgtactatgctatctactacataatatattatttaacaggggtgattgtagatgaaatttttctgttttaaacatgtctggaggctgggtgtggtgactcctgtctgtaatcccagcactttgggaggctgaggtaagtggatcacttgaggtcgggagtttgagaccagcctgactgacgtggtgaaaccctgtctctactaaaaatacaaaaattagcaaggtgtggtgatgggcacctgtaatctcagctactcgagaggctgaggcaggagaattgcttgaacccagaagatggaggttgcagtgggccgagatcacaccactgcactccaccctgggcaaaagagaaagactccgtctggaaaaataaaaaaacatgtctggaggcgggatgccgtggctcacgcctgtaatctcagcactttgggaggccaggagttgaagaccaccttaggcaacatagcaagaccccatctttacaaaaaataaaaataagaaataaacatgttcgtatcttgggccatggagtggcagcgggcatcttttgcttgtgatcagtcagtcacgtgaccccagcagcatttgtgtcctgttcccagtaccgtcctgtaattggtcagaggatgagcatgtgactcctcactgccagtcagagcctccctggaaggtggtgtttttcagcacagcccgcagatcctcatttgggacttacgtgagggcataggattcctccacgctggtggtcgagggtcagcctccgagattctgcgggagagactctcaagggtcaccagctggagctggttctgtgtctgcagggaaccacctggtgctctctccattaatagccccctaccgcgagttttctcttctttatctttgtgacttagcccagtgttcacagaaggggaggggtggagggttcaaacgctgtcatacacgtttgcttttcgaaaaacaacaggacacaaaattttataaacatgaaatgacagtacctggtacgtgactcgaatgcaactcagaagggtgccagtaagttagggaattggcctttggaaggcttccttctttttaaaaactataatgtttccatataatgtttgtgtaacaaatgagaaatgggagaccccattatcatttactaacggtaaatttacataaagtaagccagggtcagcatcagataattccgtatagcccatattttaaaccttcacattcctccatttcacagataaagaaattgagcctgagaggctaagagacaggatcacaggagcaaggaatgtgatccaggtctggttcctttgcatggtcctctagaaacattatatacattcatctcaaaaatatatatatatgtgatatatatacacacgtatataatatacatattgtgtgtgtatatatacacataatatgtgtgtgtgtatatattatatatatatatatatatatatatatatattttttttttttttttttttttttgagatggagtcttgctctgttgcccaggctggagtacagtggcatgatcttggctcactgcagcctcaatcttctgggctcaagcaatcctcccatctcagactcctgagtaagcaggaccacaggtgtgcaccaccaactgcagctaatttttttttctgtattttatgtagagatgggtttttgccatgttgcccaggctggtctcaaactcctgagctcaagcaatccacctgcctcagcttcccaaagtgctgggattacaggtgtgagccaccgcacctggctttccctgtaaaaaactttatagagatgttttcatgttgtttcataatgttcttctttcattcaggaatatgtcatcaatgtatttctgcccaacacttctactggctacatagggttctgttgtgtgagtggaccataacttatcccctattggacatttaggtcatttctctgtccttttctatttttttttttttttttttgagacggagtcttgctctgttgcccaggctggagtgcagtggtgcgatctcggctcactgcaagctctgcctcccaggttcacaccattctcctgcctcagcctcccaagtagctgggactacaggcgcctgccaccatgcccagctaattttttgtatttttagtagagacggggtttcaccgtgttggccaggattgtcttgatctcctgacctcgtgatccacctgcctcagcctcccaaagtgctgggattacaggcgtgagccaccgcgcccggccccctctctctctttttttaagagacagggtctcattatgttgcccaggctggtcttgaactcctgggctcaagctatcctcctgcctcagccccaaagtgctgtgtttataggtgtgatgccaccatgctgggcaggtcatttctcattgtcactgatagaagtgtcactgtgtacatcgtagcacaccagactccttgctcagacctgaagtcggatgtggatcccagtcagggtgaaaaggtgcttgacctacgtcaacttcatgcctctgaaggcacccacttctttgctgtcagcagtgtctgctccttggcaccaggattggatggttggtctcatccatctggagcctctcgcagaaggatcttctttggtttacttgtttgctcaccctctccaaatgtgctgggtttagtgtcaaactctgcatatatgagttcaagtcccgcagggttaagtttgaaacaaaggacagatgcacaccatatattccctatctctttttaattaccaaatacattaattgtagcaaatacaaagagaagaaaataaaaatcaacagctatctcataagtcagtcataaccactgttaacacctgggtatgtacagttgttctggaaccctgatgtaccccagaattgcttgggactcttgtttacaatctagatattccatcactccgagattctaattcagtagaactgctctagggcccacacaccggcattgcaaataagctccccaggcgatttggacacccaactttaagaacaagaatgcaaaagcgtctttctagacattacttgatttaaaagaaaataaaaatgaagttatactatatattcattttttctttttctttttttttttttttgagacagagtcttgctctatcgcccatgctggagtacagtggcatgatcttggctcactgcaacctccgcctcctgggttcaagtgattctcctgcctcagcctcctgagtagctgggattacaggtgcatgccaccatgcctggctaattttttgtaattttagttgagaaggggtttcactatgttggtcaggctggtctcgaactcctgacctcaggtgatccacccgcctcggcctcctgaagtgctgagattacaagtgtgagctgccgcgccctgccatttttgctttaaaaaaaaaatcagcaatagaatgtgaacttctgtgtctaaataccctttatacatttctgttgtgtgcatagtattccattagacgaatatatttccatttacttagtcaacactatattgttggacattagttgctcccccatcattgctattgtaaataatgctgtgtggctaaatctttgtatacattcttagttctttccttaagttaaattcctaggaaatgaattctggggtgaaagttttcattcttttgaaggtttttgatagatgctgccaatttttccagcaaaaaggttgccccactggggctgctactcccatgagctccctttgtgaggagccgtgaggtgagatcttcccatccatgatctcagccatccctgccaactacccagcgggcagcacgaggtacctgctcagtggtacctgctaagtgctcagtaattggtctacgcagtggacaggaggagtgttttcttcactgaccagaggaggacactgagagtggaggggtcaagtgactcgctcaggaactgggactcaaagccagctttgaatctagagcctgagttctcttcaccaccccatcaatttttaattgatattagagagggaagaacaggccgggcatagtggctcacgcctgctatcccagcactttgagaggcccaggcgggcggatcacttgaggtcaggagttcgagaccagcctggccaacatggcgaaaccccatctctactaaaaatacaaaaataagctgggtgtgatggcacacgcctgtaatcccagctactcaggaggctgagacaggagaatcacttgaacccgggaggcggaggttgcgtgactgcgcaagggtcctggtgagggtcccaggtcaactcctcccgtcccagtgtctgccaggccaaggggtgtgggcgctgccccacctctttccacactttcccttccagctgcgttgtgagtgacctcattttccctttacagctgcccttggagtttatttctgcagaaccggaatgtggctgaaagagtgcacaccctatctgccctgccctagcccttccagagagccccctacttttccctgacacaccctcctcttctccttgcatctccctcctgggagacctgaggaggggtccagagttcaaagacagaaacatatggtggtggcaagggactgggcacccatgggaattggggtcgggggaggactttgcatctggaccgtgggtttatttttcttttaattcttctgaagagcagcttaaacactggagtccttggccgtatttcctcttatcgtttcttcttatccagattgcagtgtctgctaagcctggttcccaggctctccgctgtgaaacacaaacagattcttgagctcgctgcccaaaactgggcagaggttcttgcttaaatatcaaaagcagtaatggccttccattactctgtgctccccaggtacagagggacaggaagccttctctccctttgtcctcccagaaacccaggagacaggtcttattcgtgtccttcccaaatcgaggcttgcggaagtgaggcagccaatcccagggcccaagggcccaagcagaggtgcgaacctgggtaggtccacattctagagctgccctcccaagccctccaccatggtgcctcctggaggtgggggcaggcccggctgatctgtcgtcacagaagttggcaggtgctcaatctttggctagaattttaaagggagatcttgaaggttcccaagcaagaaggattctaaattcacttgactttaaagagtgccatttgggcgcagtggctcatgcctgtaatcccagcactttgggaggctgaggcagatggatcacttgaggtcgggagttcaagaccaacctggccaacatggcgaaaccccgtctctactaaaaatacaaaaattagcgaggcatggtggtgggcgcctgtaatcccagctacctgggtggctgaggcaggagaatcccttgaacccaggaggtgaaggctgcagtgagccaagatttcatcactgcactccagcctgcgtgacagagagagactctgtctcaaaaagagtgccgtgccttgcttttagaaccaaacaagtgatctttgaatgttgccatgtcaggagggcagggaattgccaatgtgtggagcaacagctcattaggaaacgtgccttgatttctcagtcatattgtgctgtgcttcaaatgtgtctcatgtctcctatgcagcgtgtcacagtgtcatttgacatcaatcatgttggcagggcagcagaccccccccgccccaccgatacatacattttttgttttgttttattttgaaatagatacagccttcttgtcattcttttttttttttaattttttttaaaatttttttgagacaagagtgtcgctctattgctgaggttggagtggcactatctcggctcactgcaacctccgcctccagggttcaagtgattctcctgcctcagcctcctgagtagctgggactacaggcacccgccaccacacccggctaagttttgtatttttagtagagacggtgtctcaccatgttggccaggctggcctcgaactcttggcctcaagtgatctgcctgcctctgcctcccaaaatgctgggatcacaggcatgagccaccatgcttggccgtcattcttaatagacaccaggatactgaaaaatgctttgccactcagaaaaacaagggtacaagcatgatactatatggccactctcattccacttcagtgtgtgtgtatggtgggggcacaaaagggatgggtggagactgtggcaaaccggtgagcccacacccctcccaggtgggcagctgcaccagctcttgtggctggcaccctgcaggcatgctgcctcgactggaaatctacatttttatgtgtaatctctctcacacatcctgaagccaaacacaacttgcctgtgggctatcatctaaaattcggaggctagctggagtccgctctggaataagtctgcctggccatccttcccagctcttccccttattaactgtgttatcaagatgttaatctctcagttatttaatacctcagtttcctcatctgtcaagtgcagatcagaattttgcctacatctgagggttgttataaggattaatgaaaagccttggaacccactgtctggtgcctggacgcgcatggttaatgcctggcattctttgtgtctctttttacaagcgcagggtgaccacaggtcccagtttgccgggggcaggcctggttcatgcttgtggtgctggtgtaatgatcactaacttgccatttcattgtcggtattgacgtaaatgatatgatttgcctagagaagcaaatactggcacagtgctgtttaggactctgatgaggtttagacctaatagaaaggccatggtccaggttccttgaacatccctggacaagtcacttaacctttctgtgttcagtttcccgttctaaacgattggcaagatgaattctaagatccctccattcacagtcattctgtcatttgacaaacatctttttttttttttttttaaacgcagtctcgctctgtcgccaggctggagtgcagtagtgcgatctcggctcactgcaacctccgcctcctgggttcaagcaattcttctgcctcagcctcccaagtagctgggactacaggcgaacgccaccacgcccagcaaatttttgtacttttagtagagacagggtttcaccatgttggccagaatggtctctatctcttgacctcgtgatccacccacctcggcctcccagagtgctgggattacaggcgtgagccactgcgcccggccgacaaacatctttcacaagctcagactcatgccaggcaaaggtccagggcatggagaagccaggggcagagcctgggtcctcagtgggcacttggccagtgaaaacctccagtgcaggttgttgggaatctctaccttttttgtcaagtgttacttgaaagaccattaccaagaaattgaattgaaagttaatttttttctccttctttacggagacgagtttcactctgtcccccaggctagagtgcagtggtgcgatctgggctcactgtaacctcctcctcccgggttcaagcaattctcctgcctcagtctcctcaggcatacaccaccacatctgccaccatacccagctaatttttgtatttttattagagatggagtttcaccatgttggccaggctggtctcaaactcctggccttaagtgatccacctgcctcggcctcccaaaatgctggaattaaaggcgtgagccaccgcgccctgcctgactgttaatattttaaacctcagtttctcaggaactaatcattctggttgaatacattttgctacacatttcttttgtttgtgggggggtatctaagttgtttttttcttttaagtcagaaaacataaattttcactatggaaaattgtaaattacagaaaggcccaaagattttcttcatttctgctgactcttcaatgaaccagaggggcacgtgtaatatttccgctttcggttactccccaccccacacaccttgtagacactttgtcaaaactacattttaaaaaaatgctttcccccaaattcgtatggatgtctcctaataaagtcacgcagtcactcactggttcgtcctcaatcaaccaggcgtggagtcctaacctgtccatgattttaagaaagggcaggcgcacaacacccccgcacggagaaatgaacaggtgtggctcggcagaggccttccgcacctgcgttatggagtggtggccgctgtcggcgatggggtccccgcgagggcacaagtgatcgcgtggccacggcgaggctctgcctccgcccccgcgctccgggtcccattcccgaattctgccctcagcggcctggtcaatgaggttggatcgttggcccagaaagccacttcctgccgcgactggagccagccgtcgttgtgaaaattccccccacggggaaggaagtggctcttgggctgtttattttggctgcgggctagcagacggcgccaggggcagcggccacgcagaccgggcatttgtctagctgggcgggaggcggcggagcgggaagtgccacagattgaggggcgcacggcggtggagggaggggcgcccagtggagagggggctcacggtgacagaggaagtggcgccgcttgggggggtgcacgatggcagggggaggggtgctcggtggagggaggggcacacggtggcggggggaggggcgtgccgcagattgagggcgaacggtggcttggggaggggcgctccttggagttgggggcgcacgatggcgggggcaggggtgccccttgggagcagaagcagagaagactcggaactccaagagcaggtggatctgggagccccagatcctgggcactgggttgggggagaaaggggtcaaggtgtgcccgcccccgggatgctcggactctggggatccaggcctggtgacccccggtgaggaagagccagtgggaggaaagggccgggcgctgcccggagcagaggctggaggcgagagcgattgctgcttgctgtagcgcggccgctctccagttcctccggcaaggggggcctgggggaggactggccgaaaatactgtgatgctttggtctaagacaaaagccagcaggggatgagatcaaacaccatgacactgcgaaggggaaggccagcgcgggtgaggatttgccttcccagagaaccccaggggcggggctagcttccactcagcccgcattacccccacccccgcccccacccccggggcggggtctggaggatcagcaaaaccgcgggaaaggtgacagtcgccttccccaccaccgcctgcatttagtaggtggagggacagtcccacctctccacccattccctgccagagtgtgatttaatgtcttcctagaacacaggatatcacagtctgcagcacagagatttgaagtggggagaaaagcatctctggaaatgactggtgggggaagcccgccactgctctacaaagggtggcctgtgacagcaacagcgcaacactgggactgtgtgtgtaggttttaggcacaagcactttaagctgtgggaggaaagcagtttggcctggctgggatggaggggctgttggggaggagggaggagggtgtgaaaaggggaaggtggccggtagagggaaacccaccatggttcaggatgtcggtggggatgttgcacagttgggcggggtattgggcttcgatatcatgtaacccccgtcctccatggaagcaatgcccccctcgtccagcccaggatctaatgtgcatgcatgtacgtggacacagtctctagccaggggtggtgcagggtggggtgctgggtccccagggttggcctcttagcagggtggggctggcagcttctgctgactcaaggacatttccaggcctggcctggaggttaagcagctcattgaaaacagctgctcctcttggctggcggccctttcaggcggcctctggctctccctgggatgacccctcgcctctgcgtggcctggggaggggcagtgaagggagagaggctcagaggctggaggccaccctgccatgccctcagggatggaactgagtcccctccagggctggccagctgtcctccagcccccagctttccaggagctaagccaggtggtcgctgggccctgcgttcctgcctgcagcctttgctcctgcggttgggcagcttggaccgccttccttgtccttggaatgccttccttgtccttgtctctgcttgtgcctttcagggttttatgtgacatgcctctcattcaacacatatttgttgagagtccgctgcatgccaggcaccatgctagaattaacttctcatctgcctccttcatctgtgatgtcttgtggagagcagccgtcttatctgaggtcagacctctggctgcctctgtggatgggctgagtccggggaaactgcagcaacagctgagggttttgctccagagacacaaactgttgtcaagcctttgcactcagcctcgcgacttcaccaaagcattgtgtgccttgaagcagacaggacgggagaggaggcctcagtgaccatcaaggaggaaggagatgggttactcaatgtagggcaaattgctttaaaacccagttgcccatgagcacaggcatgtttcccaggtgatgacctggatgtctgagaaacaagaagtgtggactacatcagaaatattcctatcaaactagacacggtggctcacacctagaaatcctgcactttggaaggccaaggtaggaggatcaggaggttggggccaggagttcaagatcagcttgggcaacaaagcaagactccgtctctacaaaaagtaaaaacaattagctggggctgcgtgcggtggctcacacctgtaatcccagcactttgggaggccgaggcaggcggatcacgaggtcaggagatcgagaccatcctggctaatgcggtgaaaccctgtctctactataaatacaaaaaaattagccgggcgtggtggcgggcgcctgtagtcccagctactcgggaggctgaggcaggagaatggcatgaaccccaggggtggagcctgcagtgagtggagatcgctccactgcactccaggctgggcaacagagcagactccgtctcaaacacaaacactttgggaggccaaggtgggtagatcacgaggtcaggagattgagaccatcctggctaacatggtgaaaccccgtttctactaaaaatacaaaaaaattagccgggcatggtggcgggcgcctgtagtcccagctactcgggaggctgaggcaggagaatggtgtgaaccctggaggaggagcttgcagtgagccgagatcgcgccactgcactccagcctgggcaacagagcgagactccatctcaaaacaaacaaacaaacaaacaacaattagctgggcatggtggggtgcatgcctatagtcccagttattctgggggcttaggtgggaggatcacttgagcccaggaattcaaggctgcagtgagctatgatcatgccactgcactccagcctggacaacagaacgaaactctgtctctttaaaaaaaaaaaaaaaaagaaaaagaaaagaaaaaaagaaagaaagaaatatttctatcacacttcatttatctggaaagttcatttctatctatcaccttcacccccaaccgtgcagacttaaacttttccatggattgcttcatgagttcagcctctccagcttcattataaacgcatgagggcagaggctgtatcttcttttgattcctaggctggaatgcagtggcgtgattatagctcactactgcctcagactcctcggctcagttattcctcctgcctcagactcccgagtagctgggaccacaggcacatgccaccacacctggctaatttttttttgagatggaacatcactctgttgccaggttggagtacagtggtgcaatctcagctcactgcaacctccgcctcctgggttcaagcgattctcctacctcagcctcctgagtagttgggattacaggtgtgcgccaccatgccccgctaatttttgtacttttagtagagatggagtttcaccatgttggccaggctggtctcaaactcctgacctcgtgattcacccgcctcggcctcccaaagtgctgggattacaggtgtgagccaccacgcccagcctacacctggctaatttttaaatttttttgtagagacgggatctcaccatgttacctaggcttgtcttgaactcctgggctcaaacgatcttcccaccttagcctttcaaagtgcagggattacaggcatgagccactgtgcccagcatctggttcatttttaatttcccttacagtgcttcataaatgattatcatctgtatgggataagaaagcccccaagggagcagccagaaggatgattaggagaatgggctgagggtccagcctccaggcacctcctctgacatggccttggtgctgcagccgacccaagaggctggtctgacaggtcagaggaggtcctagagcagtaacctcccaggaaataaggagaatgggacagaaacccaatgcagacaagctaattaaaaaaggaatgcaggagcccagctggctgcagatgatcctgagtgtttcacagaatggaggaaagagcctcagggattcagggacaggacatccagcatctttgggactcacttcctctctttccatctctttgtcccgtttcactctgcttctctttatatattatcccctcatcgtaggaagcatggccattggcagccccagcctagcaatcttaacaaaaagggaaagctttatgtgttccaggggagttctctgatcagccctgtttgagttatgtgccccatcactgtggccaaggggatagacactgtcatcagctcaagagtgagccttgtgcccacccacacatccacatagccagaggtacacaatacaaggagaacaagaaggggatgggaagtttcctgggcagataaaggcagctcaatgaggtcacagattgttatcgttgccatgtcttcattgtagagcccagcgccctgccagaccatatggtgataagggctgggatcatgtctatattgttcacaataccctcagtgcctgatgccctgctggcatctatgaaattctcaatacatcattgttggatgaatgaatgaataaatgaatgaatcacacccagccagtgctcagtgcacagctccctgcctagcttcattcatttctccatttactcagtaagaacatattgagccacagcttgatgcgtagcactgtgctaggcatgggttgattgcctacaaaatgcaagaagtggtctttgtccttggggaggagaatcccaagatccttgtgaggggccctgcaccttctgtggggggcacttttgctccctgcactgacacctgatgctcccatgcatatggatgaggctggggggtgggtgaggctggaggggccctctctggatgggggtggggggcatcaggcctctgcactctgagctccctctgcccgctttgtatgtgcttgatgaagcacgggtacaaagaacacagagccagctctcaggacgccttttctctttccccagcagttccagcccagcctgagcgtgcactccccctccactaggccttggagaaagagcctcttttcattgagggcccaggcccaccggccacttcgggtggaaaatgctctcaagaaccccctgaccccggcttggcagagtccccccggatccacctacaggaatcccccctcagcttctcctttgctttgcagagctcctgcacgcctctcctctgctttttgattcctcagccttttctcatttgctgccaaatgagaaaatgcaagaggttgcctttgtcctcttttgctgccaaatgagaaaatgtgacctgtgttcttcagtgcctcctccacattgcagtcttcagatctctgcccagcggagtctctcggcccttgggaggcacagagctccttgtaacgcccccagacacctgggattttaaaggtttgtttgctcttatgcccggcatttctcctctaactggattacaaatcctgcgaggggagggacggtgctgcgagtttccttgtggtcccagcattgcctggaacatgccatgcacctagcagaaagttgggtggatttgaacccagagcctaggatgtcagagggaggaggggccttgaagctgtgcctcatgtgacaaaggaggaaactgaggcctgaaaggtacttctgttggacattctaatcccgtcccctttctggacttcagtttcctctgtcaagtgaggggaggttggcctgaccctgggaaaagcccctcccgactaacctggacctctttcagacccttcattcaccgtttgctacaactgctacagcccacctggcattgatttagcacacttgtggattttctggctggggaggagaggaagataacgtcctcttgggctattgtgggctcacgtgaagttttgggcttcgttttccagtcgtatctctgagaaacatctacaaaacaagcaataatttttttccttttccggccccaggcccctttggctacctcctgccaaggccattttctgttactactgcctggagtagagtttctgagaaccagatacctactccttaaaggaagcttgggcaggcatggatcaagtcctggagctggtttctggtttctggtgggtggctgaaaggtgacctagtctgtttgggttgttataacacaataccgttaacagatggcttgtaaacaatagaaatgtatttctcatggctttggaggctgagaagtctaagatcaaggttcctgccgattcaatgtctggtgagggccacttcctcgtggacagctgtcttctcactgtaccctcacatggtggaataggtgagggtctctctggggtgtcttttattaggacgctcatcctatccatgaaggctcttccctaatgacctaagcacctcccaaaggtctcacatcctaatgccatcaccatgggggtttaagatttcaacacaggccgggcgtggtggctcactcttgtaatcctagtactctgggaggccaaggtgggaggctcacctgaggtcaggagtttgagaccagcctggccaacatggtgaaactgcgtctgtactaaaaatacaaaaattagctgagcatggtgacgcaggcctgtagttccagctacttgggaggctgctgaggcagaagaatcgcttgaaccggggaggcggaggttgcagcagtgagcagagatcgcgccactgcactccagctggggcaacagagcgagactctgtatcaaaaaaaaaaaaaaagctttcagcacatgaatttttggaggttgacacattctgttcatggcaggagggaaggagcaggttcaaggcaggggacccagagattagctgtggcagttgtcctaggggtactttaggaagctcactttgtcctgcccagagaggaaccccatccaagtgctgttagaggacagaggggcagggtccctgccccaacctacattcccatggtggtgttttgagcatcagtgacaggagctgtgggtactatgttttaggaaaattaatctggctgttggaacaaggagcagaaagataggctggttgtttgtgattattatctaggcatgaaattcgttcattcatgcatgtgttcatttgttccttcagtatttaagcatcactgtgtgtcaggcagaactgtaggtgctggggttacagtggtggagaagacagaaaacaagagtgtcagatagcgagttctggttctggaagtggcagagtagcttgtgtcagtctaactctctggccaagaatgcttttagactaagggaaaattataaaatatggtgattatggtgatttgaaggcaatgaacagcaattaaaaccagacagaggtagagaggaaaaaatccttgaaagtaaaaagtaaacctggcacatttaaccagcttttccctgaacacactacctggtttgaggagctcatacagaaagtggcagttttaccggttagagcttggggccactagaaaggctaacattgagaggaaatgttaggaaagcaaggagccatagaaattccttctacagattctccttaaatccttggtgactcctgaactgtgcatttgcagggtagggttctaagcagcccagcaagaaacagcagctgggaggtagaggtttcagttgctatctggtgctgaggaaacagagtttaaaatttaagttccaccaagtttgaggggcttagtaatcacctaaggttttcttttgaaatcccagaagggcctcaccttatgattaaatccaaggttaagagccacatctgaagactaaaggcaaaactcaaacaaccccaccctagcaaagcttaaaaccaaacctccacaagagcaaggtaatccactggtaatttaactgcctgccataacaaaatccagcattctttagaggaagatttttttaaaatccagattccctttaacatagcatccctgacgttcagtatacaacaaaaaagtccagactccttatgacattatccacaatgtccagcatataataaaaatttgcagatagtaggcagcaggagatgacccataatcaaaagaaaaaacagtcaatagatgcagatgcacagacaacccagatggtgaaaaaaatcagataataaatgccatgaagagcattataatcaagtgatatgatagagagtaactgtggtcactttacgattgggaggccaggaggatctctctaaagatgttacccttttttgttgtttgaaatgtgaaatgccaggtagaggaaataatgcataggcccagagataggaaacagccaggcttgttcaaggaagaacaaggaggtcaaagcgacttgagcaaggtggagtaacaagatttgagatcaggccgttagacaggaccagatcatgttaaattgtggctcatagtaagaggctgaatctcatttgaagaatgatgaattatctttgaagtaattcaagcaggaggaagacttacctgccttctgttgttgttgtttttttcccttttactatgagcgaggctgtcatttcaaatatttaagaggcaattgcattttcttcttttgctgtctatgcatatctcttcttcattttctactgcattgttgatactttccctaagtttcatgtatctatttttttggtagaagctcttctttcttttcttttctttccttccttcctttctttcttttctttctttctttctctttctttttctttctttctctttctttctttctccttccttccttcattccttccttccttccttccttccttccttccttccttctttcttaagactagtaaagtcagaaaggaggaaagagtagaacaaagactttggtctgtaactgactatgaacaaccaattgagataactcaccatctttggaccagccagaggctcttttaatattgtggacttaactatttgcctctaataaggcacatatatttttgtcatttatttgaattttgaccttgctgatggcatcatatgttggcattaaaaaaatctttttatgggaaaaaagttcttatgctattgaattcatcagtgttgtctatgaccttcaacattccaataatatttttaaattttttctaggactgatttatatctttaaaagattattctaggccaggtgcggtggctcatgcctgtaatcctagcactttgggagtctgaggagggcagattccctgagctcaggagtttgagatcagactgggcaacatggagaaacactgtctctactaaaaatacaaaaaaattagccaggcatggtggcatgtgcctgtagtcccagctactcgggaggctgagacagaagaatcgcttgaacctgggaggcagaagttgcagtgagccaagattgcgccactgcactctagcctgggtgacagagcgggactctgtctcaaaaaaaaaaaaaatagattattctgactgtctttggagaatggactataataggatgatgagatcagcatcggggaagagagagaacttgagatttggacccaggagatgagggccacatttcagctccttcccttaacaatcttacgactcttgtcatcaaattgcttcaccaccctcagccaatttctttaccaataacatagcataatgctgatttatagtcttcttactgtgccagatcctgttccaaatgctttacatgtattatctcatttcatcgttaccagagtcttaagatgaggaaacggcatagagagatgaagcactttgcccaagtcccatattcagtggcagacctagaatcgaagcactagctgccgggcacggtggctcatgcctgtaatcccagcactttgggagaccaaggtgggcggatcacccgaggttgggagtttgagaccaccctgaccaacatggagaaatctcatctctactaaaaatacaaaaaaattagccaggtgtggtggtgcatgcctgtaatcccagctactcaggaggctgaggcagaattgcttgaacccaggaggcagaggttgcagtgagccgagatcgagccattgcactccagcctgggcaacacgagcaatagtccgtctcaaaaaagaaacaaaaaaaagaaacaaaagaaaataaacactagcagtcggattcatcatttcaacacatgaaaagtatcctggggagctagtggctagtggatgcaaaagttctttggagattggatgtactccatatgtgtgttgtgtacataagtggcagtcagggaacaagaggaaggtgatggatgtcacagagatctgggtttgcttactgggcgtgggacactgggagtaggtcacttcatgtctctaagactctgtctgtcttttcttaccttccttccctccctccctccctccctcattccctccttcctaccatccatccctccctccctccttcctccctagtttcttcctttctttctctttcctccttcctaccctcccttgctccttcctttctttttcttcactgaaacataactcacatatagaatttacccatttaaaacatatatttaattgggatttagttttttcacagcattgtgcaactatcatgacaatctaattttagaacattttcgtcatatccccaaaagaagcaccgtgccctttagcagccactcccctttctcgtcacccacaggcaaccattaatttgctttctgtctctatgcattgaagcttcatttcttactctgtaaagtgggtgtgctgtggtggggggcaggttggatttcccccagtgacttctacagagggtgcctgctcagcgaggagataggtggaaagaggaaaggttgaggaagccaaagaattaaattgctgcgtaaaaattaaggcgtgaaatagttcaggggtgggatgagtggtctgggaagatcctagaagaaggaaaggatggaaccagtgcaaggggcagggggaagggtgagacacaatgcctccggatcaaaggagaattttggcagaaaaaccacgccatgtaatagagattgagattgaggttgcaacgtggtgtgtggtgtaaatgcctccactcctcctcataaaatcttcagaaaagtatacaggtgatgtgccgggtggagttgggtttgctttttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgagccaccgcgcccggcctgggtttgcttttgagggtggtacagaggactgagtcctccctcctggtgagcaggggctagaacagaggtagtcctgcaggcctctgcctcatctgggcacaagggcttagcactgccactcatttccacctcagagggttggtagtgagtaccgagtgatttaacgtatgagtgacggctcaccatcctgcactgggcacatggtaggtgcctgtcagccacaccggtcatcaagggccaaccaggagagcccctgtgatgccttcgtgtcttgccttcccttccagaacccaacgtcttcctcatcttcagccatggactgcagggctgcctggaggcccagggcgggcaggtcagagtcaccccggcttgcaataccagcctccctgcccagcgctggaagtgggtctcccgaaaccggctattcaacctgggtaccatgcagtgcctgggcacaggctggccaggcaccaacaccacggcctccctgggcatgtatgagtgtgaccgggaagcactgaatcttcgctggcattgtcgtacactgggtgaccagctgtccttgctcctgggggcccgcaccagcaacatatccaagcctggcacccttgagcgtggtgaccagacccgcagtggccagtggcgcatctacggcagcgaggaggacctatgtgctctgccctaccacggtgaggggccgcttgcaggcgggagggtggggtccccgatgtccaggggactggagccatgagggacagattcccagtgacaaggcctttggcctctgtggacccttggcagtcacatgtttaattataataggaagcattttttaataattaagaaaattaacttcaggccaggtgcggtggctcacgcctgtaatcccagcactttgggaggccaaggtgggtggatcacttgaggtcaggagttcaagaccagcctggccaacatggaaaaaccccatctctactaaaaattcaaaaattagccaggtgtggtggagcatgcccatcatagtcccagctacttgggaggctgaggaaggagaatcgcttgaacccaggaggcagaggttgcactgagctgagattgcgccactgcactgtgcagcctgggtgacagagcaagactctgtccccccccccacaaaaaaaaaaagaaaagaaaagaaaaaagaaaaattaacttcaaagcatttaatttacttttgtaaaaattgcaaaatacatgcttgtaaagtcttcaaatagtatacaagtatataacataaaattaaatgaaaattcccttcccccaggcccactctccaggtgaaaccccattgtagccccccatgttgtcttaatgcctgtcttgcatgtggtgaatggggaccagagtcaccatgtgaggggctggcctcttgtggtggacagccccgtcctgggggggcctttcctgggagaccccctgagttccgagatagaactttgtggggacacagagcccatgggctgggtagctgagcagagattccaccgggctcagccctctgtgggtgacttggaggccctcttgcccagctggagtttggatgggattaggggtactagggagccccttcccagccctatgaggtgtgcagcaagacctctctctggttcctgggctgactcaacagcagaagaagccttaaagccactatggggtgccattgcctctcccacctgctgatccctgtgaacacccagcactctgggttttaggggatttctcctgagggtcgaggggcttggcagcctctggtgtccagatgccaagggcctggcccctgtccaccccctgcagaggtctacaccatccagggaaactcccacggaaagccgtgcaccatccccttcaaatatgacaaccagtggttccacggctgcaccagcacgggccgcgaggatggtcacctgtggtgtgccaccacccaggactacggcaaagacgagcgctggggcttctgccccatcaagagtgagagctgttggagccgtgggggcgggggcagtgttcctggagggaggctggtgctgaggggccccggggcccagggtgagatactgccccctcccctacctagtgtagccttttggtgggggagggtctgcactcccgagggaccctggagggggcctgaaggagagggctgtcgtggtggcaggtaacgactgcgagaccttctgggacaaggaccagctgactgacagctgctaccagtttaacttccagtccacgctgtcgtggagggaggcctgggccagctgcgagcagcagggtgcggatctgctgagcatcacggagatccacgagcagacctacatcaacggtgagccggggcctgatgcctgctcgtgcctctggagggcccgggccctttccgcttgtgggttggggagagggcgatggggagcgggggaggctggggctggggatcccctgttcagtgtccctccttgctgtcaggcctcctcactgggtacagctccaccctgtggatcggcttgaatgacttggacacgagcggaggctggcagtggtcggacaactcgcccctcaagtacctcaactgggagagtggtgaggcacaaggttgggggcgcagggcagcataggggccccgcgggctcttggcctcccatggactcctctcctcatgtcctcctgcagaggggcagtgtgggtaggggaagcaccgacctccacccccctccccagactgcgcccccctagcccatgtagggcggcgctgccccaggccgaaggtcttggtgcatatggactgggtgccagcctccactcacctcttcagccacccctgtgggcagcgggcaccagcgcacccagcagcctggacggggaggccggggcggggctggtactggttactgggtagaaggttcaaggcgacgtgcacccaggttagaaagcgcggaggaccccgtggtctggggcggagctggagctgagcaccaggcttcccgaactggctcaggcttccgagggagcagagggaggtaggaggttctgagcagggccccgggagccacggtgtgagcttctctctccgggggtgctggcgccctgccctccccacagaccagccggacaaccccagtgaggagaactgtggagtgatccgcactgagtcctcgggcggctggcagaaccgtgactgcagcatcgcgctgccctatgtgtgcaagaagaagcccaacgccacggccgagcccacccctccaggtgagccagggactgtgccgcagggtggggaggggctcccagggccagggacacagccacagagccgtggcaggcccagcctctcctccggttaccaccacagcacaaggggactctggatcctctgactcttatttcattgttcagttaaagtctgagcaaattggaatatgtcattaattcatttggggaacatgtgctgagatctgtgttaaatgctggcaatacccactgttacctcgtagagaccacagtctgctggaggtgacagacatctcaatgcagttagactatcccaggtcatgagtgattatagaaatatggatcagtgttagaggagcatagagaagaaactgattcagcttctgagagttaaggaggggtttatggaaataacatttgatttgggcctttcaaattgagtctaagtttaaaagatataaagaggtgtgggggaagggcattcctggcagagggaagagcatatgcaaaggtgtggaggcctagaactacactgcagctttaaggggtggtgagtgatggccgggcaccgtggctcacacctataatcccagcactttgggaggccaaggcaggaggattgcttgagctcagtagttcaagaccagcctgggcaacatagcgagaccctgtctctgcaaaatatttttaaaaaattagctgagtgtggtgatgcacgcctgtctactagggaggtcgaggtgggaggattgcttgagcctgcaaggttgaggctgcagtgagcctcgcttgcaccactgcactccaacctgggcaacagagcgagacgagaccctgcctcaaaaaataaataaataaataaataaataaatttaaaaaaaaaaagaaaaaagaaagaaaaagaaaaaaatggatggtgcctgttgcagtttgggtggagtatagattctaaccacttcaagtatggtcatccaaccagcagcacagcatccccggagggcttgttagaaatgcacagtcaggcccgccccagagtaattgaatcagaacctgcatttaacaagatcctcagatgattcatgtgcagacacagtttgggaagccctggcttaaagggcatggtgaagaagaggcaggttgcagccagtctctgcagggtcttggctgctgggccaagtttgggtggggccaggtaggctgtggggagcctttgaagatgtttaagccagggagtgtctggttctcgtttgtgatcatagagacagctttaggggcctcgtggaggagttggggcaatggacagggtctgctttagggattctgccccttactgctgtgtggccctgggcaagcagcttagtctctggggtgcagagtcctcattgcaaatggagaatgaagccaccccccttgcaaggttgttgtgaggaaggctggagatctggcctggcacgaaggagtttttctgtagttggctgtggatgtttactgtggtgtaggttggaggggctgtgcagtttcctgcgaagctgtggggtggtgaggagacttttacaaagaaaatatggcaacacacacacacacacacacacacacacacacacacacacaacactaactacagacagtaaagagtttacagagcaaaacttctgctcctgaccatggaagtcttgccaatgatcgactcaggactggtttttttgtttgtttgtttgagacagggtctcactctgtcacccaggctggagtgtagtggtgcgatcttggctcaccacaacctctgcctcctaggctcaagagattctcgtgcctcagcctcctgagtagctgggattacaggcgggcatcactacttcccggctaacttttgtatttttagtagagacggggtttcaccatgttggccaggctggtcttgaactcctgacctcaggtgatctgcccacctcggcctcccaaagtgctgggattacaggcgtgagccaccgcaccccgcctttttatttttatttttatttatttattttttgaaacagagtctcgctctgtcgcccaggctggagtgcagtggcacaatctccactcactgcaacctccgcctcccaggttcaagcgattctcatctccaggttcaagcgattctcatgctccagcctcccgagtagctgggattataggcatgagtgtgccaccacgcccggctaatttttttgtatttttagagatggggtttcaccatgttgcccaggctggtctcgaactcctgtcttcaagtgattcacccgcctcggcctcccaaagtgctggaattacaggcgtgagccacggctctggcctgattcaggactgctgaccactgctttatctcccattccgtaaggggccaggagcagaagcttgaaacgtcagcaacccgatctagggcacctccacctctagctggagggagccttgggccctagtccagtcctcccacaagcacctttctgaaggaaggggcccgctgggaggcccgggtgtgggcatgcctagcctatctgtggccccttaccaagtcctctgtcctctgggtcttcccggacctgcagacagatgtaaggcctcggatttccgcccctctgcccagcccatatctctctgcacgaggcatattctaggcaggttctgctttcccggtctcatcacttaagtcctgtcatttcgaaggcaggcgccccgtcgtgtccccaaagcatccacatttcccaagctcccttccggtccttgaggcccctcgggtctcagcagccactaaaatccgcttgtctggggcggcagcctcaggtcttagctcattggctaacaagagatgattgacaggtcctgagccagagagcgagcccaccgggctctcagtccgcccagctttgccgggtctccaggagcaggtgtggctggaccactggaatcccagtctgaggccgtgttctagtgggcgcgcagtttcctgcccgactaggcagaatcctgcccgagcatgacactgtgggtggtttggtgaagtcctgttttctcttgccaagaccaagtaagggcttgtttgcctcagctgccctcggatcccaacccctagaaggtgaacctgaaaggcatggaaatcggggcaagggagggcctgggacgggtggatttctcagccctggggttcagggttaggagttaggagcggggaaggagtcccagctgggggatggggccgatctgtagtgactttgagcacctcctaatgttctcggttgcccgtccaccaagacctcagattcctggtgggcttgcctgggccccattagtggctcaaggtcccgtttcccagttttctccctcctgggcttcccagacctgcacagagtgggtgctcaataaatgttatttgagagaataacagtgattccatttactaggcagggggccataagccaagcttgaatctcatagcaggaaaccccatttacagagggagaaactgaggttcatggggctggtttgtcctttctacccatcgtttattgactgattgattgatgcattgattgagataaggtcttgctgtgtcgcccaggctggagtttagtggtgcaatcatggctcactgcagccttgacctccccgggtcaagtgatcctcccacctcagcctcctgagtagcgggaccacaggcgcacatcaccatgcctggctaattttttattttttatagagatggggtctcccctttgttgcccagtctgttcttgaactactgggctcaagcaatcctccagccttagccttccaaagtgctgggattacaggcctgagccaccatgcctggcccacccatcgtattctgagagccacagccagaggaagtgttctgtcctgggtggaatctgccaccacgacttgggtctctgtcccggggctccagcagccctgtaaagttttgtgtttttttagcatttccaagacctgtggtggggaccgggattgatctgggagagtttggagaggaggtgactgggaggcctygcctgtgttgacgtcaacccagtgggttggttcccagactgggcggctcagtccctgagcagcagcctcatgggtctctgcagacaggtgggccaatgtgaaggtggagtgcgagccgagctggcagcccttccagggccactgctaccgcctgcaggccgagaagcgcagctggcaggagtccaagaaggcatgtctacggggcggtggcgacctggtcagcatccacagcatggcggagctggaattcatcaccaagcagatcaagcaaggtgaggagctgcccgcccacgtgtctgggtggagggcagggcctcccactgccccacccatcctgtccaggagcctcagagaatgttccttcctccctactggtggctgaggctgacctctcaatgttttctctcccctcctctcctgctgcacccccagaggtggaggagctgtggatcggcctcaacgatttgaaactgcagatgaattttgagtggtctgacgggagccttgtgagcttcacccactggcacccctttgagcccaacaacttccgggacagtctggaggactgtgtcaccatctggggcccggtgagatctccctctccctatcacagggccacaaaatacacccccaacctccaggtgccaccccagctgaaggacatcctagttactatcagaacctgcctggaacctcttacctctcagcagtccccctcctccccaccaatgccttcccttccatgtgagagactgccggggcttgaatcctacctccacctccacctccaagtagccaagtcaccttgggcaagttactagagggtcactggggagatcagaggagttaattctcgtgaaaccttaggcacagtgcctggcgcattcttggtctcctcccagctgcccttccctgccagaaggtggcatcagaacccacattatgtgcagatactttgcaagcagggctgcactctgccagccctcgcccccagtgctctacccagtccagggtcctgtcatctcaagagattgttgggttttatgacaatttgccatggtttgggcttagatgtggtgtaagtgaaggaattgatggagggcttcaagcagagatgaaagacccttaaaaaagggacaaagccagctgagtgtggtggctcatgcctataatcccagcacattgggaggctgaggcaggcggatcacttgaggtgaggagttcgagaccagcctggccaacatggtgaaaccccatctctactaaaaatacaaaaattagccaggcgtggtggcagatgcctgtaatcccagctactccagaggctgaggcaagagaatcgcttgaacctggaaggcagaggctgcagtgagctgagattgcaccactgcactccagcctgggtgacagagcaagaccctgtctcaaaaaaaaaaaaataaaataaaaaggagaaagccgtagggacacagaccacccttcctagggacagacaggatgttctgctgtcacatactgcaaacagtgctgtggactgtagtggcagtcatttgccttaggggatgctatttaagaattttaaaacaattatgtattgagtatctaccatgtgccagtcatttttctgggtgctaggatacacaaacacgcctgccctcagaatggagcttagagacagacatactgctggctgggggcctgtggatactggggagggcgaagacagaagcaggggccgaggactaaattcctcctcagcttcatcagctgagattctggtctgggcacactggcctctgcacactctggccctgcaggcactagctagacgccttgggaatccaagctgtctatcctgccatcagttactctaacaatcttagccagtttgtttttgatatttataaaacgagttatcaataatgtaaaaggacgcctttcctttttttttttttttttttgagatggagtttcaccctgtcgcccaggctggagtgcaatggcacgatcttggctcactggaacctctgcctcccaggttcaagcgattcttctgcctgagcctcccaaatagctgggattacaggcatgcaccaccacaaccggctaactttttgtattttcagtagagatggggtttcaccatgttggccaggctggtcttgaactcttgacctcaagtgatccgcccacctcggcctcccaaaatgctgggattacaggcatgagccaccgcgccgggccaggatgcctttcccatttagacaatgcttgttaagtttcaaggcgtttgcacatgaactgcttgtgctctcatccaccctgtgaggcttatgggttcatcccgccttccccattgcacagctgggacacagacatgtcagaatcacacagtaagtcagagacagggccagctctaggaaccagattccaagaaggatttgggagatttatggggaggtgggggttgagattcttcctcaccctgtgtccgtaggaaggccgctggaacgacagtccctgtaaccagtccttgccatccatctgcaagaaggcaggccagctgagccagggggccgccgaggaggaccatggctgccggaaggtgagggtgtttctggagctgccctgagtggggccacctgtcagaggggctaccaggggagggagaggtggatgaactcctgctgcctcgcatggcatcgccctctcgtcggcacccccttccctgagctctccaaactgctcctttattgaaggagtgatactggatgggatgggacgggtgggcgctgttgggaggaagcagagctccccacccccagcccagtcctgagtccctccccatgggggaagccacttcctcgagacagcagagaacagggaggtgggggctggtctacccccgagtgccccctcagagtccagcctagtcatgtcaagcagtggctgccgaagtgaagtgtggcccctgtgcaccgccagtcctgacttccccctcctctgccgtctgacacagggcgtgtgctggtctgtctcccctggaggagtgaggccactcagggtggggctgcaccttccccctccttgtgttcccagccccagtgggcttctagaggatgtttagggaggttgagggggggggtgtcaaggaatgaaagctgtgtaagtgagtggggggtgagtgactgagggtgtgagggggtgacagaccctctcctcctccagggagtggagcaggggtgcaaatgctgtgatgggtgcggggagcttgcttctaaaagacactgcttggcccttggccccccccagcagccccttgcagccaggctggctcctcactctgttttcctttggattaagcccctaccacacccacctggaccccaaagcctcccccagaaaatgagcaggaaacactaacaggcatggccccatgggcgaggaagcagaggggagagcagagcccagctgcggagtctgtggactggcaaggacagtgcctggcccaggggctgtgtcatggctgcgaagaggtgaagggaggagtagagaggaattaccaacttctctggcaccaaggccagcctggaatttgtgtggggagtgttgagggtccatggttaacagcccaggatcaccaggggtcttggcacagcccctgccagcagatgaggcccccaccacacataccacatctgcttctcttctgccttggctcctgccagcctcgcctgccagggtctcgtctctgagcccctcctgccatccccgggccaggagcatcctgcgccgactccagctttgccaggccctcgaggactgcccatcccctctccatcctggcagctctccccagcctttagctgtggtttcccagccaacagtaatttccccactgtgtctcttggtttaaatgcccaagagaggagcctgaagggccattcatcttcccgagccgggtcactggccggtcgctggtggcctgccaatcagccacgctggggctgggcctccaccccagcttccacagctgtgggtgatggtggagggggaatgtggagaaacgaggtgcagaacacagcccagtactcaaaccagtcccctccgtcctgagcacgttcagtgatgtccctgatggcatctcccaccacaggatttatgggtgagggtggagagtcatcttccctacagacacccctcttctgtccactcttgagccagggttggacgtggcacagcccatcctgctactggctgggagaagaccaagtgacctacagtgaggcccggcgcctgtgcactgaccatggctctcagctggtcaccatcaccaacaggtacagcaggggcgggtgccctgactagcccttgcccatgtctgggcctattgtggtcccttcagcaaacagggtagcatctgccatcatgcccagagggacctggagtcctgtgaacctcagtgggctccagggcattttaaaatcttgtcagaagaatgactctcacttgttaagtgaccccagtgtagggcagggcccgggcaggcacaggtgtgggctggatacttgagaaagagaggccttggttgtgtctgattttctctactttcttctcagcaaagcaaggggtgcagagaatagaaggggcagctgggggttccataaatcagctgcagggctcccaggcgtccctgctttcttgagcaagttattggtcgggtgcagcacccgagctcccacggtagggcgtctggcctgtgactgggggattgggaagcagggagatccctgccctgaccagcctgtctcctgaaggttcgagcaggccttcgtcagcagcctcatctacaactgggagggcgagtacttctggacggccctgcaggacctcaacagcaccggctccttcttctggctcagtggggatgaagtcatgtacacccactggaaccgggaccagcccggtgagccccttatttgacttgccttggtgaagcgaggagggaggccagggtggactggccctgccagcaccgagccccagggcttccccataaacagatcattggtgcactgtggtgtagtgtctatgagcacaagctctgaaaccaaactgcctggctccgatcatgactctgccatcttttagctgtgcatcctctgagcctcagttttctcatctgtccagtgggaataatgatcctttcctcatagggatgacatgagtcttaaatatgtgtaaagtgctaatatgtgtaaagagttaatgtgtgtaaagtgtctagattacttctctatctgggctcacaaaaaagaaacaaataatctcttctctatctggactcatttaaaaaatgtgttcagaatactgagcgcaatacaagtgcttgttattactattattaccatttcacattcattatctttctaaccccttcaataacctgatgaggtagatgatgaagcaggctcggtgaggttaagcagcttgcctgagccctcacagtgggtgaacccagagccagggctctgcctccattcccttcctccctcctggctccaaccccagagagcatgtctctgaagaggtggccaagctggttcccagtgtgagggccgtgctagtgccctgccgggatgtgagggaggaccagactatgaatccttctcagagcctgggtctcccttccctctccttcagggtacagccgtgggggctgcgtggcgctggccactggcagcgccatggggctgtgggaggtgaagaactgtacctcgttccgggcccgctacatctgccggcagagcctgggcactccagtgacgccggagctgccggggccagatcccacgcccagcctcactggctcctgtccccagggctgggcctcggacaccaaactccggtattgctataaggtagggcagcctgttggccgggtagggggcagggggaggacaggagcaaagagacagtttggatggaggtcccagtgggaccagccacaatccagagacacaccaggctgcagacgggtgatttctggtgagacttgtccatggaatctggaggcctggtgctcagtaatcatttgaggaattgagggaaccacagatcattgatttcagaggttcccaaatgttggggccagcctagctgttcttcagaattgcttggggaacctgataaaaataaaatcttggccgggcgcagtggctcacacctgtaatcccagcactttgggaggtcaaggcgggtggatcacgaggtcaggagatcgagaccatcctggctaacatggtgaaaccccatctctactaaaatacaaaaaattagccaggtgtggtggcaggcgcctgttgtcccagctactcgggaggctgaggcaggagaatggcatgaacccaggaggcggagcttgcagtgagccgagatcgtgccactgcacgccagcctgggtgacagagcgagactccgtctcaaaaataaataaataaataaataaagataaaaatctaggctgtcacaatggagatttagattcattaggtctggattgggcctggtaatctgtatttttaatcacgctaggtttgggagccactggcagagtccgaggtcttttattatgcacagaaactgaagtctagggagaagaagggacttacccacgttaagctgtggctctgctgggattagagcccagatctccctgctggtcaggggatggggctcaggggccctctccagaaggtgactgtgccagctcccctccccagacctctgcagatgaacaggattgtccttagctagccttagccccctgtccagtaaggctccaggcccctcctctgccatggtgggaaaggcagtaggtgccccctgaggggtgggccagtggggacagcttagacagctggactctgcccctgcaggtgttcagctcagagcggctgcaggacaagaagagctgggtccaggcccagggggcctgccaggagctgggggcccagctgctgagcctggccagctacgaggaggagcactttgtggccaacatgctcaacaagatcttcgggtactgagccgggctgaggcgtgttaggagggcacccgcacacgtgtaggagcaggcatggccgacagacccttggttttgttgggcgagcagggggatggcagaatgtgggtgaccccagggaggcctgcatctgctgctggtgccaggaccatcttgtcctcactcaagtagccatggtgccctggcatcttgggagaacaggctggtgtgatccatgcactcataggagactgtgttttgctcaccatcatccccccagcaatgctccaacagacccacaaacttacatatgtaccccccaccccattgcaaaatctcccacatgctggtcctggcaagtgtttcccctcaccttacaccatcctacaatgacccacactcctcaatccacagtttttcctgagagataggaggagaaaggccgcagctatgaggatttgcccatgggcctccctctgagacaccacctctgtcctgggctctgagcattagcacgtggaatcccctagcactgggaaaatcgagatggatctgggcttcctgatagcttaattgaagggatctctcctctcagcaaacattgaaattacattatttgtctcataattacaatagctcatggtcatcacttctcaccacgtaccaggcactgtgctaaacatatagtcctcactcaagcctcccgacaaccctatggagcagacgttaacccctgacccttgtgggttgatagagaggttatttgcccaagttcatgggtgaacggatacttgaatccaggtttatttaactccagagccgtagatccctgccacctcctcatctgaacttcctaaactccgtaaagtaaaagcctgcagaatcttgctggacagggcacccccacccaaaaggaacccaaacaaaggggaggggccactgccccttgagtggatggcttggtccagctctccccagctctgtcttgtggggagaactttaccgaagtgacctcaacgctgtatcgatgggtcaatgtgaagcccagatggagaggccccaggccctgggggctgccccggtcacagctgcaagggacagggggcacgtttgggttcctgcccctctcacttcccatctcttccgtccccactcctgggttgtgctgaagtgaatcagaacccgagatccacgagcagcactggttctggatcggcctgaaccgtcgggatcccagagggggtcagagttggcgctggagcgacggcgtaggggtgagggggcctggggtacttgggactgcgaggccgggagcttggtgggcggggcctgtttggggcggggcctggaggtgggcgggttttcttgagggccgggcccccagtcggagccggcttcaggaaagcaggtccgagaggggtcacccggcccccggtgagaattcgcagctcaggccaggcctcttgttcacctgttccgggcatgggggcggcctgcaccttgcgcctcacgttcctcttccctccacccgcctcctccagttctcttaccacaatttcgaccggagccggcacgacgacgacgacatccgaggctgtgcggtgctggacctggcctccctgcagtgggtggccatgcagtgcgacacacagctggactggatctgcaagatccccagaggttggccggagtggcgctgggggacgcgggatggagcgaagggtggcggggccaggaattccagggagggtctcctttcctcacaacgtctttgtccttgttcccctaggtacggacgtgcgggagcccgacgacagccctcaaggtgagcaccccccagcccatcccgctacccatcgcgtgggagagggcgtcaactctggggtaagtcccgagaggcctgaatgaaatggaggggatgaggagttccggtcgagagaagggggacggggttggctcggacggaggcagccacagccctgtttgcttccgtgtgggaggcgaggcaagcctggggcctggggcctggcacggtgcctgcctgggtccggtgtgcctgcaggctcgcctgctgccgcctggctctgccccggccctgccgcgcccgcctccggggcctggcgtgcagcctctgcctggccgccgctcccacgcccgcgctgctcctgcaggccgacgggaatggctgcgcttccaggaggccgagtacaagttctttgagcaccactccacgtgggcgcaggcgcagcgcatctgcacgtggttccaggccgagctgacctcggtgcacagccaggcggagctagacttcctgagccacaacttgcagaaggtgggcattggattgagcagggggctgcaggctgggggagggcaggcccgggatgagggcagggggctgcctacccacacctgcccgcctggcttctccagttctcccgggcccaggagcagcactggtggatcggcctgcacacctctgagagcgatgggcgcttcaggtaggaacccaggcaggcagcagatgtggaagggggctggctgtccacacacggagcacagaggcagacttgcatttgaatccagccctgccccttccttgctggctgtgtggccttgagcaagatacttaacctttctgggccttggttttctcatctgaaaatgggaatatgaggcctgcctggtaagatttttgtgaagattagaaataacatatgcgaagtgcttacgcagcatccagcacattagttactaaataatggcagctcctgttacgtctggtgtcattcatcatgactaaggccctctcggggaagtgggcagaacatatgatgatccgggggcctctttgtatttggtgcctacttaccatgtactggggggcttgaggcaggaaggaccaggagcacctgggactcagaatttgagcttgtctttttgcccttgggcttcctgccctggagtcctcaggcttcttttctggagcagcaagtgtccagggacagctggggccttatctagaggcaccaggtccttgggtctttgactgccctgagctcagtagctccacccctcagcctacagccactaaaccccagaacctgggcccttccctgcccccattcctgcggccttcattgctgcatccggtggaggcccagctgggggccggtgctggagttacctctgctccatgccattgcagatggacagatggttccattataaacttcatctcctgggcaccaggcaaacctcggcctgtcggcaaggacaagaagtgcgtgtacatgacagccagccgaggtgagggcaaggtgggggcagagtgcgcagtcagctgtgcaaagggttaatgctgggcgagcctgggcagaagtcatcagtctgttctcattctctttgttcccacaacccaataaaggctagactcagcgaggggtggacctgtctccatggtccagaagacttgaattcggagctggacacaggccctgagcctctgcccagactcctctcagggcatgaggctgagtgtgctgccagccatgccctgccctgctctgccctgggggtgactgccctcccctcccctttccgcagaggactggggggaccagaggtgcctgacagccttgccctacatctgcaagcgcagcaacgtcaccaaagaaacgcagcccccagacctgccaactacagccctggggggctgcccctctgactggatccagttcctcaacaaggtaggaggtggcaatggggcacccaagggagtcaggggagaggacgtgaacagggaaaggctgagcctcaggagtcctggccttttggcagcaccaaactcatggcctcttgcctgtacccaccaacttctctttcacctggctccagtaccagggccagactccttggaaaatgactcactgtagtagcaccttccaatcctccgggaagcaaggttgtccaaataatttggtgagtgttctagatcagtgattgccaaagattttttttttttttttttgagaaggagtttcactcttgtcacccaggctggagtgcagtggcgcgatctcggctcactgcaacctctgcttcctgggttcaaccaattctcttgcctcagcctcccgagtagctgggattacaggcacctgccaccacccctggctaatttttgtatttttagtagagatggggtttcaccatgttggccaggctagtctcgaacccctgaactcaggtgatcacctgccttggcctcccaaagtgctgggattacagatgtgagccaccgcgcccggcctgattgccggcctgattatcaaagattttgataatgaactcctattagtaaaacaaaatattgagcttacatgaccaaactatgtgtgtgtacatttaacttatatatgtatatgtgtgtgtaaataaaacctacttccatactaatatgactaatattatatatagtaaaatatgtataagaacaaaagagtataaaacattagctaaagatttttaaatacttttaagtttaaaacatttttttggctgcgcgtggtgggtcatgcctgtaatcccagccctttgggaggctgaggcgggtgaatcacctgagattaggagttcgagaccagcctgaccaacatggagaaaccccatctctactaaaaatacaaaattagctgagtgtggtggcacatgcctgtaatcccagctactcgggaggatgaggcaggagaattgcttgaatcctggaggcggaagttgcggtgagtcaagatcgtgccattgcactccagcctgggcaacaaaagcgaaactccgtctcaaaaaaataaacaaagaaaaaaaaaacaacaaaaaaaacaccttttttttttttttttttttttactagtggtatgaaaacatttttttgtgctcactcaaaagtgattaattggctgggagaggtggctcacacctgtaatcccagcactttgggaggctgaggcaggaggatgacttgagcccaggagttcgagaccagtctggacaacatggagagaccatgtctctacaaaaaataggaaaattaaccaggtgtgatggtgcacacttgtagttccagctacccaggaggtgggaagtgggaggatctcctgaacccaggaggtagaggcttcaatgagccatggtcatgccactgtataccaccctgggtaatggagtgagaccctttctccaaaaaaaaaaaagatcaattataataattattgtttaatatgctcactttggcagcacatgtacaaaaagtgaaacaatacagagaagattagcatggccctgcacaaggatgctatgcaaattcctgaagggtccatattttgcgtagttcatgaaagttgaaggttgttcaacaaaaaaattgtctaaataattttaataagagcaatgtgattacatgtacttcacgtttttaaaaaattttagtctgacactttatgactcatgactcacaggtctaacgtgttgaaaatcaaagttatttcaaaacatgcatttcatggtggctatagattaaatatgcctcacaaagacaaatagattgaagtgaaaaaagttcatccatcatttctaaataattctccccatttttagtccttctaccaattaaacaaacatttttgtttaaaaattggctagtaggctgggacgctgagcactgggaggatcacttgaagccaggagttcgagaccagcctgggcaatgtagcaagaccctatctagaaaattctttttccaagtgttttgagtgtgcaggtaggaaagtttggggcactgacattaatcactatggatgcatttctcaacacgtttttacaacaccccctccatagtgtgagtttcttccccacagtcgttatttttccatgcacaggctgatttattagacctgattagtccttcattgtttttacctggcgattgacaacgagttcataccaggaagagaaggttgtcattttccttgttatgtgccagggtaattggcattaatctcaagccatggcttctttgcaggacattttaagaacacttagccctgttgtgaagattagattagttaacactgaataatataatccacaaatccttgtcatacacaggaaggcagccagcctcttagtgtcatggatctcctgccctcctcagaatattcaaaagaacattacacggccaggcgctgtggctcatgcctgtaatcccagcactttgggaggccgaggcgggtggatcacctgagatcaggagtttgagaccaaccagggcaacatcgcaaaaccctgtctctactaaaaatacaaaaaattatctgggcatggtggtggacgcctgtaatctcacctactcgggaggctgaggcagcagaatcgcttgaacctgggaggcagaggttgcagtgagctgagatcgagccactgcactccagcctgggcgagagattgagactctctctcaaaacaaaacaaaacaaaaaccacacaaaaaaaaaattacacctcttctaccttaaaaaattaaggcatgtatattattaaaactttggaaaatacagaagagaaaccaaaactcacccaacactctctagagatcacagctatcaatatttgttattctccttcctgcctttttccatggaattgagctcatactaaataatataatttcctacccaactcttctttctctgtatcatattttgagcatctctcatgttataaagtttttccaaaaacttgttctttttccatccactttttaaacagaatgtattttatatatgtaataggtcccattcttaatcatcattgtcaagataccagttactctcctgccctgtggaaagttctgtttgtttttgtttttgagacagggtcttgctctgtctcaaaaactgctggagtatggtggtaccatcatggcttacttactgcagcctcgacctcctaggcttaagtgatcctcctgcctcagcctcctgagtagctggcactacaggcttatgccaccatgcccagctaatttttctattttttttgtagaaatggggttttgccatgttgtccaggctggttttgaacttctgggctcaagcgatcctcctgccttggccttccaaagggcagggattacaggtgtgcgccactgtgcctggcctgtggaaagctctggaagtatgctagggtgttcactccttcactgcctgccctcacacatcttgctttactagttctgtgcccttctttgtgtccctatagcatttggatgtaggtaaagcatttatggcagctgtccccaacctttttggcaccagggactggtttcttggaagacaatttttccatgtactaggggaggggagggatggttttgggatgaaactgttccacctcagatcatcaggcattagattctcataaggagcacgcaacctagatcccttgcatgtgcaattcacggtagggttcgcactcctatgagaatcttggctgggcgcggtggctcagcctataatcccagcactttgggaggcctaggtgggcagattacctgaggtcaggagtttgaaaccagcctggccaacatggtgaaaccctgtctctactaaaaatacaaaaattagccaggtgtggtggtgggcgcctgtaatcccagctactcgggaggctgaggcaggagaatagcttgaacctgggaggcggaggttgcagtgagctgagattgcgccattgcactccagcctgggcaacaaagcaagactccatctcaacaacaacaacaacaacaacaacaacaacaacaaagaatctaatgccctgctgatccaacaggaggtggagctcaggctgtaatgccccttcgccccgctgctcacctcctaagaggccggggacccctggtttgtgggacagcctcttccactagctttaagctcctggagggcagggccactagcacctgccataccccttaatacacttgcacatggcagggatgccctcagtgtcggctggactgagctgaataccctccccctttagctgtcacttcttgctggtactggtgtgaccttgctgcttccaagtttatgatgaattctactcatgatttaagttgggcctcagcatggctctgaaactacttctgtgaaaccaaatccttgaactttgtgacagacctccaaaaatctgcattatttctgggcttacaattttttaccccaaatctgccatctgcatcagtaaggatggtgctggagctaattattaatagtcacagctctcactggttaagtgcctactgtgtcatgtactgttatctttaatccttaaaatgatccccaaagacaggctttgtattaccactttacagaagagtaaattgaggccataaggttgtcagagctggaatctgacatctcccccttacctgaatcccacccctgtctttatgactccaaagcttccctacaccctttctttttttttcttttgagacagagtttcgctcttgttgcccaggctggagtgtaatggcgtgatctcagctcactgcaacctccgcctcctgggttcaagcgattctcctgcctcagcctcccaagtagctgggattacaggcatgtgccaccacgcctggctaattttatatttttagtagagatggggtttctccatgttggtcaggctggtctcgaactccagacctcaggtgatccacccgccttggcctcccaaagtgctgggattacaggtgtgagccaccgtgcctggccaacaccctttattttccatcacactgcctctgagcagaaaatcattagatattcattttcctaagaaagagtcttcacttgatgagagatttcctacccagggcttttgtttgtttgtttgtttggaaaccttatagagctaatgaaagtgggaaaagagaaatgtaaaattacagagttagaagctgctgctaatagatcattcaacaatagagataaaagtgtcctagggctgggcgcggtgaaaccccgtctctactaaaaatacaaaaaattagctgggcatggtggtgggcgcctgcagtcccagctactggggaggctgaggcaggagaatggtgtgaaccctggaggtggagcttgcagtgagccgagatggcgccactgcactccagcctgggagacagagcgagactctgtcttgaaaaaaaaaaaagtgtcctagagatgatcatgtaagttcaaagggcaagataatggatgtcagacaacattcggaagggtgtttagcagaaagcaagatagatccttgtccccatcttcctagtgcttggcctaggcactgttgtcatagcctttgacggcccaaagaaatcctcccatttgaagatcccctggagtgtgggcaggtgtgtgtgaatctgcagtgccaagctgggaggtgctggaggctgccaagcagacccagctgtcctggttctcagatactgttctctcccggccctcaaaggccccccaggacctgctgctgaggggcgcacagtggcctttctgggtttgtggatggggtggtggggctggccattacatccccacctctgccccacagtgttttcaggtccagggccaggaaccccagagccgggtgaagtggtcagaggcacagttctcctgtgaacagcaagaggcccagctggtcaccatcacaaaccccttagagcaaggtagggccagcctatggggagcccccagtatcctctgacactgtccccccaaatagctatagcagagtcactcacaactgtcttctggggaccatagcattcatcacagccagcctgcccaatgtgacctttgacctttggattggcctccatgcctcgcagagggacttccagtgggtggagcaggagcctttgatgtatgccaactgggcacctggggagccctctggccctagccctgctcccagtggcaacaaaccggtgaggatgccctccctgttcccctccgcaccgcgctgccctaagcctgtggggctgcctttgccccagcatctcttgccccagggtctcccttcttccagcctctttgctcacattgcagccttctggggacagtagcacagggaggcagctgctggggctcccctgagcagctccctccccccagaccagctgtgcagtggtcctgcacagcccctcagcccacttcactggccgctgggacgatcggagctgcacggaggagacccatggcttcatctgccagaagggcacgggtatgtgtcaccagtcacctgggaaaccccagtggggccctggcaccgggctggatgctgggaccatgccaggaggaaggaatcatggtccctggcagagcagggctctgcttgggaagcagatgggcagaaaggcaaggtgccaataagatggaaaagcaatagcacccacaggtctcaggaggaagatttgggggtatggagggctcgttgggtcggggggagagctagacgctgggctgcatgaagtgctggcacgtgtggcgtcaaggaccctgaggttccaggtccaagacacctccagctccatgcgtaggtggcctctgcgccgggttccttgggtaggaggaggtcaaggccctcacctccctgtccatttgttagcaaacatgtattattaataacaacagggctgacacctaccaagccttggtctgggccaagcgccgagtctggtggagaccgggtctttgcctggtgtgcggccttctcctggccttgtgcctgggaacggggtgagggaagcagagcccagcctgaaccctgaccccttccctgtagacccctccctgagcccgtccccagcagcgctgccccccgccccgggcactgagctctcctacctcaacggcaccttccggctgcttcagaagccgctgcgctggcacgatgccctcctgctgtgtgagagccgcaatgccagcctggcctacgtgcccgacccctacacccaggccttcctcacgcaggctgcccgagggctgcgcacgccgctctggattgggctggctggcgaggaggtgggctcccgacacttttgccctgggccccagccttgcccgggttcccctccctgccccttcctgcccccgccttatccgcaccctatcctgactattcccttcctcccaccccatggccccccatgcccagggctctcggcggtactcctgggtctcagaggagccgctgaactacgtgggctggcaggacggggagccgcagcagccggggggctgtacctacgtagatgtggacggggcctggcgcaccaccagctgtgacaccaagctgcagggggctgtgtgtggggttagcagtggtgagtgcccacctgccagggcgggggcatgggcaagctgtcggtggccccggggagggtgctgagccacttcttaatcctgtacccccacagggccccctcctccccgaagaataagctaccatggcagctgtccccagggactggcagactccgcgtggattcccttccgggagcactgctattctttccacatggagctgctgctgggccacaaggaggcgcgacagcgctgccagagaggtgggtgaccaggccagagcccacacatggcgggcaggtggcacctcctctcgtgggcactcagacccatgagtacatccccacactgctctctacccataggcagcacttacacaagatgccctcgtgctctcacatgtggagaccatacatgacactatatgtgcatatgctgcccccccacacacttgcacatgtgcacacacacacacatgaatccacagactccacaccatcctctactcaggctgcagagaagagggctggagcagctctgggggactctgccccccccggagacccatccgccctgacgtgggccttctttgcatccagcgggtggggccgtcctgtctatcctggatgagatggagaatgtgtttgtctgggagcacctgcagagctatgagggccagagtcggggcgcctggctgggcatgaacttcaaccccaaaggtgggtgccctgtgtgtggggtggagaggtcaagcctcaggctgtaaggggctgccctccactttgccctgagccttgtcctggggcttgtcgggggacagggagtcggttctagaacacacctgctctcctccacctactcctgccccaccttgtccctgctccctcagtgccttctttcctgcaggaggcactctggtctggcaggacaacacagctgtgaactactccaactgggggcccccgggcttgggccccagcatgctgagccacaacagctgctactggattcagagcaacagcgggctatggcgccccggcgcttgcaccaacatcaccatgggtgtcgtctgcaagcttcctcgtggtgagcgccgggcaggcccacgctcagcctccttccaccccgtgccgctggtcctgggctggggctgggggctgcttcacaactgcagggggcacagccgatgggaaggcctgaacagaacggactgcgggcagctgggacccccgggataaattagtcagtgggatggactggacctgaaacttatcatccaaactagaccacaaggctcgggggtcagcaaccccctgtcactttgtagtggtcaagggcatggacagagttcaagtcctggccatgtcacttatcagtggtgtgaccttgggcagagtctgtggcctcagtttcctcgtatgtaaaatgggaataatagtagttccagactgatgtggtggctcatgcctgtaatcccagcactttgggaggctgaggtgggcggatcacttgaggtcaggggttcaagaccagcctggccaacatggtgaaaccccgtctctaccaaaaagtacaaaaattagctgggcgtggtagcacatgcctgtagtcccagctacttaggaggctgaggcatgtgaattgcttcaaccgtggaggtggaggctgcagtgagccgagatcgcaccactgcattccagcctgggcaacacagcaagaccctgtctccaaaaaaaaaaaaaaaaaaaaaaagtcccgacctcctagagtgctagtgaagctagtaaagatgaggtgagttagggaaggggctgacacccaggcctcccacgagggctgggctgagacctagctgagtgcagcatggcgttttccctttctgggggaacctgaagtcttgtctcttgggcctgtgcccccacagtaaccagaggtaggatgggagggggaactagagcctctttctccccagacctcccggcccaggcctgtgtgctttgtatgtttacttaagtgattattacgatgatcactgctattattaactggccccctcctcttgcccacagctgagcagagcagcttctccccatcaggtgagtgaaaggcaatgcccccaggtgggcaggcaggaagcactgctgggcctaacgcccacttggcctttcacgcccactcgccttggcagcgcttccagagaacccagcggccctggtggtggtgctgatggcggtgctgctgctcctggccttgctgaccgcagccctcatcctttaccggaggcgccagagcatcgagcgcggggcctttgagggtgcccgctacagccgcagcagctccagccccaccgaggccactgagaagaacatcctggtgtcagacatggaaatgaatgagcaacaagaatagagccaggcgcgtgggcagggccagggcgggaggagctggggagctggggccctgggtcagtctggccccccaccagctgcctgtccagttggcctatggaagggtgcccttgggagtcgctgttgggagccggagctgggcagagcctgggctggtggggtgccaccctcccacaagggctgggctgagacccagctgagtgcagcgtggcgtttccctttctgggggggcctgaggtcttgtcacctggtcctgtgcccccacaggaaccagaggtaggatgggagggggaacgagagcctctttctccccagagcccccggcccaggcctgttgatccgcgccccaggacccccttctttgcagagcccgaggagcctcccctgtcccctcgggcagatctgttgtgtctctcttcccacctggcagcctcagctctgtgcccctcaccctgctccctctcgccccttctctcccaccccttccttctgagccgggccctggggattggggagccctcttgttcctgatgagggtcagctgagggggctgagcatccatcactcctgtgcctgctggggtggctgtggggcgtggcaggaggggcctaggtgggttgggcctgagaaccagggcacgggtgtggtgtctgctgggctggagataagactggggagagacaccccaacctcccagggtgggagctgggccgggctgggatgtcatctcctgccgggcgggggagggctctgcccctggaagagtcccctgtggggaccaaaataagttccctaacatctccagctcctggctctggtttggagcaaggggaagggttgccagagtcctgggggccccagaggagcaggagtctgggagggcccagagttcaccctctagtggatccaggaggagcagcacccgagccctggagtggcccagtacccttccaagaggccacagtcccagccaggacaaagtatgcggcccatcctggtgcgacagcgtgggacaatgtgaacatggactcgaagacatggccctttctctgtagttgattttttaaatgtgccattattgtttttaaaaaaaaaggaaaaaagaaaagcaaacaaataaaacacctttaagaggcttgaaagag

SEQ ID NO: 4 - DNA, 2772 bp, Homo sapiens
atggagagggggctgccgctcctctgcgccgtgctcgccctcgtcctcgccccggccggcgcttttcgcaacgatgaatgtggcgatactataaaaattgaaagccccgggtaccttacatctcctggttatcctcattcttatcacccaagtgaaaaatgcgaatggctgattcaggctccggacccataccagagaattatgatcaacttcaaccctcacttcgatttggaggacagagactgcaagtatgactacgtggaagtcttcgatggagaaaatgaaaatggacattttaggggaaagttctgtggaaagatagcccctcctcctgttgtgtcttcagggccatttctttttatcaaatttgtctctgactacgaaacacatggtgcaggattttccatacgttatgaaattttcaagagaggtcctgaatgttcccagaactacacaacacctagtggagtgataaagtcccccggattccctgaaaaatatcccaacagccttgaatgcacttatattgtctttgcgccaaagatgtcagagattatcctggaatttgaaagctttgacctggagcctgactcaaatcctccaggggggatgttctgtcgctacgaccggctagaaatctgggatggattccctgatgttggccctcacattgggcgttactgtggacagaaaacaccaggtcgaatccgatcctcatcgggcattctctccatggttttttacaccgacagcgcgatagcaaaagaaggtttctcagcaaactacagtgtcttgcagagcagtgtctcagaagatttcaaatgtatggaagctctgggcatggaatcaggagaaattcattctgaccagatcacagcttcttcccagtatagcaccaactggtctgcagagcgctcccgcctgaactaccctgagaatgggtggactcccggagaggattcctaccgagagtggatacaggtagacttgggccttctgcgctttgtcacggctgtcgggacacagggcgccatttcaaaagaaaccaagaagaaatattatgtcaagacttacaagatcgacgttagctccaacggggaagactggatcaccataaaagaaggaaacaaacctgttctctttcagggaaacaccaaccccacagatgttgtggttgcagtattccccaaaccactgataactcgatttgtccgaatcaagcctgcaacttgggaaactggcatatctatgagatttgaagtatacggttgcaagataacagattatccttgctctggaatgttgggtatggtgtctggacttatttctgactcccagatcacatcatccaaccaaggagacagaaactggatgcctgaaaacatccgcctggtaaccagtcgctctggctgggcacttccacccgcacctcattcctacatcaatgagtggctccaaatagacctgggggaggagaagatcgtgaggggcatcatcattcagggtgggaagcaccgagagaacaaggtgttcatgaggaagttcaagatcgggtacagcaacaacggctcggactggaagatgatcatggatgacagcaaacgcaaggcgaagtcttttgagggcaacaacaactatgatacacctgagctgcggacttttccagctctctccacgcgattcatcaggatctaccccgagagagccactcatggcggactggggctcagaatggagctgctgggctgtgaagtggaagcccctacagctggaccgaccactcccaacgggaacttggtggatgaatgtgatgacgaccaggccaactgccacagtggaacaggtgatgacttccagctcacaggtggcaccactgtgctggccacagaaaagcccacggtcatagacagcaccatacaatcagagtttccaacatatggttttaactgtgaatttggctggggctctcacaagaccttctgccactgggaacatgacaatcacgtgcagctcaagtggagtgtgttgaccagcaagacgggacccattcaggatcacacaggagatggcaacttcatctattcccaagctgacgaaaatcagaagggcaaagtggctcgcctggtgagccctgtggtttattcccagaactctgcccactgcatgaccttctggtatcacatgtctgggtcccacgtcggcacactcagggtcaaactgcgctaccagaagccagaggagtacgatcagctggtctggatggccattggacaccaaggtgaccactggaaggaagggcgtgtcttgctccacaagtctctgaaactttatcaggtgattttcgagggcgaaatcggaaaaggaaaccttggtgggattgctgtggatgacattagtattaataaccacatttcacaagaagattgtgcaaaaccagcagacctggataaaaagaacccagaaattaaaattgatgaaacagggagcacgccaggatacgaaggtgaaggagaaggtgacaagaacatctccaggaagccaggcaatgtgttgaagaccttagaacccatcctcatcaccatcatagccatgagcgccctgggggtcctcctgggggctgtctgtggggtcgtgctgtactgtgcctgttggcataatgggatgtcagaaagaaacttgtctgccctggagaactataactttgaacttgtggatggtgtgaagttgaaaaaagacaaactgaatacacagagtacttattcggaggcatga

SEQ ID NO: 5 - DNA, 5065 bp, Homo sapiens
gccttagaaaagttaacgagaaccagatgtggtggccactgccgaactttctcagagccggtgattggtccccagccgagggcctcagccaattagcttgctgggtgggcctggagtcccgccccgcccaggcgcccgcggagatccaggttcgaggctggcgcggcgcggagagtgggctggaggccggggcgggacgcgttgtgcagcgggtaagcgcacggccgagcgagcatggagggggaccgggtggccgggcggccggtgctgtcgtcgttaccagtgctactgctgctgcagttgctaatgttgcgggccgcggcgctgcacccagacgagctcttcccacacggggagtcgtggggggaccagctcctgcaggaaggcgacgacgaaagctcagccgtggtgaagctggcgaatcccctgcacttctacgaagcccgattcagcaacctctacgtgggcaccaacggcatcatctccactcaggacttccccagggaaacgcagtatgtggactatgatttccccaccgacttcccggccatcgccccttttctggcggacatcgacacgagccacggcagaggccgagtcctgtaccgagaggacacctcccccgcagtgctgggcctggccgcccgctatgtgcgcgctggcttcccgcgctctgcgcgctttacccccacccacgccttcctggccacctgggagcaggtaggcgcttacgaggaggtcaagcgcggggcgctgccctcgggagagctgaacactttccaggcagttttggcatctgatgggtctgatagctacgccctctttctttatcctgccaacggcctgcagttccttggaacccgccccaaagagtcttacaatgtccagcttcagcttccagctcgggtgggcttctgccgaggggaggctgatgatctgaagtcagaaggaccatatttcagcttgactagcactgaacagtctgtgaaaaatctctatcaactaagcaacctggggatccctggagtgtgggctttccatatcggcagcacttccccgttggacaatgtcaggccagctgcagttggagacctttccgctgcccactcttctgttcccctgggacgttccttcagccatgctacagccctggaaagtgactataatgaggacaatttggattactacgatgtgaatgaggaggaagctgaataccttccgggtgaaccagaggaggcattgaatggccacagcagcattgatgtttccttccaatccaaagtggatacaaagcctttagaggaatcttccaccttggatcctcacaccaaagaaggaacatctctgggagaggtagggggcccagatttaaaaggccaagttgagccctgggatgagagagagaccagaagcccagctccaccagaggtagacagagattcactggctccttcctgggaaaccccaccaccgtaccccgaaaacggaagcatccagccctacccagatggagggccagtgccttcggaaatggatgttcccccagctcatcctgaagaagaaattgttcttcgaagttaccctgcttcaggtcacactacacccttaagtcgagggacgtatgaggtgggactggaagacaacataggttccaacaccgaggtcttcacgtataatgctgccaacaaggaaacctgtgaacacaaccacagacaatgctcccggcatgccttctgcacggactatgccactggcttctgctgccactgccaatccaagttttatggaaatgggaagcactgtctgcctgagggggcacctcaccgagtgaatgggaaagtgagtggccacctccacgtgggccatacacccgtgcacttcactgatgtggacctgcatgcgtatatcgtgggcaatgatggcagagcctacacggccatcagccacatcccacagccagcagcccaggccctcctccccctcacaccaattggaggcctgtttggctggctctttgctttagaaaaacctggctctgagaacggcttcagcctcgcaggtgctgcctttacccatgacatggaagttacattctacccgggagaggagacggttcgtatcactcaaactgctgagggacttgacccagagaactacctgagcattaagaccaacattcaaggccaggtgccttacgtcccagcaaatttcacagcccacatctctccctacaaggagctgtaccactactccgactccactgtgacctctacaagttccagagactactctctgacttttggtgcaatcaaccaaacatggtcctaccgcatccaccagaacatcacttaccaggtgtgcaggcacgcccccagacacccgtccttccccaccacccagcagctgaacgtggaccgggtctttgccttgtataatgacgaagaaagagtgcttagatttgctgtgaccaatcaaattggcccggtcaaagaagattcagaccccactccggggaatccttgctatgatgggagccacatgtgtgacacaacagcacggtgccatccagggacaggtgtagattacacctgtgagtgcgcatctgggtaccagggagatggacggaactgtgtggatgaaaatgaatgtgcaactggctttcatcgctgtggccccaactctgtatgtatcaacttgcctggaagctacaggtgtgagtgccggagtggttatgagtttgcagatgaccggcatacttgcatcttgatcaccccacctgccaacccctgtgaggatggcagtcatacctgtgctcctgctgggcaggcccggtgtgttcaccatggaggcagcacgttcagctgtgcctgcctgcctggttatgccggcgatgggcaccagtgcactgatgtagatgaatgctcagaaaacagatgtcaccctgcagctacctgctacaatactcctggttccttctcctgccgttgtcaacccggatattatggggatggatttcagtgcatacctgactccacctcaagcctgacaccctgtgaacaacagcagcgccatgcccaggcccagtatgcctaccctggggcccggttccacatcccccaatgcgacgagcagggcaacttcctgcccctacagtgtcatggcagcactggtttctgctggtgcgtggaccctgatggtcatgaagttcctggtacccagactccacctggctccaccccgcctcactgtggaccatcaccagagcccacccagaggcccccgaccatctgtgagcgctggagggaaaacctgctggagcactacggtggcaccccccgggatgaccagtacgtgccccagtgcgatgacctgggccacttcatccccctgcagtgccacggaaagagcgacttctgctggtgtgtggacaaagatggcagagaggtgcagggcacccgctcccagccaggcaccacccctgcgtgtatacccaccgtcgctccacccatggtccggcccacgccccggccagatgtgacccctccatctgtgggcaccttcctgctctatactcagggccagcagattggctacttacccctcaatggcaccaggcttcagaaggatgcagctaagaccctgctgtctctgcatggctccataatcgtgggaattgattacgactgccgggagaggatggtgtactggacagatgttgctggacggacaatcagccgtgctggtctggaactgggagcagagcctgagacgatcgtgaattcaggtctgataagccctgaaggacttgccatagaccacatccgcagaacaatgtactggacggacagtgtcctggataagatagagagcgccctgctggatggctctgagcgcaaggtcctcttctacacagatctggtgaatccccgtgccatcgctgtggatccaatccgaggcaacttgtactggacagactggaatagagaagctcctaaaattgaaacgtcatctttagatggagaaaacagaagaattctgatcaatacagacattggattgcccaatggcttaacctttgaccctttctctaaactgctctgctgggcagatgcaggaaccaaaaaactggagtgtacactacctgatggaactggacggcgtgtcattcaaaacaacctcaagtaccccttcagcatcgtaagctatgcagatcacttctaccacacagactggaggagggatggtgttgtatcagtaaataaacatagtggccagtttactgatgagtatctcccagaacaacgatctcacctctacgggataactgcagtctacccctactgcccaacaggaagaaagtaagtacagtaatgtaaaggaagacttggagtttacaatcagaacctggaccctaaagaacagtgactgcaaaggcaaagaaagtaaaaaaggaattggccattagacgttcctgagcatccaagatgaacattttgtagtgcaaaaagacttttgtgaaaagctgatacctcaatctttactactgtatttttaaaaatgaaggttgttattgcaagtttaaaaaggtaacagaattttaactgttgcttattaaagcaacttcttgtaaacatttatcattaatatttaaaagatcaaattcattcaactaagaattagagtttaagactctaaacctgatttttgccatggattccttctggccaagaaattaaagcacatgtgatcaatataacaatataatcctaaaccttgacagttggagaagccaatgcagaactgatgggaaaggaccaattatttatagtttcccaacaaaagttctaagattttttacctctgcatcagtgcatttctatttatatcaaaaggtgctaaaatgattcaatttgcattttctgatcctgtagtgcctctatagaagtacccacagaaagtaaagtatcacatttataaataccaaagatgtaacaattttaaaattttctagattactccaataaagtgttttaagttttcctatgaaaaaaaaaaaaaaaaaaaa

SEQ ID NO: 6 - DNA, 5641 bp, Homo sapiens
cggaggaggacgcgagccccttgcgggcggtcatcacagcccagcctcggggctgccacagcgcgttgcgcctgtgcgccctcggtccccgcgtccactgagcgccgcgctcggggatggggcccggccggccggcccccgcgccctggcctcgtcacctgctgcgctgcgtcctgctcctcgggtgcctgcacctcggccgtcccggcgcccctggggacgccgccctcccggaacccaacgtcttcctcatcttcagccatggactgcagggctgcctggaggcccagggcgggcaggtcagagtcaccccggcttgcaataccagcctccctgcccagcgctggaagtgggtctcccgaaaccggctattcaacctgggtaccatgcagtgcctgggcacaggctggccaggcaccaacaccacggcctccctgggcatgtatgagtgtgaccgggaagcactgaatcttcgctggcattgtcgtacactgggtgaccagctgtccttgctcctgggggcccgcaccagcaacatatccaagcctggcacccttgagcgtggtgaccagacccgcagtggccagtggcgcatctacggcagcgaggaggacctatgtgctctgccctaccacgaggtctacaccatccagggaaactcccacggaaagccgtgcaccatccccttcaaatatgacaaccagtggttccacggctgcaccagcacgggccgcgaggatggtcacctgtggtgtgccaccacccaggactacggcaaagacgagcgctggggcttctgccccatcaagagtaacgactgcgagaccttctgggacaaggaccagctgactgacagctgctaccagtttaacttccagtccacgctgtcgtggagggaggcctgggccagctgcgagcagcagggtgcggatctgctgagcatcacggagatccacgagcagacctacatcaacggcctcctcactgggtacagctccaccctgtggatcggcttgaatgacttggacacgagcggaggctggcagtggtcggacaactcgcccctcaagtacctcaactgggagagtgaccagccggacaaccccagtgaggagaactgtggagtgatccgcactgagtcctcgggcggctggcagaaccgtgactgcagcatcgcgctgccctatgtgtgcaagaagaagcccaacgccacggccgagcccacccctccagacaggtgggccaatgtgaaggtggagtgcgagccgagctggcagcccttccagggccactgctaccgcctgcaggccgagaagcgcagctggcaggagtccaagaaggcatgtctacggggcggtggcgacctggtcagcatccacagcatggcggagctggaattcatcaccaagcagatcaagcaagaggtggaggagctgtggatcggcctcaacgatttgaaactgcagatgaattttgagtggtctgacgggagccttgtgagcttcacccactggcacccctttgagcccaacaacttccgggacagtctggaggactgtgtcaccatctggggcccggaaggccgctggaacgacagtccctgtaaccagtccttgccatccatctgcaagaaggcaggccagctgagccagggggccgccgaggaggaccatggctgccggaagggttggacgtggcacagcccatcctgctactggctgggagaagaccaagtgacctacagtgaggcccggcgcctgtgcactgaccatggctctcagctggtcaccatcaccaacaggttcgagcaggccttcgtcagcagcctcatctacaactgggagggcgagtacttctggacggccctgcaggacctcaacagcaccggctccttcttctggctcagtggggatgaagtcatgtacacccactggaaccgggaccagcccgggtacagccgtgggggctgcgtggcgctggccactggcagcgccatggggctgtgggaggtgaagaactgtacctcgttccgggcccgctacatctgccggcagagcctgggcactccagtgacgccggagctgccggggccagatcccacgcccagcctcactggctcctgtccccagggctgggcctcggacaccaaactccggtattgctataaggtgttcagctcagagcggctgcaggacaagaagagctgggtccaggcccagggggcctgccaggagctgggggcccagctgctgagcctggccagctacgaggaggagcactttgtggccaacatgctcaacaagatcttcggtgaatcagaacccgagatccacgagcagcactggttctggatcggcctgaaccgtcgggatcccagagggggtcagagttggcgctggagcgacggcgtagggttctcttaccacaatttcgaccggagccggcacgacgacgacgacatccgaggctgtgcggtgctggacctggcctccctgcagtgggtggccatgcagtgcgacacacagctggactggatctgcaagatccccagaggtacggacgtgcgggagcccgacgacagccctcaaggccgacgggaatggctgcgcttccaggaggccgagtacaagttctttgagcaccactccacgtgggcgcaggcgcagcgcatctgcacgtggttccaggccgagctgacctccgtgcacagccaggcagagctagacttcctgagccacaacttgcagaagttctcccgggcccaggagcagcactggtggatcggcctgcacacctctgagagcgatgggcgcttcagatggacagatggttccattataaacttcatctcctgggcaccaggcaaacctcggcctgtcggcaaggacaagaagtgcgtgtacatgacagccagccgagaggactggggggaccagaggtgcctgacagccttgccctacatctgcaagcgcagcaacgtcaccaaagaaacgcagcccccagacctgccaactacagccctggggggctgcccctctgactggatccagttcctcaacaagtgttttcaggtccagggccaggaaccccagagccgggtgaagtggtcagaggcacagttctcctgtgaacagcaagaggcccagctggtcaccatcacaaaccccttagagcaagcattcatcacagccagcctgcccaatgtgacctttgacctttggattggcctccatgcctcgcagagggacttccagtgggtggagcaggagcctttgatgtatgccaactgggcacctggggagccctctggccctagccctgctcccagtggcaacaaaccgaccagctgtgcggtggtcctgcacagcccctcagcccacttcactggccgctgggacgatcggagctgcacggaggagacccatggcttcatctgccagaagggcacggacccctccctgagcccgtccccagcagcgctgccccccgccccgggcactgagctctcctacctcaacggcaccttccggctgcttcagaagccgctgcgctggcacgatgccctcctgctgtgtgagagccacaatgccagcctggcctacgtgcccgacccctacacccaggccttcctcacgcaggctgcccgagggctgcgcacgccgctctggattgggctggctggcgaggagggctctcggcggtactcctgggtctcagaggagccgctgaactacgtgggctggcaggacggggagccgcagcagccggggggctgtacctacgtagatgtggacggggcctggcgcaccaccagctgtgacaccaagctgcagggggctgtgtgtggggttagcagtgggccccctcctccccgaagaataagctaccatggcagctgtccccagggactggcagactccgcgtggattcccttccgggagcactgctattctttccacatggagctgctgctgggccacaaggaggcgcgacagcgctgccagagagcgggtggggccgtcctgtctatcctggatgagatggagaatgtgtttgtctgggagcacctgcagagctatgagggccagagtcggggcgcctggctgggcatgaacttcaaccccaaaggaggcactctggtctggcaggacaacacagctgtgaactactccaactgggggcccccgggcttgggccccagcatgctgagccacaacagctgctactggattcagagcaacagcgggctatggcgccccggcgcttgcaccaacatcaccatgggtgtcgtctgcaagcttcctcgtgctgagcagagcagcttctccccatcagcgcttccagagaacccagcggccctggtggtggtgctgatggcggtgctgctgctcctggccttgctgaccgcagccctcatcctttaccggaggcgccagagcatcgagcgcggggcctttgagggtgcccgctacagccgcagcagctccagccccaccgaggccactgagaagaacatcctggtgtcagacatggaaatgaatgagcaacaagaatagagccaggcgcgtgggcagggccagggcgggaggagctggggagctggggccctgggtcagtctggccccccaccagctgcctgtccagttggcctatggaagggtgcccttgggagtcgctgttgggagccggagctgggcagagcctgggctggtggggtgccaccctcccacaagggctgggctgagacccagctgagtgcagcgtggcgtttccctttctgggggggcctgaggtcttgtcacctggtcctgtgcccccacaggaaccagaggtaggatgggagggggaacgagagcctctttctccccagagcccccggcccaggcctgttgatccgcgccccaggacccccttctttgcagagcccgaggagcctcccctgtcccctcgggcagatctgttgtgtctctcttcccacctggcagcctcagctctgtgcccctcaccctgctccctctcgccccttctctcccaccccttccttctgagccgggccctggggattggggagccctcttgttcctgatgagggtcagctgagggggctgagcatccatcactcctgtgcctgctggggtggctgtggggcgtggcaggaggggcctaggtgggttgggcctgagaaccagggcacgggtgtggtgtctgctgggctggagataagactggggagagacaccccaacctcccagggtgggagctgggccgggctgggatgtcatctcctgccgggcgggggagggctctgcccctggaagagtcccctgtggggaccaaaataagttccctaacatctccagctcctggctctggtttggagcaaggggaagggttgccagagtcctgggggccccagaggagcaggagtctgggagggcccagagttcaccctctagtggatccaggaggagcagcacccgagccctggagtggcccagtacccttccaagaggccacagtcccagccaggacaaagtatgcggcccatcctggtgcgacagcgtgggacaatgtgaacatggactcgaagacatggccctttctctgtagttgattttttaaatgtgccattattgttttt

SEQ ID NO: 7 - protein, 923 aa, Homo sapiens
MERGLPLLCAVLALVLAPAGAFRNDECGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFAPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLEPILITIIAMSALGVLLGAVCGVVLYCACWHNGMSERNLSALENYNFELVDGVKLKKDKLNTQSTYSEA

SEQ ID NO: 8 - protein, 1375 aa, Homo sapiens
MEGDRVAGRPVLSSLPVLLLLQLLMLRAAALHPDELFPHGESWGDQLLQEGDDESSAVVKLANPLHFYEARFSNLYVGTNGIISTQDFPRETQYVDYDFPTDFPAIAPFLADIDTSHGRGRVLYREDTSPAVLGLAARYVRAGFPRSARFTPTHAFLATWEQVGAYEEVKRGALPSGELNTFQAVLASDGSDSYALFLYPANGLQFLGTRPKESYNVQLQLPARVGFCRGEADDLKSEGPYFSLTSTEQSVKNLYQLSNLGIPGVWAFHIGSTSPLDNVRPAAVGDLSAAHSSVPLGRSFSHATALESDYNEDNLDYYDVNEEEAEYLPGEPEEALNGHSSIDVSFQSKVDTKPLEESSTLDPHTKEGTSLGEVGGPDLKGQVEPWDERETRSPAPPEVDRDSLAPSWETPPPYPENGSIQPYPDGGPVPSEMDVPPAHPEEEIVLRSYPASGHTTPLSRGTYEVGLEDNIGSNTEVFTYNAANKETCEHNHRQCSRHAFCTDYATGFCCHCQSKFYGNGKHCLPEGAPHRVNGKVSGHLHVGHTPVHFTDVDLHAYIVGNDGRAYTAISHIPQPAAQALLPLTPIGGLFGWLFALEKPGSENGFSLAGAAFTHDMEVTFYPGEETVRITQTAEGLDPENYLSIKTNIQGQVPYVPANFTAHISPYKELYHYSDSTVTSTSSRDYSLTFGAINQTWSYRIHQNITYQVCRHAPRHPSFPTTQQLNVDRVFALYNDEERVLRFAVTNQIGPVKEDSDPTPGNPCYDGSHMCDTTARCHPGTGVDYTCECASGYQGDGRNCVDENECATGFHRCGPNSVCINLPGSYRCECRSGYEFADDRHTCILITPPANPCEDGSHTCAPAGQARCVHHGGSTFSCACLPGYAGDGHQCTDVDECSENRCHPAATCYNTPGSFSCRCQPGYYGDGFQCIPDSTSSLTPCEQQQRHAQAQYAYPGARFHIPQCDEQGNFLPLQCHGSTGFCWCVDPDGHEVPGTQTPPGSTPPHCGPSPEPTQRPPTICERWRENLLEHYGGTPRDDQYVPQCDDLGHFIPLQCHGKSDFCWCVDKDGREVQGTRSQPGTTPACIPTVAPPMVRPTPRPDVTPPSVGTFLLYTQGQQIGYLPLNGTRLQKDAAKTLLSLHGSIIVGIDYDCRERMVYWTDVAGRTISRAGLELGAEPETIVNSGLISPEGLAIDHIRRTMYWTDSVLDKIESALLDGSERKVLFYTDLVNPRAIAVDPIRGNLYWTDWNREAPKIETSSLDGENRRILINTDIGLPNGLTFDPFSKLLCWADAGTKKLECTLPDGTGRRVIQNNLKYPFSIVSYADHFYHTDWRRDGVVSVNKHSGQFTDEYLPEQRSHLYGITAVYPYCPTGRK

SEQ ID NO: 9 - protein, 1479 aa, Homo sapiens
MGPGRPAPAPWPRHLLRCVLLLGCLHLGRPGAPGDAALPEPNVFLIFSHGLQGCLEAQGGQVRVTPACNTSLPAQRWKWVSRNRLFNLGTMQCLGTGWPGTNTTASLGMYECDREALNLRWHCRTLGDQLSLLLGARTSNISKPGTLERGDQTRSGQWRIYGSEEDLCALPYHEVYTIQGNSHGKPCTIPFKYDNQWFHGCTSTGREDGHLWCATTQDYGKDERWGFCPIKSNDCETFWDKDQLTDSCYQFNFQSTLSWREAWASCEQQGADLLSITEIHEQTYINGLLTGYSSTLWIGLNDLDTSGGWQWSDNSPLKYLNWESDQPDNPSEENCGVIRTESSGGWQNRDCSIALPYVCKKKPNATAEPTPPDRWANVKVECEPSWQPFQGHCYRLQAEKRSWQESKKACLRGGGDLVSIHSMAELEFITKQIKQEVEELWIGLNDLKLQMNFEWSDGSLVSFTHWHPFEPNNFRDSLEDCVTIWGPEGRWNDSPCNQSLPSICKKAGQLSQGAAEEDHGCRKGWTWHSPSCYWLGEDQVTYSEARRLCTDHGSQLVTITNRFEQAFVSSLIYNWEGEYFWTALQDLNSTGSFFWLSGDEVMYTHWNRDQPGYSRGGCVALATGSAMGLWEVKNCTSFRARYICRQSLGTPVTPELPGPDPTPSLTGSCPQGWASDTKLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICKIPRGTDVREPDDSPQGRREWLRFQEAEYKFFEHHSTWAQAQRICTWFQAELTSVHSQAELDFLSHNLQKFSRAQEQHWWIGLHTSESDGRFRWTDGSIINFISWAPGKPRPVGKDKKCVYMTASREDWGDQRCLTALPYICKRSNVTKETQPPDLPTTALGGCPSDWIQFLNKCFQVQGQEPQSRVKWSEAQFSCEQQEAQLVTITNPLEQAFITASLPNVTFDLWIGLHASQRDFQWVEQEPLMYANWAPGEPSGPSPAPSGNKPTSCAVVLHSPSAHFTGRWDDRSCTEETHGFICQKGTDPSLSPSPAALPPAPGTELSYLNGTFRLLQKPLRWHDALLLCESHNASLAYVPDPYTQAFLTQAARGLRTPLWIGLAGEEGSRRYSWVSEEPLNYVGWQDGEPQQPGGCTYVDVDGAWRTTSCDTKLQGAVCGVSSGPPPPRRISYHGSCPQGLADSAWIPFREHCYSFHMELLLGHKEARQRCQRAGGAVLSILDEMENVFVWEHLQSYEGQSRGAWLGMNFNPKGGTLVWQDNTAVNYSNWGPPGLGPSMLSHNSCYWIQSNSGLWRPGACTNITMGVVCKLPRAEQSSFSPSALPENPAALVVVLMAVLLLLALLTAALILYRRRQSIERGAFEGARYSRSSSSPTEATEKNILVSDMEMNEQQE

SEQ ID NO: 10 - DNA, 20 bp, Artificial Sequence
  Description of Artificial Sequence Synthetic primer
ttgtcccaactgaggctttg

SEQ ID NO: 11 - DNA, 20 bp, Artificial Sequence
  Description of Artificial Sequence Synthetic primer
aacagcctttcagctttggc

SEQ ID NO: 12 - DNA, 30 bp, Artificial Sequence
  Description of Artificial Sequence Synthetic primer
acgttggatgcctctgtttccaactcaagg

SEQ ID NO: 13 - DNA, 30 bp, Artificial Sequence
  Description of Artificial Sequence Synthetic primer
acgttggatgttcacagaggttactaaggg

SEQ ID NO: 14 - DNA, 20 bp, Artificial Sequence
  Description of Artificial Sequence Synthetic primer
aaccaacccactgggttgac

SEQ ID NO: 15 - DNA, 20 bp, Artificial Sequence
  Description of Artificial Sequence Synthetic primer
tgatctgggagagtttggag

SEQ ID NO: 16 - DNA, 19 bp, Artificial Sequence
  Description of Artificial Sequence Synthetic primer
cagctttggccaggagatg

SEQ ID NO: 17 - DNA, 20 bp, Artificial Sequence
  Description of Artificial Sequence Synthetic primer
agcacataacacagcatggc

SEQ ID NO: 18 - DNA, 19 bp, Artificial Sequence
  Description of Artificial Sequence Synthetic primer
ggttgacgtcaacacaggc

SEQ ID NO: 19 - DNA, 30 bp, Artificial Sequence
  Description of Artificial Sequence Synthetic primer
acgttggatgagaccaacattcaaggccag

SEQ ID NO: 20 - DNA, 30 bp, Artificial Sequence
  Description of Artificial Sequence Synthetic primer
acgttggatgagtggtacagctccttgtag

SEQ ID NO: 21 - DNA, 17 bp, Artificial Sequence
  Description of Artificial Sequence Synthetic primer
ggctgtgaaatttgctg

SEQ ID NO: 22 - DNA, 30 bp, Artificial Sequence
  Description of Artificial Sequence Synthetic primer
acgttggatgagaaatcgaggtgttggctg

SEQ ID NO: 23 - DNA, 30 bp, Artificial Sequence
  Description of Artificial Sequence Synthetic primer
acgttggatgctgcaagaacctgaactctg

SEQ ID NO: 24 - DNA, 30 bp, Artificial Sequence
  Description of Artificial Sequence Synthetic primer
acgttggatgcaaacacgaatctaggagcc

SEQ ID NO: 25 - DNA, 30 bp, Artificial Sequence
  Description of Artificial Sequence Synthetic primer
acgttggatgctccctttcttgaggttgac

SEQ ID NO: 26 - DNA, 36 bp, Artificial Sequence
  Description of Artificial Sequence Synthetic primer
ggcgcacgcctccacgtgtctctagacgtgtgttgg

SEQ ID NO: 27 - DNA, 30 bp, Artificial Sequence
  Description of Artificial Sequence Synthetic primer
ggcgcacgccatggattgagccacactctc

SEQ ID NO: 28 - DNA, 30 bp, Artificial Sequence
  Description of Artificial Sequence Synthetic primer
acgttggatgggttgcggttatcttagcag

SEQ ID NO: 29 - DNA, 30 bp, Artificial Sequence
  Description of Artificial Sequence Synthetic primer
acgttggatggcagtgggcactaacctaag

SEQ ID NO: 30 - DNA, 30 bp, Artificial Sequence
  Description of Artificial Sequence Synthetic primer
acgttggatgaacggacaagtactctaagc

SEQ ID NO: 31 - DNA, 31 bp, Artificial Sequence
  Description of Artificial Sequence Synthetic primer
acgttggatgcctcttctgtaatctctagtg

SEQ ID NO: 32 - DNA, 30 bp, Artificial Sequence
  Description of Artificial Sequence Synthetic primer
acgttggatgctccctttttcttattagtc

SEQ ID NO: 33 - DNA, 31 bp, Artificial Sequence
  Description of Artificial Sequence Synthetic primer
acgttggatggaaaagaacattatttgcagc

SEQ ID NO: 34 - DNA, 30 bp, Artificial Sequence
  Description of Artificial Sequence Synthetic primer
acgttggatggacatctcattttctaggtg

SEQ ID NO: 35 - DNA, 30 bp, Artificial Sequence
  Description of Artificial Sequence Synthetic primer
acgttggatggaaatcctgtggtaggaatc

SEQ ID NO: 36 - DNA, 30 bp, Artificial Sequence
  Description of Artificial Sequence Synthetic primer
acgttggatgagtggctcaatcttggctcc

SEQ ID NO: 37 - DNA, 30 bp, Artificial Sequence
  Description of Artificial Sequence Synthetic primer
acgttggatgatgcctgaaatcccagctac

SEQ ID NO: 38 - DNA, 30 bp, Artificial Sequence; identical to SEQ ID NO: 36
  Description of Artificial Sequence Synthetic primer

SEQ ID NO: 39 - DNA, 30 bp, Artificial Sequence; identical to SEQ ID NO: 37
  Description of Artificial Sequence Synthetic primer

SEQ ID NO: 40 - DNA, 30 bp, Artificial Sequence
  Description of Artificial Sequence Synthetic primer
acgttggatgcacctagggatttatagacg

SEQ ID NO: 41 - DNA, 30 bp, Artificial Sequence
  Description of Artificial Sequence Synthetic primer
acgttggatgagtctcaccaagaccttctg

SEQ ID NO: 42 - DNA, 30 bp, Artificial Sequence
  Description of Artificial Sequence Synthetic primer
acgttggatgtgcacatatagacgtatcac

SEQ ID NO: 43 - DNA, 30 bp, Artificial Sequence
  Description of Artificial Sequence Synthetic primer
acgttggatgggaatataagtcctgcagcc

SEQ ID NO: 44 - DNA, 30 bp, Artificial Sequence
  Description of Artificial Sequence Synthetic primer
acgttggatggcatacacaaatatatgcac

SEQ ID NO: 45 - DNA, 30 bp, Artificial Sequence
  Description of Artificial Sequence Synthetic primer
acgttggatgtaagtcctgcagccactttg

SEQ ID NO: 46 - DNA, 30 bp, Artificial Sequence
  Description of Artificial Sequence Synthetic primer
acgttggatgctacagtggcaaagaacacg

SEQ ID NO: 47 - DNA, 30 bp, Artificial Sequence
  Description of Artificial Sequence Synthetic primer
acgttggatgtttaaacatggtctcgtggc

SEQ ID NO: 48 - DNA, 30 bp, Artificial Sequence
  Description of Artificial Sequence Synthetic primer
acgttggatgtctgggcagtaagtttcagg

SEQ ID NO: 49 - DNA, 30 bp, Artificial Sequence
  Description of Artificial Sequence Synthetic primer
acgttggatgggatcctaatttgcgctgac

SEQ ID NO: 50 - DNA, 30 bp, Artificial Sequence
  Description of Artificial Sequence Synthetic primer
acgttggatgccaaataacatgctgatcgc

SEQ ID NO: 51 - DNA, 30 bp, Artificial Sequence
  Description of Artificial Sequence Synthetic primer
acgttggatgcacactcactgaagcgtttg

SEQ ID NO: 52 - DNA, 30 bp, Artificial Sequence
  Description of Artificial Sequence Synthetic primer
acgttggatgcctacttttccttcaaacac

SEQ ID NO: 53 - DNA, 30 bp, Artificial Sequence
  Description of Artificial Sequence Synthetic primer
acgttggatgtccttcctctcctccttctc

SEQ ID NO: 54 - DNA, 30 bp, Artificial Sequence
  Description of Artificial Sequence Synthetic primer
acgttggatgcccagcacaaataagtgctc

SEQ ID NO: 55 - DNA, 30 bp, Artificial Sequence
  Description of Artificial Sequence Synthetic primer
acgttggatgaatactctcaccaccaccac

SEQ ID NO: 56 - DNA, 30 bp, Artificial Sequence
  Description of Artificial Sequence Synthetic primer
acgttggatgtattttcctttcccaccagc

SEQ ID NO: 57 - DNA, 30 bp, Artificial Sequence
  Description of Artificial Sequence Synthetic primer
acgttggatgcaggattatctgacaggagg

SEQ ID NO: 58 - DNA, 30 bp, Artificial Sequence
  Description of Artificial Sequence Synthetic primer
acgttggatgtgtttaccgatgctcgtacc

SEQ ID NO: 59 - DNA, 30 bp, Artificial Sequence
  Description of Artificial Sequence Synthetic primer
acgttggatgcaatcccaggggattatagc

SEQ ID NO: 60 - DNA, 30 bp, Artificial Sequence
  Description of Artificial Sequence Synthetic primer
acgttggatggaggagtcgactgttgaatg

SEQ ID NO: 61 - DNA, 30 bp, Artificial Sequence
  Description of Artificial Sequence Synthetic primer
acgttggatgggatgaaatgagaatgaccc

SEQ ID NO: 62 - DNA, 30 bp, Artificial Sequence
  Description of Artificial Sequence Synthetic primer
acgttggatgctcctgggttcaagagattc

SEQ ID NO: 63 - DNA, 30 bp, Artificial Sequence
  Description of Artificial Sequence Synthetic primer
acgttggatgatacaaaaattagccgggcg

SEQ ID NO: 64 - DNA, 30 bp, Artificial Sequence
  Description of Artificial Sequence Synthetic primer
acgttggatgagcagatttactcccttccc

SEQ ID NO: 65 - DNA, 30 bp, Artificial Sequence
  Description of Artificial Sequence Synthetic primer
acgttggatggaattgggaggcatgctttg

SEQ ID NO: 66 - DNA, 30 bp, Artificial Sequence
  Description of Artificial Sequence Synthetic primer
acgttggatgcctgtacgtgtacaggtttg

SEQ ID NO: 67 - DNA, 30 bp, Artificial Sequence
  Description of Artificial Sequence Synthetic primer
acgttggatggtgcttattacccgagtgac

SEQ ID NO: 68 - DNA, 30 bp, Artificial Sequence
  Description of Artificial Sequence Synthetic primer
acgttggatggggaagaaaagacgacctac

SEQ ID NO: 69 - DNA, 30 bp, Artificial Sequence
  Description of Artificial Sequence Synthetic primer
acgttggatgtggtttatggtcttggcgtc

SEQ ID NO: 70 - DNA, 30 bp, Artificial Sequence
  Description of Artificial Sequence Synthetic primer
acgttggatgtagagcatgatctaggtccc

SEQ ID NO: 71 - DNA, 30 bp, Artificial Sequence
  Description of Artificial Sequence Synthetic primer
acgttggatgtttccagcttgcagaacctc

SEQ ID NO: 72 - DNA, 30 bp, Artificial Sequence
  Description of Artificial Sequence Synthetic primer
acgttggatgtcagctaattcccagcattc

SEQ ID NO: 73 - DNA, 31 bp, Artificial Sequence
  Description of Artificial Sequence Synthetic primer
acgttggatgggagttgacagttcaaatcct

SEQ ID NO: 74 - DNA, 31 bp, Artificial Sequence; identical to SEQ ID NO: 73
  Description of Artificial Sequence Synthetic primer

SEQ ID NO: 75 - DNA, 30 bp, Artificial Sequence; identical to SEQ ID NO: 72
  Description of Artificial Sequence Synthetic primer

SEQ ID NO: 76 - DNA, 30 bp, Artificial Sequence
  Description of Artificial Sequence Synthetic primer
acgttggatgtcagaagaccacattctcag

SEQ ID NO: 77 - DNA, 30 bp, Artificial Sequence
  Description of Artificial Sequence Synthetic primer
acgttggatgcctagaccttagtcttaccc

SEQ ID NO: 78 - DNA, 30 bp, Artificial Sequence
  Description of Artificial Sequence Synthetic primer
acgttggatgttcttggccctgatttctcc

SEQ ID NO: 79 - DNA, 30 bp, Artificial Sequence
  Description of Artificial Sequence Synthetic primer
acgttggatgaaagaagaaggccccaacac

SEQ ID NO: 80 - DNA, 30 bp, Artificial Sequence
  Description of Artificial Sequence Synthetic primer
acgttggatggaaagagcttgggatcatgg

SEQ ID NO: 81 - DNA, 30 bp, Artificial Sequence
  Description of Artificial Sequence Synthetic primer
acgttggatgcataaggccattcatgaggg

SEQ ID NO: 82 - DNA, 30 bp, Artificial Sequence
  Description of Artificial Sequence Synthetic primer
acgttggatggtggccagattctctgaatg

SEQ ID NO: 83 - DNA, 30 bp, Artificial Sequence
  Description of Artificial Sequence Synthetic primer
acgttggatgagcacttaagacagccactc

SEQ ID NO: 84 - DNA, 30 bp, Artificial Sequence
  Description of Artificial Sequence Synthetic primer
acgttggatgcctttaacccttacagagcc

SEQ ID NO: 85 - DNA, 30 bp, Artificial Sequence
  Description of Artificial Sequence Synthetic primer
acgttggatgaagtgttcttctcccacagc

SEQ ID NO: 86 - DNA, 30 bp, Artificial Sequence
  Description of Artificial Sequence Synthetic primer
acgttggatgcatttgacagctgagatccc

SEQ ID NO: 87 - DNA, 30 bp, Artificial Sequence
  Description of Artificial Sequence Synthetic primer
acgttggatggacagaaagatttgtgacgc

SEQ ID NO: 88 - DNA, 30 bp, Artificial Sequence
  Description of Artificial Sequence Synthetic primer
acgttggatggacactactaactgccctag

SEQ ID NO: 89 - DNA, 30 bp, Artificial Sequence
  Description of Artificial Sequence Synthetic primer
acgttggatgagttcgttgcaccttgagag

SEQ ID NO: 90 - DNA, 30 bp, Artificial Sequence
  Description of Artificial Sequence Synthetic primer
acgttggatgatgttggccaggctggtttc

SEQ ID NO: 91 - DNA, 30 bp, Artificial Sequence
  Description of Artificial Sequence Synthetic primer
acgttggatgtggctcactcgcaaacattg

SEQ ID NO: 92 - DNA, 31 bp, Artificial Sequence
  Description of Artificial Sequence Synthetic primer
acgttggatggttacaagtatgcgatcttcc

SEQ ID NO: 93 - DNA, 30 bp, Artificial Sequence
  Description of Artificial Sequence Synthetic primer
acgttggatgctctcctctgtgtaaccaac

SEQ ID NO: 94 - DNA, 30 bp, Artificial Sequence
  Description of Artificial Sequence Synthetic primer
acgttggatggcaaatgtgctaaggcatcg

SEQ ID NO: 95 - DNA, 30 bp, Artificial Sequence
  Description of Artificial Sequence Synthetic primer
acgttggatgacaccctgcattttctcttg

SEQ ID NO: 96 - DNA, 30 bp, Artificial Sequence
  Description of Artificial Sequence Synthetic primer
acgttggatgttgtcccaactgaggctttg

SEQ ID NO: 97 - DNA, 30 bp, Artificial Sequence
  Description of Artificial Sequence Synthetic primer
acgttggatgaacagcctttcagctttggc

SEQ ID NO: 98 - DNA, 30 bp, Artificial Sequence
  Description of Artificial Sequence Synthetic primer
acgttggatgctgggctgcctatattgttc

SEQ ID NO: 99 - DNA, 30 bp, Artificial Sequence
  Description of Artificial Sequence Synthetic primer
acgttggatgaatgtggcagcatttcaggc

SEQ ID NO: 100 - DNA, 30 bp, Artificial Sequence
  Description of Artificial Sequence Synthetic primer
acgttggatggagagattgaacagagccag

SEQ ID NO: 101 - DNA, 30 bp, Artificial Sequence
  Description of Artificial Sequence Synthetic primer
acgttggatgcacaccagagctctttaatc

SEQ ID NO: 102 - DNA, 36 bp, Artificial Sequence
  Description of Artificial Sequence Synthetic primer
ggcgcacgcctccacggggcttttccttacctctgt

SEQ ID NO: 103 - DNA, 30 bp, Artificial Sequence
  Description of Artificial Sequence Synthetic primer
ggcgcacgccaattggcatgctgatctggg

SEQ ID NO: 104 - DNA, 29 bp, Artificial Sequence
  Description of Artificial Sequence Synthetic primer
acgttggatggctcctctgcgccgtgctc

SEQ ID NO: 105 - DNA, 30 bp, Artificial Sequence
  Description of Artificial Sequence Synthetic primer
acgttggatgctgcctgtcacttaccgttg

SEQ ID NO: 106 - DNA, 30 bp, Artificial Sequence
  Description of Artificial Sequence Synthetic primer
acgttggatgtggtggtggataagccaatg

SEQ ID NO: 107 - DNA, 30 bp, Artificial Sequence
  Description of Artificial Sequence Synthetic primer
acgttggatgtacatgaaggcaatcgcacc

SEQ ID NO: 108 - DNA, 30 bp, Artificial Sequence
  Description of Artificial Sequence Synthetic primer
acgttggatgaaatgaactcctcttggccg

SEQ ID NO: 109 - DNA, 30 bp, Artificial Sequence
  Description of Artificial Sequence Synthetic primer
acgttggatgaactcctgacctcaagtgac

SEQ ID NO: 110 - DNA, 30 bp, Artificial Sequence
  Description of Artificial Sequence Synthetic primer
acgttggatgattacccaagtaggcttccc

SEQ ID NO: 111 - DNA, 30 bp, Artificial Sequence
  Description of Artificial Sequence Synthetic primer
acgttggatgacagatatgctgcagtgacc

SEQ ID NO: 112 - DNA, 30 bp, Artificial Sequence
  Description of Artificial Sequence Synthetic primer
acgttggatgaggaacagccacattttgac

SEQ ID NO: 113 - DNA, 31 bp, Artificial Sequence
  Description of Artificial Sequence Synthetic primer
acgttggatgggaagatctttgagagttagc

SEQ ID NO: 114 - DNA, 30 bp, Artificial Sequence
  Description of Artificial Sequence Synthetic primer
acgttggatgcatcattcccccagttctag

SEQ ID NO: 115 - DNA, 30 bp, Artificial Sequence
  Description of Artificial Sequence Synthetic primer
acgttggatgtgtggcatcatttgtcttcc

SEQ ID NO: 116 - DNA, 30 bp, Artificial Sequence
  Description of Artificial Sequence Synthetic primer
acgttggatgaatctgtttgcttacttttc

SEQ ID NO: 117 - DNA, 30 bp, Artificial Sequence
  Description of Artificial Sequence Synthetic primer
acgttggatggtcctctctctgttttaggc

SEQ ID NO: 118 - DNA, 30 bp, Artificial Sequence
  Description of Artificial Sequence Synthetic primer
acgttggatgaggtcctgaccatgcatgag

SEQ ID NO: 119 - DNA, 30 bp, Artificial Sequence
  Description of Artificial Sequence Synthetic primer
acgttggatgacacccaactctctatgagc

SEQ ID NO: 120 - DNA, 30 bp, Artificial Sequence
  Description of Artificial Sequence Synthetic primer
acgttggatgtgcacacatgtagtcccagc

SEQ ID NO: 121 - DNA, 30 bp, Artificial Sequence
  Description of Artificial Sequence Synthetic primer
acgttggatgctcggcttactgtaatctcc

SEQ ID NO: 122 - DNA, 30 bp, Artificial Sequence
  Description of Artificial Sequence Synthetic primer
acgttggatggaggcggagattacagtaag

SEQ ID NO: 123 - DNA, 30 bp, Artificial Sequence
  Description of Artificial Sequence Synthetic primer
acgttggatgttttcgagacggagtcttgc

SEQ ID NO: 124 - DNA, 30 bp, Artificial Sequence
  Description of Artificial Sequence Synthetic primer
acgttggatggttctgttgccttcatgctc

SEQ ID NO: 125 - DNA, 30 bp, Artificial Sequence
  Description of Artificial Sequence Synthetic primer
acgttggatgttgaaatcctttccactggc

SEQ ID NO: 126 - DNA, 30 bp, Artificial Sequence
  Description of Artificial Sequence Synthetic primer
acgttggatgaggtcttcaagacctgttcc

SEQ ID NO: 127 - DNA, 30 bp, Artificial Sequence
  Description of Artificial Sequence Synthetic primer
acgttggatgccccaagaatcttgcacttc

SEQ ID NO: 128 - DNA, 30 bp, Artificial Sequence
  Description of Artificial Sequence Synthetic primer
acgttggatgtttgtcccctatcctctctc

SEQ ID NO: 129 - DNA, 30 bp, Artificial Sequence
  Description of Artificial Sequence Synthetic primer
acgttggatgcccataaaggaagcagaagc

SEQ ID NO: 130 - DNA, 30 bp, Artificial Sequence
  Description of Artificial Sequence Synthetic primer
acgttggatggcactctctgggataatttc

SEQ ID NO: 131 - DNA, 30 bp, Artificial Sequence
  Description of Artificial Sequence Synthetic primer
acgttggatggtgaacaaaagtagactccc

SEQ ID NO: 132 - DNA, 31 bp, Artificial Sequence
  Description of Artificial Sequence Synthetic primer
acgttggatgcccatgaaagacttaaccttc

SEQ ID NO: 133 - DNA, 30 bp, Artificial Sequence
  Description of Artificial Sequence Synthetic primer
acgttggatgtaatttcctccatccctccc

SEQ ID NO: 134 - DNA, 30 bp, Artificial Sequence
  Description of Artificial Sequence Synthetic primer
acgttggatgcaaatagaactgacatctgc

SEQ ID NO: 135 - DNA, 30 bp, Artificial Sequence
  Description of Artificial Sequence Synthetic primer
acgttggatgatctggtcaggtttggtctc

SEQ ID NO: 136 - DNA, 30 bp, Artificial Sequence
  Description of Artificial Sequence Synthetic primer
acgttggatgccacccatcattacaatcgc

SEQ ID NO: 137 - DNA, 30 bp, Artificial Sequence
  Description of Artificial Sequence Synthetic primer
acgttggatggatcattggtggaaacaggg

SEQ ID NO: 138 - DNA, 30 bp, Artificial Sequence
  Description of Artificial Sequence Synthetic primer
acgttggatgctcagggtgtattttacacg

SEQ ID NO: 139 - DNA, 30 bp, Artificial Sequence
  Description of Artificial Sequence Synthetic primer
acgttggatgaactccctccttcccttaac

SEQ ID NO: 140 - DNA, 30 bp, Artificial Sequence
  Description of Artificial Sequence Synthetic primer
acgttggatgcagtgctcactcagttagtc

SEQ ID NO: 141 - DNA, 30 bp, Artificial Sequence
  Description of Artificial Sequence Synthetic primer
acgttggatggcttctattagggtgttggc

SEQ ID NO: 142 - DNA, 30 bp, Artificial Sequence
  Description of Artificial Sequence Synthetic primer
acgttggatgaattaaccctcggccaggtg

SEQ ID NO: 143 - DNA, 30 bp, Artificial Sequence
  Description of Artificial Sequence Synthetic primer
acgttggatgttcttgacctcgtgatccac

SEQ ID NO: 144 - DNA, 30 bp, Artificial Sequence
  Description of Artificial Sequence Synthetic primer
acgttggatgtggccgagggttaatttttg

SEQ ID NO: 145 - DNA, 30 bp, Artificial Sequence
  Description of Artificial Sequence Synthetic primer
acgttggatgggtgcagctaaagagaaacg

SEQ ID NO: 146 - DNA, 30 bp, Artificial Sequence
  Description of Artificial Sequence Synthetic primer
acgttggatgcatgaatccctctgcaactc

SEQ ID NO: 147 - DNA, 30 bp, Artificial Sequence
  Description of Artificial Sequence Synthetic primer
acgttggatgaagtttgagaaccactgggc

SEQ ID NO: 148 - DNA, 30 bp, Artificial Sequence
  Description of Artificial Sequence Synthetic primer
acgttggatgactttgcttcataccatgga

SEQ ID NO: 149 - DNA, 30 bp, Artificial Sequence
  Description of Artificial Sequence Synthetic primer
acgttggatggcactagtccagcattttcc

SEQ ID NO: 150 - DNA, 30 bp, Artificial Sequence
  Description of Artificial Sequence Synthetic primer
acgttggatgtggactggtttcttcctcac

SEQ ID NO: 151 - DNA, 30 bp, Artificial Sequence
  Description of Artificial Sequence Synthetic primer
acgttggatgcaagatgagatttgggtggg

SEQ ID NO: 152 - DNA, 30 bp, Artificial Sequence
  Description of Artificial Sequence Synthetic primer
acgttggatgggaagaaaccagtccaagag

SEQ ID NO: 153 - DNA, 30 bp, Artificial Sequence
  Description of Artificial Sequence Synthetic primer
acgttggatgcctcttccaaaagtaagccc

SEQ ID NO: 154 - DNA, 30 bp, Artificial Sequence
  Description of Artificial Sequence Synthetic primer
acgttggatgagaggtgcaataactggtgg

SEQ ID NO: 155 - DNA, 30 bp, Artificial Sequence
  Description of Artificial Sequence Synthetic primer
acgttggatgttcagtgaaagttgcctgtg

SEQ ID NO: 156 - DNA, 30 bp, Artificial Sequence
  Description of Artificial Sequence Synthetic primer
acgttggatgttagccaggtatggtgatgc

SEQ ID NO: 157 - DNA, 30 bp, Artificial Sequence
  Description of Artificial Sequence Synthetic primer
acgttggatgccaggttcaagcaattctcc

SEQ ID NO: 158 - DNA, 30 bp, Artificial Sequence
  Description of Artificial Sequence Synthetic primer
acgttggatgaggggtacacaattcaatcc

SEQ ID NO: 159 - DNA, 30 bp, Artificial Sequence
  Description of Artificial Sequence Synthetic primer
acgttggatgcattaatatgtggccatggg

SEQ ID NO: 160 - DNA, 30 bp, Artificial Sequence
  Description of Artificial Sequence Synthetic primer
acgttggatgtgctttctctctcttctccc

SEQ ID NO: 161 - DNA, 30 bp, Artificial Sequence
  Description of Artificial Sequence Synthetic primer
acgttggatgaagaccctcttcctggtttg

SEQ ID NO: 162 - DNA, 30 bp, Artificial Sequence
  Description of Artificial Sequence Synthetic primer
acgttggatgtgaaggatctttactcaggc

SEQ ID NO: 163 - DNA, 30 bp, Artificial Sequence
  Description of Artificial Sequence Synthetic primer
acgttggatgaccactcatgtctgcctgct

SEQ ID NO: 164 - DNA, 31 bp, Artificial Sequence
  Description of Artificial Sequence Synthetic primer
acgttggatgggatggtttcactactgtatg

SEQ ID NO: 165 - DNA, 30 bp, Artificial Sequence
  Description of Artificial Sequence Synthetic primer
acgttggatgtaacatgtccacagccttcc

SEQ ID NO: 166 - DNA, 30 bp, Artificial Sequence
  Description of Artificial Sequence Synthetic primer
acgttggatgaaaactgccattcctcccac

SEQ ID NO: 167 - DNA, 30 bp, Artificial Sequence
  Description of Artificial Sequence Synthetic primer
acgttggatgccttcttcattcatcagggc

SEQ ID NO: 168 - DNA, 30 bp, Artificial Sequence
  Description of Artificial Sequence Synthetic primer
acgttggatgataaagtgcagtacctcccc

SEQ ID NO: 169 - DNA, 30 bp, Artificial Sequence
  Description of Artificial Sequence Synthetic primer
acgttggatgtttattcatggcagaaggcg

SEQ ID NO: 170 - DNA, 30 bp, Artificial Sequence
  Description of Artificial Sequence Synthetic primer
acgttggatgttcacatggtgaaagcaggg

SEQ ID NO: 171 - DNA, 30 bp, Artificial Sequence
  Description of Artificial Sequence Synthetic primer
acgttggatgctaatttcacctgtaggatg

SEQ ID NO: 172 - DNA, 30 bp, Artificial Sequence
  Description of Artificial Sequence Synthetic primer
acgttggatgagttccacatagctggagag

SEQ ID NO: 173 - DNA, 30 bp, Artificial Sequence
  Description of Artificial Sequence Synthetic primer
acgttggatggctggcactcattttctctc

SEQ ID NO: 174 - DNA, 17 bp, Artificial Sequence
  Description of Artificial Sequence Synthetic primer
ccagtggtgtgagcttc

SEQ ID NO: 175 - DNA, 18 bp, Artificial Sequence
  Description of Artificial Sequence Synthetic primer
gaggttgactcatgattt

SEQ ID NO: 176 - DNA, 22 bp, Artificial Sequence
  Description of Artificial Sequence Synthetic primer
cctgattgaagcaaaaccagaa

SEQ ID NO: 177 - DNA, 17 bp, Artificial Sequence
  Description of Artificial Sequence Synthetic primer
tgttcaacagacccttc

SEQ ID NO: 178 - DNA, 24 bp, Artificial Sequence
  Description of Artificial Sequence Synthetic primer
gtaatctctagtgtttattgaaag

SEQ ID NO: 179 - DNA, 17 bp, Artificial Sequence
  Description of Artificial Sequence Synthetic primer
ttgcagcatttccctaa

SEQ ID NO: 180 - DNA, 18 bp, Artificial Sequence
  Description of Artificial Sequence Synthetic primer
gtggatcctaatgatcac

SEQ ID NO: 181 - DNA, 17 bp, Artificial Sequence
  Description of Artificial Sequence Synthetic primer
ggagaactgcttgaacc

SEQ ID NO: 182 - DNA, 17 bp, Artificial Sequence
  Description of Artificial Sequence Synthetic primer
agaactgcttgaacccg

SEQ ID NO: 183 - DNA, 18 bp, Artificial Sequence
  Description of Artificial Sequence Synthetic primer
tgggatttcgttacctaa

SEQ ID NO: 184 - DNA, 20 bp, Artificial Sequence
  Description of Artificial Sequence Synthetic primer
caacctgaggatgtatagaa

SEQ ID NO: 185 - DNA, 22 bp, Artificial Sequence
  Description of Artificial Sequence Synthetic primer
ggatgtatagaatgcctatatg

SEQ ID NO: 186 - DNA, 19 bp, Artificial Sequence
  Description of Artificial Sequence Synthetic primer
ttccttgatctctttcttc

SEQ ID NO: 187 - DNA, 17 bp, Artificial Sequence
  Description of Artificial Sequence Synthetic primer
ttgcgctgacaaaaacc

SEQ ID NO: 188 - DNA, 17 bp, Artificial Sequence
  Description of Artificial Sequence Synthetic primer
gagatatgtaggacccc

SEQ ID NO: 189 - DNA, 18 bp, Artificial Sequence
  Description of Artificial Sequence Synthetic primer
agagggtagtaaatcctg

SEQ ID NO: 190 - DNA, 22 bp, Artificial Sequence
  Description of Artificial Sequence Synthetic primer
tgctattactacttctacctac

SEQ ID NO: 191 - DNA, 17 bp, Artificial Sequence
  Description of Artificial Sequence Synthetic primer
gtgtgtgcacatctcaa

SEQ ID NO: 192 - DNA, 17 bp, Artificial Sequence
  Description of Artificial Sequence Synthetic primer
cgtagttcttcgcccct

SEQ ID NO: 193 - DNA, 18 bp, Artificial Sequence
  Description of Artificial Sequence Synthetic primer
accccatctaaacaaaag

SEQ ID NO: 194 - DNA, 17 bp, Artificial Sequence
  Description of Artificial Sequence Synthetic primer
tagccgggcgtggtggt

SEQ ID NO: 195 - DNA, 18 bp, Artificial Sequence
  Description of Artificial Sequence Synthetic primer
tgggatcatgaatcctag

SEQ ID NO: 196 - DNA, 18 bp, Artificial Sequence
  Description of Artificial Sequence Synthetic primer
ctaaacccttgtgacatg

SEQ ID NO: 197 - DNA, 20 bp, Artificial Sequence
  Description of Artificial Sequence Synthetic primer
ttttactgactcttcaagtg

SEQ ID NO: 198 - DNA, 17 bp, Artificial Sequence
  Description of Artificial Sequence Synthetic primer
tatcttcatctgcagcc

SEQ ID NO: 199 - DNA, 21 bp, Artificial Sequence
  Description of Artificial Sequence Synthetic primer
gacagttcaaatccttttttt

SEQ ID NO: 200 - DNA, 22 bp, Artificial Sequence
  Description of Artificial Sequence Synthetic primer
tgtctagtatttctaaacactg

SEQ ID NO: 201 - DNA, 19 bp, Artificial Sequence
  Description of Artificial Sequence Synthetic primer
cttagtcttacccatgaac

SEQ ID NO: 202 - DNA, 22 bp, Artificial Sequence
  Description of Artificial Sequence Synthetic primer
ggagttgccaattatgtataag

SEQ ID NO: 203 - DNA, 17 bp, Artificial Sequence
  Description of Artificial Sequence Synthetic primer
ggtggggataaggagct

SEQ ID NO: 204 - DNA, 17 bp, Artificial Sequence
  Description of Artificial Sequence Synthetic primer
gggtgttggagtgggtg

SEQ ID NO: 205 - DNA, 18 bp, Artificial Sequence
  Description of Artificial Sequence Synthetic primer
ccctcattcatacagttt

SEQ ID NO: 206 - DNA, 17 bp, Artificial Sequence
  Description of Artificial Sequence Synthetic primer
tttgtgacgcagcaact

SEQ ID NO: 207 - DNA, 17 bp, Artificial Sequence
  Description of Artificial Sequence Synthetic primer
ggttgattggcctgtat

SEQ ID NO: 208 - DNA, 17 bp, Artificial Sequence
  Description of Artificial Sequence Synthetic primer
aggcaggcatcacttga

SEQ ID NO: 209 - DNA, 18 bp, Artificial Sequence
  Description of Artificial Sequence Synthetic primer
tttttcctaactcagtgc

SEQ ID NO: 210 - DNA, 18 bp, Artificial Sequence
  Description of Artificial Sequence Synthetic primer
gtatagtcaacttccacc

SEQ ID NO: 211 - DNA, 19 bp, Artificial Sequence
  Description of Artificial Sequence Synthetic primer
cagctttggccaggagatg

SEQ ID NO: 212 - DNA, 21 bp, Artificial Sequence
  Description of Artificial Sequence Synthetic primer
tccatcataaaatgtgacaat

SEQ ID NO: 213 - DNA, 18 bp, Artificial Sequence
  Description of Artificial Sequence Synthetic primer
tgacttctgagaaagtga

SEQ ID NO: 214 - DNA, 20 bp, Artificial Sequence
  Description of Artificial Sequence Synthetic primer
atgctgatctgggaaggtag

SEQ ID NO: 215 - DNA, 17 bp, Artificial Sequence
  Description of Artificial Sequence Synthetic primer
ttaccgttgcgaaaagc

SEQ ID NO: 216 - DNA, 18 bp, Artificial Sequence
  Description of Artificial Sequence Synthetic primer
tgaatctttggtaacacc

SEQ ID NO: 217 - DNA, 17 bp, Artificial Sequence
  Description of Artificial Sequence Synthetic primer
gtgctaggattacaggc

SEQ ID NO: 218 - DNA, 17 bp, Artificial Sequence
  Description of Artificial Sequence Synthetic primer
cacagagctcacccacc

SEQ ID NO: 219 - DNA, 19 bp, Artificial Sequence
  Description of Artificial Sequence Synthetic primer
aggaaaaaggcttatcaag

SEQ ID NO: 220 - DNA, 17 bp, Artificial Sequence
  Description of Artificial Sequence Synthetic primer
acttgacctagcaccag

SEQ ID NO: 221 - DNA, 18 bp, Artificial Sequence
  Description of Artificial Sequence Synthetic primer
ggctaaaaagtgtgtcaa

SEQ ID NO: 222 - DNA, 17 bp, Artificial Sequence
  Description of Artificial Sequence Synthetic primer
gatgtgctcagctctta

SEQ ID NO: 223 - DNA, 17 bp, Artificial Sequence
  Description of Artificial Sequence Synthetic primer
ctgtaatctccgcctcc

SEQ ID NO: 224 - DNA, 17 bp, Artificial Sequence
  Description of Artificial Sequence Synthetic primer
ggctggagtgcagtggc

SEQ ID NO: 225 - DNA, 18 bp, Artificial Sequence
  Description of Artificial Sequence Synthetic primer
ctggctagccttttagaa

SEQ ID NO: 226 - DNA, 18 bp, Artificial Sequence
  Description of Artificial Sequence Synthetic primer
acaaaaaccaccatcaaa

SEQ ID NO: 227 - DNA, 17 bp, Artificial Sequence
  Description of Artificial Sequence Synthetic primer
atgccaactgagaagga

SEQ ID NO: 228 - DNA, 17 bp, Artificial Sequence
  Description of Artificial Sequence Synthetic primer
ctcccttctgggaggat

SEQ ID NO: 229 - DNA, 17 bp, Artificial Sequence
  Description of Artificial Sequence Synthetic primer
cccaatcctcccagaag

SEQ ID NO: 230 - DNA, 17 bp, Artificial Sequence
  Description of Artificial Sequence Synthetic primer
ggtctccaaatgagtgg

SEQ ID NO: 231 - DNA, 18 bp, Artificial Sequence
  Description of Artificial Sequence Synthetic primer
gaaacagggatggagata

SEQ ID NO: 232 - DNA, 17 bp, Artificial Sequence
  Description of Artificial Sequence Synthetic primer
cagcaacaggtctcacc

SEQ ID NO: 233 - DNA, 18 bp, Artificial Sequence
  Description of Artificial Sequence Synthetic primer
gagaggaatggagaaaaa

SEQ ID NO: 234 - DNA, 17 bp, Artificial Sequence
  Description of Artificial Sequence Synthetic primer
gtactgggattacaggc

SEQ ID NO: 235 - DNA, 17 bp, Artificial Sequence
  Description of Artificial Sequence Synthetic primer
aaacgtcagagccagcc

SEQ ID NO: 236 - DNA, 17 bp, Artificial Sequence
  Description of Artificial Sequence Synthetic primer
tctcactggggacccac

SEQ ID NO: 237 - DNA, 17 bp, Artificial Sequence
  Description of Artificial Sequence Synthetic primer
gcattttcctctctctg

SEQ ID NO: 238 - DNA, 18 bp, Artificial Sequence
  Description of Artificial Sequence Synthetic primer
gatagaggtcaaggagag

SEQ ID NO: 239 - DNA, 17 bp, Artificial Sequence
  Description of Artificial Sequence Synthetic primer
gtaagccctacctatgc

SEQ ID NO: 240 - DNA, 17 bp, Artificial Sequence
  Description of Artificial Sequence Synthetic primer
gttgcctgtgttctgga

SEQ ID NO: 241 - DNA, 17 bp, Artificial Sequence
  Description of Artificial Sequence Synthetic primer
gcaattctcctgcctca

SEQ ID NO: 242 - DNA, 20 bp, Artificial Sequence
  Description of Artificial Sequence Synthetic primer
gtgaatatggaaaaaaccaa

SEQ ID NO: 243 - DNA, 17 bp, Artificial Sequence
  Description of Artificial Sequence Synthetic primer
tgccatcttctcattgt

SEQ ID NO: 244 - DNA, 18 bp, Artificial Sequence
  Description of Artificial Sequence Synthetic primer
ttgtgtagaatagagccc

SEQ ID NO: 245 - DNA, 17 bp, Artificial Sequence
  Description of Artificial Sequence Synthetic primer
ttgtcctctgtgctaga

SEQ ID NO: 246 - DNA, 17 bp, Artificial Sequence
  Description of Artificial Sequence Synthetic primer
tgctggtgagcagggag

SEQ ID NO: 247 - DNA, 17 bp, Artificial Sequence
  Description of Artificial Sequence Synthetic primer
gcagaaggcgaagtggt

SEQ ID NO: 248 - DNA, 17 bp, Artificial Sequence
  Description of Artificial Sequence Synthetic primer
gtgcagtacctcccccc

SEQ ID NO: 249 - DNA, 20 bp, Artificial Sequence
  Description of Artificial Sequence Synthetic primer
ctcattttctctctattccc

SEQ ID NO: 250 - DNA, 19 bp, Artificial Sequence
  Description of Artificial Sequence Synthetic primer
agagaggtcctgaatgttc

SEQ ID NO: 251 - DNA, 19 bp, Artificial Sequence
  Description of Artificial Sequence Synthetic primer
gattatccttgctctggaa

SEQ ID NO: 252 - DNA, 19 bp, Artificial Sequence
  Description of Artificial Sequence Synthetic primer
gcaccatacaatcagagtt

SEQ ID NO: 253 - DNA, 19 bp, Artificial Sequence
  Description of Artificial Sequence Synthetic primer
gccaggcaatgtgttgaag

SEQ ID NO: 254 - protein, 6 aa, Artificial Sequence
  Description of Artificial Sequence Synthetic 6xHis tag
HHHHHH

SEQ ID NO: 255 - DNA, 23 bp, Artificial Sequence
  Description of Artificial Sequence Synthetic nucleotide sequence motif
aannnnnnnnnnnnnnnnnnntt